(19) United States
(12) Patent Application Publication
(10) Pub. No.: US 2022/0226375 A1
(43) Pub. Date: Jul. 21, 2022

(54) ANTI-CD33 IMMUNE CELL CANCER THERAPY

(71) Applicant: CRISPR Therapeutics AG, Zug (CH)

(72) Inventors: Jonathan Alexander Terrett, Cambridge, MA (US); Jason Sagert, Cambridge, MA (US); Demetrios Kalaitzidis, Cambridge, MA (US)

(73) Assignee: CRISPR Therapeutics AG, Zug (CH)

(21) Appl. No.: 17/291,181

(22) PCT Filed: Nov. 7, 2019
(86) PCT No.: PCT/IB2019/001194

§ 371 Date: May 4, 2021

Related U.S. Application Data
(60) Provisional application No. 62/756,718, filed on Nov. 7, 2018.
(60) Provisional application No. 62/767,395, filed on Nov. 14, 2018.
(60) Provisional application No. 62/767,388, filed on Nov. 14, 2018.
(60) Provisional application No. 62/826,648, filed on Mar. 29, 2019.
(60) Provisional application No. 62/826,643, filed on Mar. 29, 2019.

(51) Int. Cl. A61K 35/17; C07K 16/28; C07K 14/705; C07K 14/725; C07K 14/715; A61P 35/02

(52) U.S. Cl. CPC A61K 35/17; C07K 16/2803; C07K 14/70521; C07K 14/70517; A61K 39/00; C07K 14/7151; A61P 35/02; C07K 2317/622; C07K 2317/56; C07K 14/7051

(57) ABSTRACT

Provided herein, in some embodiments, are methods and compositions (e.g., cell compositions) for the treatment of cancer, such as CD33⁺ malignancies.

### CROSS REFERENCE TO RELATED APPLICATIONS

This application claims the benefit of the filing dates of U.S. Provisional Application No. 62/756,718, filed Nov. 7, 2018, U.S. Provisional Application No. 62/767,388, filed Nov. 14, 2018, U.S. Provisional Application No. 62/767,395, filed Nov. 14, 2018, U.S. Provisional Application No. 62/826,643, filed Mar. 29, 2019, and U.S. Provisional Application No. 62/826,648, filed Mar. 29, 2019. The entire contents of each of the prior applications are incorporated by reference herein.

### BACKGROUND

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

### SUMMARY

Acute myeloid leukemia (AML) is a blood neoplasm arising from mutations accumulated in myeloid progenitors and results in excess proliferation and blocked differentiation which leads to accumulation of myeloid blasts in hematopoietic tissue. Although responses to standard induction chemotherapy are initially high, relapse is common and prognosis is poor for the majority of AML patients (Talati and Sweet, 2018). Few new therapeutics for AML have been approved in recent years.

CD33 (also known as Siglec3, sialic acid binding Ig-like lectin 3, gp67, or p67) is an attractive target for treatment of AML and other leukemias, e.g., T cell leukemias. It is expressed on the majority of AML blasts and subpopulations (immunophenotypically-defined leukemia stem cells) at both presentation and relapse (Haubner et al., 2018). Its expression is thought to be restricted to normal monocytes, granulocytes, hematopoietic progenitors and some cells in the immunophenotypically-defined hematopoietic stem cell population (Haubner et al., 2018). Knockout of CD33 in mice does not result in any apparent phenotype (Brikman-Van der Linden et al., 2003). Further, the anti-CD33 antibody-drug conjugate gemtuzumab ozogmicin (GO) is approved for human use in AML and has an acceptable safety profile. However, GO shows only modest improvements in overall survival (Talati and Sweet, 2018). Targeting CD33-expressing cells with a more potent payload, such as anti-CD33CAR-T cells may demonstrated improved efficacy in AML relative to GO. Further, anti-CD33CAR-T cells represent effective therapeutic option for other CD33-expressing malignancies.

Some aspects of the present disclosure provide an engineered T cell comprising a nucleic acid encoding a chimeric antigen receptor (CAR), wherein the CAR comprise an ectodomain that binds specifically to CD33. In some embodiments, the engineered T cell further comprises a disrupted T cell receptor alpha chain constant region (TRAC) gene. For example, the TRAC gene may be disrupted by insertion of the nucleic acid encoding a CAR. In some embodiments, the engineered T cell further comprises a disrupted beta-2-microglobulin (β2M) gene. In some embodiments, the engineered T cell further comprises a disrupted CD33 gene.

In some embodiments, the engineered T cell comprises a disrupted TRAC gene, a disrupted β2M gene, a disrupted CD33 gene, and a nucleic acid encoding a CAR comprising an anti-CD33 antigen-binding fragment. In some embodiments, the engineered T cell comprises a disrupted TRAC gene, wherein the disrupted TRAC gene comprises a nucleic acid encoding a CAR comprising an anti-CD33 antigen-binding fragment, a disrupted β2M gene and a disrupted CD33 gene. In some embodiments, the CAR comprises (a) an ectodomain that comprises an anti-CD33 antigen-binding fragment, (b) a CD8 transmembrane domain, and (c) an endodomain that comprises a 41BB co-stimulatory domain and a CD3z co-stimulatory domain.

In some embodiments, the engineered T cell comprises: (i) a disrupted TRAC gene, wherein the disrupted TRAC gene comprises a nucleic acid encoding a CAR comprising the amino acid sequence of SEQ ID NO: 104; and (ii) a disrupted β2M gene. In some examples, such engineered T cells comprise a wild-type CD33 gene.

In some embodiments, the engineered T cell comprises: (i) a disrupted TRAC gene, wherein the disrupted TRAC gene comprises a nucleic acid encoding a CAR comprising the amino acid sequence of SEQ ID NO: 104; (ii) a disrupted β2M gene; and a disrupted CD33 gene.

In some embodiments, the engineered T cell comprises: (i) a disrupted TRAC gene, wherein the disrupted TRAC gene comprises a nucleic acid encoding a CAR, wherein the nucleic acid sequence is at least 90% identical to SEQ ID NO: 56 and encodes the CAR of SEQ ID NO:104; and (ii) a disrupted β2M gene.

In some embodiments, the engineered T cell comprises: (i) a disrupted TRAC gene, wherein the disrupted TRAC gene comprises a nucleic acid encoding a CAR, wherein the nucleic acid sequence is at least 90% identical to SEQ ID NO: 56 and encodes the CAR of SEQ ID NO: 104; (ii) a disrupted β2M gene; and (iii) a disrupted CD33 gene.

In some embodiments, the engineered T cell comprises: (i) a disrupted TRAC gene, wherein the disrupted TRAC gene comprises the nucleic acid sequence of SEQ ID NO: 55; and (ii) a disrupted β2M gene. In some embodiments, the engineered T cell comprises a wild-type CD33.

In some embodiments, the engineered T cell comprises: (i) a disrupted TRAC gene, wherein the disrupted TRAC gene comprises the nucleic acid sequence of SEQ ID NO: 55; (ii) a disrupted β2M gene; and (iii) a disrupted CD33 gene.

In some embodiments, the disclosure provides a population of cells comprising engineered T cells, wherein the engineered T cells comprise: (i) a disrupted TRAC gene, wherein the disrupted TRAC gene comprises a nucleic acid encoding a CAR comprising (a) an ectodomain that comprises an anti-CD33 antigen-binding fragment, (b) a CD8 transmembrane domain, and (c) an endodomain that comprises a 41BB co-stimulatory domain and a CD3ζ co-stimulatory domain; and (ii) a disrupted β2M gene. In some examples, the engineered T cells comprise a wild-type CD33.

In some embodiments, the disclosure provides a population of cells comprising engineered T cells, wherein the engineered T cells comprise: (i) a disrupted TRAC gene, wherein the disrupted TRAC gene comprises a nucleic acid encoding a CAR comprising (a) an ectodomain that comprises an anti-CD33 antigen-binding fragment, (b) a CD8 transmembrane domain, and (c) an endodomain that comprises a 41BB co-stimulatory domain and a CD3ζ co-stimulatory domain; (ii) a disrupted β2M gene; and (iii) a disrupted CD33 gene.

In some embodiments, the disclosure provides a population of cells comprising engineered T cells, wherein the engineered T cells comprise: (i) a disrupted TRAC gene, wherein the disrupted TRAC gene comprises a nucleic acid encoding a CAR comprising the amino acid sequence of SEQ ID NO: 104; and (ii) a disrupted β2M gene. In some examples, the engineered T cells comprise a wild-type CD33.

In some embodiments, the disclosure provides a population of cells comprising engineered T cells, wherein the engineered T cells comprise: (i) a disrupted TRAC gene, wherein the disrupted TRAC gene comprises a nucleic acid encoding a CAR comprising the amino acid sequence of SEQ ID NO: 104; (ii) a disrupted β2M gene; and (iii) a disrupted CD33 gene.

In some embodiments, the disclosure provides a population of cells comprising engineered T cells, wherein the engineered T cells comprise: (i) a disrupted TRAC gene, wherein the disrupted TRAC gene comprises a nucleic acid encoding a CAR, wherein the nucleic acid sequence is at least 90% identical to SEQ ID NO: 56 and encodes the CAR of SEQ ID NO:104; and (ii) a disrupted β2M gene. In some examples, the engineered T cells comprise a wild-type CD33.

In some embodiments, the disclosure provides a population of cells comprising engineered T cells, wherein the engineered T cells comprise: (i) a disrupted TRAC gene, wherein the disrupted TRAC gene comprises a nucleic acid encoding a CAR, wherein the nucleic acid sequence is at least 90% identical to SEQ ID NO: 56 and encodes the CAR of SEQ ID NO:104; (ii) a disrupted β2M gene; and (iii) a disrupted CD33 gene.

In some embodiments, the disclosure provides a population of cells comprising engineered T cells, wherein the engineered T cells comprise: (i) a disrupted TRAC gene, wherein the disrupted TRAC gene comprises the nucleic acid sequence of SEQ ID NO: 55; and (ii) a disrupted β2M gene. In some examples, the engineered T cells comprise a wild-type CD33.

In some embodiments, the disclosure provides a population of cells comprising engineered T cells, wherein the engineered T cells comprise: (i) a disrupted TRAC gene, wherein the disrupted TRAC gene comprises the nucleic acid sequence of SEQ ID NO: 55; (ii) a disrupted β2M gene; and (iii) a disrupted CD33 gene.

Any engineered T cells described herein may be human T cells.

The ectodomain of the CAR, in some embodiments, comprises an anti-CD33 antibody. In some embodiments, the anti-CD33 antibody is an anti-CD33 single-chain variable fragment (scFv). The anti-CD33 scFv, in some embodiments, comprises an amino acid sequence of any one of SEQ ID NO: 73, 75, 85, 87, 97, or 99. In some embodiments, the anti-CD33 scFv comprises a heavy chain variable region (VH) comprising an amino acid sequence of any one of SEQ ID NO: 65, 77 or 89 and/or a light chain variable region (VL) comprising an amino acid sequence of any one of SEQ ID NO: 66, 78 or 90. In some embodiments, the anti-CD33 scFv comprises a VH comprising CDR amino acid sequences of SEQ ID NO: 67, SEQ ID NO: 68, and/or SEQ ID NO: 69; and/or the anti-CD33 scFv comprises a VL sequence comprising CDR amino acid sequences of SEQ ID NO: 70, SEQ ID NO: 71, and/or SEQ ID NO: 72. In some embodiments, the anti-CD33 scFv comprises a VH comprising CDR amino acid sequences of SEQ ID NO: 79, SEQ ID NO: 80, and/or SEQ ID NO: 81; and/or the anti-CD33 scFv comprises a VL sequence comprising CDR amino acid sequences of SEQ ID NO: 82, SEQ ID NO: 83, and/or SEQ ID NO: 84. In some embodiments, the anti-CD33 scFv comprises a VH comprising CDR amino acid sequences of SEQ ID NO: 91, SEQ ID NO: 92, and/or SEQ ID NO: 93; and/or the anti-CD33 scFv comprises a VL sequence comprising CDR amino acid sequences of SEQ ID NO: 94, SEQ ID NO: 95, and/or SEQ ID NO: 96.

The CAR, in some embodiments, comprises a CD3ζ cytoplasmic signaling domain. In some embodiments, the CAR comprises a CD28 co-stimulatory domain or a 41BB co-stimulatory domain. In specific examples, the CAR disclosed herein comprises an anti-CD33 scFv, a CD28 co-stimulatory domain, and a CD3ζ cytoplasmic signaling domain. In other examples, the CAR disclosed herein comprises an anti-CD33 scFv, a 4-1BB co-stimulatory domain, and a CD3ζ cytoplasmic signaling domain.

In some embodiments, the TRAC gene comprises the nucleotide sequence of any one of SEQ ID NOs: 49, 51, 53, 55, 57, 59, 61, 63, 109, 112, 115, or 118 and/or wherein the CAR comprises the nucleotide sequence of any one of SEQ ID NOs: 50, 52, 54, 56, 58, 60, 62, 64, 110, 113, 116 or 119. In some embodiments, the disrupted β2M gene comprises at least one nucleotide sequence selected from any one of SEQ ID NOs: 9-14.

In some embodiments, the T cells comprise a wild-type CD33 gene. In some embodiments, the T cells comprise a disrupted CD33 gene.

In some embodiments, the disrupted CD33 gene comprises a nucleotide sequence of

(SEQ ID NO: 187)
AGTTCATGGTACTGGTTCC,
(SEQ ID NO: 188)
AGTTCATGGTTCC,
(SEQ ID NO: 189)
AGTTCATGTACTGGTTCC,
(SEQ ID NO: 190)
AGTTCATGGTTTACTGGTTCC,
(SEQ ID NO: 191)
AGTTCC, AGTACTGGTTCC,
(SEQ ID NO: 192)
AGTTCATACTGGTTCC,
(SEQ ID NO: 193)
AGTTCATGGTATACTGGTTCC,
and/or
(SEQ ID NO: 194)
AGTTACTGGTTCC.

In some embodiments, the disrupted CD33 gene lacks a fragment comprising

(SEQ ID NO: 186)
AGTTCATGGTTACTGGTTCC.

In some embodiments, the disrupted CD33 gene comprises a nucleotide sequence of

(SEQ ID NO: 300)
AAATCCTGGCACT,
(SEQ ID NO: 301)
AAATCCCTGGCACT,
(SEQ ID NO: 302)
AAATCCTCATTCCCTGGCACT,
(SEQ ID NO: 304)
AAATCCTCACCCTGGCACT,
(SEQ ID NO: 305)
AAATCCTCCCCTGGCACT,
(SEQ ID NO: 306)
AAATCCTCCCTGGCACT,
(SEQ ID NO: 307)
AAATCCCCTGGCACT,
(SEQ ID NO: 308)
ACATCCTCATTCCCTGGCACT,
(SEQ ID NO: 309)
ACATCCTGGCACT,
(SEQ ID NO: 310)
AAATCCTCTCCCTGGCACT,
(SEQ ID NO: 311)
AAATCCTCATCTGGCACT,
(SEQ ID NO: 312)
AAATCCT, AAACCCTGGCACT,
(SEQ ID NO: 313)
AAATCCTCTGGCACT,
(SEQ ID NO: 314)
AAATCCCCCTGGCACT,
(SEQ ID NO: 315)
AAATCCTCACT,
(SEQ ID NO: 316)
ACATCCCTGGCACT,
and/or
AAAT.

In some embodiments, the disrupted CD33 gene lacks a fragment comprising

(SEQ ID NO: 299)
AAATCCTCATCCCTGGCACT.

In some embodiments, the disrupted CD33 gene lacks a fragment, the 3′ segment of which comprises the nucleotide sequence of AAATCCTCAT (SEQ ID NO: 317), AAATCCTCATCCCT (SEQ ID NO: 318), AAATCCTCATCCCTGG (SEQ ID NO: 320), AAATCCTCATC (SEQ ID NO: 322), or AAATCCTCATCCCTGGCA (SEQ ID NO: 324).

In some embodiments, the disrupted CD33 gene lacks a fragment, the 5′ segment of which comprises the nucleotide sequence of CTCATCCCTGGCACT (SEQ ID NO: 323).

Also provided herein, in some aspects, is a population of engineered T cells (e.g., comprising a nucleic acid encoding an anti-CD33 CAR), wherein at least 25% or at least 50% of engineered T cells of the population express the CAR. For example, at least 70% of engineered T cells of the population express the CAR.

In some embodiments, at least 25% of engineered T cells of the population express the CAR following at least 7 days or at least 14 days of in vitro proliferation.

In some embodiments, at least 50% of engineered T cells of the population do not express a detectable level of T cell receptor (TCR) protein. For example, at least 90% of engineered T cells of the population may not express a detectable level of TCR protein.

In some embodiments, at least 50% of engineered T cells of the population do not express a detectable level of β2M protein. For example, at least 70% of engineered T cells of the population may not express a detectable level of β2M protein.

In some embodiments, at least 20% of engineered T cells of the population do not express a detectable level of CD33 protein. For example, at least 50% of engineered T cells of the population may not express a detectable level of CD33 protein.

In some embodiments, engineered T cells of the population, when co-cultured in vitro with a population of cancer cells that express CD33, induce cell lysis of at least 50% of the cancer cells of the population. For example, engineered T cells of the population may induce cell lysis of at least 70%, at least 80%, or at least 90% of the cancer cells of the population. In some embodiments, engineered T cells of the population, when co-cultured in vitro with a population of cancer cells, secrete IFNγ. In some embodiments, the ratio of engineered T cells to cancer cells is 1:1 to 2:1. The cancer cells may be, for example, leukemia, such as acute lymphoblastic leukemia (ALL), acute myeloid leukemia (AML), chronic lymphocytic leukemia (CLL) and chronic myeloid leukemia (CML). Other cancer cells may be targeted.

In some embodiments, proliferative capacity of engineered T cells of the population is within 10% of proliferative capacity of control cells.

Other aspects of the present disclosure provide a method that comprises administering the population of engineered T cells as described herein. In some embodiments, percent body weight of the subject, following 5-10 days of administration, is within 10% of initial body weight of the subject, wherein initial body weight of the subject is body weight of the subject at the time of administration. In some embodiments, the subject is a human subject. In some embodiments, the subject has a cancer. The cancer may express CD33, for example. The cancer may be, for example, leukemia, such as ALL, AML, CLL and CML.

Further aspects of the present disclosure provide a method for producing an engineered T cell, the method comprising (a) delivering to a T cell a RNA-guided nuclease, a gRNA targeting a TRAC gene, and a vector comprising a donor template that comprises a nucleic acid encoding a CAR that comprise an ectodomain that binds specifically to CD33, wherein the nucleic acid encoding the CAR is flanked by left and right homology arms to the TRAC gene, and (b) producing an engineered T cell. In some embodiments, the gRNA targeting the TRAC gene comprises the nucleotide sequence of SEQ ID NO: 18 or SEQ ID NO: 19, or targets the nucleotide sequence of SEQ ID NO: 40.

In some embodiments, the method further comprises delivering to the T cell a gRNA targeting the β2M gene. In some embodiments, the gRNA targeting the β2M gene comprises the nucleotide sequence of SEQ ID NO: 20 or SEQ ID NO: 21, or targets the nucleotide sequence of SEQ ID NO: 41.

In some embodiments, the method further comprises delivering to the T cell a gRNA targeting the CD33 gene. In some embodiments, the gRNA targeting the CD33 gene comprises a nucleotide sequence as provided in Table 10.

In some embodiments, the RNA-guided nuclease is a Cas9 nuclease, optionally a S. pyogenes Cas9 nuclease.

In some embodiments, the donor template comprises the nucleotide sequence of any one of SEQ ID NOs: 49, 51, 53, 55, 57, 59, 61, 63, 109, 112, 115, or 118.

In some embodiments, the CAR comprises the nucleotide sequence of any one of SEQ ID NOs: 50, 52, 54, 56, 58, 60, 62, 64, 110, 113, 116 or 119.

Further aspects of the present disclosure provide a method for reducing volume of a tumor in a subject, comprising administering to a subject having cancer, e.g., leukemia, a population of engineered T cells as described herein. In some embodiments, the volume of the tumor in the subject is reduced by at least 50% relative to a baseline control, optionally wherein 1×10⁵ cells to 1×10⁷ cells of the population are administered.

### BRIEF DESCRIPTION OF THE DRAWINGS

FIG. 1A includes flow cytometry plots depicting surface expression of an anti-CD33 CAR on T cells edited with select anti-CD33 CAR constructs at two weeks post electroporation. Transfection with CTX-965b CAR resulted in a high proportion of T cells expressing an anti-CD33 CAR. All CAR T cells are also TRAC−/β2M− (2KO).

FIG. 1B includes flow cytometry plots depicting surface expression of an anti-CD33 CAR on T cells edited with select anti-CD33 CAR constructs at two weeks post electroporation. Transfection with CTX-970 CAR and CTX-965b CAR resulted in a high proportion of T cells expressing an anti-CD33 CAR. All CAR T cells are also TRAC−/β2M− (2KO).

FIG. 1C includes flow cytometry plots depicting surface expression of an anti-CD33 CAR on T cells edited with select anti-CD33 CAR constructs at one week post electroporation. Transfection with CTX-981 CAR, CTX-981b CAR, CTX-982 CAR, and CTX-982b CAR all resulted in a high proportion of T cells expressing an anti-CD33 CAR. All CAR T cells are also TRAC−/β2M− (2KO).

FIG. 2A includes flow cytometry plots demonstrating the effects of gene editing on donor T cell populations. Shown is the proportion of edited T cells with expression of cell-surface TCR, β2M and anti-CD33 CAR. Additionally, the proportion of T cells that are CD4+ and CD8+ are shown. Notably, CTX-965b cells retain expression of high levels of CD4/CD8 for at least one week after gene editing.

FIG. 2B shows the % of cells expressing CAR in TRAC−/β2M− T cells over time. All anti-CD33 CAR-T cells expanded over the two week period.

FIG. 2C shows the % of CD4+ and CD8+ T cells within the TRAC−/β2M−/anti-CD33 CAR+ edited T cell population over time. The top panel shows CD4+ and CD8+ cells in CTX-965b CAR T cells from seven primary T cell donors at 1 and 2 weeks post editing. The bottom panel shows CD4+ and CD8+ cells in CTX-970 CAR T cells from four primary T cell donors at 1 and 2 weeks post editing.

FIG. 2D shows the % of CD4+ and CD8+ cells in the TRAC−/β2M−/anti-CD33 CAR+ T cell population over time. Specifically, the figure shows CD4+ and CD8+ cells in CTX-982b CAR T cells from three primary T cell donors at 1 and 2 weeks post editing.

FIG. 3 includes a graph showing cell expansion of a population of anti-CD33 CAR-T cells (CTX-965b), and populations of control T cells (No RNP; TRAC−/β2M−).

FIG. 4 includes a graph showing surface expression levels of CD33 in varying blood-derived cell lines. The left panel shows that THP-1 (AML) and PBMC have high surface expression of CD33, the right panel shows high surface expression of CD33 in AML cancer cell lines: MV4-11, THP-1 and KG-1. The right panel also shows that MV4-11 cells engineered with a CD33 knockout (MV4-11 CD33 knock-out) do not express CD33.

FIGS. 5A-5D include graphs demonstrating that TRAC−/β2M−/anti-CD33 CAR+ T cells (CTX-965b and CTX-970) are capable of killing AML cells that express CD33 (THP-1, KG-1 and MV4-11). FIG. 5A shows that CTX-965b CAR T cells generated from 4 different primary T cell donors are effective at inducing cell lysis in THP-1 cells at cell ratios of 0.05:1 to 1:1 CTX-965b:THP-1. FIG. 5B shows that CTX-965b and CTX-970 CAR T cells, each generated from T cells isolated from one donor, are effective at inducing cell lysis of THP-1 cells at cell ratios of 0.05:1 to 1:1 CAR-T cells:THP-1. FIG. 5C shows that CTX-965b, generated from T cells isolated from 4 different donors, and CTX-970 CAR T cells, generated from one primary T cell donor, are effective at inducing cell lysis in KG-1 cells at cell ratios of 0.05:1 to 1:1 CAR-T cells:KG-1. FIG. 5D shows that CTX-965b and CTX-970 CAR T cells, each generated from T cells isolated from one donor, are effective at inducing cell lysis in MV4-11 cells at cell ratios of 0.05:1 to 1:1 CAR-T cells:MV4-11.

FIGS. 6A-6E include graphs demonstrating that TRAC−/β2M−/anti-CD33 CAR⁺ T cells (CTX-965b and CTX-970) are capable of secreting IFNγ in the presence of AML cells that express CD33 (THP-1, KG-1 and MV4-11). FIG. 6A shows that CTX-965b CAR T cells generated from 4 donors are effective at secreting IFNγ in the presence of THP-1 cells at cell ratios of 0.05:1 to 1:1 CTX-965b:THP-1. FIG. 6B shows that CTX-965b and CTX-970 CAR T cells are effective at secreting IFNγ in the presence of THP-1 cells at cell ratios of 0.05:1 to 1:1 CAR-T cells:THP-1. FIG. 6C shows that CTX-965b and CTX-970 CAR T cells are effective at secreting IFNγ in the presence of MV-411 cells at cell ratios of 0.05:1 to 1:1 CAR-T cells:MV-411. FIG. 6D shows that CTX-965b and CTX-970 CAR T cells are effective at secreting IFNγ in the presence of KG1 cells at cell ratios of 0.05:1 to 1:1 CAR-T cells:KG1. FIG. 6E shows that CTX-965b CAR T cell growth is cytokine dependent.

FIG. 7 includes a graph showing that TRAC−/β2M−/anti-CD33 CAR+ T cells (CTX-965b CAR T cells) are effective at reducing tumor volume in a subcutaneous THP-1 AML cancer in vivo mouse model.

FIG. 8 includes a graph showing that TRAC−/β2M−/anti-CD33 CAR+ T cells (CTX-965b CAR T cells) are effective at reducing tumor volume of well-established tumors (starting tumor volume of approximately 150 mm³) in a subcutaneous THP-1 AML cancer in vivo mouse model.

FIG. 9 shows the percent of edited CD8+(left panel) and CD4+(right panel) T cells after gene editing by various sgRNAs that target CD33.

FIG. 10 shows the % CAR-expressing cells in TRAC−/β2M− edited T cells with and without CD33 disruption at day 7 and day 14 post gene editing. The data is shown for T cells expressing CAR from one of six different constructs: CTX-981 (981), CTX-981b (981b), CTX-982 (982), CTX-982b (982b), CTX-970 (970), or CTX-965b (965b).

FIG. 11 shows % CAR-expressing cells in TRAC−/β2M-edited T cells and TRAC−/β2M−/CD33-/anti-CD33 CAR+ edited T cells over time. The data is shown for CAR+ T cells expressing CAR from one of three different constructs: CTX-965b (965b), CTX-970 (970), or CTX-982b (982b). black bars=7 days; grey bars=14 days.

FIG. 12 shows the % of CD4+ and CD8+ cells in the TRAC−/β2M−/anti-CD33 CAR+ edited T cell and TRAC−/β2M−/CD33-/anti-CD33 CAR+ edited T cell populations over time. The left panel shows CD8+ cells in CAR T cells at 1 and 2 weeks post editing. The right panel shows CD4+CD8+ cells in CAR T cells at 1 and 2 weeks post editing.

FIG. 13 shows that TRAC−/β2M−/CD33-/anti-CD33 CAR+ T cells retain the ability to kill AML cancer cells. Left panel shows % cell lysis of AML cells by CAR+ T cells that were generated from a single primary T cell donor. The right panel shows % cell lysis of AML cells by CAR+ T cells that were generated from a different primary T cell donor.

FIG. 14 demonstrates that 2× KO (TRAC−/β2M−) anti-CD33 CAR T cells and 3× KO (TRAC−/β2M−/CD33−) anti-CD33 CAR T cells are capable of inducing IFNγ secretion in AML cells expressing CD33 (MV4-11; MV4-11) at similar levels. CAR T cells generated from 2 different primary T cell donors are effective at inducing IFNγ secretion at cell ratios of 0.05:1 to 1:1 CAR T cell:MV4-11.

FIG. 15 demonstrates that 2× KO (TRAC−/β2M−) anti-CD33 CAR T cells and 3× KO (TRAC−/β2M−/CD33−) anti-CD33 CAR T cells are capable of inducing IL-2 secretion in AML cells expressing CD33 (MV4-11; MV4-11). Data is for CAR T cells generated from 2 different primary T cell donors.

FIG. 16 shows the % cell lysis for wild-type MV4-11 cells (left panel) or CD33 knock-out MV4-11 cells (CD33˜ MV4-11) (right panel) in response to 2× KO (TRAC−/β2M−) anti-CD33 CAR T cells or 3× KO (TRAC−/β2M−/CD33−) anti-CD33 CAR T cells when seeded at a 1:1 ratio (CAR+ T cell:target cell).

FIG. 17 shows % cell lysis for CD33 knock-out MV4-11 cells (CD33˜ MV4-11) in response to 2× KO (TRAC−/β2M−) anti-CD33 CAR T cells or 3× KO (TRAC−/β2M−/CD33−) anti-CD33 CAR T cells when seed at a 0.25:1, 0.5:1, 1:1, and 2:1 ratio of CAR+ T cell:CD33-/MV4-11 cells.

FIG. 18 shows IL-2 secretion (left panel) and IFNγ secretion (right panel) in 2× KO (TRAC−/β2M−) anti-CD33 CAR T cells or 3× KO (TRAC−/β2M−/CD33−) anti-CD33 CAR T cells in response to cancer cells deficient in CD33 (CD33 KO, MV4-11 cells).

FIGS. 19A-19E include graphs demonstrating that TRAC−/β2M−/anti-CD33 CAR+ T cells (CTX-965b and CTX-970) provided therapeutic effects in a MV-4-11 NSG mouse model of AML. FIG. 19A shows that three different doses of CTX-965b CAR T cells were effective in reducing tumor burden in the mouse model of AML. FIG. 19A shows that three different doses of CTX-965b CAR T cells were effective in increasing median survival in the mouse model of AML. FIG. 19C shows that CTX-965b CAR T cells with and without knockout of the CD33 gene were effective in increasing median survival in the mouse model of AML. FIG. 19D shows that CTX-970 CAR T cells with and without knockout of the CD33 gene were effective in increasing median survival in the mouse model of AML. FIG. 19E shows the tumor burden as measured by bioluminescence imaging at Day 33 following treatment of mice with CTX-965b or CTX-970 CAR T cells with or without CD33 knockout.

### DETAILED DESCRIPTION

The present disclosure is based, at least in part, on the discovery that anti-CD33 CAR+ T cells reduced tumor burden and increased median survival in mouse models of acute myeloid leukemia (AML). It has also been demonstrated that CD33 is highly expressed on activated T cells, which may be susceptible to self-reactive killing by anti-CD33 CAR+ T cells. Such self-reactive killing may be reduced or eliminated by disrupting the endogenous CD33 gene in anti-CD33 CAR+ T cells using gene editing methods provided herein. Accordingly, the present disclosure provides, in some aspects, anti-CD33 CAR+ T cells having a disrupted endogenous CD33 gene. In other aspects, the present disclosure provides anti-CD33 CAR+ T cells having a wild-type endogenous CD33 gene.

Aspects of the present disclosure provide anti-CD33 CAR+ T cells with or without a disrupted CD33 gene, methods of producing such anti-CD33 CAR+ T cells, and methods of using such anti-CD33 CAR+ T cells for treating cancer (e.g., AML) in a subject. Components and processes (e.g., the CRISPR approach for gene editing and components used therein) for making anti-CD33 CAR+ T cells disclosed herein are also within the scope of the present disclosure.

### CD33 Cancer Antigen

In some embodiments, the T cells of the present disclosure are engineered with a chimeric antigen receptor (CAR) designed to target CD33. CD33, also known as Siglec3, is a transmembrane receptor expressed on cells of myeloid lineage that is known to bind sialic acids. As CD33 is expressed in cancer cells (e.g., acute myeloid leukemia), it is thought that CD33 represents a cell surface marker for targeting these malignancies.

Thus, in some embodiments, T cells of the present disclosure are engineered to express a CAR comprising an anti-CD33 antibody (e.g., anti-CD33 scFv). In some embodiments, the anti-CD33 antibody is an anti-CD33 scFv encoded by the sequence of any one of SEQ ID NOS: 74, 76, 86, 88, 98, or 100. In some embodiments, the anti-CD33 antibody is an anti-CD33 scFv comprising the sequence of any one of SEQ ID NOS: 73, 75, 85, 87, 97, or 99. In some embodiments, the anti-CD33 antibody is an anti-CD33 scFv comprising a VH comprising an amino acid sequence of any one of SEQ ID NO: 65, 77 or 89. In some embodiments, the anti-CD33 antibody is an anti-CD33 scFv comprising a VL comprising an amino acid sequence of any one of SEQ ID NO: 66, 78 or 90. In some embodiments, a CAR comprising an anti-CD33 antibody is encoded by the sequence of any one of SEQ ID NOs: 50, 52, 54, 56, 58, 60, 62, 64, 110, 113, 116 or 119. In some embodiments, a CAR comprising an anti-CD33 antibody comprises the sequence of any one of SEQ ID NOS: 101-108, 111, 114, 117, or 120. In some embodiments, a CAR comprising an anti-CD33 antibody comprises an anti-CD33 antibody as described in U.S. Pat. Nos. 9,359,442, 9,587,019, or U.S. Pat. No. 5,773,001.

### Multi-Gene Editing

The engineered T cells of the present disclosure, in some embodiments, include more than one gene edit, for example, in more than one gene. For example, an engineered T cell may comprise a disrupted T cell receptor alpha chain constant region (TRAC) gene, a disrupted beta-2-microglobulin (β2M) gene, a disrupted programmed cell death-1 (PD-1 or PDCD1) gene, a disrupted CD70 gene, or any combination of two or more of the foregoing disrupted genes. In some embodiments, an engineered T cell comprises a disrupted TRAC gene, a disrupted β2M gene, and a disrupted CD70 gene. In some embodiments, an engineered T cell comprises a disrupted TRAC gene, a disrupted β2M gene, and a disrupted PD-1 gene. In some embodiments, an engineered T cell comprises a disrupted TRAC gene, a disrupted β2M gene, a disrupted CD70 gene and a disrupted PD-1 gene.

It should be understood that gene disruption encompasses gene modification through gene editing (e.g., using CRISPR/Cas gene editing to insert or delete one or more nucleotides). As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region.

Provided herein, in some embodiments, are populations of cells in which a certain percentage of the cells has been edited (e.g., β2M gene edited), resulting in a certain percentage of cells not expressing a particular gene and/or protein. In some embodiments, at least 50% (e.g., 50%, 55%, 60%, 65%, 70%, 75%, 80%, 85%, 90%, or 85%) of the cells of a gene-edited population of cells are β2M knockout cells. In some embodiments, at least 50% of the cells (e.g. T cells) of the population do not express detectable levels of β2M protein. In some embodiments, at least 55%, at least 60%, at least 65%, at least 70%, at least 75%, at least 80%, at least 85%, at least 90%, or at least 95% of the cells of a gene-edited population of cells may be β2M knockout cells.

Methods of using CRISPR-Cas gene editing technology to create a genomic deletion in a cell (e.g., to knock out a gene in a cell) are known (Bauer D E et al., Vis. Exp. 2015; 95;e52118).

TRAC Gene Edit

In some embodiments, an engineered T cell comprises a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. In some embodiments, gRNAs targeting the TRAC genomic region create Indels in the TRAC gene disrupting expression of the mRNA or protein. In some embodiments, a disruption in the TRAC gene expression is created by gRNAs targeting the TRAC genomic region. In some embodiments, a disruption in the TRAC gene expression is created by knocking an exogenous sequence (e.g., a nucleic acid encoding a chimeric antigen receptor) into the TRAC gene (e.g., using an adeno-associated viral (AAV) vector and donor template). In some embodiments, a genomic deletion in the TRAC gene is created by a gRNA and/or knocking an exogenous sequence (e.g., a nucleic acid encoding a chimeric antigen receptor) into the TRAC gene (e.g., using an AAV vector and donor template). In some embodiments, a disruption in the TRAC gene expression is created by gRNAs targeting the TRAC genomic region and knocking a chimeric antigen receptor (CAR) into the TRAC gene.

Non-limiting examples of modified and unmodified TRAC gRNA sequences that may be used as provided herein to create a genomic disruption in the TRAC gene are listed in Table 4 (e.g., SEQ ID NOS: 18 and 19). See also International Application No. PCT/US2018/032334, filed May 11, 2018, incorporated herein by reference. Other gRNA sequences may be designed using the TRAC gene sequence located on chromosome 14 (GRCh38: chromosome 14: 22,547,506-22,552,154; Ensembl; ENSG00000277734).

In some embodiments, at least 50% of a population of engineered T cells do not express a detectable level of T cell receptor (TCR) surface protein. For example, at least 55%, at least 60%, at least 70%, at least 75%, at least 80%, at least 85%, at least 90%, or at least 95% of a population may not express a detectable level of TCR surface protein. In some embodiments, 50%-100%, 50%-90%, 50%-80%, 50%-70%, 50%-600%, 60%-100%, 60%-90%, 60%-80%, 60%-70%, 70%-100%, 70%-90%, 70%-80%, 80%-100%, 80%-90%, or 90%-100% of the population of engineered T cells do not express a detectable level of TCR surface protein.

In some embodiments, a ribonucleoprotein particle (RNP) containing an RNA-guided nuclease (e.g., a Cas nuclease, such as a Cas9 nuclease) and a gRNA targeting the TRAC gene (or any other gene of interest) are delivered to T cells (e.g., primary T cells). In other embodiments, the RNA-guided nuclease and gRNA are delivered separately to T cells. A ribonucleoprotein particle (RNP) is simply a RNA-guided nuclease (e.g., Cas9) pre-complexed/complexed with a gRNA.

In some embodiments, gRNAs targeting the TRAC genomic region create Indels in the TRAC gene comprising at least one nucleotide sequence selected from the following sequences in Table 1:

TABLE 1

| | SEQ ID |
|---|---|
| Sequence | NO: |
| AAGAGCAACAAATCTGACT | 1 |
| AAGAGCAACAGTGCTGTGC | 2 |
| CTGGAGCAACAAATCTGAC | |
| TAAGAGCAACAAATCTGAC | |
| T | |
| AAGAGCAACAGTGCTGGAG | 3 |
| CAACAAATCTGACTAAGAG | |
| CAACAAATCTGACT | |
| AAGAGCAACAGTGCCTGGA | 4 |
| GCAACAAATCTGACTAAGA | |
| GCAACAAATCTGACT | |
| AAGAGCAACAGTGCTGACT | 5 |
| AAGAGCAACAAATCTGACT | |
| AAGAGCAACAGTGCTGTGG | 6 |
| GCCTGGAGCAACAAATCTG | |
| ACTAAGAGCAACAAATCTG | |
| ACT | |
| AAGAGCAACAGTGCTGGCC | 7 |
| TGGAGCAACAAATCTGACT | |
| AAGAGCAACAAATCTGACT | |
| AAGAGCAACAGTGCTGTGT | 8 |
| GCCTGGAGCAACAAATCTG | |
| ACTAAGAGCAACAAATCTG | |
| ACT | |

In some embodiments, an engineered T cell comprises a deletion in the TRAC gene relative to unmodified T cells. In some embodiments, an engineered T cell comprises a deletion of 15-30 base pairs in the TRAC gene relative to unmodified T cells. In some embodiments, an engineered T cell comprises a deletion of 15, 16, 17, 18, 19, 20, 21, 22, 23, 24, 25, 26, 27, 28, 29 or 30 base pairs in the TRAC gene relative to unmodified T cells. In some embodiments, an engineered T cell comprises a deletion of more than 30 base pairs in the TRAC gene relative to unmodified T cells. In some embodiments, an engineered T cell comprises a deletion of 20 base pairs in the TRAC gene relative to unmodified T cells. In some embodiments, an engineered T cell comprises a deletion of AGAGCAACAGTGCTGTGGCC (SEQ ID NO: 325) in the TRAC gene relative to unmodified T cells. In some embodiments, an engineered T cell comprises a deletion comprising AGAGCAACAGTGCTGTGGCC (SEQ ID NO: 325) in the TRAC gene relative to unmodified T cells. In some embodiments, an engineered T cell comprises a deletion of SEQ ID NO: 40 in the TRAC gene relative to unmodified T cells. In some embodiments, an engineered T cell comprises a deletion comprising SEQ ID NO: 40 in the TRAC gene relative to unmodified T cells.

### β2M Gene Edit

In some embodiments, an engineered T cell comprises a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

Non-limiting examples of modified and unmodified β2M gRNA sequences that may be used as provided herein to create a genomic disruption in the β2M gene are listed in Table 4 (e.g., SEQ ID NOs: 20 and 21). See also International Application No. PCT/US2018/032334, filed May 11, 2018, incorporated herein by reference. Other gRNA sequences may be designed using the β2M gene sequence located on Chromosome 15 (GRCh38 coordinates: Chromosome 15: 44,711,477-44,718,877; Ensembl: ENSG00000166710).

In some embodiments, gRNAs targeting the β2M genomic region create Indels in the β2M gene disrupting expression of the mRNA or protein.

In some embodiments, at least 50% of the engineered T cells of a population of engineered T cells does not express a detectable level of β2M surface protein. For example, at least 55%, at least 60%, at least 70%, at least 75%, at least 80%, at least 85%, at least 90%, or at least 95% of the engineered T cells of a population may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

In some embodiments, a ribonucleoprotein particle (RNP) containing an RNA-guided nuclease (e.g., a Cas nuclease, such as a Cas9 nuclease) and a gRNA targeting the B2M gene (or any other gene of interest) are delivered to T cells (e.g., primary T cells). In other embodiments, the RNA-guided nuclease and gRNA are delivered separately to T cells. A ribonucleoprotein particle (RNP) is simply a RNA-guided nuclease (e.g., Cas9) pre-complexed/complexed with a gRNA.

In some embodiments, an edited β2M gene comprises at least one nucleotide sequence selected from the following sequences in Table 2:

TABLE 2

| | SEQ ID |
|---|---|
| Sequences | NO: |
| CGTGGCCTTAGCTGTGCTCGCGC | 9 |
| TACTCTCTCTTTCTGCCTGGAGG | |
| CTATCCAGCGTGAGTCTCTCCTA | |
| CCCTCCCGCT | |
| CGTGGCCTTAGCTGTGCTCGCGC | 10 |
| TACTCTCTCTTTCGCCTGGAGGC | |
| TATCCAGCGTGAGTCTCTCCTAC | |
| CCTCCCGCT | |
| CGTGGCCTTAGCTGTGCTCGCGC | 11 |
| TACTCTCTCTTTCTGGAGGCTAT | |
| CCAGCGTGAGTCTCTCCTACCCT | |
| CCCGCT | |
| CGTGGCCTTAGCTGTGCTCGCGC | 12 |
| TACTCTCTCTTTCTGGATAGCCT | |
| GGAGGCTATCCAGCGTGAGTCTC | |
| TCCTACCCTCCCGCT | |
| CGTGGCCTTAGCTGTGCTCGCGC | 13 |
| TATCCAGCGTGAGTCTCTCCTAC | |
| CCTCCCGCT | |
| CGTGGCCTTAGCTGTGCTCGCGC | 14 |
| TACTCTCTCTTTCTGTGGCCTGG | |
| AGGCTATCCAGCGTGAGTCTCTC | |
| CTACCCTCCCGCT | |

CD33 Gene Edit

CD33 (also known as Siglec3, sialic acid binding Ig-like lectin 3, gp67, or p67) is a transmembrane receptor expressed on cells of myeloid lineage. CD33 binds sialic acids, therefore is a member of the SIGLEC family of lectins. It is usually considered myeloid-specific, but it can also be found on some lymphoid cells, including activated-T cells (Hernandez-Caselles et al., 2006).

In some embodiments, an engineered T cell comprises a disrupted CD33 gene. In some embodiments, expression of the endogenous CD33 gene is eliminated to enhance anti-tumor efficacy and decrease fratricide of the CAR T cells of the present disclosure. In some embodiments, gRNAs targeting the CD33 genomic region create Indels in, around, or nearby the CD33 gene disrupting expression of CD33 mRNA and/or CD33 protein.

Non-limiting examples of modified and unmodified CD33 gRNA sequences that may be used as provided herein to create a genomic disruption in the CD33 gene are listed in Table 10, e.g., CD33-1 gRNA; UGGCUAUGGAUCCAAAUUUCguuuuagagcuagaaauagcaaguuaaaauaa ggcuaguccguuaucaacuugaaaaaguggcaccgagucggugcUUUU (SEQ ID NO: 132). In some examples, CD33-2 or CD33-10 guide RNAs may be used to create genomic disruptions in a CD33 gene. In some examples, the guide RNA used to disrupt the CD33 gene comprises a spacer sequence listed in Table 10. Other gRNA sequences may be designed using the CD33 gene sequence located on Chromosome 19 (GRCh38 coordinates: Chromosome 19: 51,225,064-51,243,860; Ensembl: ENSG00000105383.14).

In some embodiments, an engineered T cell comprises a disrupted CD33 gene. In some embodiments, at least 20% of the engineered T cells of a population of engineered T cells does not express a detectable level of CD33 surface protein. For example, at least 20%, at least 25%, at least 30%, at least 35%, at least 40%, at least 45%, at least 50%, at least 55%, at least 60%, at least 70%, at least 75%, at least 80%, at least 85%, at least 90%, or at least 95% of the engineered T cells of a population may not express a detectable level of CD33 surface protein. In some embodiments, 20%-75%, 20-50%, 30-50%, 30%-75%, 50%-100% a, 50%-90%, 50%-80%, 50%-70%, 50%-60%, 60%-100%, 60%-90%, 60%-80%, 60%-70%, 70%-100%, 70%-90%, 70%-80%, 80%-100%, 80-90%, or 90%-100% of the engineered T cells of a population does not express a detectable level of CD33 surface protein.

In some embodiments, a ribonucleoprotein particle (RNP) containing an RNA-guided nuclease (e.g., a Cas nuclease, such as a Cas9 nuclease) and a gRNA targeting the CD33 gene (or any other gene of interest) are delivered to T cells (e.g., primary T cells). In other embodiments, the RNA-guided nuclease and gRNA are delivered separately to T cells. A ribonucleoprotein particle (RNP) is simply a RNA-guided nuclease (e.g., Cas9) pre-complexed/complexed with a gRNA.

In some embodiments, the edited CD33 gene may comprise a mutated fragment, e.g., the edited CD33 gene comprises one or more of the mutated fragments provided in Tables 13-22 (column “Gene Edited Sequences”), e.g., those provided in Table 14 and/or Table 22). For example, the CD33 gene may comprise a mutated fragment having a deletion relative to the wild-type counterpart, e.g., the edited CD33 gene may comprise a nucleotide sequence set forth as GGATCCAAA-TTCTGGCTGC (SEQ ID NO: 175), where a single nucleotide deletion is represented by a dash (-). In another example, the edited CD33 gene may comprise a mutated fragment having an insertion relative to the wild-type counterpart, e.g., GGATCCAAATTTTCTGGCTGC (SEQ ID NO: 176), where the insertion is indicated in boldface. In yet another example, the CD33 gene may comprise a mutated fragment having both a deletion and an insertion relative to the wild-type counterpart, e.g., the deletion shown in SEQ ID NO: 175 and the insertion shown in SEQ ID NO: 176 as relative to the wild-type counterpart sequence SEQ ID NO:174.

In some embodiments, the edited CD33 gene may be described in terms of a fragment that is deleted from the wild-type (or unedited) gene.

For example, the edited CD33 gene may lack a fragment comprising GGATCCAAATTTCTGGCTGC (SEQ ID NO: 174), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising AGTTCATGGTTACTGGTTCC (SEQ ID NO: 186), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising ACTCCCCAGTTCATGGTTAC (SEQ ID NO: 196), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising AGCCATTATATCCAGGGACT (SEQ ID NO: 207), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising TCAGTGACGGTACAGGAGGG (SEQ ID NO: 220), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising AGGTGAAGTTCGCTGGAGCT (SEQ ID NO: 243), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising AGTTCGCTGGAGCTGGTGTG (SEQ ID NO: 263), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising ACTACTCACTCCTCGGTGCT (SEQ ID NO: 268), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising CCCGATCTTCTCCTGGTTGT (SEQ ID NO: 285), or a portion thereof, which may comprise 1, 2, 3, 4, 5, 6, 7, 8, 9, 10, 11, 12, 13, 14, 15, or more nucleotides.

For example, the edited CD33 gene may lack a fragment comprising AAATCCTCATCCCTGGCACT (SEQ ID NO: 299), or a portion thereof, which may comprise 1, 2, 3, 4, 5, 6, 7, 8, 9, 10, 11, 12, 13, 14, 15, or more nucleotides.

In some embodiments, the edited CD33 gene may have one or more of the following features:

(a) comprise a nucleotide sequence of GGATCCAAATTCTGGCTGC (SEQ ID NO:175), GGATCCAAATTTTCTGGCTGC (SEQ ID NO:176), GGATCCTGGCTGC (SEQ ID NO: 177), GGATCCAATTCTGGCTGC (SEQ ID NO: 178), TCCTGGCTGC (SEQ ID NO: 179), GGATCTGGCTGC (SEQ ID NO: 180), GGATCC, and/or GGATCCATTCTGGCTGC (SEQ ID NO: 181);

(b) lack a fragment comprising GGATCCAAATTTCTGGCTGC (SEQ ID NO: 174); and

(c) lack a fragment, the 3′ segment of which comprises the nucleotide sequence of GGATCCAAATTTC (SEQ ID NO: 182), GGATCCAAATT (SEQ ID NO: 183), or GGATCCAAATTT (SEQ ID NO: 185).

Such an edited CD33 gene may be produced using a guide RNA comprising a spacer sequence of SEQ ID NO: 164 (e.g., the gRNA of SEQ ID NO: 142).

In some embodiments, the edited CD33 gene may have one or more of the following features:

(a) comprise a nucleotide sequence of AGTTCATGGTACTGGTTCC (SEQ ID NO: 187), AGTTCATGGTTCC (SEQ ID NO: 188), AGTTCATGTACTGGTTCC (SEQ ID NO: 189), AGTTCATGGTTTACTGGTTCC (SEQ ID NO: 190), AGTTCC, AGTACTGGTTCC (SEQ ID NO: 191), AGTTCATACTGGTTCC (SEQ ID NO: 192), AGTTCATGGTATACTGGTTCC (SEQ ID NO: 193), and/or AGTTACTGGTTCC (SEQ ID NO: 194); and

(b) lack a fragment comprising AGTTCATGGTTACTGGTTCC (SEQ ID NO: 186);

Such an edited CD33 gene may be produced using a guide RNA comprising a spacer sequence of SEQ ID NO: 165 (e.g., the gRNA of SEQ ID NO: 143).

In some embodiments, the edited CD33 gene may have one or more of the following features:

(a) comprise a nucleotide sequence of ACTCCCCAGTTTCATGGTTAC (SEQ ID NO: 197), ACTCCCCAGTCATGGTTAC (SEQ ID NO: 198), ACTCCCCATGGTTAC (SEQ ID NO: 199), ACTCCCCAGTTAC (SEQ ID NO: 200), ACTCATGGTTAC (SEQ ID NO: 201), ACTCCCCATCATGGTTAC (SEQ ID NO: 202), ACTCCCCATTCATGGTTAC (SEQ ID NO: 203), ACTCCCCAGTGTCATGGTTAC (SEQ ID NO: 204), and/or ACTCCCCAGTCTCATGGTTAC (SEQ ID NO: 205);

(b) lack a fragment comprising ACTCCCCAGTTCATGGTTAC (SEQ ID NO: 196); and

(c) lack a fragment, the 3′ segment of which comprises the nucleotide sequence of ACTCCCCAGTTCATGGTT (SEQ ID NO: 206).

Such an edited CD33 gene may be produced using a guide RNA comprising a spacer sequence of SEQ ID NO: 166 (e.g., the gRNA of SEQ ID NO: 144).

In some embodiments, the edited CD33 gene may have one or more of the following features:

(a) comprise a nucleotide sequence of AGCCATTATCCAGGGACT (SEQ ID NO: 208), AGCCAGGGACT (SEQ ID NO: 209), AGCCATTATTCCAGGGACT (SEQ ID NO: 210), AGTCCAGGGACT (SEQ ID NO: 211), AGCCATTATAATCCAGGGACT (SEQ ID NO: 212), AGCCATTATCCGGGGACT (SEQ ID NO: 213), AGCCATTATACAGGGACT (SEQ ID NO: 214), AGCCATTATTCCGGGGACT (SEQ ID NO: 216), and/or AGCCATTATAATCCGGGGACT (SEQ ID NO: 217);

(b) lack a fragment comprising AGCCATTATATCCAGGGACT (SEQ ID NO: 207); and

(c) lack a fragment, the 3′ segment of which comprises the nucleotide sequence of AGCCATTATATCCA (SEQ ID NO: 218) or AGCCATTATA (SEQ ID NO: 219).

Such an edited CD33 gene may be produced using a guide RNA comprising a spacer sequence of SEQ ID NO: 167 (e.g., the gRNA of SEQ ID NO: 145).

In some embodiments, the edited CD33 gene may have one or more of the following features:

(a) comprise a nucleotide sequence of TCAGTGACAGGAGGG (SEQ ID NO: 221), TCAGTGACGTACAGGAGGG (SEQ ID NO: 222), TCAGGAGGG (SEQ ID NO: 223), TCAGTGACGGAGGG (SEQ ID NO: 224), TCAGTGACGGGAGGG (SEQ ID NO: 226), TCAGTGACGGTTACAGGAGGG (SEQ ID NO: 227), TCAGTGACGGACAGGAGGG (SEQ ID NO: 228), TCAGTGACGGGTACAGGAGGG (SEQ ID NO: 229), TCAGTACAGGAGGG (SEQ ID NO: 230), TCAGTGACTACAGGAGGG (SEQ ID NO: 231), TCAGTGACGGG (SEQ ID NO: 232), TCAGTGACGG (SEQ ID NO: 233), TCAGTGACGGCAGGAGGG (SEQ ID NO: 234),TCAGTGACGGAGGAGGG (SEQ ID NO: 235), TCAGTGATACAGGAGGG (SEQ ID NO: 236), TCAGTGTACAGGAGGG (SEQ ID NO: 237), and/or TCATACAGGAGGG (SEQ ID NO: 238);

(b) lack a fragment comprising TCAGTGACGGTACAGGAGGG (SEQ ID NO: 220);

(c) lack a fragment, the 3′ segment of which comprises the nucleotide sequence of TCAGTGACGGTA (SEQ ID NO: 239) or TCAGTGACG; and

(d) lack a fragment, the 5′ segment of which comprises the nucleotide sequence of GTGACGGTACAGGAGGG (SEQ ID NO: 242).

Such an edited CD33 gene may be produced using a guide RNA comprising a spacer sequence of SEQ ID NO: 168 (e.g., the gRNA of SEQ ID NO: 146).

In some embodiments, the edited CD33 gene may have one or more of the following features:
(a) comprise a nucleotide sequence of AGCTGGAGCT (SEQ ID NO: 244), AGGTGAAGCTGGAGCT (SEQ ID NO: 245), AGGTGAAGCT (SEQ ID NO: 246), AGGTGAAGTTGGAGCT (SEQ ID NO: 247), AGGTGAAGTCGCTGGAGCT (SEQ ID NO: 248), AGGTGGAGCT (SEQ ID NO: 249), AGGTGAAGCGCTGGAGCT (SEQ ID NO: 250), AGGTGACGCTGGAGCT (SEQ ID NO: 252), and/or AGGTGAAGTTTCGCTGGAGCT (SEQ ID NO: 253);

(b) lack a fragment comprising AGGTGAAGTTCGCTGGAGCT (SEQ ID NO: 243);

(c) lack a fragment, the 3′ segment of which comprises the nucleotide sequence of AGGTGAAGTTCG (SEQ ID NO: 256), AGGTGAAGTTCGCTGGAG (SEQ ID NO: 259), AGGTGAAGTTCGCTGG (SEQ ID NO: 260), or AGGTGAAGTT (SEQ ID NO: 261); and

(d) lack a fragment, the 5′ segment of which comprises the nucleotide sequence of GGTGAAGTTCGCTGGAGCT (SEQ ID NO: 262).

Such an edited CD33 gene may be produced using a guide RNA comprising a spacer sequence of SEQ ID NO: 169 (e.g., the gRNA of SEQ ID NO: 147).

In some embodiments, the edited CD33 gene may have one or more of the following features:

(a) comprise a nucleotide sequence of AGTTCGCTGGTGTG (SEQ ID NO: 264) and/or AGTTCGCTGAGCTGGTGTG (SEQ ID NO: 266);

(b) lack a fragment comprising AGTTCGCTGGAGCTGGTGTG (SEQ ID NO: 263); and

(c) lack a fragment, the 3′ segment of which comprises the nucleotide sequence of AGTTCGCTGG (SEQ ID NO: 267).

Such an edited CD33 gene may be produced using a guide RNA comprising a spacer sequence of SEQ ID NO: 170 (e.g., the gRNA of SEQ ID NO: 148).

In some embodiments, the edited CD33 gene may have one or more of the following features:

(a) comprise a nucleotide sequence of ACTACTCACTTCCTCGGTGCT (SEQ ID NO: 269), ACTACTCGGTGCT (SEQ ID NO: 270), ACTACTCATCCTCGGTGCT (SEQ ID NO: 271), ACTACT, ACTACTCACCCTCGGTGCT (SEQ ID NO: 272), ACTACTCCTCGGTGCT (SEQ ID NO: 273), ACTACTCACCTCGGTGCT (SEQ ID NO: 275), ACTACTCACTCGGTGCT (SEQ ID NO: 276), ACTACTCTCCTCGGTGCT (SEQ ID NO: 277), ACTACTTCCTCGGTGCT (SEQ ID NO: 278), ACTACTCACTTCGGTGCT (SEQ ID NO: 279), and/or ACTATCCTCGGTGCT (SEQ ID NO: 280);

(b) lack a fragment comprising ACTACTCACTCCTCGGTGCT (SEQ ID NO: 268); and

(c) lack a fragment, the 3′ segment of which comprises the nucleotide sequence of ACTACTCACT (SEQ ID NO: 282), ACTACTCACTCCTC (SEQ ID NO: 283), or ACTACTCACTCCTCGGT (SEQ ID NO: 284).

Such an edited CD33 gene may be produced using a guide RNA comprising a spacer sequence of SEQ ID NO: 171 (e.g., the gRNA of SEQ ID NO: 149).

In some embodiments, the edited CD33 gene may have one or more of the following features:

(a) comprise a nucleotide sequence of CCCGATCTTCCTGGTTGT (SEQ ID NO: 286),

CCCGATCCTGGTTGT (SEQ ID NO: 287), CCCGATCTGGTTGT (SEQ ID NO: 288), CCCTGGTTGT (SEQ ID NO: 289), CCCGATCTTCTGGTTGT (SEQ ID NO: 290), CCCGATCTTGGTTGT (SEQ ID NO: 291), CCCGATCTCCTGGTTGT (SEQ ID NO: 292), CCCGATCTTCCCTGGTTGT (SEQ ID NO: 293), and/or CCCGAT;

(b) lack a fragment comprising CCCGATCTTCTCCTGGTTGT (SEQ ID NO: 285); (c) lack a fragment, the 3′ segment of which comprises the nucleotide sequence of CCCGATCTTCT (SEQ ID NO: 295); and

(d) lack a fragment, the 5′ segment of which comprises the nucleotide sequence of TCCTGGTTGT (SEQ ID NO: 298).

Such an edited CD33 gene may be produced using a guide RNA comprising a spacer sequence of SEQ ID NO: 172 (e.g., the gRNA of SEQ ID NO: 150).

In some embodiments, the edited CD33 gene may have one or more of the following features:

(a) comprise a nucleotide sequence of AAATCCTGGCACT (SEQ ID NO: 300), AAATCCCTGGCACT (SEQ ID NO: 301), AAATCCTCATTCCCTGGCACT (SEQ ID NO: 302), AAATCCTCACCCTGGCACT (SEQ ID NO: 304), AAATCCTCCCCTGGCACT (SEQ ID NO: 305), AAATCCTCCCTGGCACT (SEQ ID NO: 306), AAATCCCCTGGCACT (SEQ ID NO: 307), ACATCCTCATTCCCTGGCACT (SEQ ID NO: 308), ACATCCTGGCACT (SEQ ID NO: 309), AAATCCTCTCCCTGGCACT (SEQ ID NO: 310), AAATCCTCATCTGGCACT (SEQ ID NO: 311), AAATCCT, AAACCCTGGCACT (SEQ ID NO: 312), AAATCCTCTGGCACT (SEQ ID NO: 313), AAATCCCCCTGGCACT (SEQ ID NO: 314), AAATCCTCACT (SEQ ID NO: 315), ACATCCCTGGCACT (SEQ ID NO: 316), and/or AAAT;

(b) lack a fragment comprising AAATCCTCATCCCTGGCACT (SEQ ID NO: 299);

(c) lack a fragment, the 3′ segment of which comprises the nucleotide sequence of AAATCCTCAT (SEQ ID NO: 317), AAATCCTCATCCCT (SEQ ID NO: 318), AAATCCTCATCCCTGG (SEQ ID NO: 320), AAATCCTCATC (SEQ ID NO: 322), or AAATCCTCATCCCTGGCA (SEQ ID NO: 324); and

(d) lack a fragment, the 5′ segment of which comprises the nucleotide sequence of CTCATCCCTGGCACT (SEQ ID NO: 323).

Such an edited CD33 gene may be produced using a guide RNA comprising a spacer sequence of SEQ ID NO: 173 (e.g., the gRNA of SEQ ID NO: 151).

PD-1 Gene Edit

PD-1 is an immune checkpoint molecule that is upregulated in activated T cells and serves to dampen or stop T cell responses. Disrupting PD-1 by gene editing could lead to more persistent and/or potent therapeutic T cell responses and/or reduce immune suppression in a subject. In some embodiments, an engineered T cell comprises a disrupted PD-1 gene. In some embodiments, expression of the endogenous PD-1 gene is eliminated to enhance anti-tumor efficacy of the CAR T cells of the present disclosure.

Non-limiting examples of modified and unmodified PD-1 gRNA sequences that may be used as provided herein to create a genomic deletion in the PD-1 gene are listed in Table 4 (e.g., SEQ ID NOS: 22 and 23). See also International Application No. PCT/US2018/032334, filed May 11, 2018, incorporated herein by reference. Other gRNA sequences may be designed using the PD-1 gene sequence located on Chromosome 2 (GRCh38 coordinates: Chromosome 2: 241,849,881-241,858,908; Ensembl: ENSG00000188389).

In some embodiments, gRNAs targeting the PD-1 genomic region create Indels in the PD-1 gene disrupting expression of the PD-1 mRNA or protein.

In some embodiments, an engineered T cell comprises a disrupted PD-1 gene. In some embodiments, at least 50% of the engineered T cells of a population of engineered T cells does not express a detectable level of PD-1 surface protein. For example, at least 55%, at least 60%, at least 70%, at least 75%, at least 80%, at least 85%, at least 90%, or at least 95% of the engineered T cells of a population may not express a detectable level of PD-1 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of PD-1 surface protein.

In some embodiments, a ribonucleoprotein particle (RNP) containing an RNA-guided nuclease (e.g., a Cas nuclease, such as a Cas9 nuclease) and a gRNA targeting the PD-1 gene (or any other gene of interest) are delivered to T cells (e.g., primary T cells). In other embodiments, the RNA-guided nuclease and gRNA are delivered separately to T cells. A ribonucleoprotein particle (RNP) is simply a RNA-guided nuclease (e.g., Cas9) pre-complexed/complexed with a gRNA.

CD70 Gene Edit

Cluster of Differentiation 70 (CD70) is a member of the tumor necrosis factor superfamily and its expression is restricted to activated T and B lymphocytes and mature dendritic cells. CD70 has also been detected on hematological tumors and on carcinomas. CD70 is implicated in tumor cell and regulatory T cell survival through interaction with its ligand, CD27. Disrupting CD70 by gene editing increases cell expansion and reduces cell exhaustion. In some embodiments, an engineered T cell comprises a disrupted CD70 gene. In some embodiments, expression of the endogenous CD70 gene is eliminated to enhance anti-tumor efficacy of the CAR T cells of the present disclosure. In some embodiments, gRNAs targeting the CD70 genomic region create Indels in, or around, the CD70 gene disrupting expression of the CD70 mRNA and/or protein.

Non-limiting examples of modified and unmodified CD70 gRNA sequences that may be used as provided herein to create a genomic disruption in the CD70 gene are listed in Table 4 (e.g., SEQ ID NOS: 24-27). See also International Application No. PCT/IB2019/000500, filed May 10, 2019, incorporated herein by reference. Other gRNA sequences may be designed using the CD70 gene sequence located on Chromosome 19 (GRCh38 coordinates: Chromosome 19: 6,583,183-6,604,103; Ensembl: ENSG00000125726).

In some embodiments, an engineered T cell comprises a disrupted CD70 gene. In some embodiments, at least 50% of the engineered T cells of a population of engineered T cells does not express a detectable level of CD70 surface protein. For example, at least 55%, at least 60%, at least 70%, at least 75%, at least 80%, at least 85%, at least 90%, or at least 95% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of CD70 surface protein.

In some embodiments, a ribonucleoprotein particle (RNP) containing an RNA-guided nuclease (e.g., a Cas nuclease, such as a Cas9 nuclease) and a gRNA targeting the CD70 gene (or any other gene of interest) are delivered to T cells (e.g., primary T cells). In other embodiments, the RNA-guided nuclease and gRNA are delivered separately to T cells. A ribonucleoprotein particle (RNP) is simply a RNA-guided nuclease (e.g., Cas9) pre-complexed/complexed with a gRNA.

### Cellular Phenotypes

In some embodiments, one or more gene edits within a population of cells results in a phenotype associated with changes in cellular proliferative capacity, cellular exhaustion, cellular viability, cellular lysis capability (e.g., increase cytokine production and/or release), or any combination thereof.

In some embodiments, engineered T cells of the present disclosure exhibit at least 20% greater cellular proliferative capacity, relative to control T cells. For example, engineered T cells may exhibit at least 25%, at least 30%, at least 35%, at least 40%, at least 45%, at least 50%, at least 55%, at least 60%, at least 65%, at least 70%, at least 75%, at least 80%, or at least 90% greater cellular proliferative capacity, relative to control T cells. In some embodiments, engineered T cells of the present disclosure exhibit 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% greater cellular proliferative capacity, relative to control T cells.

In some embodiments, engineered T cells of the present disclosure exhibit an at least 20% increase in cellular viability, relative to control cells. For example, engineered T cells of the present disclosure may exhibit at least 25%, at least 30%, at least 35%, at least 40%, at least 45%, at least 50%, at least 55%, at least 60%, at least 65%, at least 70%, at least 75%, at least 80%, or at least 90% increase in cellular viability, relative to control cells. In some embodiments, engineered T cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular viability, relative to control cells.

In some embodiments, engineered T cells of the present disclosure exhibit an at least 20% increase in cellular lysis capability (kill at least 20% more target cells), relative to control cells. For example, engineered T cells of the present disclosure may exhibit an at least at least 25%, at least 30%, at least 35%, at least 40%, at least 45%, at least 50%, at least 55%, at least 60%, at least 65%, at least 70%, at least 75%, at least 80%, or at least 90% increase in cellular lysis capability, relative to control cells. In some embodiments, engineered T cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular lysis capability, relative to control cells. For example, the level of cytokines (e.g., IL-2 and/or IFN-gamma) secreted by the engineered T cells may at least 2-fold (e.g., at least 3-fold, at least 4-fold, or at least 5-fold) greater than the level of cytokines secreted by control T cells.

Control T cells, in some embodiments, are engineered T cells (e.g., gene edited T cells). In some embodiments, control T cells are engineered T cells that comprise a disrupted TRAC gene, a nucleic acid encoding a CAR (e.g., an anti-CD33 CAR) inserted into the TRAC gene, and/or a disrupted β2M gene. In some embodiments, control T cells are unedited T cells.

### Gene Editing Methods

Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When an sequence of an endogenous gene is edited, for example by deletion, insertion or substitution of nucleotide(s)/nucleic acid(s), the endogenous gene comprising the affected sequence may be knocked-out or knocked-down due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and WP/SPBc/TP901-1, whether used individually or in combination.

Other non-limiting examples of targeted nucleases include naturally-occurring and recombinant nucleases, e.g., CRISPR/Cas9, restriction endonucleases, meganucleases homing endonucleases, and the like.

### CRISPR-Cas9 Gene Editing

The CRISPR-Cas9 system is a naturally-occurring defense mechanism in prokaryotes that has been repurposed as a RNA-guided DNA-targeting platform used for gene editing. It relies on the DNA nuclease Cas9, and two noncoding RNAs-crisprRNA (crRNA) and trans-activating RNA (tracrRNA)—to target the cleavage of DNA.

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

In some embodiments, the Cas9 (CRISPR associated protein 9) endonuclease is from Streptococcus pyogenes, although other Cas9 homologs may be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

### Guide RNAs

The present disclosure provides a genome-targeting nucleic acid that can direct the activities of an associated polypeptide (e.g., a site-directed polypeptide) to a specific target sequence within a target nucleic acid. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid is a double-molecule guide RNA. In some embodiments, the genome-targeting nucleic acid is a single-molecule guide RNA.

A double-molecule guide RNA comprises two strands of RNA. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (sgRNA) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins.

A single-molecule guide RNA (referred to as a “sgRNA” or “gRNA”) in a Type V system comprises, in the 5′ to 3′ direction, a minimum CRISPR repeat sequence and a spacer sequence.

The sgRNA can comprise a 20 nucleotide spacer sequence at the 5′ end of the sgRNA sequence. The sgRNA can comprise a less than 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence (see Table 3).

The sgRNA can comprise no uracil at the 3′ end of the sgRNA sequence. The sgRNA can comprise one or more uracil at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence.

The sgRNA can be unmodified or modified. For example, modified sgRNAs can comprise one or more 2′-O-methyl phosphorothioate nucleotides.

TABLE 3
Exemplary sgRNA sequences.

| SEQ ID | |
|---|---|
| NO. | sgRNA sequence |
| 15 | nnnnnnnnnnnnnnnnnnnnguuuu |
| | agagcuagaaauagcaaguuaaaau |
| | aaggcuaguccguuaucaacuugaa |
| | aaaguggcaccgagucggugcuuuu |
| 16 | nnnnnnnnnnnnnnnnnnnnguuuu |
| | agagcuagaaauagcaaguuaaaau |
| | aaggcuaguccguuaucaacuugaa |
| | aaaguggcaccgagucggugc |
| 17 | n₍₁₇₋₃₀₎guuuuagagcuagaaau |
| | agcaaguuaaaauaaggcuaguccg |
| | uuaucaacuugaaaaaguggcaccg |
| | agucggugcu₍₁₋₈₎ |

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

Spacer Sequence

A gRNA comprises a spacer sequence. A spacer sequence is a sequence (e.g., a 20 nucleotide sequence) that defines the target sequence (e.g., a DNA target sequences, such as a genomic target sequence) of a target nucleic acid of interest. In some embodiments, the spacer sequence is 15 to 30 nucleotides. In some embodiments, the spacer sequence is 15, 16, 17, 18, 19, 29, 21, 22, 23, 24, 25, 26, 27, 28, 29, or 30 nucleotides. In some embodiments, a spacer sequence is 20 nucleotides.

The “target sequence” is adjacent to a PAM sequence and is the sequence modified by an RNA-guided nuclease (e.g., Cas9). The “target nucleic acid” is a double-stranded molecule: one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid of interest. Thus, the gRNA spacer sequence is the RNA equivalent of the target sequence. For example, if the target sequence is 5′-AGAGCAACAGTGCTGTGGCC-3′ (SEQ ID NO: 325), then the gRNA spacer sequence is 5′-AGAGCAACAGUGCUGUGGCC-3′ (SEQ ID NO: 19). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM of the Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence comprises 20 nucleotides. In some embodiments, the target nucleic acid comprises less than 20 nucleotides. In some embodiments, the target nucleic acid comprises more than 20 nucleotides. In some embodiments, the target nucleic acid comprises at least: 5, 10, 15, 16, 17, 18, 19, 20, 21, 22, 23, 24, 25, 30 or more nucleotides. In some embodiments, the target nucleic acid comprises at most: 5, 10, 15, 16, 17, 18, 19, 20, 21, 22, 23, 24, 25, 30 or more nucleotides. In some embodiments, the target nucleic acid sequence comprises 20 bases immediately 5′ of the first nucleotide of the PAM. For example, in a sequence comprising 5′-NNNNNNNNNNNNNNNNNNNNNRG-3′, the target nucleic acid comprises the sequence that corresponds to the Ns, wherein N is any nucleotide, and the underlined NRG sequence is the S. pyogenes PAM.

Non-limiting examples of gRNAs that may be used as provided herein are provided in Table 4, Table 10, and PCT/US2018/032334, filed May 11, 2018.

TABLE 4
gRNA Sequences/Target Sequences
gRNA Sequences

| Name | Unmodified | Modified |
|---|---|---|
| | Sequence | Sequence |
| TRAC sgRNA | AGAGCAACAG | A*G*A*GCAA |
| | UGCUGUGGCC | CAGUGCUGUG |
| | guuuuagagc | CiCC gu uu |
| | uagaaauagc | ua gage u |
| | aaguuaaaau | a gaaauagc |
| | aaggcuaguc | aaguuaaaau |
| | cguuaucaac | aaggcuaguc |
| | uugaaaaagu | cguuaucaac |
| | ggcaccgagu | uugaaaaagu |
| | cggugcUUUU | ggcaccgagu |
| | (SEQ ID | cggugcU*U* |
| | NO: 18) | U*U(SEQ ID |
| | | NO: 28) |
| TRAC sgRNA | AGAGCAACAG | A*G*A*GCAA |
| spacer | UGCUGUGGCC | CAGUGCUGUG |
| | (SEQ ID | GCC (SEQ I |
| | NO: 19) | D NO: 29) |
| β2M sgRNA | GCUACUCUCU | G*C*U*ACUC |
| | CUUUCUGGCC | UCUCUUUCUG |
| | guuuuagagc | GCCguuuuag |
| | uagaaauagc | agcuagaaau |
| | aaguuaaaau | agcaaguuaa |
| | aaggcuaguc | aauaaggcua |
| | cguuaucaac | guccguuauc |
| | uugaaaaagu | aacuugaaaa |
| | ggcaccgagu | aguggcaccg |
| | cggugcUUUU | agucggugcU |
| | (SEQ ID | *U*U*U(SEQ |
| | NO: 20) | ID NO: 30) |
| β2M sgRNA spacer | GCUACUCUCU | G*C*U*ACUC |
| | CUUUCUGGCC | UCUCUUUCUG |
| | (SEQ ID | GCC (SEQ I |
| | NO: 21) | D NO: 31) |
| PD-1 sgRNA | CUGCAGCUUC | C*U*G*CAGC |
| | UCCAACACAU | UUCUCCAACA |
| | guuuuagagc | CAUguuuuag |
| | uagaaauagc | agcuagaaau |
| | aaguuaaaau | agcaaguuaa |
| | aaggcuaguc | aauaaggcua |
| | cguuaucaac | guccguuauc |
| | uugaaaaagu | aacuugaaaa |
| | ggcaccgagu | aguggcaccg |
| | cggugcUUUU | agucggugcU |
| | (SEQ ID | *U*U*U |
| | NO: 22) | (SEQ ID |
| | | NO: 32) |
| PD-1 sgRNA | CUGCAGCUUC | C*U*G*CAGC |
| spacer | UCCAACACAU | UUCUCCAACA |
| | (SEQ ID | CAU |
| | NO: 23) | (SEQ ID |
| | | NO: 33) |
| CD70 sgRNA | GCUUUGGUCC | G*C*U*UUGG |
| (E1_T7) | CAUUGGUCGC | UCCCAUUGGU |
| | guuuuagagc | CGCguuuuag |
| | uagaaauagc | agcuagaaau |
| | aaguuaaaau | agcaaguuaa |
| | aaggc uagu | aauaaggcua |
| | ccguuaucaa | guccguuauc |
| | cuugaaaaag | aacuugaaaa |
| | uggcaccgag | aguggcaccg |
| | ucggugcUUU | agucggugcU |
| | U | *U*U*U |
| | (SEQ ID | (SEQ |
| | NO: 24) | ID NO: |
| | | 34), T7 |
| CD70 sgRNA | GCUUUGGUCC | G*C*U*UUGG |
| (E1_T7) | CAUUGGUCGC | UCCCAUUGCU |
| spacer | (SEQ ID | CGC (SEQ |
| | NO: 25) | ID NO: |
| | | 35) |
| CD70 sgRNA | GCCCGCAGGA | G*C*C*CGCA |
| (E1_T8) | CGCACCCAUA | GGACGCACCC |
| | guuuuagagc | AUAguuuuag |
| | uagaaauagc | agcuagaaau |
| | aaguuaaaau | agcaaguuaa |
| | aaggcuaguc | aauaaggcua |
| | cguuaucaac | guccguuauc |
| | uugaaaaagu | aacuugaaaa |
| | ggcaccgagu | aguggcaccg |
| | cggugcUUUU | agucggugcU |
| | (SEQ ID | *U*U*U |
| | NO: 26) | (SEQ ID |
| | | NO: 36), |
| | | T8 |
| CD70 sgRNA | GCCCGCAGGA | g*c*c*cgca |
| (E1_T8) spacer | CGCACCCAUA | ggacgcaccc |
| | (SEQ ID | AUA (SEQ |
| | NO: 27) | ID NO: |
| | | 37) |

Target Sequences

| Guide Name | Target Sequence (PAM) |
|---|---|
| CD70 sgRNA | GCTTTGGTCCCATTGGTCGC (GGG) |
| (E1_T7) | (SEQ ID NO: 38) |
| CD70 sgRNA | GCCCGCAGGACGCACCCATA (GGG) |
| (E1_T8) | (SEQ ID NO: 39) |
| TRAC sgRNA | AGAGCAACAGTGCTGTGGCC (TGG) |
| | (SEQ ID NO: 40) |
| β2M sgRNA | GCTACTCTCTCTTTCTGGCC (TGG) |
| | (SEQ ID NO: 41) |
| PD-1 sgRNA | CTGCAGCTTCTCCAACACAT (CGG) |
| | (SEQ ID NO: 42) |

*2′-O-methyl phosphorothioate residue

### Chimeric Antigen Receptor (CAR) T Cells

A chimeric antigen receptor refers to an artificial immune cell receptor that is engineered to recognize and bind to an antigen expressed by tumor cells. Generally, a CAR is designed for a T cell and is a chimera of a signaling domain of the T-cell receptor (TCR) complex and an antigen-recognizing domain (e.g., a single chain fragment (scFv) of an antibody or other antibody fragment) (Enblad et al., Human Gene Therapy. 2015; 26(8):498-505). A T cell that expresses a CAR is referred to as a CAR T cell. CARs have the ability to redirect T-cell specificity and reactivity toward a selected target in a non-MHC-restricted manner. The non-MHC-restricted antigen recognition gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCR) alpha and beta chains.

There are four generations of CARs, each of which contains different components. First generation CARs join an antibody-derived scFv to the CD3zeta ((or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains may include, e.g., a combination of CD3ζ, CD27, CD28, 4-1BB, ICOS, or OX40. CARs, in some embodiments, contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125(26):4017-4023; Kakarla and Gottschalk, Cancer J. 2014; 20(2):151-155).

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency.

In some embodiments, a chimeric antigen receptor is a first generation CAR. In other embodiments, a chimeric antigen receptor is a second generation CAR. In yet other embodiments, a chimeric antigen receptor is a third generation CAR.

A CAR, in some embodiments, comprises an extracellular (ecto) domain comprising an antigen binding domain (e.g., an antibody, such as an scFv), a transmembrane domain, and a cytoplasmic (endo) domain.

Ectodomain

The ectodomain is the region of the CAR that is exposed to the extracellular fluid and, in some embodiments, includes an antigen binding domain, and optionally a signal peptide, a spacer domain, and/or a hinge domain. In some instances the antigen binding domain is a fragment of an antibody. See discussions below.

In some embodiments, the antigen binding domain is a single-chain variable fragment (scFv) that include the VL and VH of immunoglobins connected with a short linker peptide. The linker, in some embodiments, includes hydrophilic residues with stretches of glycine and serine for flexibility as well as stretches of glutamate and lysine for added solubility. A single-chain variable fragment (scFv) is not actually a fragment of an antibody, but instead is a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. Non-limiting examples of VH and VL protein sequences that may be used to create an anti-CD33 scFv may include the amino acid sequence of SEQ ID NOs: 65, 77 or 89 (VH) and SEQ ID NOs: 66, 78 or 90 (VL). In some embodiments, the scFv of the present disclosure is humanized. In other embodiments, the scFv is fully human. In yet other embodiments, the scFv is a chimera (e.g., of mouse and human sequence). In some embodiments, the scFv is an anti-CD33 scFv (binds specifically to CD33). Non-limiting examples of anti-CD33 scFv proteins that may be used as provided herein may include the amino acid sequence of any one of SEQ ID NOS: 54, 68, 75, 82. Other scFv proteins may be used.

The signal peptide can enhance the antigen specificity of CAR binding. Signal peptides can be derived from antibodies, such as, but not limited to, CD8, as well as epitope tags such as, but not limited to, GST or FLAG. Examples of signal peptides include MLLLVTSLLLCELPHPAFLLIP (SEQ ID NO: 162) and MALPVTALLLPLALLLHAARP (SEQ ID NO: 121). Other signal peptides may be used.

In some embodiments, a spacer domain or hinge domain is located between an extracellular domain (comprising the antigen binding domain) and a transmembrane domain of a CAR, or between a cytoplasmic domain and a transmembrane domain of the CAR. A spacer domain is any oligopeptide or polypeptide that functions to link the transmembrane domain to the extracellular domain and/or the cytoplasmic domain in the polypeptide chain. A hinge domain is any oligopeptide or polypeptide that functions to provide flexibility to the CAR, or domains thereof, or to prevent steric hindrance of the CAR, or domains thereof. In some embodiments, a spacer domain or a hinge domain may comprise up to 300 amino acids (e.g., 10 to 100 amino acids, or 5 to 20 amino acids). In some embodiments, one or more spacer domain(s) may be included in other regions of a CAR. In some embodiments, the hinge domain is a CD8 hinge domain. Other hinge domains may be used.

Transmembrane Domain

The transmembrane domain is a hydrophobic alpha helix that spans the membrane.

The transmembrane domain provides stability of the CAR. In some embodiments, the transmembrane domain of a CAR as provided herein is a CD8 transmembrane domain. In other embodiments, the transmembrane domain is a CD28 transmembrane domain. In yet other embodiments, the transmembrane domain is a chimera of a CD8 and CD28 transmembrane domain. Other transmembrane domains may be used. In some embodiments, the transmembrane domain is a CD8a transmembrane domain: FVPVFLPAKPTTTPAPRPPTPAPTIASQPLSLRPEACRPAAGGAVHTRGLDFACDIYIW APLAGTCGVLLLSLVITLYCNHRNR (SEQ ID NO: 125). Other transmembrane domains may be used.

In some embodiments, the transmembrane domain is a CD8a transmembrane domain comprising the amino acid sequence: IYIWAPLAGTCGVLLLSLVITLY (SEQ ID NO: 163).

Endodomain

The endodomain is the functional end of the receptor. Following antigen recognition, receptors cluster and a signal is transmitted to the cell. The most commonly used endodomain component is CD3-zeta, which contains three (3) immunoreceptor tyrosine-based activation motif (ITAM)s. This transmits an activation signal to the T cell after the antigen is bound. In many cases, CD3-zeta may not provide a fully competent activation signal and, thus, a co-stimulatory signaling is used. For example, CD28 and/or 4-1BB may be used with CD3-zeta (CD3ζ) to transmit a proliferative/survival signal. Thus, in some embodiments, the co-stimulatory molecule of a CAR as provided herein is a CD28 co-stimulatory molecule. In other embodiments, the co-stimulatory molecule is a 4-1BB co-stimulatory molecule. In some embodiments, a CAR includes CD3˜ and CD28. In other embodiments, a CAR includes CD3-zeta and 4-1BB. In still other embodiments, a CAR includes CD3˜, CD28, and 4-11BB. Table 5 provides examples of signaling molecules that may be used as provided herein.

TABLE 5
Exemplary sequences of CAR components.

| Name | Sequence |
|---|---|
| 4-1BB | AAACGGGGCAGAAAGAAACTCCTGTATATATTCAAACAACCATTTATGAGACCAGT |
| | CAAACTACTCAAGAGGAAGATGGCTGTAGCTGCCGATTTCCAGAAGAAGAAGAAA |
| | GGAGGATGTGAACTG (SEQ ID NO: 43) |
| | KRGRKKLLYIFKQPFMRPVQTTQEEDGCSCRFPEEEEGGCEL (SEQ ID NO:) |
| | 44 |
| CD28 | TCAAAGCGGAGTAGGTTGTTGCATTCCGATTACATGAATATGACTCCTCGCCGGCC |
| | TGGGCCGACAAGAAAACATTACCAACCCTATGCCCCCCCACGAGACTTCGCTGCGT |
| | ACAGGTCC (SEQ ID NO: 45) |
| | SKRSRLLHSDYMNMTPRRPGPTRKHYQPYAPPRDFAAYRS (SEQ ID NO: |
| | 46) |
| CD3- | CGAGTGAAGTTTTCCCGAAGCGCAGACGCTCCGGCATATCAGCAAGGACAGAATCA |
| zeta | GCTGTATAACGAACTGAATTTGGGACGCCGCGAGGAGTATGACGTGCTTGATAAAC |
| | GCCGGGGGAGAGACCCGGAAATGGGGGGTAAACCCCGAAGAAAGAATCCCCAAGAA |
| | GGACTCTACAATGAACTCCAGAAGGATAAGATGGCGGAGGCCTACTCAGAAATAGG |
| | TATGAAGGGCGAACGACGACGGGGAAAAGGTCACGATGGCCTCTACCAAGGGTTGA |
| | GTACGGCAACCAAAGATACGTACGATGCACTGCATATGCAGGCCCTGCCTCCCAGA |
| | (SEQ ID NO: 47) |
| | RVKFSRSADAPAYQQGQNQLYNELNLGRREEYDVLDKRRGRDPEMGGKPRRKNPQE |
| | GLYNELQKDKMAEAYSEIGMKGERRRGKGHDGLYQGLSTATKDTYDALHMQALPPR |
| | (SEQ ID NO: 48) |

Exemplary CAR sequences are provided in Table 26 below.

TABLE 26
CAR Structure:
CD8[signal peptide]-anti-CD33[scFV]-CD8[hinge]-CD8[tm]-CD28[co-stimulatory domain]-CD3z;
or
CD8[signal peptide]-anti-CD33[scFV]-CD8[hinge]-CD8[tm]-41BB[co-stimulatory domain]-CD3z

| | | SEQ |
|---|---|---|
| | | ID |
| Name | Sequence | NO: |
| CTX-964 | GAGATGTAAGGAGCTGCTGTGACTTGCTCAAGGCCTTATATCGAGTAAACGG | 49 |
| Donor | TAGTGCTGGGGCTTAGACGCAGGTGTTCTGATTTATAGTTCAAAACCTCTAT | |
| LHA to RHA | CAATGAGAGAGCAATCTCCTGGTAATGTGATAGATTTCCCAACTTAATGCCA | |
| CD28 costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGCAGGTACAACTCCAACAACCCGGAGCTGAGGTTG | |
| | TAAAACCAGGTGCGTCAGTCAAGATGAGTTGCAAAGCCAGTGGATATACTTT | |
| | TACTTCCTATTACATTCATTGGATCAAGCAGACTCCAGGTCAGGGGCTCGAG | |
| | TGGGTAGGCGTGATCTACCCCGGTAACGACGACATTTCATACAACCAAAAAT | |
| | TTCAGGGGAAAGCGACGCTGACTGCTGACAAGAGTAGCACGACCGCATATAT | |
| | GCAACTCTCATCACTTACGTCTGAGGATTCTGCAGTTTATTATTGCGCTCGG | |
| | GAAGTTCGGCTTCGATATTTCGATGTGTGGGGTCAGGGCACGACCGTAACGG | |
| | TGAGCAGTGGTGGCGGTGGCGGGTCCGGGGGCGGTGGATCAGGTGGTGGGGG | |
| | GAGTGAGATAGTGTTGACCCAGTCACCGGGGTCCCTCGCAGTTTCACCGGGA | |
| | GAGAGGGTCACAATGTCCTGCAAATCCTCCCAATCAGTGTTCTTCTCTTCCA | |
| | GCCAAAAAAACTACCTTGCGTGGTATCAACAGATACCGGGACAGTCTCCTCG | |
| | CCTCCTGATCTACTGGGCATCTACCCGAGAAAGCGGTGTTCCGGATAGGTTT | |
| | ACCGGTTCCGGGTCTGGGACCGATTTTACGTTGACAATATCCAGCGTACAGC | |
| | CGGAAGACCTTGCTATCTATTACTGTCACCAGTACCTTTCCAGCCGGACGTT | |
| | CGGGCAGGGCACGAAGCTGGAGATTAAAAGTGCTGCTGCCTTTGTCCCGGTA | |
| | TTTCTCCCAGCCAAACCGACCACGACTCCCGCCCCGCGCCCTCCGACACCCG | |
| | CTCCCACCATCGCCTCTCAACCTCTTAGTCTTCGCCCCGAGGCATGCCGACC | |
| | CGCCGCCGGGGGTGCTGTTCATACGAGGGGCTTGGACTTCGCTTGTGATATT | |
| | TACATTTGGGCTCCGTTGGCGGGTACGTGCGGCGTCCTTTTGTTGTCACTCG | |
| | TTATTACTTTGTATTGTAATCACAGGAATCGCTCAAAGCGGAGTAGGTTGTT | |
| | GCATTCCGATTACATGAATATGACTCCTCGCCGGCCTGGGCCGACAAGAAAA | |
| | CATTACCAACCCTATGCCCCCCCACGAGACTTCGCTGCGTACAGGTCCCGAG | |
| | TGAAGTTTTCCCGAAGCGCAGACGCTCCGGCATATCAGCAAGGACAGAATCA | |
| | GCTGTATAACGAACTGAATTTGGGACGCCGCGAGGAGTATGACGTGCTTGAT | |
| | AAACGCCGGGGGAGAGACCCGGAAATGGGGGGTAAACCCCGAAGAAAGAATC | |
| | CCCAAGAAGGACTCTACAATGAACTCCAGAAGGATAAGATGGCGGAGGCCTA | |
| | CTCAGAAATAGGTATGAAGGGCGAACGACGACGGGGAAAAGGTCACGATGGC | |
| | CTCTACCAAGGGTTGAGTACGGCAACCAAAGATACGTACGATGCACTGCATA | |
| | TGCAGGCCCTGCCTCCCAGATAATAATAAAATCGCTATCCATCGAAGATGGA | |
| | TGTGTGTTGGTTTTTTGTGTGTGGAGCAACAAATCTGACTTTGCATGTGCAA | |
| | ACGCCTTCAACAACAGCATTATTCCAGAAGACACCTTCTTCCCCAGCCCAGG | |
| | TAAGGGCAGCTTTGGTGCCTTCGCAGGCTGTTTCCTTGCTTCAGGAATGGCC | |
| | AGGTTCTGCCCAGAGCTCTGGTCAATGATGTCTAAAACTCCTCTGATTGGTG | |
| | GTCTCGGCCTTATCCATTGCCACCAAAACCCTCTTTTTACTAAGAAACAGTG | |
| | AGCCTTGTTCTGGCAGTCCAGAGAATGACACGGGAAAAAAGCAGATGAAGAG | |
| | AAGGTGGCAGGAGAGGGCACGTGGCCCAGCCTCAGTCTCTCCAACTGAGTTC | |
| | CTGCCTGCCTGCCTTTGCTCAGACTGTTTGCCCCTTACTGCTCTTCTAGGCC | |
| | TCATTCTAAGCCCCTTCTCCAAGTTGCCTCTCCTTATTTCTCCCTGTCTGCC | |
| | AAAAAATCTTTCCCAGCTCACTAAGTCAGTCTCACGCAGTCACTCATTAACC | |
| | CACCAATCACTGATTGTGCCGGCACATGAATGCACCAGGTGTTGAAGTGGAG | |
| | GAATTAAAAAGTCAGATGAGGGGTGTGCCCAGAGGAAGCACCATTCTAGTTG | |
| | GGGGAGCCCATCTGTCAGCTGGGAAAAGTCCAAATAACTTCAGATTGGAATG | |
| | TGTTTTAACTCAGGGTTGAGAAAACAGCTACCTTCAGGACAAAAGTCAGGGA | |
| | AGGGCTCTCTGAAGAAATGCTACTTGAAGATACCAGCCCTACCAAGGGCAGG | |
| | GAGAGGACCCTATAGAGGCCTGGGACAGGAGCTCAATGAGAAAGG | |
| CTX-964 | CCACCATGGCGCTTCCGGTGACAGCACTGCTCCTCCCCTTGGCGCTGTTGCT | 50 |
| CAR | CCACGCAGCAAGGCCGCAGGTACAACTCCAACAACCCGGAGCTGAGGTTGTA | |
| CD28 costim. | AAACCAGGTGCGTCAGTCAAGATGAGTTGCAAAGCCAGTGGATATACTTTTA | |
| | CTTCCTATTACATTCATTGGATCAAGCAGACTCCAGGTCAGGGGCTCGAGTG | |
| | GGTAGGCGTGATCTACCCCGGTAACGACGACATTTCATACAACCAAAAATTT | |
| | CAGGGGAAAGCGACGCTGACTGCTGACAAGAGTAGCACGACCGCATATATGC | |
| | AACTCTCATCACTTACGTCTGAGGATTCTGCAGTTTATTATTGCGCTCGGGA | |
| | AGTTCGGCTTCGATATTTCGATGTGTGGGGTCAGGGCACGACCGTAACGGTG | |
| | AGCAGTGGTGGCGGTGGCGGGTCCGGGGGCGGTGGATCAGGTGGTGGGGGGA | |
| | GTGAGATAGTGTTGACCCAGTCACCGGGGTCCCTCGCAGTTTCACCGGGAGA | |
| | GAGGGTCACAATGTCCTGCAAATCCTCCCAATCAGTGTTCTTCTCTTCCAGC | |
| | CAAAAAAACTACCTTGCGTGGTATCAACAGATACCGGGACAGTCTCCTCGCC | |
| | TCCTGATCTACTGGGCATCTACCCGAGAAAGCGGTGTTCCGGATAGGTTTAC | |
| | CGGTTCCGGGTCTGGGACCGATTTTACGTTGACAATATCCAGCGTACAGCCG | |
| | GAAGACCTTGCTATCTATTACTGTCACCAGTACCTTTCCAGCCGGACGTTCG | |
| | GGCAGGGCACGAAGCTGGAGATTAAAAGTGCTGCTGCCTTTGTCCCGGTATT | |
| | TCTCCCAGCCAAACCGACCACGACTCCCGCCCCGCGCCCTCCGACACCCGCT | |
| | CCCACCATCGCCTCTCAACCTCTTAGTCTTCGCCCCGAGGCATGCCGACCCG | |
| | CCGCCGGGGGTGCTGTTCATACGAGGGGCTTGGACTTCGCTTGTGATATTTA | |
| | CATTTGGGCTCCGTTGGCGGGTACGTGCGGCGTCCTTTTGTTGTCACTCGTT | |
| | ATTACTTTGTATTGTAATCACAGGAATCGCTCAAAGCGGAGTAGGTTGTTGC | |
| | ATTCCGATTACATGAATATGACTCCTCGCCGGCCTGGGCCGACAAGAAAACA | |
| | TTACCAACCCTATGCCCCCCCACGAGACTTCGCTGCGTACAGGTCCCGAGTG | |
| | AAGTTTTCCCGAAGCGCAGACGCTCCGGCATATCAGCAAGGACAGAATCAGC | |
| | TGTATAACGAACTGAATTTGGGACGCCGCGAGGAGTATGACGTGCTTGATAA | |
| | ACGCCGGGGGAGAGACCCGGAAATGGGGGGTAAACCCCGAAGAAAGAATCCC | |
| | CAAGAAGGACTCTACAATGAACTCCAGAAGGATAAGATGGCGGAGGCCTACT | |
| | CAGAAATAGGTATGAAGGGCGAACGACGACGGGGAAAAGGTCACGATGGCCT | |
| | CTACCAAGGGTTGAGTACGGCAACCAAAGATACGTACGATGCACTGCATATG | |
| | CAGGCCCTGCCTCCCAGATAATAATAAAATCGCTATCCATCGAAGATGGATG | |
| | TGTGTTGGTTTTTTGTGTG | |
| CTX-964b | GAGATGTAAGGAGCTGCTGTGACTTGCTCAAGGCCTTATATCGAGTAAACGG | 51 |
| Donor | TAGTGCTGGGGCTTAGACGCAGGTGTTCTGATTTATAGTTCAAAACCTCTAT | |
| LHA to RHA | CAATGAGAGAGCAATCTCCTGGTAATGTGATAGATTTCCCAACTTAATGCCA | |
| 41BB costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGCAGGTACAACTCCAACAACCCGGAGCTGAGGTTG | |
| | TAAAACCAGGTGCGTCAGTCAAGATGAGTTGCAAAGCCAGTGGATATACTTT | |
| | TACTTCCTATTACATTCATTGGATCAAGCAGACTCCAGGTCAGGGGCTCGAG | |
| | TGGGTAGGCGTGATCTACCCCGGTAACGACGACATTTCATACAACCAAAAAT | |
| | TTCAGGGGAAAGCGACGCTGACTGCTGACAAGAGTAGCACGACCGCATATAT | |
| | GCAACTCTCATCACTTACGTCTGAGGATTCTGCAGTTTATTATTGCGCTCGG | |
| | GAAGTTCGGCTTCGATATTTCGATGTGTGGGGTCAGGGCACGACCGTAACGG | |
| | TGAGCAGTGGTGGCGGTGGCGGGTCCGGGGGCGGTGGATCAGGTGGTGGGGG | |
| | GAGTGAGATAGTGTTGACCCAGTCACCGGGGTCCCTCGCAGTTTCACCGGGA | |
| | GAGAGGGTCACAATGTCCTGCAAATCCTCCCAATCAGTGTTCTTCTCTTCCA | |
| | GCCAAAAAAACTACCTTGCGTGGTATCAACAGATACCGGGACAGTCTCCTCG | |
| | CCTCCTGATCTACTGGGCATCTACCCGAGAAAGCGGTGTTCCGGATAGGTTT | |
| | ACCGGTTCCGGGTCTGGGACCGATTTTACGTTGACAATATCCAGCGTACAGC | |
| | CGGAAGACCTTGCTATCTATTACTGTCACCAGTACCTTTCCAGCCGGACGTT | |
| | CGGGCAGGGCACGAAGCTGGAGATTAAAAGTGCTGCTGCCTTTGTCCCGGTA | |
| | TTTCTCCCAGCCAAACCGACCACGACTCCCGCCCCGCGCCCTCCGACACCCG | |
| | CTCCCACCATCGCCTCTCAACCTCTTAGTCTTCGCCCCGAGGCATGCCGACC | |
| | CGCCGCCGGGGGTGCTGTTCATACGAGGGGCTTGGACTTCGCTTGTGATATT | |
| | TACATTTGGGCTCCGTTGGCGGGTACGTGCGGCGTCCTTTTGTTGTCACTCG | |
| | TTATTACTTTGTATTGTAATCACAGGAATCGCAAACGGGGCAGAAAGAAACT | |
| | CCTGTATATATTCAAACAACCATTTATGAGACCAGTACAAACTACTCAAGAG | |
| | GAAGATGGCTGTAGCTGCCGATTTCCAGAAGAAGAAGAAGGAGGATGTGAAC | |
| | TGCGAGTGAAGTTTTCCCGAAGCGCAGACGCTCCGGCATATCAGCAAGGACA | |
| | GAATCAGCTGTATAACGAACTGAATTTGGGACGCCGCGAGGAGTATGACGTG | |
| | CTTGATAAACGCCGGGGGAGAGACCCGGAAATGGGGGGTAAACCCCGAAGAA | |
| | AGAATCCCCAAGAAGGACTCTACAATGAACTCCAGAAGGATAAGATGGCGGA | |
| | GGCCTACTCAGAAATAGGTATGAAGGGCGAACGACGACGGGGAAAAGGTCAC | |
| | GATGGCCTCTACCAAGGGTTGAGTACGGCAACCAAAGATACGTACGATGCAC | |
| | TGCATATGCAGGCCCTGCCTCCCAGATAATAATAAAATCGCTATCCATCGAA | |
| | GATGGATGTGTGTTGGTTTTTTGTGTGTGGAGCAACAAATCTGACTTTGCAT | |
| | GTGCAAACGCCTTCAACAACAGCATTATTCCAGAAGACACCTTCTTCCCCAG | |
| | CCCAGGTAAGGGCAGCTTTGGTGCCTTCGCAGGCTGTTTCCTTGCTTCAGGA | |
| | ATGGCCAGGTTCTGCCCAGAGCTCTGGTCAATGATGTCTAAAACTCCTCTGA | |
| | TTGGTGGTCTCGGCCTTATCCATTGCCACCAAAACCCTCTTTTTACTAAGAA | |
| | ACAGTGAGCCTTGTTCTGGCAGTCCAGAGAATGACACGGGAAAAAAGCAGAT | |
| | GAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTC | |
| | TAGGCCTCATTCTAAGCCCCTTCTCCAAGTTGCCTCTCCTTATTTCTCCCTG | |
| | TCTGCCAAAAAATCTTTCCCAGCTCACTAAGTCAGTCTCACGCAGTCACTCA | |
| | TTAACCCACCAATCACTGATTGTGCCGGCACATGAATGCACCAGGTGTTGAA | |
| | GTGGAGGAATTAAAAAGTCAGATGAGGGGTGTGCCCAGAGGAAGCACCATTC | |
| | TAGTTGGGGGAGCCCATCTGTCAGCTGGGAAAAGTCCAAATAACTTCAGATT | |
| | GGAATGTGTTTTAACTCAGGGTTGAGAAAACAGCTACCTTCAGGACAAAAGT | |
| | CAGGGAAGGGCTCTCTGAAGAAATGCTACTTGAAGATACCAGCCCTACCAAG | |
| | GGCAGGGAGAGGACCCTATAGAGGCCTGGGACAGGAGCTCAATGAGAAAGG | |
| CTX-964b | CCACCATGGCGCTTCCGGTGACAGCACTGCTCCTCCCCTTGGCGCTGTTGCT | 52 |
| CAR | CCACGCAGCAAGGCCGCAGGTACAACTCCAACAACCCGGAGCTGAGGTTGTA | |
| 41BB costim. | AAACCAGGTGCGTCAGTCAAGATGAGTTGCAAAGCCAGTGGATATACTTTTA | |
| | CTTCCTATTACATTCATTGGATCAAGCAGACTCCAGGTCAGGGGCTCGAGTG | |
| | GGTAGGCGTGATCTACCCCGGTAACGACGACATTTCATACAACCAAAAATTT | |
| | CAGGGGAAAGCGACGCTGACTGCTGACAAGAGTAGCACGACCGCATATATGC | |
| | AACTCTCATCACTTACGTCTGAGGATTCTGCAGTTTATTATTGCGCTCGGGA | |
| | AGTTCGGCTTCGATATTTCGATGTGTGGGGTCAGGGCACGACCGTAACGGTG | |
| | AGCAGTGGTGGCGGTGGCGGGTCCGGGGGCGGTGGATCAGGTGGTGGGGGGA | |
| | GTGAGATAGTGTTGACCCAGTCACCGGGGTCCCTCGCAGTTTCACCGGGAGA | |
| | GAGGGTCACAATGTCCTGCAAATCCTCCCAATCAGTGTTCTTCTCTTCCAGC | |
| | CAAAAAAACTACCTTGCGTGGTATCAACAGATACCGGGACAGTCTCCTCGCC | |
| | TCCTGATCTACTGGGCATCTACCCGAGAAAGCGGTGTTCCGGATAGGTTTAC | |
| | CGGTTCCGGGTCTGGGACCGATTTTACGTTGACAATATCCAGCGTACAGCCG | |
| | GAAGACCTTGCTATCTATTACTGTCACCAGTACCTTTCCAGCCGGACGTTCG | |
| | GGCAGGGCACGAAGCTGGAGATTAAAAGTGCTGCTGCCTTTGTCCCGGTATT | |
| | TCTCCCAGCCAAACCGACCACGACTCCCGCCCCGCGCCCTCCGACACCCGCT | |
| | CCCACCATCGCCTCTCAACCTCTTAGTCTTCGCCCCGAGGCATGCCGACCCG | |
| | CCGCCGGGGGTGCTGTTCATACGAGGGGCTTGGACTTCGCTTGTGATATTTA | |
| | CATTTGGGCTCCGTTGGCGGGTACGTGCGGCGTCCTTTTGTTGTCACTCGTT | |
| | ATTACTTTGTATTGTAATCACAGGAATCGCAAACGGGGCAGAAAGAAACTCC | |
| | TGTATATATTCAAACAACCATTTATGAGACCAGTACAAACTACTCAAGAGGA | |
| | AGATGGCTGTAGCTGCCGATTTCCAGAAGAAGAAGAAGGAGGATGTGAACTG | |
| | CGAGTGAAGTTTTCCCGAAGCGCAGACGCTCCGGCATATCAGCAAGGACAGA | |
| | ATCAGCTGTATAACGAACTGAATTTGGGACGCCGCGAGGAGTATGACGTGCT | |
| | TGATAAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGAGG | |
| | CCTACTCAGAAATAGGTATGAAGGGCGAACGACGACGGGGAAAAGGTCACGA | |
| | TGGCCTCTACCAAGGGTTGAGTACGGCAACCAAAGATACGTACGATGCACTG | |
| | CATATGCAGGCCCTGCCTCCCAGATAATAATAAAATCGCTATCCATCGAAGA | |
| | TGGATGTGTGTTGGTTTTTTGTGTG | |
| CTX-965 | GAGATGTAAGGAGCTGCTGTGACTTGCTCAAGGCCTTATATCGAGTAAACGG | 53 |
| Donor | TAGTGCTGGGGCTTAGACGCAGGTGTTCTGATTTATAGTTCAAAACCTCTAT | |
| LHA to RHA | CAATGAGAGAGCAATCTCCTGGTAATGTGATAGATTTCCCAACTTAATGCCA | |
| CD28 costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGAAATCGTCCTCACACAATCCCCGGGGAGCCTCG | |
| | CAGTCAGTCCTGGGGAACGAGTCACTATGAGCTGCAAATCCAGTCAGAGTGT | |
| | TTTTTTCTCAAGTAGCCAGAAGAACTACCTCGCATGGTACCAACAAATACCG | |
| | GGGCAATCTCCCCGCTTGCTTATATACTGGGCAAGTACCCGCGAATCCGGCG | |
| | TACCGGATCGATTCACGGGATCTGGGTCAGGTACTGATTTCACTTTGACTAT | |
| | CAGCTCTGTTCAGCCTGAAGATTTGGCAATTTACTACTGTCACCAATACTTG | |
| | AGTAGCCGAACTTTCGGCCAGGGCACGAAGCTCGAAATCAAGGGCGGAGGGG | |
| | GAGGTTCTGGTGGGGGCGGTTCTGGCGGTGGAGGAAGCCAAGTACAGTTGCA | |
| | ACAGCCAGGGGCGGAGGTCGTAAAACCTGGGGCGTCTGTCAAGATGAGCTGT | |
| | AAAGCAAGTGGATACACCTTCACCTCCTACTATATACATTGGATTAAGCAAA | |
| | CTCCGGGTCAGGGGCTGGAATGGGTTGGCGTTATATACCCCGGGAACGATGA | |
| | TATATCATACAACCAAAAATTTCAAGGCAAGGCGACTCTGACTGCCGATAAG | |
| | AGTAGCACAACAGCTTACATGCAGCTTTCTTCCCTGACCAGCGAAGATTCAG | |
| | CAGTTTACTACTGCGCTCGGGAAGTGCGCCTGCGATACTTTGATGTCTGGGG | |
| | TCAAGGAACTACAGTTACTGTATCAAGCAGTGCTGCTGCCTTTGTCCCGGTA | |
| | TTTCTCCCAGCCAAACCGACCACGACTCCCGCCCCGCGCCCTCCGACACCCG | |
| | CTCCCACCATCGCCTCTCAACCTCTTAGTCTTCGCCCCGAGGCATGCCGACC | |
| | CGCCGCCGGGGGTGCTGTTCATACGAGGGGCTTGGACTTCGCTTGTGATATT | |
| | TACATTTGGGCTCCGTTGGCGGGTACGTGCGGCGTCCTTTTGTTGTCACTCG | |
| | TTATTACTTTGTATTGTAATCACAGGAATCGCTCAAAGCGGAGTAGGTTGTT | |
| | GCATTCCGATTACATGAATATGACTCCTCGCCGGCCTGGGCCGACAAGAAAA | |
| | CATTACCAACCCTATGCCCCCCCACGAGACTTCGCTGCGTACAGGTCCCGAG | |
| | TGAAGTTTTCCCGAAGCGCAGACGCTCCGGCATATCAGCAAGGACAGAATCA | |
| | GCTGTATAACGAACTGAATTTGGGACGCCGCGAGGAGTATGACGTGCTTGAT | |
| | AAACGCCGGGGGAGAGACCCGGAAATGGGGGGTAAACCCCGAAGAAAGAATC | |
| | CCCAAGAAGGACTCTACAATGAACTCCAGAAGGATAAGATGGCGGAGGCCTA | |
| | CTCAGAAATAGGTATGAAGGGCGAACGACGACGGGGAAAAGGTCACGATGGC | |
| | CTCTACCAAGGGTTGAGTACGGCAACCAAAGATACGTACGATGCACTGCATA | |
| | TGCAGGCCCTGCCTCCCAGATAATAATAAAATCGCTATCCATCGAAGATGGA | |
| | TGTGTGTTGGTTTTTTGTGTGTGGAGCAACAAATCTGACTTTGCATGTGCAA | |
| | ACGCCTTCAACAACAGCATTATTCCAGAAGACACCTTCTTCCCCAGCCCAGG | |
| | TAAGGGCAGCTTTGGTGCCTTCGCAGGCTGTTTCCTTGCTTCAGGAATGGCC | |
| | AGGTTCTGCCCAGAGCTCTGGTCAATGATGTCTAAAACTCCTCTGATTGGTG | |
| | GTCTCGGCCTTATCCATTGCCACCAAAACCCTCTTTTTACTAAGAAACAGTG | |
| | AGCCTTGTTCTGGCAGTCCAGAGAATGACACGGGAAAAAAGCAGATGAAGAG | |
| | AAGGTGGCAGGAGAGGGCACGTGGCCCAGCCTCAGTCTCTCCAACTGAGTTC | |
| | CTGCCTGCCTGCCTTTGCTCAGACTGTTTGCCCCTTACTGCTCTTCTAGGCC | |
| | TCATTCTAAGCCCCTTCTCCAAGTTGCCTCTCCTTATTTCTCCCTGTCTGCC | |
| | AAAAAATCTTTCCCAGCTCACTAAGTCAGTCTCACGCAGTCACTCATTAACC | |
| | CACCAATCACTGATTGTGCCGGCACATGAATGCACCAGGTGTTGAAGTGGAG | |
| | GAATTAAAAAGTCAGATGAGGGGTGTGCCCAGAGGAAGCACCATTCTAGTTG | |
| | GGGGAGCCCATCTGTCAGCTGGGAAAAGTCCAAATAACTTCAGATTGGAATG | |
| | TGTTTTAACTCAGGGTTGAGAAAACAGCTACCTTCAGGACAAAAGTCAGGGA | |
| | AGGGCTCTCTGAAGAAATGCTACTTGAAGATACCAGCCCTACCAAGGGCAGG | |
| | GAGAGGACCCTATAGAGGCCTGGGACAGGAGCTCAATGAGAAAGG | |
| CTX-965 | CCACCATGGCGCTTCCGGTGACAGCACTGCTCCTCCCCTTGGCGCTGTTGCT | 54 |
| CAR | CCACGCAGCAAGGCCGGAAATCGTCCTCACACAATCCCCGGGGAGCCTCGCA | |
| CD28 costim. | GTCAGTCCTGGGGAACGAGTCACTATGAGCTGCAAATCCAGTCAGAGTGTTT | |
| | TTTTCTCAAGTAGCCAGAAGAACTACCTCGCATGGTACCAACAAATACCGGG | |
| | GCAATCTCCCCGCTTGCTTATATACTGGGCAAGTACCCGCGAATCCGGCGTA | |
| | CCGGATCGATTCACGGGATCTGGGTCAGGTACTGATTTCACTTTGACTATCA | |
| | GCTCTGTTCAGCCTGAAGATTTGGCAATTTACTACTGTCACCAATACTTGAG | |
| | TAGCCGAACTTTCGGCCAGGGCACGAAGCTCGAAATCAAGGGCGGAGGGGGA | |
| | GGTTCTGGTGGGGGCGGTTCTGGCGGTGGAGGAAGCCAAGTACAGTTGCAAC | |
| | AGCCAGGGGCGGAGGTCGTAAAACCTGGGGCGTCTGTCAAGATGAGCTGTAA | |
| | AGCAAGTGGATACACCTTCACCTCCTACTATATACATTGGATTAAGCAAACT | |
| | CCGGGTCAGGGGCTGGAATGGGTTGGCGTTATATACCCCGGGAACGATGATA | |
| | TATCATACAACCAAAAATTTCAAGGCAAGGCGACTCTGACTGCCGATAAGAG | |
| | TAGCACAACAGCTTACATGCAGCTTTCTTCCCTGACCAGCGAAGATTCAGCA | |
| | GTTTACTACTGCGCTCGGGAAGTGCGCCTGCGATACTTTGATGTCTGGGGTC | |
| | AAGGAACTACAGTTACTGTATCAAGCAGTGCTGCTGCCTTTGTCCCGGTATT | |
| | TCTCCCAGCCAAACCGACCACGACTCCCGCCCCGCGCCCTCCGACACCCGCT | |
| | CCCACCATCGCCTCTCAACCTCTTAGTCTTCGCCCCGAGGCATGCCGACCCG | |
| | CCGCCGGGGGTGCTGTTCATACGAGGGGCTTGGACTTCGCTTGTGATATTTA | |
| | CATTTGGGCTCCGTTGGCGGGTACGTGCGGCGTCCTTTTGTTGTCACTCGTT | |
| | ATTACTTTGTATTGTAATCACAGGAATCGCTCAAAGCGGAGTAGGTTGTTGC | |
| | ATTCCGATTACATGAATATGACTCCTCGCCGGCCTGGGCCGACAAGAAAACA | |
| | TTACCAACCCTATGCCCCCCCACGAGACTTCGCTGCGTACAGGTCCCGAGTG | |
| | AAGTTTTCCCGAAGCGCAGACGCTCCGGCATATCAGCAAGGACAGAATCAGC | |
| | TGTATAACGAACTGAATTTGGGACGCCGCGAGGAGTATGACGTGCTTGATAA | |
| | ACGCCGGGGGAGAGACCCGGAAATGGGGGGTAAACCCCGAAGAAAGAATCCC | |
| | CAAGAAGGACTCTACAATGAACTCCAGAAGGATAAGATGGCGGAGGCCTACT | |
| | CAGAAATAGGTATGAAGGGCGAACGACGACGGGGAAAAGGTCACGATGGCCT | |
| | CTACCAAGGGTTGAGTACGGCAACCAAAGATACGTACGATGCACTGCATATG | |
| | CAGGCCCTGCCTCCCAGATAATAATAAAATCGCTATCCATCGAAGATGGATG | |
| | TGTGTTGGTTTTTTGTGTG | |
| CTX-965b | GAGATGTAAGGAGCTGCTGTGACTTGCTCAAGGCCTTATATCGAGTAAACGG | 55 |
| Donor | TAGTGCTGGGGCTTAGACGCAGGTGTTCTGATTTATAGTTCAAAACCTCTAT | |
| LHA or RHA | CAATGAGAGAGCAATCTCCTGGTAATGTGATAGATTTCCCAACTTAATGCCA | |
| 41BB costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGAAATCGTCCTCACACAATCCCCGGGGAGCCTCG | |
| | CAGTCAGTCCTGGGGAACGAGTCACTATGAGCTGCAAATCCAGTCAGAGTGT | |
| | TTTTTTCTCAAGTAGCCAGAAGAACTACCTCGCATGGTACCAACAAATACCG | |
| | GGGCAATCTCCCCGCTTGCTTATATACTGGGCAAGTACCCGCGAATCCGGCG | |
| | TACCGGATCGATTCACGGGATCTGGGTCAGGTACTGATTTCACTTTGACTAT | |
| | CAGCTCTGTTCAGCCTGAAGATTTGGCAATTTACTACTGTCACCAATACTTG | |
| | AGTAGCCGAACTTTCGGCCAGGGCACGAAGCTCGAAATCAAGGGCGGAGGGG | |
| | GAGGTTCTGGTGGGGGCGGTTCTGGCGGTGGAGGAAGCCAAGTACAGTTGCA | |
| | ACAGCCAGGGGCGGAGGTCGTAAAACCTGGGGCGTCTGTCAAGATGAGCTGT | |
| | AAAGCAAGTGGATACACCTTCACCTCCTACTATATACATTGGATTAAGCAAA | |
| | CTCCGGGTCAGGGGCTGGAATGGGTTGGCGTTATATACCCCGGGAACGATGA | |
| | TATATCATACAACCAAAAATTTCAAGGCAAGGCGACTCTGACTGCCGATAAG | |
| | AGTAGCACAACAGCTTACATGCAGCTTTCTTCCCTGACCAGCGAAGATTCAG | |
| | CAGTTTACTACTGCGCTCGGGAAGTGCGCCTGCGATACTTTGATGTCTGGGG | |
| | TCAAGGAACTACAGTTACTGTATCAAGCAGTGCTGCTGCCTTTGTCCCGGTA | |
| | TTTCTCCCAGCCAAACCGACCACGACTCCCGCCCCGCGCCCTCCGACACCCG | |
| | CTCCCACCATCGCCTCTCAACCTCTTAGTCTTCGCCCCGAGGCATGCCGACC | |
| | CGCCGCCGGGGGTGCTGTTCATACGAGGGGCTTGGACTTCGCTTGTGATATT | |
| | TACATTTGGGCTCCGTTGGCGGGTACGTGCGGCGTCCTTTTGTTGTCACTCG | |
| | TTATTACTTTGTATTGTAATCACAGGAATCGCAAACGGGGCAGAAAGAAACT | |
| | CCTGTATATATTCAAACAACCATTTATGAGACCAGTACAAACTACTCAAGAG | |
| | GAAGATGGCTGTAGCTGCCGATTTCCAGAAGAAGAAGAAGGAGGATGTGAAC | |
| | TGCGAGTGAAGTTTTCCCGAAGCGCAGACGCTCCGGCATATCAGCAAGGACA | |
| | GAATCAGCTGTATAACGAACTGAATTTGGGACGCCGCGAGGAGTATGACGTG | |
| | CTTGATAAACGCCGGGGGAGAGACCCGGAAATGGGGGGTAAACCCCGAAGAA | |
| | AGAATCCCCAAGAAGGACTCTACAATGAACTCCAGAAGGATAAGATGGCGGA | |
| | GGCCTACTCAGAAATAGGTATGAAGGGCGAACGACGACGGGGAAAAGGTCAC | |
| | GATGGCCTCTACCAAGGGTTGAGTACGGCAACCAAAGATACGTACGATGCAC | |
| | TGCATATGCAGGCCCTGCCTCCCAGATAATAATAAAATCGCTATCCATCGAA | |
| | GATGGATGTGTGTTGGTTTTTTGTGTGTGGAGCAACAAATCTGACTTTGCAT | |
| | GTGCAAACGCCTTCAACAACAGCATTATTCCAGAAGACACCTTCTTCCCCAG | |
| | CCCAGGTAAGGGCAGCTTTGGTGCCTTCGCAGGCTGTTTCCTTGCTTCAGGA | |
| | ATGGCCAGGTTCTGCCCAGAGCTCTGGTCAATGATGTCTAAAACTCCTCTGA | |
| | TTGGTGGTCTCGGCCTTATCCATTGCCACCAAAACCCTCTTTTTACTAAGAA | |
| | ACAGTGAGCCTTGTTCTGGCAGTCCAGAGAATGACACGGGAAAAAAGCAGAT | |
| | GAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTC | |
| | TAGGCCTCATTCTAAGCCCCTTCTCCAAGTTGCCTCTCCTTATTTCTCCCTG | |
| | TCTGCCAAAAAATCTTTCCCAGCTCACTAAGTCAGTCTCACGCAGTCACTCA | |
| | TTAACCCACCAATCACTGATTGTGCCGGCACATGAATGCACCAGGTGTTGAA | |
| | GTGGAGGAATTAAAAAGTCAGATGAGGGGTGTGCCCAGAGGAAGCACCATTC | |
| | TAGTTGGGGGAGCCCATCTGTCAGCTGGGAAAAGTCCAAATAACTTCAGATT | |
| | GGAATGTGTTTTAACTCAGGGTTGAGAAAACAGCTACCTTCAGGACAAAAGT | |
| | CAGGGAAGGGCTCTCTGAAGAAATGCTACTTGAAGATACCAGCCCTACCAAG | |
| | GGCAGGGAGAGGACCCTATAGAGGCCTGGGACAGGAGCTCAATGAGAAAGG | |
| CTX-965b | CCACCATGGCGCTTCCGGTGACAGCACTGCTCCTCCCCTTGGCGCTGTTGCT | 56 |
| CAR | CCACGCAGCAAGGCCGGAAATCGTCCTCACACAATCCCCGGGGAGCCTCGCA | |
| 41BB costim. | GTCAGTCCTGGGGAACGAGTCACTATGAGCTGCAAATCCAGTCAGAGTGTTT | |
| | TTTTCTCAAGTAGCCAGAAGAACTACCTCGCATGGTACCAACAAATACCGGG | |
| | GCAATCTCCCCGCTTGCTTATATACTGGGCAAGTACCCGCGAATCCGGCGTA | |
| | CCGGATCGATTCACGGGATCTGGGTCAGGTACTGATTTCACTTTGACTATCA | |
| | GCTCTGTTCAGCCTGAAGATTTGGCAATTTACTACTGTCACCAATACTTGAG | |
| | TAGCCGAACTTTCGGCCAGGGCACGAAGCTCGAAATCAAGGGCGGAGGGGGA | |
| | GGTTCTGGTGGGGGCGGTTCTGGCGGTGGAGGAAGCCAAGTACAGTTGCAAC | |
| | AGCCAGGGGCGGAGGTCGTAAAACCTGGGGCGTCTGTCAAGATGAGCTGTAA | |
| | AGCAAGTGGATACACCTTCACCTCCTACTATATACATTGGATTAAGCAAACT | |
| | CCGGGTCAGGGGCTGGAATGGGTTGGCGTTATATACCCCGGGAACGATGATA | |
| | TATCATACAACCAAAAATTTCAAGGCAAGGCGACTCTGACTGCCGATAAGAG | |
| | TAGCACAACAGCTTACATGCAGCTTTCTTCCCTGACCAGCGAAGATTCAGCA | |
| | GTTTACTACTGCGCTCGGGAAGTGCGCCTGCGATACTTTGATGTCTGGGGTC | |
| | AAGGAACTACAGTTACTGTATCAAGCAGTGCTGCTGCCTTTGTCCCGGTATT | |
| | TCTCCCAGCCAAACCGACCACGACTCCCGCCCCGCGCCCTCCGACACCCGCT | |
| | CCCACCATCGCCTCTCAACCTCTTAGTCTTCGCCCCGAGGCATGCCGACCCG | |
| | CCGCCGGGGGTGCTGTTCATACGAGGGGCTTGGACTTCGCTTGTGATATTTA | |
| | CATTTGGGCTCCGTTGGCGGGTACGTGCGGCGTCCTTTTGTTGTCACTCGTT | |
| | ATTACTTTGTATTGTAATCACAGGAATCGCAAACGGGGCAGAAAGAAACTCC | |
| | TGTATATATTCAAACAACCATTTATGAGACCAGTACAAACTACTCAAGAGGA | |
| | AGATGGCTGTAGCTGCCGATTTCCAGAAGAAGAAGAAGGAGGATGTGAACTG | |
| | CGAGTGAAGTTTTCCCGAAGCGCAGACGCTCCGGCATATCAGCAAGGACAGA | |
| | ATCAGCTGTATAACGAACTGAATTTGGGACGCCGCGAGGAGTATGACGTGCT | |
| | TGATAAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGAGG | |
| | CCTACTCAGAAATAGGTATGAAGGGCGAACGACGACGGGGAAAAGGTCACGA | |
| | TGGCCTCTACCAAGGGTTGAGTACGGCAACCAAAGATACGTACGATGCACTG | |
| | CATATGCAGGCCCTGCCTCCCAGATAATAATAAAATCGCTATCCATCGAAGA | |
| | TGGATGTGTGTTGGTTTTTTGTGTG | |
| CTX-969 | GAGATGTAAGGAGCTGCTGTGACTTGCTCAAGGCCTTATATCGAGTAAACGG | 57 |
| Donor | TAGTGCTGGGGCTTAGACGCAGGTGTTCTGATTTATAGTTCAAAACCTCTAT | |
| LHA or RHA | CAATGAGAGAGCAATCTCCTGGTAATGTGATAGATTTCCCAACTTAATGCCA | |
| CD28 costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGCAAGTGCAGCTTGTGCAATCTGGCGCCGAAGTTA | |
| | AGAAACCAGGCGCATCTGTGAAGGTGAGTTGTAAAGCGTCCGGCTATACTTT | |
| | TACGAACTACGACATTAACTGGGTTAGGCAAGCTCCAGGGCAGGGTCTGGAG | |
| | TGGATAGGTTGGATATATCCAGGTGACGGGTCTACTAAATATAATGAAAAAT | |
| | TCAAGGCTAAGGCCACTTTGACCGCCGACACCTCTACATCAACTGCATACAT | |
| | GGAGTTGAGAAGCCTTCGCAGCGATGACACGGCGGTATATTATTGTGCCAGT | |
| | GGTTATGAAGACGCTATGGACTATTGGGGGCAGGGGACGACGGTAACCGTAT | |
| | CAAGCGGAGGCGGAGGGGGATCAGGCGGGGGCGGATCAGGGGGGGGCGGTAG | |
| | TGATATACAAATGACACAATCTCCAAGCTCACTTTCTGCCAGCGTTGGTGAT | |
| | CGCGTAACTATTAACTGCAAGGCCTCACAGGACATTAACAGTTATCTTAGCT | |
| | GGTTCCAGCAAAAACCTGGGAAAGCTCCAAAAACTTTGATCTATCGAGCGAA | |
| | TAGGCTGGTCGACGGAGTCCCATCTCGCTTCTCCGGCAGTGGCTCAGGCCAG | |
| | GACTATACGCTGACAATAAGTAGTTTGCAACCAGAGGACTTCGCAACCTATT | |
| | ACTGTTTGCAATACGATGAGTTCCCACTCACATTCGGTGGGGGTACTAAAGT | |
| | AGAAATAAAAAGTGCTGCTGCCTTTGTCCCGGTATTTCTCCCAGCCAAACCG | |
| | ACCACGACTCCCGCCCCGCGCCCTCCGACACCCGCTCCCACCATCGCCTCTC | |
| | AACCTCTTAGTCTTCGCCCCGAGGCATGCCGACCCGCCGCCGGGGGTGCTGT | |
| | TCATACGAGGGGCTTGGACTTCGCTTGTGATATTTACATTTGGGCTCCGTTG | |
| | GCGGGTACGTGCGGCGTCCTTTTGTTGTCACTCGTTATTACTTTGTATTGTA | |
| | ATCACAGGAATCGCTCAAAGCGGAGTAGGTTGTTGCATTCCGATTACATGAA | |
| | TATGACTCCTCGCCGGCCTGGGCCGACAAGAAAACATTACCAACCCTATGCC | |
| | CCCCCACGAGACTTCGCTGCGTACAGGTCCCGAGTGAAGTTTTCCCGAAGCG | |
| | CAGACGCTCCGGCATATCAGCAAGGACAGAATCAGCTGTATAACGAACTGAA | |
| | TTTGGGACGCCGCGAGGAGTATGACGTGCTTGATAAACGCCGGGGGAGAGAC | |
| | CCGGAAATGGGGGGTAAACCCCGAAGAAAGAATCCCCAAGAAGGACTCTACA | |
| | ATGAACTCCAGAAGGATAAGATGGCGGAGGCCTACTCAGAAATAGGTATGAA | |
| | GGGCGAACGACGACGGGGAAAAGGTCACGATGGCCTCTACCAAGGGTTGAGT | |
| | ACGGCAACCAAAGATACGTACGATGCACTGCATATGCAGGCCCTGCCTCCCA | |
| | GATAATAATAAAATCGCTATCCATCGAAGATGGATGTGTGTTGGTTTTTTGT | |
| | GTGTGGAGCAACAAATCTGACTTTGCATGTGCAAACGCCTTCAACAACAGCA | |
| | TTATTCCAGAAGACACCTTCTTCCCCAGCCCAGGTAAGGGCAGCTTTGGTGC | |
| | CTTCGCAGGCTGTTTCCTTGCTTCAGGAATGGCCAGGTTCTGCCCAGAGCTC | |
| | TGGTCAATGATGTCTAAAACTCCTCTGATTGGTGGTCTCGGCCTTATCCATT | |
| | GCCACCAAAACCCTCTTTTTACTAAGAAACAGTGAGCCTTGTTCTGGCAGTC | |
| | CAGAGAATGACACGGGAAAAAAGCAGATGAAGAGAAGGTGGCAGGAGAGGGC | |
| | ACGTGGCCCAGCCTCAGTCTCTCCAACTGAGTTCCTGCCTGCCTGCCTTTGC | |
| | TCAGACTGTTTGCCCCTTACTGCTCTTCTAGGCCTCATTCTAAGCCCCTTCT | |
| | CCAAGTTGCCTCTCCTTATTTCTCCCTGTCTGCCAAAAAATCTTTCCCAGCT | |
| | CACTAAGTCAGTCTCACGCAGTCACTCATTAACCCACCAATCACTGATTGTG | |
| | CCGGCACATGAATGCACCAGGTGTTGAAGTGGAGGAATTAAAAAGTCAGATG | |
| | AGGGGTGTGCCCAGAGGAAGCACCATTCTAGTTGGGGGAGCCCATCTGTCAG | |
| | CTGGGAAAAGTCCAAATAACTTCAGATTGGAATGTGTTTTAACTCAGGGTTG | |
| | AGAAAACAGCTACCTTCAGGACAAAAGTCAGGGAAGGGCTCTCTGAAGAAAT | |
| | GCTACTTGAAGATACCAGCCCTACCAAGGGCAGGGAGAGGACCCTATAGAGG | |
| | CCTGGGACAGGAGCTCAATGAGAAAGG | |
| CTX-969 | CCACCATGGCGCTTCCGGTGACAGCACTGCTCCTCCCCTTGGCGCTGTTGCT | 58 |
| CAR | CCACGCAGCAAGGCCGCAAGTGCAGCTTGTGCAATCTGGCGCCGAAGTTAAG | |
| CD28 costim. | AAACCAGGCGCATCTGTGAAGGTGAGTTGTAAAGCGTCCGGCTATACTTTTA | |
| | CGAACTACGACATTAACTGGGTTAGGCAAGCTCCAGGGCAGGGTCTGGAGTG | |
| | GATAGGTTGGATATATCCAGGTGACGGGTCTACTAAATATAATGAAAAATTC | |
| | AAGGCTAAGGCCACTTTGACCGCCGACACCTCTACATCAACTGCATACATGG | |
| | AGTTGAGAAGCCTTCGCAGCGATGACACGGCGGTATATTATTGTGCCAGTGG | |
| | TTATGAAGACGCTATGGACTATTGGGGGCAGGGGACGACGGTAACCGTATCA | |
| | AGCGGAGGCGGAGGGGGATCAGGCGGGGGCGGATCAGGGGGGGGCGGTAGTG | |
| | ATATACAAATGACACAATCTCCAAGCTCACTTTCTGCCAGCGTTGGTGATCG | |
| | CGTAACTATTAACTGCAAGGCCTCACAGGACATTAACAGTTATCTTAGCTGG | |
| | TTCCAGCAAAAACCTGGGAAAGCTCCAAAAACTTTGATCTATCGAGCGAATA | |
| | GGCTGGTCGACGGAGTCCCATCTCGCTTCTCCGGCAGTGGCTCAGGCCAGGA | |
| | CTATACGCTGACAATAAGTAGTTTGCAACCAGAGGACTTCGCAACCTATTAC | |
| | TGTTTGCAATACGATGAGTTCCCACTCACATTCGGTGGGGGTACTAAAGTAG | |
| | AAATAAAAAGTGCTGCTGCCTTTGTCCCGGTATTTCTCCCAGCCAAACCGAC | |
| | CACGACTCCCGCCCCGCGCCCTCCGACACCCGCTCCCACCATCGCCTCTCAA | |
| | CCTCTTAGTCTTCGCCCCGAGGCATGCCGACCCGCCGCCGGGGGTGCTGTTC | |
| | ATACGAGGGGCTTGGACTTCGCTTGTGATATTTACATTTGGGCTCCGTTGGC | |
| | GGGTACGTGCGGCGTCCTTTTGTTGTCACTCGTTATTACTTTGTATTGTAAT | |
| | CACAGGAATCGCTCAAAGCGGAGTAGGTTGTTGCATTCCGATTACATGAATA | |
| | TGACTCCTCGCCGGCCTGGGCCGACAAGAAAACATTACCAACCCTATGCCCC | |
| | CCCACGAGACTTCGCTGCGTACAGGTCCCGAGTGAAGTTTTCCCGAAGCGCA | |
| | GACGCTCCGGCATATCAGCAAGGACAGAATCAGCTGTATAACGAACTGAATT | |
| | TGGGACGCCGCGAGGAGTATGACGTGCTTGATAAACGCCGGGGGAGAGACCC | |
| | GGAAATGGGGGGTAAACCCCGAAGAAAGAATCCCCAAGAAGGACTCTACAAT | |
| | GAACTCCAGAAGGATAAGATGGCGGAGGCCTACTCAGAAATAGGTATGAAGG | |
| | GCGAACGACGACGGGGAAAAGGTCACGATGGCCTCTACCAAGGGTTGAGTAC | |
| | GGCAACCAAAGATACGTACGATGCACTGCATATGCAGGCCCTGCCTCCCAGA | |
| | TAATAATAAAATCGCTATCCATCGAAGATGGATGTGTGTTGGTTTTTTGTGT | |
| | G | |
| CTX-969b | GAGATGTAAGGAGCTGCTGTGACTTGCTCAAGGCCTTATATCGAGTAAACGG | 59 |
| Donor | TAGTGCTGGGGCTTAGACGCAGGTGTTCTGATTTATAGTTCAAAACCTCTAT | |
| LHA to RHA | CAATGAGAGAGCAATCTCCTGGTAATGTGATAGATTTCCCAACTTAATGCCA | |
| 41BB costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGCAAGTGCAGCTTGTGCAATCTGGCGCCGAAGTTA | |
| | AGAAACCAGGCGCATCTGTGAAGGTGAGTTGTAAAGCGTCCGGCTATACTTT | |
| | TACGAACTACGACATTAACTGGGTTAGGCAAGCTCCAGGGCAGGGTCTGGAG | |
| | TGGATAGGTTGGATATATCCAGGTGACGGGTCTACTAAATATAATGAAAAAT | |
| | TCAAGGCTAAGGCCACTTTGACCGCCGACACCTCTACATCAACTGCATACAT | |
| | GGAGTTGAGAAGCCTTCGCAGCGATGACACGGCGGTATATTATTGTGCCAGT | |
| | GGTTATGAAGACGCTATGGACTATTGGGGGCAGGGGACGACGGTAACCGTAT | |
| | CAAGCGGAGGCGGAGGGGGATCAGGCGGGGGCGGATCAGGGGGGGGCGGTAG | |
| | TGATATACAAATGACACAATCTCCAAGCTCACTTTCTGCCAGCGTTGGTGAT | |
| | CGCGTAACTATTAACTGCAAGGCCTCACAGGACATTAACAGTTATCTTAGCT | |
| | GGTTCCAGCAAAAACCTGGGAAAGCTCCAAAAACTTTGATCTATCGAGCGAA | |
| | TAGGCTGGTCGACGGAGTCCCATCTCGCTTCTCCGGCAGTGGCTCAGGCCAG | |
| | GACTATACGCTGACAATAAGTAGTTTGCAACCAGAGGACTTCGCAACCTATT | |
| | ACTGTTTGCAATACGATGAGTTCCCACTCACATTCGGTGGGGGTACTAAAGT | |
| | AGAAATAAAAAGTGCTGCTGCCTTTGTCCCGGTATTTCTCCCAGCCAAACCG | |
| | ACCACGACTCCCGCCCCGCGCCCTCCGACACCCGCTCCCACCATCGCCTCTC | |
| | AACCTCTTAGTCTTCGCCCCGAGGCATGCCGACCCGCCGCCGGGGGTGCTGT | |
| | TCATACGAGGGGCTTGGACTTCGCTTGTGATATTTACATTTGGGCTCCGTTG | |
| | GCGGGTACGTGCGGCGTCCTTTTGTTGTCACTCGTTATTACTTTGTATTGTA | |
| | ATCACAGGAATCGCAAACGGGGCAGAAAGAAACTCCTGTATATATTCAAACA | |
| | ACCATTTATGAGACCAGTACAAACTACTCAAGAGGAAGATGGCTGTAGCTGC | |
| | CGATTTCCAGAAGAAGAAGAAGGAGGATGTGAACTGCGAGTGAAGTTTTCCC | |
| | GAAGCGCAGACGCTCCGGCATATCAGCAAGGACAGAATCAGCTGTATAACGA | |
| | ACTGAATTTGGGACGCCGCGAGGAGTATGACGTGCTTGATAAACGCCGGGGG | |
| | AGAGACCCGGAAATGGGGGGTAAACCCCGAAGAAAGAATCCCCAAGAAGGAC | |
| | TCTACAATGAACTCCAGAAGGATAAGATGGCGGAGGCCTACTCAGAAATAGG | |
| | TATGAAGGGCGAACGACGACGGGGAAAAGGTCACGATGGCCTCTACCAAGGG | |
| | TTGAGTACGGCAACCAAAGATACGTACGATGCACTGCATATGCAGGCCCTGC | |
| | CTCCCAGATAATAATAAAATCGCTATCCATCGAAGATGGATGTGTGTTGGTT | |
| | TTTTGTGTGTGGAGCAACAAATCTGACTTTGCATGTGCAAACGCCTTCAACA | |
| | ACAGCATTATTCCAGAAGACACCTTCTTCCCCAGCCCAGGTAAGGGCAGCTT | |
| | TGGTGCCTTCGCAGGCTGTTTCCTTGCTTCAGGAATGGCCAGGTTCTGCCCA | |
| | GAGCTCTGGTCAATGATGTCTAAAACTCCTCTGATTGGTGGTCTCGGCCTTA | |
| | TCCATTGCCACCAAAACCCTCTTTTTACTAAGAAACAGTGAGCCTTGTTCTG | |
| | GCAGTCCAGAGAATGACACGGGAAAAAAGCAGATGAAGAGAAGGTGGCAGGA | |
| | GAGGGCACGTGGCCCAGCCTCAGTCTCTCCAACTGAGTTCCTGCCTGCCTGC | |
| | CTTTGCTCAGACTGTTTGCCCCTTACTGCTCTTCTAGGCCTCATTCTAAGCC | |
| | CCTTCTCCAAGTTGCCTCTCCTTATTTCTCCCTGTCTGCCAAAAAATCTTTC | |
| | CCAGCTCACTAAGTCAGTCTCACGCAGTCACTCATTAACCCACCAATCACTG | |
| | ATTGTGCCGGCACATGAATGCACCAGGTGTTGAAGTGGAGGAATTAAAAAGT | |
| | CAGATGAGGGGTGTGCCCAGAGGAAGCACCATTCTAGTTGGGGGAGCCCATC | |
| | TGTCAGCTGGGAAAAGTCCAAATAACTTCAGATTGGAATGTGTTTTAACTCA | |
| | GGGTTGAGAAAACAGCTACCTTCAGGACAAAAGTCAGGGAAGGGCTCTCTGA | |
| | AGAAATGCTACTTGAAGATACCAGCCCTACCAAGGGCAGGGAGAGGACCCTA | |
| | TAGAGGCCTGGGACAGGAGCTCAATGAGAAAGG | |
| CTX-969b | CCACCATGGCGCTTCCGGTGACAGCACTGCTCCTCCCCTTGGCGCTGTTGCT | 60 |
| CAR | CCACGCAGCAAGGCCGCAAGTGCAGCTTGTGCAATCTGGCGCCGAAGTTAAG | |
| 41BB costim. | AAACCAGGCGCATCTGTGAAGGTGAGTTGTAAAGCGTCCGGCTATACTTTTA | |
| | CGAACTACGACATTAACTGGGTTAGGCAAGCTCCAGGGCAGGGTCTGGAGTG | |
| | GATAGGTTGGATATATCCAGGTGACGGGTCTACTAAATATAATGAAAAATTC | |
| | AAGGCTAAGGCCACTTTGACCGCCGACACCTCTACATCAACTGCATACATGG | |
| | AGTTGAGAAGCCTTCGCAGCGATGACACGGCGGTATATTATTGTGCCAGTGG | |
| | TTATGAAGACGCTATGGACTATTGGGGGCAGGGGACGACGGTAACCGTATCA | |
| | AGCGGAGGCGGAGGGGGATCAGGCGGGGGCGGATCAGGGGGGGGCGGTAGTG | |
| | ATATACAAATGACACAATCTCCAAGCTCACTTTCTGCCAGCGTTGGTGATCG | |
| | CGTAACTATTAACTGCAAGGCCTCACAGGACATTAACAGTTATCTTAGCTGG | |
| | TTCCAGCAAAAACCTGGGAAAGCTCCAAAAACTTTGATCTATCGAGCGAATA | |
| | GGCTGGTCGACGGAGTCCCATCTCGCTTCTCCGGCAGTGGCTCAGGCCAGGA | |
| | CTATACGCTGACAATAAGTAGTTTGCAACCAGAGGACTTCGCAACCTATTAC | |
| | TGTTTGCAATACGATGAGTTCCCACTCACATTCGGTGGGGGTACTAAAGTAG | |
| | AAATAAAAAGTGCTGCTGCCTTTGTCCCGGTATTTCTCCCAGCCAAACCGAC | |
| | CACGACTCCCGCCCCGCGCCCTCCGACACCCGCTCCCACCATCGCCTCTCAA | |
| | CCTCTTAGTCTTCGCCCCGAGGCATGCCGACCCGCCGCCGGGGGTGCTGTTC | |
| | ATACGAGGGGCTTGGACTTCGCTTGTGATATTTACATTTGGGCTCCGTTGGC | |
| | GGGTACGTGCGGCGTCCTTTTGTTGTCACTCGTTATTACTTTGTATTGTAAT | |
| | CACAGGAATCGCAAACGGGGCAGAAAGAAACTCCTGTATATATTCAAACAAC | |
| | CATTTATGAGACCAGTACAAACTACTCAAGAGGAAGATGGCTGTAGCTGCCG | |
| | ATTTCCAGAAGAAGAAGAAGGAGGATGTGAACTGCGAGTGAAGTTTTCCCGA | |
| | AGCGCAGACGCTCCGGCATATCAGCAAGGACAGAATCAGCTGTATAACGAAC | |
| | TGAATTTGGGACGCCGCGAGGAGTATGACGTGCTTGATAAACGCCGGGGGAG | |
| | AGACCCGGAAATGGGGGGTAAACCCCGAAGAAAGAATCCCCAAGAAGGACTC | |
| | TACAATGAACTCCAGAAGGATAAGATGGCGGAGGCCTACTCAGAAATAGGTA | |
| | TGAAGGGCGAACGACGACGGGGAAAAGGTCACGATGGCCTCTACCAAGGGTT | |
| | GAGTACGGCAACCAAAGATACGTACGATGCACTGCATATGCAGGCCCTGCCT | |
| | CCCAGATAATAATAAAATCGCTATCCATCGAAGATGGATGTGTGTTGGTTTT | |
| | TTGTGTG | |
| CTX-970 | GAGATGTAAGGAGCTGCTGTGACTTGCTCAAGGCCTTATATCGAGTAAACGG | 61 |
| Donor | TAGTGCTGGGGCTTAGACGCAGGTGTTCTGATTTATAGTTCAAAACCTCTAT | |
| LHA or RHA | CAATGAGAGAGCAATCTCCTGGTAATGTGATAGATTTCCCAACTTAATGCCA | |
| CD28 costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGACATCCAAATGACCCAGTCACCGAGTTCACTGT | |
| | CTGCCAGTGTAGGAGATCGCGTCACCATTAATTGCAAGGCCAGTCAGGACAT | |
| | AAATAGTTACCTGAGCTGGTTCCAGCAAAAACCAGGTAAGGCCCCGAAGACT | |
| | CTTATCTATCGAGCGAACCGACTTGTAGACGGTGTTCCTTCCAGATTTTCCG | |
| | GCAGTGGCAGCGGCCAAGATTACACTCTTACTATCTCTTCACTGCAACCTGA | |
| | AGATTTCGCGACCTATTACTGCCTCCAATACGACGAGTTCCCACTGACGTTT | |
| | GGCGGCGGAACGAAGGTAGAAATCAAGGGCGGGGGAGGGGGGTCAGGTGGAG | |
| | GCGGCTCTGGCGGAGGTGGTAGTCAGGTCCAACTCGTTCAGAGCGGGGCGGA | |
| | GGTAAAGAAGCCAGGGGCCAGTGTCAAGGTTAGTTGTAAAGCATCTGGCTAT | |
| | ACCTTCACGAATTACGATATAAACTGGGTACGACAAGCCCCTGGGCAAGGAC | |
| | TTGAATGGATTGGATGGATCTATCCAGGCGATGGATCAACCAAATACAATGA | |
| | GAAGTTTAAGGCTAAAGCCACACTCACCGCCGATACCTCCACCAGTACAGCG | |
| | TATATGGAGTTGAGGTCACTTCGCTCTGATGATACTGCGGTGTACTATTGCG | |
| | CAAGTGGTTACGAGGACGCTATGGACTACTGGGGGCAAGGGACAACAGTGAC | |
| | CGTTTCTTCTAGTGCTGCTGCCTTTGTCCCGGTATTTCTCCCAGCCAAACCG | |
| | ACCACGACTCCCGCCCCGCGCCCTCCGACACCCGCTCCCACCATCGCCTCTC | |
| | AACCTCTTAGTCTTCGCCCCGAGGCATGCCGACCCGCCGCCGGGGGTGCTGT | |
| | TCATACGAGGGGCTTGGACTTCGCTTGTGATATTTACATTTGGGCTCCGTTG | |
| | GCGGGTACGTGCGGCGTCCTTTTGTTGTCACTCGTTATTACTTTGTATTGTA | |
| | ATCACAGGAATCGCTCAAAGCGGAGTAGGTTGTTGCATTCCGATTACATGAA | |
| | TATGACTCCTCGCCGGCCTGGGCCGACAAGAAAACATTACCAACCCTATGCC | |
| | CCCCCACGAGACTTCGCTGCGTACAGGTCCCGAGTGAAGTTTTCCCGAAGCG | |
| | CAGACGCTCCGGCATATCAGCAAGGACAGAATCAGCTGTATAACGAACTGAA | |
| | TTTGGGACGCCGCGAGGAGTATGACGTGCTTGATAAACGCCGGGGGAGAGAC | |
| | CCGGAAATGGGGGGTAAACCCCGAAGAAAGAATCCCCAAGAAGGACTCTACA | |
| | ATGAACTCCAGAAGGATAAGATGGCGGAGGCCTACTCAGAAATAGGTATGAA | |
| | GGGCGAACGACGACGGGGAAAAGGTCACGATGGCCTCTACCAAGGGTTGAGT | |
| | ACGGCAACCAAAGATACGTACGATGCACTGCATATGCAGGCCCTGCCTCCCA | |
| | GATAATAATAAAATCGCTATCCATCGAAGATGGATGTGTGTTGGTTTTTTGT | |
| | GTGTGGAGCAACAAATCTGACTTTGCATGTGCAAACGCCTTCAACAACAGCA | |
| | TTATTCCAGAAGACACCTTCTTCCCCAGCCCAGGTAAGGGCAGCTTTGGTGC | |
| | CTTCGCAGGCTGTTTCCTTGCTTCAGGAATGGCCAGGTTCTGCCCAGAGCTC | |
| | TGGTCAATGATGTCTAAAACTCCTCTGATTGGTGGTCTCGGCCTTATCCATT | |
| | GCCACCAAAACCCTCTTTTTACTAAGAAACAGTGAGCCTTGTTCTGGCAGTC | |
| | CAGAGAATGACACGGGAAAAAAGCAGATGAAGAGAAGGTGGCAGGAGAGGGC | |
| | ACGTGGCCCAGCCTCAGTCTCTCCAACTGAGTTCCTGCCTGCCTGCCTTTGC | |
| | TCAGACTGTTTGCCCCTTACTGCTCTTCTAGGCCTCATTCTAAGCCCCTTCT | |
| | CCAAGTTGCCTCTCCTTATTTCTCCCTGTCTGCCAAAAAATCTTTCCCAGCT | |
| | CACTAAGTCAGTCTCACGCAGTCACTCATTAACCCACCAATCACTGATTGTG | |
| | CCGGCACATGAATGCACCAGGTGTTGAAGTGGAGGAATTAAAAAGTCAGATG | |
| | AGGGGTGTGCCCAGAGGAAGCACCATTCTAGTTGGGGGAGCCCATCTGTCAG | |
| | CTGGGAAAAGTCCAAATAACTTCAGATTGGAATGTGTTTTAACTCAGGGTTG | |
| | AGAAAACAGCTACCTTCAGGACAAAAGTCAGGGAAGGGCTCTCTGAAGAAAT | |
| | GCTACTTGAAGATACCAGCCCTACCAAGGGCAGGGAGAGGACCCTATAGAGG | |
| | CCTGGGACAGGAGCTCAATGAGAAAGG | |
| CTX-970 | CCACCATGGCGCTTCCGGTGACAGCACTGCTCCTCCCCTTGGCGCTGTTGCT | 62 |
| CAR | CCACGCAGCAAGGCCGGACATCCAAATGACCCAGTCACCGAGTTCACTGTCT | |
| CD28 costim. | GCCAGTGTAGGAGATCGCGTCACCATTAATTGCAAGGCCAGTCAGGACATAA | |
| | ATAGTTACCTGAGCTGGTTCCAGCAAAAACCAGGTAAGGCCCCGAAGACTCT | |
| | TATCTATCGAGCGAACCGACTTGTAGACGGTGTTCCTTCCAGATTTTCCGGC | |
| | AGTGGCAGCGGCCAAGATTACACTCTTACTATCTCTTCACTGCAACCTGAAG | |
| | ATTTCGCGACCTATTACTGCCTCCAATACGACGAGTTCCCACTGACGTTTGG | |
| | CGGCGGAACGAAGGTAGAAATCAAGGGCGGGGGAGGGGGGTCAGGTGGAGGC | |
| | GGCTCTGGCGGAGGTGGTAGTCAGGTCCAACTCGTTCAGAGCGGGGCGGAGG | |
| | TAAAGAAGCCAGGGGCCAGTGTCAAGGTTAGTTGTAAAGCATCTGGCTATAC | |
| | CTTCACGAATTACGATATAAACTGGGTACGACAAGCCCCTGGGCAAGGACTT | |
| | GAATGGATTGGATGGATCTATCCAGGCGATGGATCAACCAAATACAATGAGA | |
| | AGTTTAAGGCTAAAGCCACACTCACCGCCGATACCTCCACCAGTACAGCGTA | |
| | TATGGAGTTGAGGTCACTTCGCTCTGATGATACTGCGGTGTACTATTGCGCA | |
| | AGTGGTTACGAGGACGCTATGGACTACTGGGGGCAAGGGACAACAGTGACCG | |
| | TTTCTTCTAGTGCTGCTGCCTTTGTCCCGGTATTTCTCCCAGCCAAACCGAC | |
| | CACGACTCCCGCCCCGCGCCCTCCGACACCCGCTCCCACCATCGCCTCTCAA | |
| | CCTCTTAGTCTTCGCCCCGAGGCATGCCGACCCGCCGCCGGGGGTGCTGTTC | |
| | ATACGAGGGGCTTGGACTTCGCTTGTGATATTTACATTTGGGCTCCGTTGGC | |
| | GGGTACGTGCGGCGTCCTTTTGTTGTCACTCGTTATTACTTTGTATTGTAAT | |
| | CACAGGAATCGCTCAAAGCGGAGTAGGTTGTTGCATTCCGATTACATGAATA | |
| | TGACTCCTCGCCGGCCTGGGCCGACAAGAAAACATTACCAACCCTATGCCCC | |
| | CCCACGAGACTTCGCTGCGTACAGGTCCCGAGTGAAGTTTTCCCGAAGCGCA | |
| | GACGCTCCGGCATATCAGCAAGGACAGAATCAGCTGTATAACGAACTGAATT | |
| | TGGGACGCCGCGAGGAGTATGACGTGCTTGATAAACGCCGGGGGAGAGACCC | |
| | GGAAATGGGGGGTAAACCCCGAAGAAAGAATCCCCAAGAAGGACTCTACAAT | |
| | GAACTCCAGAAGGATAAGATGGCGGAGGCCTACTCAGAAATAGGTATGAAGG | |
| | GCGAACGACGACGGGGAAAAGGTCACGATGGCCTCTACCAAGGGTTGAGTAC | |
| | GGCAACCAAAGATACGTACGATGCACTGCATATGCAGGCCCTGCCTCCCAGA | |
| | TAATAATAAAATCGCTATCCATCGAAGATGGATGTGTGTTGGTTTTTTGTGT | |
| | G | |
| CTX-970b | GAGATGTAAGGAGCTGCTGTGACTTGCTCAAGGCCTTATATCGAGTAAACGG | 63 |
| Donor | TAGTGCTGGGGCTTAGACGCAGGTGTTCTGATTTATAGTTCAAAACCTCTAT | |
| LHA or RHA | CAATGAGAGAGCAATCTCCTGGTAATGTGATAGATTTCCCAACTTAATGCCA | |
| 41BB costim. | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcagttagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGACATCCAAATGACCCAGTCACCGAGTTCACTGT | |
| | CTGCCAGTGTAGGAGATCGCGTCACCATTAATTGCAAGGCCAGTCAGGACAT | |
| | AAATAGTTACCTGAGCTGGTTCCAGCAAAAACCAGGTAAGGCCCCGAAGACT | |
| | CTTATCTATCGAGCGAACCGACTTGTAGACGGTGTTCCTTCCAGATTTTCCG | |
| | GCAGTGGCAGCGGCCAAGATTACACTCTTACTATCTCTTCACTGCAACCTGA | |
| | AGATTTCGCGACCTATTACTGCCTCCAATACGACGAGTTCCCACTGACGTTT | |
| | GGCGGCGGAACGAAGGTAGAAATCAAGGGCGGGGGAGGGGGGTCAGGTGGAG | |
| | GCGGCTCTGGCGGAGGTGGTAGTCAGGTCCAACTCGTTCAGAGCGGGGCGGA | |
| | GGTAAAGAAGCCAGGGGCCAGTGTCAAGGTTAGTTGTAAAGCATCTGGCTAT | |
| | ACCTTCACGAATTACGATATAAACTGGGTACGACAAGCCCCTGGGCAAGGAC | |
| | TTGAATGGATTGGATGGATCTATCCAGGCGATGGATCAACCAAATACAATGA | |
| | GAAGTTTAAGGCTAAAGCCACACTCACCGCCGATACCTCCACCAGTACAGCG | |
| | TATATGGAGTTGAGGTCACTTCGCTCTGATGATACTGCGGTGTACTATTGCG | |
| | CAAGTGGTTACGAGGACGCTATGGACTACTGGGGGCAAGGGACAACAGTGAC | |
| | CGTTTCTTCTAGTGCTGCTGCCTTTGTCCCGGTATTTCTCCCAGCCAAACCG | |
| | ACCACGACTCCCGCCCCGCGCCCTCCGACACCCGCTCCCACCATCGCCTCTC | |
| | AACCTCTTAGTCTTCGCCCCGAGGCATGCCGACCCGCCGCCGGGGGTGCTGT | |
| | TCATACGAGGGGCTTGGACTTCGCTTGTGATATTTACATTTGGGCTCCGTTG | |
| | GCGGGTACGTGCGGCGTCCTTTTGTTGTCACTCGTTATTACTTTGTATTGTA | |
| | ATCACAGGAATCGCAAACGGGGCAGAAAGAAACTCCTGTATATATTCAAACA | |
| | ACCATTTATGAGACCAGTACAAACTACTCAAGAGGAAGATGGCTGTAGCTGC | |
| | CGATTTCCAGAAGAAGAAGAAGGAGGATGTGAACTGCGAGTGAAGTTTTCCC | |
| | GAAGCGCAGACGCTCCGGCATATCAGCAAGGACAGAATCAGCTGTATAACGA | |
| | ACTGAATTTGGGACGCCGCGAGGAGTATGACGTGCTTGATAAACGCCGGGGG | |
| | AGAGACCCGGAAATGGGGGGTAAACCCCGAAGAAAGAATCCCCAAGAAGGAC | |
| | TCTACAATGAACTCCAGAAGGATAAGATGGCGGAGGCCTACTCAGAAATAGG | |
| | TATGAAGGGCGAACGACGACGGGGAAAAGGTCACGATGGCCTCTACCAAGGG | |
| | TTGAGTACGGCAACCAAAGATACGTACGATGCACTGCATATGCAGGCCCTGC | |
| | CTCCCAGATAATAATAAAATCGCTATCCATCGAAGATGGATGTGTGTTGGTT | |
| | TTTTGTGTGTGGAGCAACAAATCTGACTTTGCATGTGCAAACGCCTTCAACA | |
| | ACAGCATTATTCCAGAAGACACCTTCTTCCCCAGCCCAGGTAAGGGCAGCTT | |
| | TGGTGCCTTCGCAGGCTGTTTCCTTGCTTCAGGAATGGCCAGGTTCTGCCCA | |
| | GAGCTCTGGTCAATGATGTCTAAAACTCCTCTGATTGGTGGTCTCGGCCTTA | |
| | TCCATTGCCACCAAAACCCTCTTTTTACTAAGAAACAGTGAGCCTTGTTCTG | |
| | GCAGTCCAGAGAATGACACGGGAAAAAAGCAGATGAAGAGAAGGTGGCAGGA | |
| | GAGGGCACGTGGCCCAGCCTCAGTCTCTCCAACTGAGTTCCTGCCTGCCTGC | |
| | CTTTGCTCAGACTGTTTGCCCCTTACTGCTCTTCTAGGCCTCATTCTAAGCC | |
| | CCTTCTCCAAGTTGCCTCTCCTTATTTCTCCCTGTCTGCCAAAAAATCTTTC | |
| | CCAGCTCACTAAGTCAGTCTCACGCAGTCACTCATTAACCCACCAATCACTG | |
| | ATTGTGCCGGCACATGAATGCACCAGGTGTTGAAGTGGAGGAATTAAAAAGT | |
| | CAGATGAGGGGTGTGCCCAGAGGAAGCACCATTCTAGTTGGGGGAGCCCATC | |
| | TGTCAGCTGGGAAAAGTCCAAATAACTTCAGATTGGAATGTGTTTTAACTCA | |
| | GGGTTGAGAAAACAGCTACCTTCAGGACAAAAGTCAGGGAAGGGCTCTCTGA | |
| | AGAAATGCTACTTGAAGATACCAGCCCTACCAAGGGCAGGGAGAGGACCCTA | |
| | TAGAGGCCTGGGACAGGAGCTCAATGAGAAAGG | |
| CTX-970b | CCACCATGGCGCTTCCGGTGACAGCACTGCTCCTCCCCTTGGCGCTGTTGCT | 64 |
| CAR | CCACGCAGCAAGGCCGGACATCCAAATGACCCAGTCACCGAGTTCACTGTCT | |
| 41BB costim. | GCCAGTGTAGGAGATCGCGTCACCATTAATTGCAAGGCCAGTCAGGACATAA | |
| | ATAGTTACCTGAGCTGGTTCCAGCAAAAACCAGGTAAGGCCCCGAAGACTCT | |
| | TATCTATCGAGCGAACCGACTTGTAGACGGTGTTCCTTCCAGATTTTCCGGC | |
| | AGTGGCAGCGGCCAAGATTACACTCTTACTATCTCTTCACTGCAACCTGAAG | |
| | ATTTCGCGACCTATTACTGCCTCCAATACGACGAGTTCCCACTGACGTTTGG | |
| | CGGCGGAACGAAGGTAGAAATCAAGGGCGGGGGAGGGGGGTCAGGTGGAGGC | |
| | GGCTCTGGCGGAGGTGGTAGTCAGGTCCAACTCGTTCAGAGCGGGGCGGAGG | |
| | TAAAGAAGCCAGGGGCCAGTGTCAAGGTTAGTTGTAAAGCATCTGGCTATAC | |
| | CTTCACGAATTACGATATAAACTGGGTACGACAAGCCCCTGGGCAAGGACTT | |
| | GAATGGATTGGATGGATCTATCCAGGCGATGGATCAACCAAATACAATGAGA | |
| | AGTTTAAGGCTAAAGCCACACTCACCGCCGATACCTCCACCAGTACAGCGTA | |
| | TATGGAGTTGAGGTCACTTCGCTCTGATGATACTGCGGTGTACTATTGCGCA | |
| | AGTGGTTACGAGGACGCTATGGACTACTGGGGGCAAGGGACAACAGTGACCG | |
| | TTTCTTCTAGTGCTGCTGCCTTTGTCCCGGTATTTCTCCCAGCCAAACCGAC | |
| | CACGACTCCCGCCCCGCGCCCTCCGACACCCGCTCCCACCATCGCCTCTCAA | |
| | CCTCTTAGTCTTCGCCCCGAGGCATGCCGACCCGCCGCCGGGGGTGCTGTTC | |
| | ATACGAGGGGCTTGGACTTCGCTTGTGATATTTACATTTGGGCTCCGTTGGC | |
| | GGGTACGTGCGGCGTCCTTTTGTTGTCACTCGTTATTACTTTGTATTGTAAT | |
| | CACAGGAATCGCAAACGGGGCAGAAAGAAACTCCTGTATATATTCAAACAAC | |
| | CATTTATGAGACCAGTACAAACTACTCAAGAGGAAGATGGCTGTAGCTGCCG | |
| | ATTTCCAGAAGAAGAAGAAGGAGGATGTGAACTGCGAGTGAAGTTTTCCCGA | |
| | AGCGCAGACGCTCCGGCATATCAGCAAGGACAGAATCAGCTGTATAACGAAC | |
| | TGAATTTGGGACGCCGCGAGGAGTATGACGTGCTTGATAAACGCCGGGGGAG | |
| | AGACCCGGAAATGGGGGGTAAACCCCGAAGAAAGAATCCCCAAGAAGGACTC | |
| | TACAATGAACTCCAGAAGGATAAGATGGCGGAGGCCTACTCAGAAATAGGTA | |
| | TGAAGGGCGAACGACGACGGGGAAAAGGTCACGATGGCCTCTACCAAGGGTT | |
| | GAGTACGGCAACCAAAGATACGTACGATGCACTGCATATGCAGGCCCTGCCT | |
| | CCCAGATAATAATAAAATCGCTATCCATCGAAGATGGATGTGTGTTGGTTTT | |
| | TTGTGTG | |
| anti-CD33 antibody ‘Antibody A’ VH CDRs underlined and in bold | 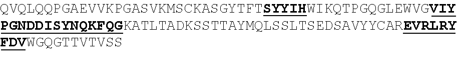 | 65 |
| anti-CD33 antibody ‘Antibody A’ VL CDRs underlined and in bold | 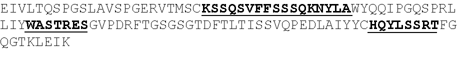 | 66 |
| anti-CD33 | SYYIH | 67 |
| antibody | | |
| ‘Antibody A’ | | |
| VH CDR1 | | |
| anti-CD33 | VIYPGNDDISYNQKFQG | 68 |
| antibody | | |
| ‘Antibody A’ | | |
| VH CDR2 | | |
| anti-CD33 | EVRLRYFDV | 69 |
| antibody | | |
| ‘Antibody A’ | | |
| VH CDR3 | | |
| anti-CD33 | KSSQSVFFSSSQKNYLA | 70 |
| antibody | | |
| ‘Antibody A’ | | |
| VL CDR1 | | |
| anti-CD33 | WASTRES | 71 |
| antibody | | |
| ‘Antibody A’ | | |
| VL CDR2 | | |
| anti-CD33 | HQYLSSRT | 72 |
| antibody | | |
| ‘Antibody A’ | | |
| VL CDR3 | | |
| CTX-964 and | QVQLQQPGAEVVKPGASVKMSCKASGYTFTSYYIHWIKQTPGQGLEWVGVIY | 73 |
| CTX-964b | PGNDDISYNQKFQGKATLTADKSSTTAYMQLSSLTSEDSAVYYCAREVRLRY | |
| scFv | FDVWGQGTTVTVSSGGGGGSGGGGSGGGGSEIVLTQSPGSLAVSPGERVTMS | |
| Linker underlined | CKSSQSVFFSSSQKNYLAWYQQIPGQSPRLLIYWASTRESGVPDRFTGSGSG | |
| | TDFTLTISSVQPEDLAIYYCHQYLSSRTFGQGTKLEIK | |
| CTX-964 and | CAGGTACAACTCCAACAACCCGGAGCTGAGGTTGTAAAACCAGGTGCGTCAG | 74 |
| CTX-964b | TCAAGATGAGTTGCAAAGCCAGTGGATATACTTTTACTTCCTATTACATTCA | |
| scFv | TTGGATCAAGCAGACTCCAGGTCAGGGGCTCGAGTGGGTAGGCGTGATCTAC | |
| | CCCGGTAACGACGACATTTCATACAACCAAAAATTTCAGGGGAAAGCGACGC | |
| | TGACTGCTGACAAGAGTAGCACGACCGCATATATGCAACTCTCATCACTTAC | |
| | GTCTGAGGATTCTGCAGTTTATTATTGCGCTCGGGAAGTTCGGCTTCGATAT | |
| | TTCGATGTGTGGGGTCAGGGCACGACCGTAACGGTGAGCAGTGGTGGCGGTG | |
| | GCGGGTCCGGGGGCGGTGGATCAGGTGGTGGGGGGAGTGAGATAGTGTTGAC | |
| | CCAGTCACCGGGGTCCCTCGCAGTTTCACCGGGAGAGAGGGTCACAATGTCC | |
| | TGCAAATCCTCCCAATCAGTGTTCTTCTCTTCCAGCCAAAAAAACTACCTTG | |
| | CGTGGTATCAACAGATACCGGGACAGTCTCCTCGCCTCCTGATCTACTGGGC | |
| | ATCTACCCGAGAAAGCGGTGTTCCGGATAGGTTTACCGGTTCCGGGTCTGGG | |
| | ACCGATTTTACGTTGACAATATCCAGCGTACAGCCGGAAGACCTTGCTATCT | |
| | ATTACTGTCACCAGTACCTTTCCAGCCGGACGTTCGGGCAGGGCACGAAGCT | |
| | GGAGATTAAA | |
| CTX-965 and | EIVLTQSPGSLAVSPGERVTMSCKSSQSVFFSSSQKNYLAWYQQIPGQSPRL | 75 |
| CTX-965b | LIYWASTRESGVPDRFTGSGSGTDFTLTISSVQPEDLAIYYCHQYLSSRTFG | |
| scFv | QGTKLEIKGGGGGSGGGGSGGGGSQVQLQQPGAEVVKPGASVKMSCKASGYT | |
| Linker underlined | FTSYYIHWIKQTPGQGLEWVGVIYPGNDDISYNQKFQGKATLTADKSSTTAY | |
| | MQLSSLTSEDSAVYYCAREVRLRYFDVWGQGTTVTVSS | |
| CTX-965 and | GAAATCGTCCTCACACAATCCCCGGGGAGCCTCGCAGTCAGTCCTGGGGAAG | 76 |
| CTX-965b | GAGTCACTATGAGCTGCAAATCCAGTCAGAGTGTTTTTTTCTCAAGTAGCCA | |
| scFv | GAAGAACTACCTCGCATGGTACCAACAAATACCGGGGCAATCTCCCCGCTTG | |
| | CTTATATACTGGGCAAGTACCCGCGAATCCGGCGTACCGGATCGATTCACGG | |
| | GATCTGGGTCAGGTACTGATTTCACTTTGACTATCAGCTCTGTTCAGCCTGA | |
| | AGATTTGGCAATTTACTACTGTCACCAATACTTGAGTAGCCGAACTTTCGGC | |
| | CAGGGCACGAAGCTCGAAATCAAGGGCGGAGGGGGAGGTTCTGGTGGGGGCG | |
| | CGTAAAACCTGGGGCGTCTGTCAAGATGAGCTGTAAAGCAAGTGGATACACC | |
| | TTCACCTCCTACTATATACATTGGATTAAGCAAACTCCGGGTCAGGGGCTGG | |
| | AATGGGTTGGCGTTATATACCCCGGGAACGATGATATATCATACAACCAAAA | |
| | ATTTCAAGGCAAGGCGACTCTGACTGCCGATAAGAGTAGCACAACAGCTTAC | |
| | ATGCAGCTTTCTTCCCTGACCAGCGAAGATTCAGCAGTTTACTACTGCGCTC | |
| | GGGAAGTGCGCCTGCGATACTTTGATGTCTGGGGTCAAGGAACTACAGTTAC | |
| | TGTATCAAGC | |
| anti-CD33 antibody ‘Antibody B’ VH CDRs underlined and in bold | 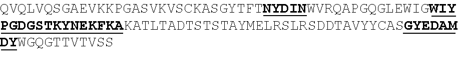 | 77 |
| anti-CD33 antibody ‘Antibody B’ VL CDRs underlined and in bold | 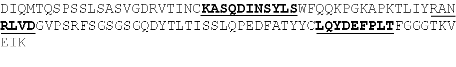 | 78 |
| anti-CD33 | NYDIN | 79 |
| antibody | | |
| ‘Antibody B’ | | |
| VH CDR1 | | |
| anti-CD33 antibody ‘Antibody B’ VH CDR2 | 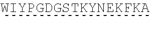 | 80 |
| anti-CD33 antibody ‘Antibody B’ VH CDR3 | 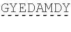 | 81 |
| anti-CD33 | KASQDINSYLS | 82 |
| antibody | | |
| ‘Antibody B’ | | |
| VL CDR1 | | |
| anti-CD33 | RANRLVD | 83 |
| antibody | | |
| ‘Antibody B’ | | |
| VL CDR2 | | |
| anti-CD33 | LQYDEFPLT | 84 |
| antibody | | |
| ‘Antibody B’ | | |
| VL CDR3 | | |
| CTX-969 and CTX-969b scFv Linker underlined | 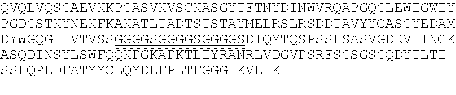 | 85 |
| CTX-969 | CAAGTGCAGCTTGTGCAATCTGGCGCCGAAGTTAAGAAACCAGGCGCATCTG | 86 |
| CTX-969b | TGAAGGTGAGTTGTAAAGCGTCCGGCTATACTTTTACGAACTACGACATTAA | |
| scFv | CTGGGTTAGGCAAGCTCCAGGGCAGGGTCTGGAGTGGATAGGTTGGATATAT | |
| | CCAGGTGACGGGTCTACTAAATATAATGAAAAATTCAAGGCTAAGGCCACTT | |
| | TGACCGCCGACACCTCTACATCAACTGCATACATGGAGTTGAGAAGCCTTCG | |
| | CAGCGATGACACGGCGGTATATTATTGTGCCAGTGGTTATGAAGACGCTATG | |
| | GACTATTGGGGGCAGGGGACGACGGTAACCGTATCAAGCGGAGGCGGAGGGG | |
| | GATCAGGCGGGGGCGGATCAGGGGGGGGCGGTAGTGATATACAAATGACACA | |
| | ATCTCCAAGCTCACTTTCTGCCAGCGTTGGTGATCGCGTAACTATTAACTGC | |
| | AAGGCCTCACAGGACATTAACAGTTATCTTAGCTGGTTCCAGCAAAAACCTG | |
| | GGAAAGCTCCAAAAACTTTGATCTATCGAGCGAATAGGCTGGTCGACGGAGT | |
| | CCCATCTCGCTTCTCCGGCAGTGGCTCAGGCCAGGACTATACGCTGACAATA | |
| | AGTAGTTTGCAACCAGAGGACTTCGCAACCTATTACTGTTTGCAATACGATG | |
| | AGTTCCCACTCACATTCGGTGGGGGTACTAAAGTAGAAATAAAA | |
| CTX-970 and CTX-970b scFv linker underlined | 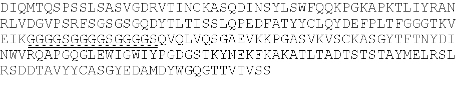 | 87 |
| CTX-970 and | GACATCCAAATGACCCAGTCACCGAGTTCACTGTCTGCCAGTGTAGGAGATC | 88 |
| CTX-970b | GCGTCACCATTAATTGCAAGGCCAGTCAGGACATAAATAGTTACCTGAGCTG | |
| svFv | GTTCCAGCAAAAACCAGGTAAGGCCCCGAAGACTCTTATCTATCGAGCGAAC | |
| | CGACTTGTAGACGGTGTTCCTTCCAGATTTTCCGGCAGTGGCAGCGGCCAAG | |
| | ATTACACTCTTACTATCTCTTCACTGCAACCTGAAGATTTCGCGACCTATTA | |
| | CTGCCTCCAATACGACGAGTTCCCACTGACGTTTGGCGGCGGAACGAAGGTA | |
| | GAAATCAAGGGCGGGGGAGGGGGGTCAGGTGGAGGCGGCTCTGGCGGAGGTG | |
| | GTAGTCAGGTCCAACTCGTTCAGAGCGGGGCGGAGGTAAAGAAGCCAGGGGC | |
| | CAGTGTCAAGGTTAGTTGTAAAGCATCTGGCTATACCTTCACGAATTACGAT | |
| | ATAAACTGGGTACGACAAGCCCCTGGGCAAGGACTTGAATGGATTGGATGGA | |
| | TCTATCCAGGCGATGGATCAACCAAATACAATGAGAAGTTTAAGGCTAAAGC | |
| | CACACTCACCGCCGATACCTCCACCAGTACAGCGTATATGGAGTTGAGGTCA | |
| | CTTCGCTCTGATGATACTGCGGTGTACTATTGCGCAAGTGGTTACGAGGACG | |
| | CCATGGACTACTGGGGGCAAGGGACAACAGTGACCGTTTCTTCT | |
| anti-CD33 antibody ‘Antibody C’ VL CDRs underlined and in bold | 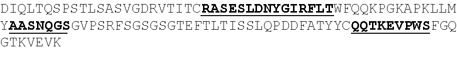 | 90 |
| anti-CD33 | DSNIH | 91 |
| antibody | | |
| ‘Antibody C’ | | |
| VH CDR1 | | |
| anti-CD33 | YIYPYNGGTDYNQKFKN | 92 |
| antibody | | |
| ‘Antibody C’ | | |
| VH CDR2 | | |
| anti-CD33 | GNPWLAY | 93 |
| antibody | | |
| ‘Antibody C’ | | |
| VH CDR3 | | |
| anti-CD33 | RASESLDNYGIRFLT | 94 |
| antibody | | |
| ‘Antibody C’ | | |
| VL CDR1 | | |
| anti-CD33 | AASNQGS | 95 |
| antibody | | |
| ‘Antibody C’ | | |
| VL CDR2 | | |
| anti-CD33 | QQTKEVPWS | 96 |
| antibody | | |
| ‘Antibody C’ | | |
| VL CDR3 | | |
| CTX-981 and CTX-981b ScFv linker underlined | 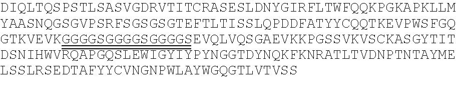 | 97 |
| CTX-981 and | GATATACAGCTCACGCAGAGTCCATCAACACTGTCCGCCAGTGTCGGTGACC | 98 |
| CTX-981b | GGGTTACTATTACGTGCCGCGCAAGCGAATCTCTGGATAATTATGGTATCCG | |
| scFv | GTTTCTGACATGGTTTCAGCAAAAACCGGGGAAAGCTCCCAAGCTGCTTATG | |
| | TACGCCGCCTCTAATCAGGGGTCAGGTGTCCCTAGCCGGTTCTCCGGTTCCG | |
| | GTAGTGGCACGGAATTCACTCTCACAATCAGTTCACTCCAGCCGGATGACTT | |
| | TGCAACGTATTATTGTCAACAAACGAAGGAGGTTCCTTGGTCTTTCGGTCAG | |
| | GGAACTAAGGTTGAGGTTAAGGGAGGAGGTGGTTCTGGCGGAGGCGGATCTG | |
| | GTGGCGGAGGTTCCGAGGTACAACTTGTGCAAAGTGGGGCTGAGGTTAAAAA | |
| | ACCCGGCAGCTCTGTCAAAGTTTCCTGTAAGGCTAGTGGTTACACCATCACT | |
| | GACTCCAATATACACTGGGTTAGACAGGCTCCAGGGCAGTCACTTGAGTGGA | |
| | TAGGCTACATCTATCCATACAACGGAGGTACAGACTACAACCAAAAATTTAA | |
| | AAACAGGGCGACGCTTACAGTCGATAACCCCACAAATACAGCATATATGGAG | |
| | CTGTCATCTTTGCGCAGCGAAGATACAGCTTTCTACTATTGTGTGAATGGTA | |
| | ATCCCTGGCTGGCCTATTGGGGGCAGGGAACTCTTGTCACTGTTTCCAGT | |
| CTX-982 and | EVQLVQSGAEVKKPGSSVKVSCKASGYTITDSNIHWVRQAPGQSLEWIGYIY | 99 |
| CTX-982b | PYNGGTDYNQKFKNRATLTVDNPTNTAYMELSSLRSEDTAFYYCVNGNPWLA | |
| scFv | YWGQGTLVTVSSGGGGSGGGGSGGGGSDIQLTQSPSTLSASVGDRVTITCRA | |
| linker underlined | SESLDNYGIRFLTWFQQKPGKAPKLLMYAASNQGSGVPSRFSGSGSGTEFTL | |
| | TISSLQPDDFATYYCQQTKEVPWSFGQGTKVEVK | |
| CTX-982 and | GAGGTCCAACTTGTTCAATCCGGCGCTGAAGTGAAAAAGCCAGGAAGTAGCG | 100 |
| CTX-982b | TAAAAGTAAGCTGTAAAGCTAGCGGTTACACCATTACCGACAGCAACATCCA | |
| scFv | TTGGGTGCGGCAGGCGCCAGGACAATCCCTCGAGTGGATAGGTTACATCTAT | |
| | CCTTACAACGGGGGAACAGATTATAATCAGAAGTTCAAGAACCGGGCAACGC | |
| | TCACTGTTGACAATCCCACTAATACTGCCTATATGGAGCTCTCCAGCCTCCG | |
| | CAGTGAGGACACTGCGTTTTATTATTGCGTGAATGGCAACCCGTGGCTTGCT | |
| | TATTGGGGACAGGGCACATTGGTTACAGTAAGTTCTGGTGGCGGAGGTTCCG | |
| | GGGGAGGGGGTAGTGGTGGTGGTGGGTCAGACATTCAACTTACACAAAGTCC | |
| | ATCAACCCTCAGTGCGTCTGTAGGGGATCGGGTCACAATAACCTGCCGAGCC | |
| | AGCGAGTCTTTGGACAACTACGGAATAAGGTTCCTCACGTGGTTTCAGCAGA | |
| | AACCGGGCAAAGCACCCAAGCTCCTTATGTATGCCGCGAGCAACCAGGGTTC | |
| | CGGAGTCCCGAGCCGGTTTTCTGGTTCCGGGAGCGGTACGGAGTTCACACTC | |
| | ACAATATCTTCCCTGCAGCCTGATGACTTTGCCACCTACTATTGCCAGCAGA | |
| | CTAAAGAGGTTCCCTGGTCCTTTGGTCAGGGCACGAAAGTGGAAGTCAAA | |
| CTX-964 | MALPVTALLLPLALLLHAARPQVQLQQPGAEVVKPGASVKMSCKASGYTFTS | 101 |
| CAR | YYIHWIKQTPGQGLEWVGVIYPGNDDISYNQKFQGKATLTADKSSTTAYMQL | |
| CD28 costim. | SSLTSEDSAVYYCAREVRLRYFDVWGQGTTVTVSSGGGGGSGGGGSGGGGSE | |
| | IVLTQSPGSLAVSPGERVTMSCKSSQSVFFSSSQKNYLAWYQQIPGQSPRLL | |
| | IYWASTRESGVPDRFTGSGSGTDFTLTISSVQPEDLAIYYCHQYLSSRTFGQ | |
| | GTKLEIKSAAAFVPVFLPAKPTTTPAPRPPTPAPTIASQPLSLRPEACRPAA | |
| | GGAVHTRGLDFACDIYIWAPLAGTCGVLLLSLVITLYCNHRNRSKRSRLLHS | |
| | DYMNMTPRRPGPTRKHYQPYAPPRDFAAYRSRVKFSRSADAPAYQQGQNQLY | |
| | NELNLGRREEYDVLDKRRGRDPEMGGKPRRKNPQEGLYNELQKDKMAEAYSE | |
| | IGMKGERRRGKGHDGLYQGLSTATKDTYDALHMQALPPR | |
| CTX-964b | MALPVTALLLPLALLLHAARPQVQLQQPGAEVVKPGASVKMSCKASGYTFTS | 102 |
| CAR | YYIHWIKQTPGQGLEWVGVIYPGNDDISYNQKFQGKATLTADKSSTTAYMQL | |
| 41BB costim. | SSLTSEDSAVYYCAREVRLRYFDVWGQGTTVTVSSGGGGGSGGGGSGGGGSE | |
| | IVLTQSPGSLAVSPGERVTMSCKSSQSVFFSSSQKNYLAWYQQIPGQSPRLL | |
| | IYWASTRESGVPDRFTGSGSGTDFTLTISSVQPEDLAIYYCHQYLSSRTFGQ | |
| | GTKLEIKSAAAFVPVFLPAKPTTTPAPRPPTPAPTIASQPLSLRPEACRPAA | |
| | GGAVHTRGLDFACDIYIQAPLAGTCGVLLLSLVITLYCNHRNRKRGRKKLLY | |
| | IFKQPFMRPVQTTQEEDGCSCRFPEEEEGGCELRVKFSRSADAPAYQQGQNQ | |
| | LYNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| CTX-965 | MALPVTALLLPLALLLHAARPEIVLTQSPGSLAVSPGERVTMSCKSSQSVFF | 103 |
| CAR | SSSQKNYLAWYQQIPGQSPRLLIYWASTRESGVPDRFTGSGSGTDFTLTISS | |
| CD28 costim. | VQPEDLAIYYCHQYLSSRTFGQGTKLEIKGGGGGSGGGGSGGGGSQVQLQQP | |
| | GAEVVKPGASVKMSCKASGYTFTSYYIHQIKQTPGQGLEWVGVIYPGNDDIS | |
| | YNQKFQGKATLTADKSSTTAYMQLSSLTSEDSAVYYCAREVRLRYFDVQGQG | |
| | TTVTVSSSAAAFVPVFLPAKPTTTPAPRPPTPAPTIASQPLSLRPEACRPAA | |
| | GGAVHTRGLDFACDIYIWAPLAGTCGVLLLSLVITLYCNHRNRSKRSRLLHS | |
| | DYMNMTPRRPGPTRKHYQPYAPPRDFAAYRSRVKFSRSADAPAYQQGQNQLY | |
| | NELNLGRREEYDVLDKRRGRDPEMGGKPRRKNPQEGLYNELQKDKMAEAYSE | |
| | IGMKGERRRGKGHDGLYQGLSTATKDTYDALHMQALPPR | |
| CTX-965b | MALPVTALLLPLALLLHAARPEIVLTQSPGSLAVSPGERVTMSCKSSQSVFF | 104 |
| CAR | SSSQKNYLAWYQQIPGQSPRLLIYWASTRESGVPDRFTGSGSGTDFTLTISS | |
| 41BB costim. | VQPEDLAIYYCHQYLSSRTFGQGTKLEIKGGGGGSGGGGSGGGGSQVQLQQP | |
| | GAEVVKPGASVKMSCKASGYTFTSYYIHWIKQTPGQGLEWVGVIYPGNDDIS | |
| | YNQKFQGKATLTADKSSTTAYMQLSSLTSEDSAVYYCAREVRLRYFDVWGQG | |
| | TTVTVSSSAAAFVPVFLPAKPTTTPAPRPPTPAPTIASQPLSLRPEACRPAA | |
| | GGAVHTRGLDFACDIYIWAPLAGTCGVLLLSLVITLYCNHRNRKRGRKKLLY | |
| | IFKQPFMRPVQTTQEEDGCSCRFPEEEEGGCELRVKFSRSADAPAYQQGQNQ | |
| | LYNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| CTX-969 | MALPVTALLLPLALLLHAARPQVQLVQSGAEVKKPGASVKVSCKASGYTFTN | 105 |
| CAR | YDINWVRQAPGQGLEWIGWIYPGDGSTKYNEKFKAKATLTADTSTSTAYMEL | |
| CD28 costim. | RSLRSDDTAVYYCASGYEDAMDYWGQGTTVTVSSGGGGGSGGGGSGGGGSDI | |
| | QMTQSPSSLSASVGDRVTINCKASQDINSYLSWFQQKPGKAPKTLIYRANRL | |
| | VDGVPSRFSGSGSGQDYTLTISSLQPEDFAT | |
| | YYCLQYDEFPLTFGGGTKVEIKSAAAFVPVFLPAKPTTTPAPRPPTPAPTIA | |
| | SQPLSLRPEACRPAAGGAVHTRGLDFACDIYIWAPLAGTCGVLLLSLVITLY | |
| | CNHRNRSKRSRLLHSDYMNMTPRRPGPTRKHYQPYAPPRDFAAYRSRVKFSR | |
| | SADAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQALP | |
| | PR | |
| CTX-969b | MALPVTALLLPLALLLHAARPQVQLVQSGAEVKKPGASVKVSCKASGYTFTN | 106 |
| CAR | YDINWVRQAPGQGLEWIGWIYPGDGSTKYNEKFKAKATLTADTSTSTAYMEL | |
| 41BB costim. | RSLRSDDTAVYYCASGYEDAMDYWGQGTTVTVSSGGGGGSGGGGSGGGGSDI | |
| | QMTQSPSSLSASVGDRVTINCKASQDINSYLSWFQQKPGKAPKTLIYRANRL | |
| | VDGVPSRFSGSGSGQDYTLTISSLQPEDFAT | |
| | YYCLQYDEFPLTFGGGTKVEIKSAAAFVPVFLPAKPTTTPAPRPPTPAPTIA | |
| | SQPLSLRPEACRPAAGGAVHTRGLDFACDIYIWAPLAGTCGVLLLSLVITLY | |
| | CNHRNRKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKNPQE | |
| | GLYNELQKDKMAEAYSEIGMKGERRRGKGHDGLYQGLSTATKDTYDALHMQA | |
| | LPPR | |
| CTX-970 | MALPVTALLLPLALLLHAARPDIQMTQSPSSLSASVGDRVTINCKASQDINS | 107 |
| CAR | YLSWFQQKPGKAPKTLIYRANRLVDGVPSRFSGSGSGQDYTLTISSLQPEDF | |
| CD28 costim. | ATYYCLQYDEFPLTFGGGTKVEIKGGGGGSGGGGSGGGGSQVQLVQSGAEVK | |
| | KPGASVKVSCKASGYTFTNYDINWVRQAPGQGLEWIGWIYPGDGSTKYNEKF | |
| | KAKATLTADTSTSTAYMELRSLRSDDTAVYYCASGYEDAMDYWGQGTTVTVS | |
| | SSAAAFVPVFLPAKPTTTPAPRPPTPAPTIASQOLSLRPEACRPAAGGAVHT | |
| | RGLDFACDIYIWAPLAGTCGVLLLSLVITLYCNHRNRSKRSRLLHSDYMNMT | |
| | PRRPGPTRKHYQPYAPPRDFAAYRSRVKFSRSADAPAYQQGQNQLYNELNLG | |
| | RREEYDVLDKRRGRDPEMGGKPRRKNPQEGLYNELQKDKMAEAYSEIGMKGE | |
| | RRRGKGHDGLYQGLSTATKDTYDALHMQALPPR | |
| CTX-970b | MALPVTALLLPLALLLHAARPDIQMTQSPSSLSASVGDRVTINCKASQDINS | 108 |
| CAR | YLSWFQQKPGKAPKTLIYRANRLVDGVPSRFSGSGSGQDYTLTISSLQPEDF | |
| 41BB costim. | ATYYCLQYDEFPLTFGGGTKVEIKGGGGGSGGGGSGGGGSQVQLVQSGAEVK | |
| | KPGASVKVSCKASGYTFTNYDINWVRQAPGQGLEWIGWIYPGDGSTKYNEKF | |
| | KAKATLTADTSTSTAYMELRSLRSDDTAVYYCASGYEDAMDYWGQGTTVTVS | |
| | SSAAAFVPVFLPAKPTTTPAPRPPTPAPTIASQPLSLRPEACRPAAGGAVHT | |
| | RGLDFACDIYIWAPLAGTCGVLLLSLVITLYCNHRNRKRGRKKLLYIFKQPF | |
| | MRPVQTTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAYSEIGMK | |
| | GERRRGKGHDGLYQGLSTATKDTYDALHMQALPPR | |
| CTX-981 Donor | GAGATGTAAGGAGCTGCTGTGACTTGCTCAAGGCCTTATATCGAGTAAACGG | 109 |
| template | TAGTGCTGGGGCTTAGACGCAGGTGTTCTGATTTATAGTTCAAAACCTCTAT | |
| LHA or RHA | CAATGAGAGAGCAATCTCCTGGTAATGTGATAGATTTCCCAACTTAATGCCA | |
| CD28 costim | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGATATACAGCTCACGCAGAGTCCATCAACACTGT | |
| | CCGCCAGTGTCGGTGACCGGGTTACTATTACGTGCCGCGCAAGCGAATCTCT | |
| | GGATAATTATGGTATCCGGTTTCTGACATGGTTTCAGCAAAAACCGGGGAAA | |
| | GCTCCCAAGCTGCTTATGTACGCCGCCTCTAATCAGGGGTCAGGTGTCCCTA | |
| | GCCGGTTCTCCGGTTCCGGTAGTGGCACGGAATTCACTCTCACAATCAGTTC | |
| | ACTCCAGCCGGATGACTTTGCAACGTATTATTGTCAACAAACGAAGGAGGTT | |
| | CCTTGGTCTTTCGGTCAGGGAACTAAGGTTGAGGTTAAGGGAGGAGGTGGTT | |
| | CTGGCGGAGGCGGATCTGGTGGCGGAGGTTCCGAGGTACAACTTGTGCAAAG | |
| | TGGGGCTGAGGTTAAAAAACCCGGCAGCTCTGTCAAAGTTTCCTGTAAGGCT | |
| | AGTGGTTACACCATCACTGACTCCAATATACACTGGGTTAGACAGGCTCCAG | |
| | GGCAGTCACTTGAGTGGATAGGCTACATCTATCCATACAACGGAGGTACAGA | |
| | CTACAACCAAAAATTTAAAAACAGGGCGACGCTTACAGTCGATAACCCCACA | |
| | AATACAGCATATATGGAGCTGTCATCTTTGCGCAGCGAAGATACAGCTTTCT | |
| | ACTATTGTGTGAATGGTAATCCCTGGCTGGCCTATTGGGGGCAGGGAACTCT | |
| | TGTCACTGTTTCCAGTAGTGCTGCTGCCTTTGTCCCGGTATTTCTCCCAGCC | |
| | AAACCGACCACGACTCCCGCCCCGCGCCCTCCGACACCCGCTCCCACCATCG | |
| | CCTCTCAACCTCTTAGTCTTCGCCCCGAGGCATGCCGACCCGCCGCCGGGGG | |
| | TGCTGTTCATACGAGGGGCTTGGACTTCGCTTGTGATATTTACATTTGGGCT | |
| | CCGTTGGCGGGTACGTGCGGCGTCCTTTTGTTGTCACTCGTTATTACTTTGT | |
| | ATTGTAATCACAGGAATCGCTCAAAGCGGAGTAGGTTGTTGCATTCCGATTA | |
| | CATGAATATGACTCCTCGCCGGCCTGGGCCGACAAGAAAACATTACCAACCC | |
| | TATGCCCCCCCACGAGACTTCGCTGCGTACAGGTCCCGAGTGAAGTTTTCCC | |
| | GAAGCGCAGACGCTCCGGCATATCAGCAAGGACAGAATCAGCTGTATAACGA | |
| | ACTGAATTTGGGACGCCGCGAGGAGTATGACGTGCTTGATAAACGCCGGGGG | |
| | AGAGACCCGGAAATGGGGGGTAAACCCCGAAGAAAGAATCCCCAAGAAGGAC | |
| | TCTACAATGAACTCCAGAAGGATAAGATGGCGGAGGCCTACTCAGAAATAGG | |
| | TATGAAGGGCGAACGACGACGGGGAAAAGGTCACGATGGCCTCTACCAAGGG | |
| | TTGAGTACGGCAACCAAAGATACGTACGATGCACTGCATATGCAGGCCCTGC | |
| | CTCCCAGATAATAATAAAATCGCTATCCATCGAAGATGGATGTGTGTTGGTT | |
| | TTTTGTGTGTGGAGCAACAAATCTGACTTTGCATGTGCAAACGCCTTCAACA | |
| | ACAGCATTATTCCAGAAGACACCTTCTTCCCCAGCCCAGGTAAGGGCAGCTT | |
| | TGGTGCCTTCGCAGGCTGTTTCCTTGCTTCAGGAATGGCCAGGTTCTGCCCA | |
| | GAGCTCTGGTCAATGATGTCTAAAACTCCTCTGATTGGTGGTCTCGGCCTTA | |
| | TCCATTGCCACCAAAACCCTCTTTTTACTAAGAAACAGTGAGCCTTGTTCTG | |
| | GCAGTCCAGAGAATGACACGGGAAAAAAGCAGATGAAGAGAAGGTGGCAGGA | |
| | GAGGGCACGTGGCCCAGCCTCAGTCTCTCCAACTGAGTTCCTGCCTGCCTGC | |
| | CTTTGCTCAGACTGTTTGCCCCTTACTGCTCTTCTAGGCCTCATTCTAAGCC | |
| | CCTTCTCCAAGTTGCCTCTCCTTATTTCTCCCTGTCTGCCAAAAAATCTTTC | |
| | CCAGCTCACTAAGTCAGTCTCACGCAGTCACTCATTAACCCACCAATCACTG | |
| | ATTGTGCCGGCACATGAATGCACCAGGTGTTGAAGTGGAGGAATTAAAAAGT | |
| | CAGATGAGGGGTGTGCCCAGAGGAAGCACCATTCTAGTTGGGGGAGCCCATC | |
| | TGTCAGCTGGGAAAAGTCCAAATAACTTCAGATTGGAATGTGTTTTAACTCA | |
| | GGGTTGAGAAAACAGCTACCTTCAGGACAAAAGTCAGGGAAGGGCTCTCTGA | |
| | AGAAATGCTACTTGAAGATACCAGCCCTACCAAGGGCAGGGAGAGGACCCTA | |
| | TAGAGGCCTGGGACAGGAGCTCAATGAGAAAGG | |
| CTX-981 | ATGGCGCTTCCGGTGACAGCACTGCTCCTCCCCTTGGCGCTGTTGCTCCACG | 110 |
| CAR (nucleotide) | CAGCAAGGCCGGATATACAGCTCACGCAGAGTCCATCAACACTGTCCGCCAG | |
| CD28 costim. | TGTCGGTGACCGGGTTACTATTACGTGCCGCGCAAGCGAATCTCTGGATAAT | |
| | TATGGTATCCGGTTTCTGACATGGTTTCAGCAAAAACCGGGGAAAGCTCCCA | |
| | AGCTGCTTATGTACGCCGCCTCTAATCAGGGGTCAGGTGTCCCTAGCCGGTT | |
| | CTCCGGTTCCGGTAGTGGCACGGAATTCACTCTCACAATCAGTTCACTCCAG | |
| | CCGGATGACTTTGCAACGTATTATTGTCAACAAACGAAGGAGGTTCCTTGGT | |
| | CTTTCGGTCAGGGAACTAAGGTTGAGGTTAAGGGAGGAGGTGGTTCTGGCGG | |
| | AGGCGGATCTGGTGGCGGAGGTTCCGAGGTACAACTTGTGCAAAGTGGGGCT | |
| | GAGGTTAAAAAACCCGGCAGCTCTGTCAAAGTTTCCTGTAAGGCTAGTGGTT | |
| | ACACCATCACTGACTCCAATATACACTGGGTTAGACAGGCTCCAGGGCAGTC | |
| | ACTTGAGTGGATAGGCTACATCTATCCATACAACGGAGGTACAGACTACAAC | |
| | CAAAAATTTAAAAACAGGGCGACGCTTACAGTCGATAACCCCACAAATACAG | |
| | CATATATGGAGCTGTCATCTTTGCGCAGCGAAGATACAGCTTTCTACTATTG | |
| | TGTGAATGGTAATCCCTGGCTGGCCTATTGGGGGCAGGGAACTCTTGTCACT | |
| | GTTTCCAGTAGTGCTGCTGCCTTTGTCCCGGTATTTCTCCCAGCCAAACCGA | |
| | CCACGACTCCCGCCCCGCGCCCTCCGACACCCGCTCCCACCATCGCCTCTCA | |
| | ACCTCTTAGTCTTCGCCCCGAGGCATGCCGACCCGCCGCCGGGGGTGCTGTT | |
| | CATACGAGGGGCTTGGACTTCGCTTGTGATATTTACATTTGGGCTCCGTTGG | |
| | CGGGTACGTGCGGCGTCCTTTTGTTGTCACTCGTTATTACTTTGTATTGTAA | |
| | TCACAGGAATCGCTCAAAGCGGAGTAGGTTGTTGCATTCCGATTACATGAAT | |
| | ATGACTCCTCGCCGGCCTGGGCCGACAAGAAAACATTACCAACCCTATGCCC | |
| | CCCCACGAGACTTCGCTGCGTACAGGTCCCGAGTGAAGTTTTCCCGAAGCGC | |
| | AGACGCTCCGGCATATCAGCAAGGACAGAATCAGCTGTATAACGAACTGAAT | |
| | TTGGGACGCCGCGAGGAGTATGACGTGCTTGATAAACGCCGGGGGAGAGACC | |
| | CGGAAATGGGGGGTAAACCCCGAAGAAAGAATCCCCAAGAAGGACTCTACAA | |
| | TGAACTCCAGAAGGATAAGATGGCGGAGGCCTACTCAGAAATAGGTATGAAG | |
| | GGCGAACGACGACGGGGAAAAGGTCACGATGGCCTCTACCAAGGGTTGAGTA | |
| | CGGCAACCAAAGATACGTACGATGCACTGCATATGCAGGCCCTGCCTCCCAG | |
| | A | |
| CTX-981 | MALPVTALLLPLALLLHAARPDIQLTQSPSTLSASVGDRVTITCRASESLDN | 111 |
| CAR | YGIRFLTWFQQKPGKAPKLLMYAASNQGSGVPSRFSGSGSGTEFTLTISSLQ | |
| (amino acid) | PDDFATYYCQQTKEVPWSFGQGTKVEVKGGGGSGGGGSGGGGSEVQLVQSGA | |
| CD28 costim. | EVKKPGSSVKVSCKASGYTITDSNIHWVRQAPGQSLEWIGYIYPYNGGTDYN | |
| | QKFKNRATLTVDNPTNTAYMELSSLRSEDTAFYYCVNGNPWLAYWGQGTLVT | |
| | VSSSAAAFVPVFLPAKPTTTPAPRPPTPAPTIASQPLSLRPEACRPAAGGAV | |
| | HTRGLDFACDIYIWAPLAGTCGVLLLSLVITLYCNHRNRSKRSRLLHSDYMN | |
| | MTPRRPGPTRKHYQPYAPPRDFAAYRSRVKFSRSADAPAYQQGQNQLYNELN | |
| | LGRREEYDVLDKRRGRDPEMGGKPRRKNPQEGLYNELQKDKMAEAYSEIGMK | |
| | GERRRGKGHDGLYQGLSTATKDTYDALHMQALPPR | |
| CTX-981b Donor | GAGATGTAAGGAGCTGCTGTGACTTGCTCAAGGCCTTATATCGAGTAAACGG | 112 |
| template | TAGTGCTGGGGCTTAGACGCAGGTGTTCTGATTTATAGTTCAAAACCTCTAT | |
| LHA or RHA | CAATGAGAGAGCAATCTCCTGGTAATGTGATAGATTTCCCAACTTAATGCCA | |
| 41BB costim | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGATATACAGCTCACGCAGAGTCCATCAACACTGT | |
| | CCGCCAGTGTCGGTGACCGGGTTACTATTACGTGCCGCGCAAGCGAATCTCT | |
| | GGATAATTATGGTATCCGGTTTCTGACATGGTTTCAGCAAAAACCGGGGAAA | |
| | GCTCCCAAGCTGCTTATGTACGCCGCCTCTAATCAGGGGTCAGGTGTCCCTA | |
| | GCCGGTTCTCCGGTTCCGGTAGTGGCACGGAATTCACTCTCACAATCAGTTC | |
| | ACTCCAGCCGGATGACTTTGCAACGTATTATTGTCAACAAACGAAGGAGGTT | |
| | CCTTGGTCTTTCGGTCAGGGAACTAAGGTTGAGGTTAAGGGAGGAGGTGGTT | |
| | CTGGCGGAGGCGGATCTGGTGGCGGAGGTTCCGAGGTACAACTTGTGCAAAG | |
| | TGGGGCTGAGGTTAAAAAACCCGGCAGCTCTGTCAAAGTTTCCTGTAAGGCT | |
| | AGTGGTTACACCATCACTGACTCCAATATACACTGGGTTAGACAGGCTCCAG | |
| | GGCAGTCACTTGAGTGGATAGGCTACATCTATCCATACAACGGAGGTACAGA | |
| | CTACAACCAAAAATTTAAAAACAGGGCGACGCTTACAGTCGATAACCCCACA | |
| | AATACAGCATATATGGAGCTGTCATCTTTGCGCAGCGAAGATACAGCTTTCT | |
| | ACTATTGTGTGAATGGTAATCCCTGGCTGGCCTATTGGGGGCAGGGAACTCT | |
| | TGTCACTGTTTCCAGTAGTGCTGCTGCCTTTGTCCCGGTATTTCTCCCAGCC | |
| | AAACCGACCACGACTCCCGCCCCGCGCCCTCCGACACCCGCTCCCACCATCG | |
| | CCTCTCAACCTCTTAGTCTTCGCCCCGAGGCATGCCGACCCGCCGCCGGGGG | |
| | TGCTGTTCATACGAGGGGCTTGGACTTCGCTTGTGATATTTACATTTGGGCT | |
| | CCGTTGGCGGGTACGTGCGGCGTCCTTTTGTTGTCACTCGTTATTACTTTGT | |
| | ATTGTAATCACAGGAATCGCAAACGGGGCAGAAAGAAACTCCTGTATATATT | |
| | CAAACAACCATTTATGAGACCAGTACAAACTACTCAAGAGGAAGATGGCTGT | |
| | AGCTGCCGATTTCCAGAAGAAGAAGAAGGAGGATGTGAACTGCGAGTGAAGT | |
| | TTTCCCGAAGCGCAGACGCTCCGGCATATCAGCAAGGACAGAATCAGCTGTA | |
| | TAACGAACTGAATTTGGGACGCCGCGAGGAGTATGACGTGCTTGATAAACGC | |
| | CGGGGGAGAGACCCGGAAATGGGGGGTAAACCCCGAAGAAAGAATCCCCAAG | |
| | AAGGACTCTACAATGAACTCCAGAAGGATAAGATGGCGGAGGCCTACTCAGA | |
| | AATAGGTATGAAGGGCGAACGACGACGGGGAAAAGGTCACGATGGCCTCTAC | |
| | CAAGGGTTGAGTACGGCAACCAAAGATACGTACGATGCACTGCATATGCAGG | |
| | CCCTGCCTCCCAGATAATAATAAAATCGCTATCCATCGAAGATGGATGTGTG | |
| | TTGGTTTTTTGTGTGTGGAGCAACAAATCTGACTTTGCATGTGCAAACGCCT | |
| | TCAACAACAGCATTATTCCAGAAGACACCTTCTTCCCCAGCCCAGGTAAGGG | |
| | CAGCTTTGGTGCCTTCGCAGGCTGTTTCCTTGCTTCAGGAATGGCCAGGTTC | |
| | TGCCCAGAGCTCTGGTCAATGATGTCTAAAACTCCTCTGATTGGTGGTCTCG | |
| | GCCTTATCCATTGCCACCAAAACCCTCTTTTTACTAAGAAACAGTGAGCCTT | |
| | GTTCTGGCAGTCCAGAGAATGACACGGGAAAAAAGCAGATGAAGAGAAGGTG | |
| | GCAGGAGAGGGCACGTGGCCCAGCCTCAGTCTCTCCAACTGAGTTCCTGCCT | |
| | GCCTGCCTTTGCTCAGACTGTTTGCCCCTTACTGCTCTTCTAGGCCTCATTC | |
| | TAAGCCCCTTCTCCAAGTTGCCTCTCCTTATTTCTCCCTGTCTGCCAAAAAA | |
| | TCTTTCCCAGCTCACTAAGTCAGTCTCACGCAGTCACTCATTAACCCACCAA | |
| | TCACTGATTGTGCCGGCACATGAATGCACCAGGTGTTGAAGTGGAGGAATTA | |
| | AAAAGTCAGATGAGGGGTGTGCCCAGAGGAAGCACCATTCTAGTTGGGGGAG | |
| | CCCATCTGTCAGCTGGGAAAAGTCCAAATAACTTCAGATTGGAATGTGTTTT | |
| | AACTCAGGGTTGAGAAAACAGCTACCTTCAGGACAAAAGTCAGGGAAGGGCT | |
| | CTCTGAAGAAATGCTACTTGAAGATACCAGCCCTACCAAGGGCAGGGAGAGG | |
| | ACCCTATAGAGGCCTGGGACAGGAGCTCAATGAGAAAGG | |
| CTX-981b | ATGGCGCTTCCGGTGACAGCACTGCTCCTCCCCTTGGCGCTGTTGCTCCACG | 113 |
| CAR (nucleotide) | CAGCAAGGCCGGATATACAGCTCACGCAGAGTCCATCAACACTGTCCGCCAG | |
| 41BB costim. | TGTCGGTGACCGGGTTACTATTACGTGCCGCGCAAGCGAATCTCTGGATAAT | |
| | TATGGTATCCGGTTTCTGACATGGTTTCAGCAAAAACCGGGGAAAGCTCCCA | |
| | AGCTGCTTATGTACGCCGCCTCTAATCAGGGGTCAGGTGTCCCTAGCCGGTT | |
| | CTCCGGTTCCGGTAGTGGCACGGAATTCACTCTCACAATCAGTTCACTCCAG | |
| | CCGGATGACTTTGCAACGTATTATTGTCAACAAACGAAGGAGGTTCCTTGGT | |
| | CTTTCGGTCAGGGAACTAAGGTTGAGGTTAAGGGAGGAGGTGGTTCTGGCGG | |
| | AGGCGGATCTGGTGGCGGAGGTTCCGAGGTACAACTTGTGCAAAGTGGGGCT | |
| | GAGGTTAAAAAACCCGGCAGCTCTGTCAAAGTTTCCTGTAAGGCTAGTGGTT | |
| | ACACCATCACTGACTCCAATATACACTGGGTTAGACAGGCTCCAGGGCAGTC | |
| | ACTTGAGTGGATAGGCTACATCTATCCATACAACGGAGGTACAGACTACAAC | |
| | CAAAAATTTAAAAACAGGGCGACGCTTACAGTCGATAACCCCACAAATACAG | |
| | CATATATGGAGCTGTCATCTTTGCGCAGCGAAGATACAGCTTTCTACTATTG | |
| | TGTGAATGGTAATCCCTGGCTGGCCTATTGGGGGCAGGGAACTCTTGTCACT | |
| | GTTTCCAGTAGTGCTGCTGCCTTTGTCCCGGTATTTCTCCCAGCCAAACCGA | |
| | CCACGACTCCCGCCCCGCGCCCTCCGACACCCGCTCCCACCATCGCCTCTCA | |
| | ACCTCTTAGTCTTCGCCCCGAGGCATGCCGACCCGCCGCCGGGGGTGCTGTT | |
| | CATACGAGGGGCTTGGACTTCGCTTGTGATATTTACATTTGGGCTCCGTTGG | |
| | CGGGTACGTGCGGCGTCCTTTTGTTGTCACTCGTTATTACTTTGTATTGTAA | |
| | TCACAGGAATCGCAAACGGGGCAGAAAGAAACTCCTGTATATATTCAAACAA | |
| | CCATTTATGAGACCAGTACAAACTACTCAAGAGGAAGATGGCTGTAGCTGCC | |
| | GATTTCCAGAAGAAGAAGAAGGAGGATGTGAACTGCGAGTGAAGTTTTCCCG | |
| | AAGCGCAGACGCTCCGGCATATCAGCAAGGACAGAATCAGCTGTATAACGAA | |
| | CTGAATTTGGGACGCCGCGAGGAGTATGACGTGCTTGATAAACGCCGGGGGA | |
| | GAGACCCGGAAATGGGGGGTAAACCCCGAAGAAAGAATCCCCAAGAAGGACT | |
| | CTACAATGAACTCCAGAAGGATAAGATGGCGGAGGCCTACTCAGAAATAGGT | |
| | ATGAAGGGCGAACGACGACGGGGAAAAGGTCACGATGGCCTCTACCAAGGGT | |
| | TGAGTACGGCAACCAAAGATACGTACGATGCACTGCATATGCAGGCCCTGCC | |
| | TCCCAGA | |
| CTX-981b | MALPVTALLLPLALLLHAARPDIQLTQSPSTLSASVGDRVTITCRASESLDN | 114 |
| CAR | YGIRFLTWFQQKPGKAPKLLMYAASNQGAGVPSRFSGSGSGTEFTLTISSLQ | |
| (amino acid) | PDDFATYYCQQTKEVPWSFGQGTKVEVKGGGGSGGGGSGGGGSEVQLVQSGA | |
| 41BB costim. | EVKKPGSSVKVSCKASGYTITDSNIHWVRQAPGQSLEWIGYIYPYNGGTDYN | |
| | QKFKNRATLTVDNPTNTAYMELSSLRSEDTAFYYCVNGNPQLAYWGQGTLVT | |
| | VSSSAAAFVPVFLPAKPTTTPAPRPPTPAPTIASQPLSLRPEACRPAAGGAV | |
| | HTRGLDFACDIYIWAPLAGTCGVLLLSLVITLYCNHRNRKRGRKKLLYIFKQ | |
| | PFMRPVQTTQEEDGCSCRFPEEEEGGCELRVKFSRSADAPAYQQGQNQLYNE | |
| | LNLGRREEYDVLDKRRGRDPEMGGKPRRKNPQEGLYNELQKDKMAEAYSEIG | |
| | MKGERRRGKGHDGLYQGLSTATKDTYDALHMQALPPR | |
| CTX-982 | GAGATGTAAGGAGCTGCTGTGACTTGCTCAAGGCCTTATATCGAGTAAACGG | 115 |
| Donor | TAGTGCTGGGGCTTAGACGCAGGTGTTCTGATTTATAGTTCAAAACCTCTAT | |
| LHA or RHA | CAATGAGAGAGCAATCTCCTGGTAATGTGATAGATTTCCCAACTTAATGCCA | |
| CD28 costim | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGAGGTCCAACTTGTTCAATCCGGCGCTGAAGTGA | |
| | AAAAGCCAGGAAGTAGCGTAAAAGTAAGCTGTAAAGCTAGCGGTTACACCAT | |
| | TACCGACAGCAACATCCATTGGGTGCGGCAGGCGCCAGGACAATCCCTCGAG | |
| | TGGATAGGTTACATCTATCCTTACAACGGGGGAACAGATTATAATCAGAAGT | |
| | TCAAGAACCGGGCAACGCTCACTGTTGACAATCCCACTAATACTGCCTATAT | |
| | GGAGCTCTCCAGCCTCCGCAGTGAGGACACTGCGTTTTATTATTGCGTGAAT | |
| | GGCAACCCGTGGCTTGCTTATTGGGGACAGGGCACATTGGTTACAGTAAGTT | |
| | CTGGTGGCGGAGGTTCCGGGGGAGGGGGTAGTGGTGGTGGTGGGTCAGACAT | |
| | TCAACTTACACAAAGTCCATCAACCCTCAGTGCGTCTGTAGGGGATCGGGTC | |
| | ACAATAACCTGCCGAGCCAGCGAGTCTTTGGACAACTACGGAATAAGGTTCC | |
| | TCACGTGGTTTCAGCAGAAACCGGGCAAAGCACCCAAGCTCCTTATGTATGC | |
| | CGCGAGCAACCAGGGTTCCGGAGTCCCGAGCCGGTTTTCTGGTTCCGGGAGC | |
| | GGTACGGAGTTCACACTCACAATATCTTCCCTGCAGCCTGATGACTTTGCCA | |
| | CCTACTATTGCCAGCAGACTAAAGAGGTTCCCTGGTCCTTTGGTCAGGGCAC | |
| | GAAAGTGGAAGTCAAAAGTGCTGCTGCCTTTGTCCCGGTATTTCTCCCAGCC | |
| | AAACCGACCACGACTCCCGCCCCGCGCCCTCCGACACCCGCTCCCACCATCG | |
| | CCTCTCAACCTCTTAGTCTTCGCCCCGAGGCATGCCGACCCGCCGCCGGGGG | |
| | TGCTGTTCATACGAGGGGCTTGGACTTCGCTTGTGATATTTACATTTGGGCT | |
| | CCGTTGGCGGGTACGTGCGGCGTCCTTTTGTTGTCACTCGTTATTACTTTGT | |
| | ATTGTAATCACAGGAATCGCTCAAAGCGGAGTAGGTTGTTGCATTCCGATTA | |
| | CATGAATATGACTCCTCGCCGGCCTGGGCCGACAAGAAAACATTACCAACCC | |
| | TATGCCCCCCCACGAGACTTCGCTGCGTACAGGTCCCGAGTGAAGTTTTCCC | |
| | GAAGCGCAGACGCTCCGGCATATCAGCAAGGACAGAATCAGCTGTATAACGA | |
| | ACTGAATTTGGGACGCCGCGAGGAGTATGACGTGCTTGATAAACGCCGGGGG | |
| | AGAGACCCGGAAATGGGGGGTAAACCCCGAAGAAAGAATCCCCAAGAAGGAC | |
| | TCTACAATGAACTCCAGAAGGATAAGATGGCGGAGGCCTACTCAGAAATAGG | |
| | TATGAAGGGCGAACGACGACGGGGAAAAGGTCACGATGGCCTCTACCAAGGG | |
| | TTGAGTACGGCAACCAAAGATACGTACGATGCACTGCATATGCAGGCCCTGC | |
| | CTCCCAGATAATAATAAAATCGCTATCCATCGAAGATGGATGTGTGTTGGTT | |
| | TTTTGTGTGTGGAGCAACAAATCTGACTTTGCATGTGCAAACGCCTTCAACA | |
| | ACAGCATTATTCCAGAAGACACCTTCTTCCCCAGCCCAGGTAAGGGCAGCTT | |
| | TGGTGCCTTCGCAGGCTGTTTCCTTGCTTCAGGAATGGCCAGGTTCTGCCCA | |
| | GAGCTCTGGTCAATGATGTCTAAAACTCCTCTGATTGGTGGTCTCGGCCTTA | |
| | TCCATTGCCACCAAAACCCTCTTTTTACTAAGAAACAGTGAGCCTTGTTCTG | |
| | GCAGTCCAGAGAATGACACGGGGAAAAAGCAGATGAAGAGAAGGTGGCAGGA | |
| | GAGGGCACGTGGCCCAGCCTCAGTCTCTCCAACTGAGTTCCTGCCTGCCTGC | |
| | CTTTGCTCAGACTGTTTGCCCCTTACTGCTCTTCTAGGCCTCATTCTAAGCC | |
| | CCTTCTCCAAGTTGCCTCTCCTTATTTCTCCCTGTCTGCCAAAAAATCTTTC | |
| | CCAGCTCACTAAGTCAGTCTCACGCAGTCACTCATTAACCCACCAATCACTG | |
| | ATTGTGCCGGCACATGAATGCACCAGGTGTTGAAGTGGAGGAATTAAAAAGT | |
| | CAGATGAGGGGTGTGCCCAGAGGAAGCACCATTCTAGTTGGGGGAGCCCATC | |
| | TGTCAGCTGGGAAAAGTCCAAATAACTTCAGATTGGAATGTGTTTTAACTCA | |
| | GGGTTGAGAAAACAGCTACCTTCAGGACAAAAGTCAGGGAAGGGCTCTCTGA | |
| | AGAAATGCTACTTGAAGATACCAGCCCTACCAAGGGCAGGGAGAGGACCCTA | |
| | TAGAGGCCTGGGACAGGAGCTCAATGAGAAAGG | |
| CTX-982 | ATGGCGCTTCCGGTGACAGCACTGCTCCTCCCCTTGGCGCTGTTGCTCCACG | 116 |
| CAR (nucleotide) | CAGCAAGGCCGGAGGTCCAACTTGTTCAATCCGGCGCTGAAGTGAAAAAGCC | |
| CD28 costim. | AGGAAGTAGCGTAAAAGTAAGCTGTAAAGCTAGCGGTTACACCATTACCGAC | |
| | AGCAACATCCATTGGGTGCGGCAGGCGCCAGGACAATCCCTCGAGTGGATAG | |
| | GTTACATCTATCCTTACAACGGGGGAACAGATTATAATCAGAAGTTCAAGAA | |
| | CCGGGCAACGCTCACTGTTGACAATCCCACTAATACTGCCTATATGGAGCTC | |
| | TCCAGCCTCCGCAGTGAGGACACTGCGTTTTATTATTGCGTGAATGGCAACC | |
| | CGTGGCTTGCTTATTGGGGACAGGGCACATTGGTTACAGTAAGTTCTGGTGG | |
| | CGGAGGTTCCGGGGGAGGGGGTAGTGGTGGTGGTGGGTCAGACATTCAACTT | |
| | ACACAAAGTCCATCAACCCTCAGTGCGTCTGTAGGGGATCGGGTCACAATAA | |
| | CCTGCCGAGCCAGCGAGTCTTTGGACAACTACGGAATAAGGTTCCTCACGTG | |
| | GTTTCAGCAGAAACCGGGCAAAGCACCCAAGCTCCTTATGTATGCCGCGAGC | |
| | AACCAGGGTTCCGGAGTCCCGAGCCGGTTTTCTGGTTCCGGGAGCGGTACGG | |
| | AGTTCACACTCACAATATCTTCCCTGCAGCCTGATGACTTTGCCACCTACTA | |
| | TTGCCAGCAGACTAAAGAGGTTCCCTGGTCCTTTGGTCAGGGCACGAAAGTG | |
| | GAAGTCAAAAGTGCTGCTGCCTTTGTCCCGGTATTTCTCCCAGCCAAACCGA | |
| | CCACGACTCCCGCCCCGCGCCCTCCGACACCCGCTCCCACCATCGCCTCTCA | |
| | ACCTCTTAGTCTTCGCCCCGAGGCATGCCGACCCGCCGCCGGGGGTGCTGTT | |
| | CATACGAGGGGCTTGGACTTCGCTTGTGATATTTACATTTGGGCTCCGTTGG | |
| | CGGGTACGTGCGGCGTCCTTTTGTTGTCACTCGTTATTACTTTGTATTGTAA | |
| | TCACAGGAATCGCTCAAAGCGGAGTAGGTTGTTGCATTCCGATTACATGAAT | |
| | ATGACTCCTCGCCGGCCTGGGCCGACAAGAAAACATTACCAACCCTATGCCC | |
| | CCCCACGAGACTTCGCTGCGTACAGGTCCCGAGTGAAGTTTTCCCGAAGCGC | |
| | AGACGCTCCGGCATATCAGCAAGGACAGAATCAGCTGTATAACGAACTGAAT | |
| | TTGGGACGCCGCGAGGAGTATGACGTGCTTGATAAACGCCGGGGGAGAGACC | |
| | CGGAAATGGGGGGTAAACCCCGAAGAAAGAATCCCCAAGAAGGACTCTACAA | |
| | TGAACTCCAGAAGGATAAGATGGCGGAGGCCTACTCAGAAATAGGTATGAAG | |
| | GGCGAACGACGACGGGGAAAAGGTCACGATGGCCTCTACCAAGGGTTGAGTA | |
| | CGGCAACCAAAGATACGTACGATGCACTGCATATGCAGGCCCTGCCTCCCAG | |
| | A | |
| CTX-982 | MALPVTALLLPLALLLHAARPEVQLVQSGAEVKKPGSSVKVSCKASGYTITD | 117 |
| CAR | SNIHWVRQAPGQSLEWIGYIYPYNGGTDYNQKFKNRATLTVDNPTNTAYMEL | |
| (amino acid) | SSLRSEDTAFYYCVNGNPWLAYWGQGTLVTVSSGGGGSGGGGSGGGGSDIQL | |
| CD28 costim. | TQSPSTLSASVGDRVTITCRASESLDNYGIRFLTWFQQKPGKAPKLLMYAAS | |
| | NQGSGVPSRFSGSGSGTEFTLTISSLQPDDFATYYCQQTKEVPWSFGQGTKV | |
| | EVKSAAAFVPVFLPAKPTTTPAPRPPTPAPTIASQPLSLRPEACRPAAGGAV | |
| | HTRGLDFACDIYIWAPLAGTCGVLLLSLVITLYCNHRNRSKRSRLLHSDYMN | |
| | MTPRRPGPTRKHYQPYAPPRDFAAYRSRVKFSRSADAPAYQQGQNQLYNELN | |
| | LGRREEYDVLDKRRGRDPEMGGKPRRKNPQEGLYNELQKDKMAEAYSEIGMK | |
| | GERRRGKGHDGLYQGLSTATKDTYDALHMQALPPR | |
| CTX-982b | GAGATGTAAGGAGCTGCTGTGACTTGCTCAAGGCCTTATATCGAGTAAACGG | 118 |
| Donor | TAGTGCTGGGGCTTAGACGCAGGTGTTCTGATTTATAGTTCAAAACCTCTAT | |
| LHA to RHA | CAATGAGAGAGCAATCTCCTGGTAATGTGATAGATTTCCCAACTTAATGCCA | |
| 41BB costim | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCAggctccggtgcccgtcagtgggcagagcgcac | |
| | atcgcccacagtccccgagaagttggggggaggggtcggcaattgaaccggt | |
| | gcctagagaaggtggcgcggggtaaactgggaaagtgatgtcgtgtactggc | |
| | tccgcctttttcccgagggtgggggagaaccgtatataagtgcagtagtcgc | |
| | cgtgaacgttctttttcgcaacgggtttgccgccagaacacaggtaagtgcc | |
| | gtgtgtggttcccgcgggcctggcctctttacgggttatggcccttgcgtgc | |
| | cttgaattacttccactggctgcagtacgtgattcttgatcccgagcttcgg | |
| | gttggaagtgggtgggagagttcgaggccttgcgcttaaggagccccttcgc | |
| | ctcgtgcttgagttgaggcctggcctgggcgctggggccgccgcgtgcgaat | |
| | ctggtggcaccttcgcgcctgtctcgctgctttcgataagtctctagccatt | |
| | taaaatttttgatgacctgctgcgacgctttttttctggcaagatagtcttg | |
| | taaatgcgggccaagatctgcacactggtatttcggtttttggggccgcggg | |
| | cggcgacggggcccgtgcgtcccagcgcacatgttcggcgaggcggggcctg | |
| | cgagcgcggccaccgagaatcggacgggggtagtctcaagctggccggcctg | |
| | ctctggtgcctggcctcgcgccgccgtgtatcgccccgccctgggcggcaag | |
| | gctggcccggtcggcaccagttgcgtgagcggaaagatggccgcttcccggc | |
| | cctgctgcagggagctcaaaatggaggacgcggcgctcgggagagcgggcgg | |
| | gtgagtcacccacacaaaggaaaagggcctttccgtcctcagccgtcgcttc | |
| | atgtgactccacggagtaccgggcgccgtccaggcacctcgattagttctcg | |
| | agcttttggagtacgtcgtctttaggttggggggaggggttttatgcgatgg | |
| | agtttccccacactgagtgggtggagactgaagttaggccagcttggcactt | |
| | gatgtaattctccttggaatttgccctttttgagtttggatcttggttcatt | |
| | ctcaagcctcagacagtggttcaaagtttttttcttccatttcaggtgtcgt | |
| | gaCCACCATGGCGCTTCCGGTGACAGCACTGCTCCTCCCCTTGGCGCTGTTG | |
| | CTCCACGCAGCAAGGCCGGAGGTCCAACTTGTTCAATCCGGCGCTGAAGTGA | |
| | AAAAGCCAGGAAGTAGCGTAAAAGTAAGCTGTAAAGCTAGCGGTTACACCAT | |
| | TACCGACAGCAACATCCATTGGGTGCGGCAGGCGCCAGGACAATCCCTCGAG | |
| | TGGATAGGTTACATCTATCCTTACAACGGGGGAACAGATTATAATCAGAAGT | |
| | TCAAGAACCGGGCAACGCTCACTGTTGACAATCCCACTAATACTGCCTATAT | |
| | GGAGCTCTCCAGCCTCCGCAGTGAGGACACTGCGTTTTATTATTGCGTGAAT | |
| | GGCAACCCGTGGCTTGCTTATTGGGGACAGGGCACATTGGTTACAGTAAGTT | |
| | CTGGTGGCGGAGGTTCCGGGGGAGGGGGTAGTGGTGGTGGTGGGTCAGACAT | |
| | TCAACTTACACAAAGTCCATCAACCCTCAGTGCGTCTGTAGGGGATCGGGTC | |
| | ACAATAACCTGCCGAGCCAGCGAGTCTTTGGACAACTACGGAATAAGGTTCC | |
| | TCACGTGGTTTCAGCAGAAACCGGGCAAAGCACCCAAGCTCCTTATGTATGC | |
| | CGCGAGCAACCAGGGTTCCGGAGTCCCGAGCCGGTTTTCTGGTTCCGGGAGC | |
| | GGTACGGAGTTCACACTCACAATATCTTCCCTGCAGCCTGATGACTTTGCCA | |
| | CCTACTATTGCCAGCAGACTAAAGAGGTTCCCTGGTCCTTTGGTCAGGGCAC | |
| | GAAAGTGGAAGTCAAAAGTGCTGCTGCCTTTGTCCCGGTATTTCTCCCAGCC | |
| | AAACCGACCACGACTCCCGCCCCGCGCCCTCCGACACCCGCTCCCACCATCG | |
| | CCTCTCAACCTCTTAGTCTTCGCCCCGAGGCATGCCGACCCGCCGCCGGGGG | |
| | TGCTGTTCATACGAGGGGCTTGGACTTCGCTTGTGATATTTACATTTGGGCT | |
| | CCGTTGGCGGGTACGTGCGGCGTCCTTTTGTTGTCACTCGTTATTACTTTGT | |
| | ATTGTAATCACAGGAATCGCAAACGGGGCAGAAAGAAACTCCTGTATATATT | |
| | CAAACAACCATTTATGAGACCAGTACAAACTACTCAAGAGGAAGATGGCTGT | |
| | AGCTGCCGATTTCCAGAAGAAGAAGAAGGAGGATGTGAACTGCGAGTGAAGT | |
| | TTTCCCGAAGCGCAGACGCTCCGGCATATCAGCAAGGACAGAATCAGCTGTA | |
| | TAACGAACTGAATTTGGGACGCCGCGAGGAGTATGACGTGCTTGATAAACGC | |
| | CGGGGGAGAGACCCGGAAATGGGGGGTAAACCCCGAAGAAAGAATCCCCAAG | |
| | AAGGACTCTACAATGAACTCCAGAAGGATAAGATGGCGGAGGCCTACTCAGA | |
| | AATAGGTATGAAGGGCGAACGACGACGGGGAAAAGGTCACGATGGCCTCTAC | |
| | CAAGGGTTGAGTACGGCAACCAAAGATACGTACGATGCACTGCATATGCAGG | |
| | CCCTGCCTCCCAGATAATAATAAAATCGCTATCCATCGAAGATGGATGTGTG | |
| | TTGGTTTTTTGTGTGTGGAGCAACAAATCTGACTTTGCATGTGCAAACGCCT | |
| | TCAACAACAGCATTATTCCAGAAGACACCTTCTTCCCCAGCCCAGGTAAGGG | |
| | CAGCTTTGGTGCCTTCGCAGGCTGTTTCCTTGCTTCAGGAATGGCCAGGTTC | |
| | TGCCCAGAGCTCTGGTCAATGATGTCTAAAACTCCTCTGATTGGTGGTCTCG | |
| | GCCTTATCCATTGCCACCAAAACCCTCTTTTTACTAAGAAACAGTGAGCCTT | |
| | GTTCTGGCAGTCCAGAGAATGACACGGGAAAAAAGCAGATGAAGAGAAGGTG | |
| | GCAGGAGAGGGCACGTGGCCCAGCCTCAGTCTCTCCAACTGAGTTCCTGCCT | |
| | GCCTGCCTTTGCTCAGACTGTTTGCCCCTTACTGCTCTTCTAGGCCTCATTC | |
| | TAAGCCCCTTCTCCAAGTTGCCTCTCCTTATTTCTCCCTGTCTGCCAAAAAA | |
| | TCTTTCCCAGCTCACTAAGTCAGTCTCACGCAGTCACTCATTAACCCACCAA | |
| | TCACTGATTGTGCCGGCACATGAATGCACCAGGTGTTGAAGTGGAGGAATTA | |
| | AAAAGTCAGATGAGGGGTGTGCCCAGAGGAAGCACCATTCTAGTTGGGGGAG | |
| | CCCATCTGTCAGCTGGGAAAAGTCCAAATAACTTCAGATTGGAATGTGTTTT | |
| | AACTCAGGGTTGAGAAAACAGCTACCTTCAGGACAAAAGTCAGGGAAGGGCT | |
| | CTCTGAAGAAATGCTACTTGAAGATACCAGCCCTACCAAGGGCAGGGAGAGG | |
| | ACCCTATAGAGGCCTGGGACAGGAGCTCAATGAGAAAGG | |
| CTX-982b | ATGGCGCTTCCGGTGACAGCACTGCTCCTCCCCTTGGCGCTGTTGCTCCACG | 119 |
| CAR (nucleotide) | CAGCAAGGCCGGAGGTCCAACTTGTTCAATCCGGCGCTGAAGTGAAAAAGCC | |
| 41BB costim. | AGGAAGTAGCGTAAAAGTAAGCTGTAAAGCTAGCGGTTACACCATTACCGAC | |
| | AGCAACATCCATTGGGTGCGGCAGGCGCCAGGACAATCCCTCGAGTGGATAG | |
| | GTTACATCTATCCTTACAACGGGGGAACAGATTATAATCAGAAGTTCAAGAA | |
| | CCGGGCAACGCTCACTGTTGACAATCCCACTAATACTGCCTATATGGAGCTC | |
| | TCCAGCCTCCGCAGTGAGGACACTGCGTTTTATTATTGCGTGAATGGCAACC | |
| | CGTGGCTTGCTTATTGGGGACAGGGCACATTGGTTACAGTAAGTTCTGGTGG | |
| | CGGAGGTTCCGGGGGAGGGGGTAGTGGTGGTGGTGGGTCAGACATTCAACTT | |
| | ACACAAAGTCCATCAACCCTCAGTGCGTCTGTAGGGGATCGGGTCACAATAA | |
| | CCTGCCGAGCCAGCGAGTCTTTGGACAACTACGGAATAAGGTTCCTCACGTG | |
| | GTTTCAGCAGAAACCGGGCAAAGCACCCAAGCTCCTTATGTATGCCGCGAGC | |
| | AACCAGGGTTCCGGAGTCCCGAGCCGGTTTTCTGGTTCCGGGAGCGGTACGG | |
| | AGTTCACACTCACAATATCTTCCCTGCAGCCTGATGACTTTGCCACCTACTA | |
| | TTGCCAGCAGACTAAAGAGGTTCCCTGGTCCTTTGGTCAGGGCACGAAAGTG | |
| | GAAGTCAAAAGTGCTGCTGCCTTTGTCCCGGTATTTCTCCCAGCCAAACCGA | |
| | CCACGACTCCCGCCCCGCGCCCTCCGACACCCGCTCCCACCATCGCCTCTCA | |
| | ACCTCTTAGTCTTCGCCCCGAGGCATGCCGACCCGCCGCCGGGGGTGCTGTT | |
| | CATACGAGGGGCTTGGACTTCGCTTGTGATATTTACATTTGGGCTCCGTTGG | |
| | CGGGTACGTGCGGCGTCCTTTTGTTGTCACTCGTTATTACTTTGTATTGTAA | |
| | TCACAGGAATCGCAAACGGGGCAGAAAGAAACTCCTGTATATATTCAAACAA | |
| | CCATTTATGAGACCAGTACAAACTACTCAAGAGGAAGATGGCTGTAGCTGCC | |
| | GATTTCCAGAAGAAGAAGAAGGAGGATGTGAACTGCGAGTGAAGTTTTCCCG | |
| | AAGCGCAGACGCTCCGGCATATCAGCAAGGACAGAATCAGCTGTATAACGAA | |
| | CTGAATTTGGGACGCCGCGAGGAGTATGACGTGCTTGATAAACGCCGGGGGA | |
| | GAGACCCGGAAATGGGGGGTAAACCCCGAAGAAAGAATCCCCAAGAAGGACT | |
| | CTACAATGAACTCCAGAAGGATAAGATGGCGGAGGCCTACTCAGAAATAGGT | |
| | ATGAAGGGCGAACGACGACGGGGAAAAGGTCACGATGGCCTCTACCAAGGGT | |
| | TGAGTACGGCAACCAAAGATACGTACGATGCACTGCATATGCAGGCCCTGCC | |
| | TCCCAGA | |
| CTX-982b | MALPVTALLLPLALLLHAARPEVQLVQSGAEVKKPGSSVKVSCKASGYTITD | 120 |
| CAR | SNIHWVRQAPGQSLEWIGYIYPYNGGTDYNQKFKNRATLTVDNPTNTAYMEL | |
| (amino acid) | SSLRSEDTAFYYCVNGNPWLAYWGQGTLVTVSSGGGGSGGGGSGGGGSDIQL | |
| 41BB costim. | TQSPSTLSASVGDRVTITCRASESLDNYGIRFLTWFQQKPGKAPKLLMYAAS | |
| | NQGSGVPSRFSGSGSGTEFTLTISSLQPDDFATYYCQQTKEVPWSFGQGTKV | |
| | EVKSAAAFVPVFLPAKPTTTPAPRPPTPAPTIASQPLSLRPEACRPAAGGAV | |
| | HTRGLDFACDIYIWAPLAGTCGVLLLSLVITLYCNHRNRKRGRKKLLYIFKQ | |
| | PFMRPVQTTQEEDGCSCRFPEEEEGGCELRVKFSRSADAPAYQQGQNQLYNE | |
| | LNLGRREEYDVLDKRRGRDPEMGGKPRRKNPQEGLYNELQKDKMAEAYSEIG | |
| | MKGERRRGKGHDGLYQGLSTATKDTYDALHMQALPPR | |
| CD8 signal | MALPVTALLLPLALLLHAARP | 121 |
| peptide | | |
| CD8a extracellular + | GCTGCTGCCTTTGTCCCGGTATTTCTCCCAGCCAAACCGACCACGACTCCCG | 122 |
| transmembrane + | CCCCGCGCCCTCCGACACCCGCTCCCACCATCGCCTCTCAACCTCTTAGTCT | |
| 5′Linker | TCGCCCCGAGGCATGCCGACCCGCCGCCGGGGGTGCTGTTCATACGAGGGGC | |
| (underlined) | TTGGACTTCGCTTGTGATATTTACATTTGGGCTCCGTTGGCGGGTACGTGCG | |
| | GCGTCCTTTTGTTGTCACTCGTTATTACTTTGTATTGTAATCACAGGAATCG | |
| | C | |
| CD8a extracellular + | SAAAFVPVFLPAKPTTTPAPRPPTPAPTIASQPLSLRPEACRPAAGGAVHTR | 123 |
| transmembrane + | GLDFACDIYIWAPLAGTCGVLLLSLVITLYCNHRNR | |
| 5′Linker | | |
| (underlined) | | |
| CD8a extracellular + | TTTGTCCCGGTATTTCTCCCAGCCAAACCGACCACGACTCCCGCCCCGCGCC | 124 |
| transmembrane + | CTCCGACACCCGCTCCCACCATCGCCTCTCAACCTCTTAGTCTTCGCCCCGA | |
| (without linker) | GGCATGCCGACCCGCCGCCGGGGGTGCTGTTCATACGAGGGGCTTGGACTTC | |
| | GCTTGTGATATTTACATTTGGGCTCCGTTGGCGGGTACGTGCGGCGTCCTTT | |
| | TGTTGTCACTCGTTATTACTTTGTATTGTAATCACAGGAATCGC | |
| CD8a extracellular + | FVPVFLPAKPTTTPAPRPPTPAPTIASQPLSLRPEACRPAAGGAVHTRGLDF | 125 |
| transmembrane | ACDIYIWAPLAGTCGVLLLSLVITLYCNHRNR | |
| (without linker) | | |
| CD28 co- | TCAAAGCGGAGTAGGTTGTTGCATTCCGATTACATGAATATGACTCCTCGCC | 45 |
| stimulatory | GGCCTGGGCCGACAAGAAAACATTACCAACCCTATGCCCCCCCACGAGACTT | |
| | CGCTGCGTACAGGTCC | |
| CD28 co- | SKRSRLLHSDYMNMTPRRPGPTRKHYQPYAPPRDFAAYRS | 46 |
| stimulatory | | |
| 41BB co- | AAACGGGGCAGAAAGAAACTCCTGTATATATTCAAACAACCATTTATGAGAC | 43 |
| stimulatory | CAGTACAAACTACTCAAGAGGAAGATGGCTGTAGCTGCCGATTTCCAGAAGA | |
| | AGAAGAAGGAGGATGTGAACTG | |
| 41BB co- | KRGRKKLLYIFKQPFMRPVQTTQEEDGCSCRFPEEEEGGCEL | 44 |
| stimulatory | | |
| CD3z | CGAGTGAAGTTTTCCCGAAGCGCAGACGCTCCGGCATATCAGCAAGGACAGA | 47 |
| | ATCAGCTGTATAACGAACTGAATTTGGGACGCCGCGAGGAGTATGACGTGCT | |
| | TGATAAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGAGG | |
| | CCTACTCAGAAATAGGTATGAAGGGCGAACGACGACGGGGAAAAGGTCACGA | |
| | TGGCCTCTACCAAGGGTTGAGTACGGCAACCAAAGATACGTACGATGCACTG | |
| | CATATGCAGGCCCTGCCTCCCAGA | |
| CD3a | RVKFSRSADAPAYQQGQNQLYNELNLGRREEYDVLDKRRGRDPEMGGKPRRK | 48 |
| | NPQEGLYNELQKDKMAEAYSEIGMKGERRRGKGHDGLYQGLSTATKDTYDAL | |
| | HMQALPPR | |

TABLE 27
Donor Components
Donor structure: TRAC[LHA]-EF1a[promoter]-CAR-polyA-TRAC[RHA]

| | | SEQ |
|---|---|---|
| | | ID |
| Name | Sequence | NO: |
| TRAC-LHA | GAGATGTAAGGAGCTGCTGTGACTTGCTCAAGGCCTTATATCGAGTAAACGGTAGT | 128 |
| | GCTGGGGCTTAGACGCAGGTGTTCTGATTTATAGTTCAAAACCTCTATCAATGAGA | |
| | GAGCAATCTCCTGGTAATGTGATAGATTTCCCAACTTAATGCCAACATACCATAAA | |
| | CCTCCCATTCTGCTAATGCCCAGCCTAAGTTGGGGAGACCACTCCAGATTCCAAGA | |
| | TGTACAGTTTGCTTTGCTGGGCCTTTTTCCCATGCCTGCCTTTACTCTGCCAGAGT | |
| | TATATTGCTGGGGTTTTGAAGAAGATCCTATTAAATAAAAGAATAAGCAGTATTAT | |
| | TAAGTAGCCCTGCATTTCAGGTTTCCTTGAGTGGCAGGCCAGGCCTGGCCGTGAAC | |
| | GTTCACTGAAATCATGGCCTCTTGGCCAAGATTGATAGCTTGTGCCTGTCCCTGAG | |
| | TCCCAGTCCATCACGAGCAGCTGGTTTCTAAGATGCTATTTCCCGTATAAAGCATG | |
| | AGACCGTGACTTGCCAGCCCCACAGAGCCCCGCCCTTGTCCATCACTGGCATCTGG | |
| | ACTCCAGCCTGGGTTGGGGCAAAGAGGGAAATGAGATCATGTCCTAACCCTGATCC | |
| | TCTTGTCCCACAGATATCCAGAACCCTGACCCTGCCGTGTACCAGCTGAGAGACTC | |
| | TAAATCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGACATG | |
| | AGGTCTATGGACTTCA | |
| EF1α | GGCTCCGGTGCCCGTCAGTGGGCAGAGCGCACATCGCCCACAGTCCCCGAGAAGTT | 129 |
| promoter | GGGGGGAGGGGTCGGCAATTGAACCGGTGCCTAGAGAAGGTGGCGCGGGGTAAACT | |
| | GGGAAAGTGATGTCGTGTACTGGCTCCGCCTTTTTCCCGAGGGTGGGGGAGAACCG | |
| | TATATAAGTGCAGTAGTCGCCGTGAACGTTCTTTTTCGCAACGGGTTTGCCGCCAG | |
| | AACACAGGTAAGTGCCGTGTGTGGTTCCCGCGGGCCTGGCCTCTTTACGGGTTATG | |
| | GCCCTTGCGTGCCTTGAATTACTTCCACTGGCTGCAGTACGTGATTCTTGATCCCG | |
| | AGCTTCGGGTTGGAAGTGGGTGGGAGAGTTCGAGGCCTTGCGCTTAAGGAGCCCCT | |
| | TCGCCTCGTGCTTGAGTTGAGGCCTGGCCTGGGCGCTGGGGCCGCCGCGTGCGAAT | |
| | CTGGTGGCACCTTCGCGCCTGTCTCGCTGCTTTCGATAAGTCTCTAGCCATTTAAA | |
| | ATTTTTGATGACCTGCTGCGACGCTTTTTTTCTGGCAAGATAGTCTTGTAAATGCG | |
| | GGCCAAGATCTGCACACTGGTATTTCGGTTTTTGGGGCCGCGGGCGGCGACGGGGC | |
| | CCGTGCGTCCCAGCGCACATGTTCGGCGAGGCGGGGCCTGCGAGCGCGGCCACCGA | |
| | GAATCGGACGGGGGTAGTCTCAAGCTGGCCGGCCTGCTCTGGTGCCTGGCCTCGCG | |
| | CCGCCGTGTATCGCCCCGCCCTGGGCGGCAAGGCTGGCCCGGTCGGCACCAGTTGC | |
| | GTGAGCGGAAAGATGGCCGCTTCCCGGCCCTGCTGCAGGGAGCTCAAAATGGAGGA | |
| | CGCGGCGCTCGGGAGAGCGGGCGGGTGAGTCACCCACACAAAGGAAAAGGGCCTTT | |
| | CCGTCCTCAGCCGTCGCTTCATGTGACTCCACGGAGTACCGGGCGCCGTCCAGGCA | |
| | CCTCGATTAGTTCTCGAGCTTTTGGAGTACGTCGTCTTTAGGTTGGGGGGAGGGGT | |
| | TTTATGCGATGGAGTTTCCCCACACTGAGTGGGTGGAGACTGAAGTTAGGCCAGCT | |
| | TGGCACTTGATGTAATTCTCCTTGGAATTTGCCCTTTTTGAGTTTGGATCTTGGTT | |
| | CATTCTCAAGCCTCAGACAGTGGTTCAAAGTTTTTTTCTTCCATTTCAGGTGTCGT | |
| | GA | |
| Synthetic | AATAAAATCGCTATCCATCGAAGATGGATGTGTGTTGGTTTTTTGTGTG | 130 |
| poly(A) | | |
| signal | | |
| TRAC-RHA | TGGAGCAACAAATCTGACTTTGCATGTGCAAACGCCTTCAACAACAGCATTATTCC | 131 |
| | AGAAGACACCTTCTTCCCCAGCCCAGGTAAGGGCAGCTTTGGTGCCTTCGCAGGCT | |
| | GTTTCCTTGCTTCAGGAATGGCCAGGTTCTGCCCAGAGCTCTGGTCAATGATGTCT | |
| | AAAACTCCTCTGATTGGTGGTCTCGGCCTTATCCATTGCCACCAAAACCCTCTTTT | |
| | TACTAAGAAACAGTGAGCCTTGTTCTGGCAGTCCAGAGAATGACACGGGAAAAAAG | |
| | CAGATGAAGAGAAGGTGGCAGGAGAGGGCACGTGGCCCAGCCTCAGTCTCTCCAAC | |
| | TGAGTTCCTGCCTGCCTGCCTTTGCTCAGACTGTTTGCCCCTTACTGCTCTTCTAG | |
| | GCCTCATTCTAAGCCCCTTCTCCAAGTTGCCTCTCCTTATTTCTCCCTGTCTGCCA | |
| | AAAAATCTTTCCCAGCTCACTAAGTCAGTCTCACGCAGTCACTCATTAACCCACCA | |
| | ATCACTGATTGTGCCGGCACATGAATGCACCAGGTGTTGAAGTGGAGGAATTAAAA | |
| | AGTCAGATGAGGGGTGTGCCCAGAGGAAGCACCATTCTAGTTGGGGGAGCCCATCT | |
| | GTCAGCTGGGAAAAGTCCAAATAACTTCAGATTGGAATGTGTTTTAACTCAGGGTT | |
| | GAGAAAACAGCTACCTTCAGGACAAAAGTCAGGGAAGGGCTCTCTGAAGAAATGCT | |
| | ACTTGAAGATACCAGCCCTACCAAGGGCAGGGAGAGGACCCTATAGAGGCCTGGGA | |
| | CAGGAGCTCAATGAGAAAGG | |

### Antibodies

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv), single chain variable fragment (scFv), mutants thereof, fusion proteins comprising an antibody portion, humanized antibodies, chimeric antibodies, diabodies, linear antibodies, single chain antibodies, single domain antibodies (e.g., camel or llama VHH antibodies), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

A typical antibody molecule comprises a heavy chain variable region (VH) and a light chain variable region (VL), which are usually involved in antigen binding. These regions/residues that are responsible for antigen-binding can be identified from amino acid sequences of the VH/VL sequences of a reference antibody (e.g., an anti-CD33 antibody as described herein) by methods known in the art. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. As used herein, a CDR may refer to the CDR defined by any method known in the art. Two antibodies having the same CDR means that the two antibodies have the same amino acid sequence of that CDR as determined by the same method. See, e.g., Kabat, E. A., et al., (1991) Sequences of Proteins of Immunological Interest, Fifth Edition, U.S. Department of Health and Human Services, NIH Publication No. 91-3242, Chothia et al., (1989) Nature 342:877; Chothia, C. et al., (1987) J. Mol. Biol. 196:901-917, Al-lazikani et al., (1997) J. Molec. Biol. 273:927-948; and Almagro, J. Mol. Recognit. 17:132-143 (2004). See also hgmp.mrc.ac.uk and bioinf.org.uk/abs.

In some embodiments, an antibody is a scFv, such as an anti-CD33 scFv. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used as provided herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC).

In some embodiments, an antibody of the present disclosure is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In some embodiments, an antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure specifically binds a target antigen, such as human CD33. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a CD33 epitope is an antibody that binds this CD33 epitope with greater affinity, avidity, more readily, and/or with greater duration than it binds to other CD33 epitopes or non-CD33 epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In some embodiments, the equilibrium dissociation constant (K_{D}) between the antibody and CD33 is 100 pM to 1 μM. In some embodiments, the K_{D} between the antibody and CD33 is 1 nM to 100 nM.

Also within the scope of the present disclosure are functional variants of any of the exemplary anti-CD33 antibodies as disclosed herein. A functional variant may contain one or more amino acid residue variations in the VH and/or VL, or in one or more of the HC CDRs and/or one or more of the VL CDRs as relative to a reference antibody, while retaining substantially similar binding and biological activities (e.g., substantially similar binding affinity, binding specificity, inhibitory activity, anti-tumor activity, or a combination thereof) as the reference antibody.

In some examples, an anti-CD33 antibody disclosed herein comprises a VH CDR1, a VH CDR2, and a VH CDR3, which collectively contains no more than 10 amino acid variations (e.g., no more than 9, 8, 7, 6, 5, 4, 3, 2, or 1 amino acid variation) as compared with the VH CDR1, VH CDR2, and VH CDR3 of a reference antibody such as Antibody A (VH: SEQ ID NO: 65; VL: SEQ ID NO: 66). “Collectively” means that the total number of amino acid variations in all of the three VH CDRs is within the defined range. Alternatively or in addition, the anti-CD33 antibody may comprise a VL CDR1, a VL CDR2, and a VL CDR3, which collectively contains no more than 10 amino acid variations (e.g., no more than 9, 8, 7, 6, 5, 4, 3, 2 or 1 amino acid variation) as compared with the VL CDR1, VL CDR2, and VL CDR3 of the reference antibody.

In some examples, the anti-CD33 antibody disclosed herein may comprise a VH CDR1, a VH CDR2, and a VH CDR3, at least one of which contains no more than 5 amino acid variations (e.g., no more than 4, 3, 2, or 1 amino acid variation) as the counterpart VH CDR of a reference antibody such as Antibody A (VH: SEQ ID NO: 65; VL: SEQ ID NO: 66). In specific examples, the antibody comprises a VH CDR3, which contains no more than 5 amino acid variations (e.g., no more than 4, 3, 2, or 1 amino acid variation) as the VH CDR3 of a reference antibody such as Antibody A (VH: SEQ ID NO: 65; VL: SEQ ID NO: 66). Alternatively or in addition, an anti-CD33 antibody may comprise a VL CDR1, a VL CDR2, and a VL CDR3, at least one of which contains no more than 5 amino acid variations (e.g., no more than 4, 3, 2, or 1 amino acid variation) as the counterpart VL CDR of the reference antibody. In specific examples, the antibody comprises a VL CDR3, which contains no more than 5 amino acid variations (e.g., no more than 4, 3, 2, or 1 amino acid variation) as the VL CDR3 of the reference antibody.

In some examples, an anti-CD33 antibody disclosed herein comprises a VH CDR1, a VH CDR2, and a VH CDR3, which collectively contains no more than 10 amino acid variations (e.g., no more than 9, 8, 7, 6, 5, 4, 3, 2, or 1 amino acid variation) as compared with the VH CDR1, VH CDR2, and VH CDR3 of a reference antibody such as Antibody B (VH: SEQ ID NO: 77; VL: SEQ ID NO: 78). In some examples, the anti-CD33 antibody disclosed herein may comprise a VH CDR1, a VH CDR2, and a VH CDR3, at least one of which contains no more than 5 amino acid variations (e.g., no more than 4, 3, 2, or 1 amino acid variation) as the counterpart VH CDR of a reference antibody such as Antibody B (VH: SEQ ID NO: 77; VL: SEQ ID NO: 78). In specific examples, the antibody comprises a VH CDR3, which contains no more than 5 amino acid variations (e.g., no more than 4, 3, 2, or 1 amino acid variation) as the VH CDR3 of a reference antibody such as Antibody B (VH: SEQ ID NO: 77; VL: SEQ ID NO: 78).

In some examples, an anti-CD33 antibody disclosed herein comprises a VH CDR1, a VH CDR2, and a VH CDR3, which collectively contains no more than 10 amino acid variations (e.g., no more than 9, 8, 7, 6, 5, 4, 3, 2, or 1 amino acid variation) as compared with the VH CDR1, VH CDR2, and VH CDR3 of a reference antibody such as Antibody C (VH: SEQ ID NO: 89; VL: SEQ ID NO: 90). In some examples, the anti-CD33 antibody disclosed herein may comprise a VH CDR1, a VH CDR2, and a VH CDR3, at least one of which contains no more than 5 amino acid variations (e.g., no more than 4, 3, 2, or 1 amino acid variation) as the counterpart VH CDR of a reference antibody such as Antibody C (VH: SEQ ID NO: 89; VL: SEQ ID NO: 90). In specific examples, the antibody comprises a VH CDR3, which contains no more than 5 amino acid variations (e.g., no more than 4, 3, 2, or 1 amino acid variation) as the VH CDR3 of a reference antibody such as Antibody C (VH: SEQ ID NO: 89; VL: SEQ ID NO: 90).

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: ((a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

In some embodiments, an antibody disclosed herein may comprise VH CDRs that collectively are at least 80% (e.g., 85%, 90%, 95%, or 98%) identical to the VH CDRs of a reference antibody such as Antibody A (VH: SEQ ID NO: 65; VL: SEQ ID NO: 66). Alternatively or in addition, the antibody may comprise VL CDRs that collectively are at least 80% (e.g., 85%, 90%, 95%, or 98%) identical to the VL CDRs of the reference antibody. In some embodiments, an antibody may comprise a VH that is at least 80% (e.g., 85%, 90%, 95%, or 98%) identical to the VH of a reference antibody such as Antibody A (VH: SEQ ID NO: 65; VL: SEQ ID NO: 66) and/or a VL that is at least 80% (e.g., 85%, 90%, 95%, or 98%) identical to the VL variable region of the reference antibody.

### Donor Template

The nucleic acid encoding a CAR may be delivered to a T cell using a vector (e.g., an AAV vector) that comprises what is referred to herein as a donor template (also referred to as a donor polynucleotide). A donor template can contain a non-homologous sequence, such as the nucleic acid encoding a CAR, flanked by two regions of homology to allow for efficient HDR at a genomic location of interest. Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, is inserted so that its expression is driven by the endogenous promoter at the integration site, namely the promoter that drives expression of the endogenous gene into which the donor is inserted. However, in some embodiments, the donor template comprises an exogenous promoter and/or enhancer, for example a constitutive promoter, an inducible promoter, or tissue-specific promoter. In some embodiments, the exogenous promoter is an EF1α promoter comprising a sequence of SEQ ID NO: 129. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

### Delivery Methods and Constructs

Nucleases and/or donor templates may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

Adeno-Associated Viral Delivery

The donor nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

Homology-Directed Repair (HDR)

The donor nucleic acid encoding a CAR is inserted by homology directed repair (HDR) into the target gene locus. Both strands of the DNA at the target locus are cut by a CRISPR Cas9 enzyme. HDR then occurs to repair the double-strand break (DSB) and insert the donor DNA. For this to occur correctly, the donor sequence is designed with flanking residues which are complementary to the sequence surrounding the DSB site in the target gene (hereinafter “homology arms”). These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

The target gene can be associated with an immune response in a subject, wherein permanently deleting at least a portion of the target gene will modulate the immune response. For example, to generate a CAR T cell, the target gene can be the TCRα constant region (TRAC). Disruption of TRAC leads to loss of function of the endogenous TCR.

In some embodiments, the target gene is in a safe harbor locus.

### Engineered T Cells

Engineered (gene edited) CAR T cells of the present disclosure may be autologous (“self”) or non-autologous (“non-self,” e.g., allogeneic, syngeneic or xenogeneic). “Autologous” refers to cells from the same subject. “Allogeneic” refers to cells of the same species as a subject, but that differ genetically to the cells in the subject. In some embodiments, the T cells are obtained from a mammal. In some embodiments, the T cells are obtained from a human.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some embodiments, an isolated population of T cells is used. In some embodiments, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRab, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, the engineered T cell populations do not express or do not substantially express one or more of the following markers: CD70, CD57, CD244, CD160, PD-1, CTLA4, HM3, and LAG3. In some embodiments, subpopulations of T cells may be isolated by positive or negative selection prior to genetic engineering and/or post genetic engineering.

In some embodiments, an isolated population of T cells expresses one or more of the markers including, but not limited to a CD3+, CD4+, CD8+, or a combination thereof. In some embodiments, the T cells are isolated from a donor, or subject, and first activated and stimulated to proliferate in vitro prior to undergoing gene editing.

To achieve sufficient therapeutic doses of T cell compositions, T cells are often subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells.

In some embodiments, T cells are activated at the same time that genome editing compositions are introduced into the T cells.

### Treatment Methods and Compositions

Provided herein, in some embodiments, are methods for treating cancer (e.g., leukemias, e.g., acute myeloid leukemia). Non-limiting examples of leukemias that may be treated as provided herein include acute lymphoblastic leukemia (ALL), acute myeloid leukemia (AML), chronic lymphocytic leukemia (CLL) and chronic myeloid leukemia (CML). In some embodiment, the methods comprise delivering the CAR T cells (e.g., anti-CD33 CAR T cells) of the present disclosure to a subject having cancer (e.g., leukemias) including acute lymphoblastic leukemia (ALL), acute myeloid leukemia (AML), chronic lymphocytic leukemia (CLL) and chronic myeloid leukemia (CML).

The step of administering may include the placement (e.g., transplantation) of cells, e.g., engineered T cells, into a subject, by a method or route that results in at least partial localization of the introduced cells at a desired site, such as tumor, such that a desired effect(s) is produced. Engineered T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of engineered T cells is administered via a systemic route of administration, such as an intraperitoneal or intravenous route.

A subject may be any subject for whom diagnosis, treatment, or therapy is desired. In some embodiments, the subject is a mammal. In some embodiments, the subject is a human.

A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used.

In some embodiments, an engineered T cell population being administered according to the methods described herein comprises allogeneic T cells obtained from one or more donors. Allogeneic refers to a cell, cell population, or biological samples comprising cells, obtained from one or more different donors of the same species, where the genes at one or more loci are not identical to the recipient (e.g., subject). For example, an engineered T cell population, being administered to a subject can be derived from one or more unrelated donors, or from one or more non-identical siblings. In some embodiments, syngeneic cell populations may be used, such as those obtained from genetically identical donors, (e.g., identical twins). In some embodiments, the cells are autologous cells; that is, the engineered T cells are obtained or isolated from a subject and administered to the same subject, i.e., the donor and recipient are the same.

In some embodiments, an engineered T cell population being administered according to the methods described herein does not induce toxicity in the subject, e.g., the engineered T cells do not induce toxicity in non-cancer cells. In some embodiments, an engineered T cell population being administered does not trigger complement mediated lysis, or does not stimulate antibody-dependent cell mediated cytotoxicity (ADCC).

An effective amount refers to the amount of a population of engineered T cells needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

For use in the various aspects described herein, an effective amount of cells (e.g., engineered T cells) comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The cells are derived from one or more donors, or are obtained from an autologous source. In some examples described herein, the cells are expanded in culture prior to administration to a subject in need thereof.

Modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

In some embodiments, engineered T cells are administered systemically, which refers to the administration of a population of cells other than directly into a target site, tissue, or organ, such that it enters, instead, the subject's circulatory system and, thus, is subject to metabolism and other like processes.

The efficacy of a treatment comprising a composition for the treatment of a medical condition can be determined by the skilled clinician. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

### Other Embodiments

The disclosure relates to the following embodiments. Throughout this section, the term embodiment is abbreviated as ‘E’ followed by an ordinal. For example, E1 is equivalent to Embodiment 1.

E1. An engineered T cell comprising a nucleic acid encoding a chimeric antigen receptor (CAR), wherein the CAR comprises an ectodomain that binds specifically to CD33.
E2. The engineered T cell of embodiment 1 further comprising a disrupted T cell receptor alpha chain constant region (TRAC) gene.
E3. The engineered T cell of embodiment 2, wherein the nucleic acid encoding the CAR is inserted into the TRAC gene.
E4. The engineered T cell of any one of embodiments 1-3 further comprising a disrupted beta-2-microglobulin (p82M) gene.
E5. The engineered T cell of any one of embodiments 1-4, wherein the ectodomain of the CAR comprises an anti-CD33 antibody.
E6. The engineered T cell of embodiment 5, wherein the anti-CD33 antibody is an anti-CD33 single-chain variable fragment (scFv).
E7. The engineered T cell of embodiment 6, wherein the anti-CD33 scFv comprises the same heavy chain variable domain (VH) complementarity determining regions (CDRs) and the same light chain variable domain (VL) CDRs as a reference antibody, wherein the reference antibody comprises:

(i) a VH set forth as SEQ ID NO: 65 and a VL set forth as SEQ ID NO: 66,

(ii) a VH set forth as SEQ ID NO: 77 and a VL set forth as SEQ ID NO: 78, or

(iii) a VH set forth as SEQ ID NO: 89 and a VL set forth as SEQ ID NO: 90.

E8. The engineered T cell of embodiment 7, wherein the anti-CD33 scFv comprises the same VH and VL chains as the reference antibody.
E9. The engineered T cell of embodiment 7, wherein the anti-CD33 scFv comprises the amino acid sequence of any one of SEQ ID NOs: 73, 75, 85, 87, 97, or 99.
E10. The engineered T cell of any one of embodiments 1-9, wherein the CAR further comprises a CD28 co-stimulatory domain or a 41BB co-stimulatory domain.
E11. The engineered T cell of embodiment 10, wherein the CAR further comprises a CD3ζ cytoplasmic signaling domain.
E12. The engineered T cell of any one of embodiment 3-11, wherein the TRAC gene comprises the nucleotide sequence of any one of SEQ ID NOs: 49, 51, 53, 55, 57, 59, 61, 63, 109, 112, 115, or 118, and/or wherein the CAR is encoded by the nucleotide sequence of any one of SEQ ID NOs: 50, 52, 54, 56, 58, 60, 62, 64, 110, 113, 116 or 119.
E13. The engineered T cell of any one of embodiments 4-14, wherein the disrupted β2M gene comprises at least one nucleotide sequence selected from any one of SEQ ID NOs: 9-14.
E14. The engineered T cell of any one of embodiments 1-15, wherein the T cells comprise a wild-type CD33 gene.
E15. The engineered T cell of any one of embodiments 1-15, wherein the T cells further comprise a disrupted CD33 gene.
E16. The engineered T cell of embodiment 17, wherein the disrupted CD33 gene comprises a nucleotide sequence of AGTTCATGGTACTGGTTCC (SEQ ID NO: 187), AGTTCATGGTTCC (SEQ ID NO: 188), AGTTCATGTACTGGTTCC (SEQ ID NO: 189), AGTTCATGGTTTACTGGTTCC (SEQ ID NO: 190), AGTTCC, AGTACTGGTTCC (SEQ ID NO: 191), AGTTCATACTGGTTCC (SEQ ID NO: 192), AGTTCATGGTATACTGGTTCC (SEQ ID NO: 193), and/or AGTTACTGGTTCC (SEQ ID NO: 194).
E17. The engineered T cell of embodiment 17 or embodiment 18, wherein the disrupted CD33 gene lacks a fragment comprising AGTTCATGGTTACTGGTTCC (SEQ ID NO: 186).
E18. The engineered T cell of embodiment 17, wherein the disrupted CD33 gene comprises a nucleotide sequence of AAATCCTGGCACT (SEQ ID NO: 300), AAATCCCTGGCACT (SEQ ID NO: 301), AAATCCTCATTCCCTGGCACT (SEQ ID NO: 302), AAATCCTCACCCTGGCACT (SEQ ID NO: 304), AAATCCTCCCCTGGCACT (SEQ ID NO: 305), AAATCCTCCCTGGCACT (SEQ ID NO: 306), AAATCCCCTGGCACT (SEQ ID NO: 307), ACATCCTCATTCCCTGGCACT (SEQ ID NO: 308), ACATCCTGGCACT (SEQ ID NO: 309), AAATCCTCTCCCTGGCACT (SEQ ID NO: 310), AAATCCTCATCTGGCACT (SEQ ID NO: 311), AAATCCT, AAACCCTGGCACT (SEQ ID NO: 312), AAATCCTCTGGCACT (SEQ ID NO: 313), AAATCCCCCTGGCACT (SEQ ID NO: 314), AAATCCTCACT (SEQ ID NO: 315), ACATCCCTGGCACT (SEQ ID NO: 316), and/or AAAT.
E19. The engineered T cell of embodiment 20, wherein the disrupted CD33 gene lacks a fragment comprising AAATCCTCATCCCTGGCACT (SEQ ID NO: 299).
E20. The engineered T cell of embodiment 20 or embodiment 21, wherein the disrupted CD33 gene lacks a fragment, the 3′ segment of which comprises the nucleotide sequence of AAATCCTCAT (SEQ ID NO: 317), AAATCCTCATCCCT (SEQ ID NO: 318), AAATCCTCATCCCTGG (SEQ ID NO: 320), AAATCCTCATC (SEQ ID NO: 322), or AAATCCTCATCCCTGGCA (SEQ ID NO: 324).
E21. The engineered T cell of any one of embodiments 20-22, wherein the disrupted CD33 gene lacks a fragment, the 5′ segment of which comprises the nucleotide sequence of CTCATCCCTGGCACT (SEQ ID NO: 323).
E22. A population of engineered T cells comprising the engineered T cell of any one of embodiments 1-21, wherein at least 25% or at least 50% of engineered T cells of the population express the CAR.
E23. The population of embodiment 22, wherein at least 70% of engineered T cells of the population express the CAR.
E24. The population of embodiment 22, wherein at least 25% of engineered T cells of the population express the CAR following at least 7 days or at least 14 days of in vitro proliferation.
E25. The population of any one of embodiments 22-24, wherein at least 50% of engineered T cells of the population do not express a detectable level of T cell receptor (TCR) protein.
E26. The population of embodiment 25, wherein at least 90% of engineered T cells of the population do not express a detectable level of TCR protein.
E27. The population of any one of embodiments 22-26, wherein at least 50% of engineered T cells of the population do not express a detectable level of β2M protein.
E28. The population of embodiment 27, wherein at least 70% of engineered T cells of the population do not express a detectable level of β2M protein.
E29. The population of any one of embodiments 22-28, wherein at least 20% of engineered T cells of the population do not express a detectable level of CD33 protein.
E30. The population of embodiment 29, wherein at least 50% of engineered T cells of the population do not express a detectable level of CD33 protein.
E31. The population of any one of embodiments 22-30, wherein engineered T cells of the population, when co-cultured in vitro with a population of cancer cells that express CD33, induce cell lysis of at least 10%, at least 25%, or at least 50% of the cancer cells of the population.
E32. The population of embodiment 31, wherein engineered T cells of the population, when co-cultured in vitro with a population of cancer cells that express CD33, induce cell lysis of at least 70%, at least 80%, or at least 90% of the population of cancer cells.
E33. The population of embodiment 31 or embodiment 32, wherein engineered T cells of the population, when co-cultured in vitro with a population of cancer cells, secrete IFNγ.
E34. The population of any one of embodiments 31-33, wherein the ratio of engineered T cells to cancer cells is 1:1 to 2:1.
E35. The population of any one of embodiments 31-34, wherein the cancer cells comprise leukemia.
E36. The population of any one of embodiments 31-34, wherein the cancer cells comprise acute lymphoblastic leukemia (ALL), acute myeloid leukemia (AML), chronic lymphocytic leukemia (CLL) and chronic myeloid leukemia (CML).
E37. A method comprising administering the population of engineered T cells of any one of embodiments 22-36 to a subject.
E38. The method of embodiment 37, wherein the subject is a human subject.
E39. The method of embodiment 37 or 38, wherein the subject has a cancer.
E40. The method of embodiment 39, wherein the cancer is a leukemia, optionally acute lymphoblastic leukemia (ALL), acute myeloid leukemia (AML), chronic lymphocytic leukemia (CLL) and chronic myeloid leukemia (CML).
E41. The method of embodiment 39 or embodiment 40 wherein the cancer comprises cancer cells expressing CD33.
E42. The method of any one of embodiments 39-41, wherein administering the population of engineered T cells to a subject causes a reduction in cancerous tumor volume(s) relative to a baseline control.
E43. A method for producing an engineered T cell, the method comprising

(a) delivering to a T cell
(i) a RNA-guided nuclease,
(ii) a gRNA targeting a TRAC gene, and
(iii) a vector comprising a donor template that comprises a nucleic acid encoding a CAR that comprise an ectodomain that binds specifically to CD33; and

(b) producing an engineered T cell having a disrupted TRAC gene and expressing the CAR.

E44. The method of embodiment 43, wherein the gRNA targeting the TRAC gene comprises the nucleotide sequence of SEQ ID NO: 18 or SEQ ID NO: 19, or targets the nucleotide sequence of SEQ ID NO: 40.
E45. The method of embodiment 43 or 44 wherein the nucleic acid encoding the CAR is flanked by left and right homology arms to the TRAC gene.
E46. The method of any one of embodiments 43-45 further comprising delivering to the T cell a gRNA targeting the β2M gene.
E47. The method of embodiment 46, wherein the gRNA targeting the β2M gene comprises the nucleotide sequence of SEQ ID NO: 20 or SEQ ID NO: 21, or targets the nucleotide sequence of SEQ ID NO: 41.
E48. The method of any one of embodiments 43-47, wherein the RNA-guided nuclease is a Cas9 nuclease, optionally a S. pyogenes Cas9 nuclease.
E49. The method of any one of embodiments 43-48 further comprising delivering to the T cell a gRNA targeting the CD33 gene.
E50. The method of embodiment 49, wherein the gRNA targeting the CD33 gene comprises a nucleotide sequence as provided in Table 10.
E51. The method of any one of embodiments 43-50, wherein the ectodomain of the CAR is an anti-CD33 antibody.
E52. The method of embodiment 51, wherein the anti-CD33 antibody is an anti-CD33 single-chain variable fragment (scFv).
E53. The method of embodiment 52, wherein the anti-CD33 scFv comprises the same heavy chain variable domain (VH) complementarity determining regions (CDRs) and the same light chain variable domain (VL) CDRs as a reference antibody, wherein the reference antibody comprises (i) a VH set forth as SEQ ID NO: 65 and a VL set forth as SEQ ID NO: 66, (ii) a VH set forth as SEQ ID NO: 77 and a VL set forth as SEQ ID NO: 78, or (iii) a VH set forth as SEQ ID NO: 89 and a VL set forth as SEQ ID NO: 90.
E54. The method of embodiment 52, wherein the anti-CD33 scFv comprises the same VH and VL chains as the reference antibody.
E55. The method of embodiment 54, wherein the anti-CD33 scFv comprises the amino acid sequence of any one of SEQ ID NOs: 73, 75, 85, 87, 97, or 99.
E56. The method of any one of embodiments 43-55, wherein the CAR comprises a CD28 co-stimulatory domain or a 41BB co-stimulatory domain.
E57. The method of embodiment 56, wherein the CAR further comprises a CD3ζ cytoplasmic signaling domain.
E58. The method of any one of embodiments 43-57, wherein the donor template comprises the nucleotide sequence of any one of SEQ ID NOs: 49, 51, 53, 55, 57, 59, 61, 63, 109, 112, 115, or 118.
E59. The method of any one of embodiments 43-58, wherein the CAR is encoded by a nucleotide sequence of any one of SEQ ID NOs: 50, 52, 54, 56, 58, 60, 62, 64, 110, 113, 116 or 119.
E60. A method for reducing volume of a tumor in a subject having cancer, the method comprising administering to the subject a population of engineered T cells any one of embodiments 22-36.
E61. The method of embodiment 60, wherein the volume of the tumor in the subject is reduced by at least 50% relative to a baseline control, optionally wherein 1×10⁵ cells to 1×10⁷ cells of the population are administered.
E62. A population of cells comprising engineered T cells, wherein the engineered T cells comprise:

(i) a disrupted TRAC gene;

(ii) a disrupted 12M gene; and

(iii) a nucleic acid encoding a CAR comprising an anti-CD33 antigen-binding fragment.

E63. The population of cells of embodiment 62, wherein the CAR comprises (a) an ectodomain that comprises an anti-CD33 antigen-binding fragment, (b) a CD8 transmembrane domain, and (c) an endodomain that comprises a 41BB co-stimulatory domain and a CD3ζ co-stimulatory domain.
E64. The population of cells of embodiment 62 or embodiment 63, wherein the disrupted TRAC gene comprises the nucleic acid encoding the CAR.
E65. The population of cells of any one of embodiments 62-64 further comprising a disrupted CD33 gene.
E66. A population of cells comprising engineered T cells, wherein the engineered T cells comprise:

(i) a disrupted TRAC gene, wherein the disrupted TRAC gene comprises a nucleic acid encoding a CAR comprising (a) an ectodomain that comprises an anti-CD33 antigen-binding fragment, (b) a CD8 transmembrane domain, and (c) an endodomain that comprises a 41BB co-stimulatory domain and a CD3ζ co-stimulatory domain; and

(ii) a disrupted β2M gene.

E67. The population of cells of embodiment 63 further comprising a disrupted CD33 gene.
E68. A population of cells comprising engineered T cells, wherein the engineered T cells comprise:

(i) a disrupted TRAC gene, wherein the disrupted TRAC gene comprises a nucleic acid encoding a CAR comprising the amino acid sequence of SEQ ID NO: 104; and

(ii) a disrupted β2M gene.

E69. The population of cells of embodiment 68 further comprising a disrupted CD33 gene.
E70. A population of cells comprising engineered T cells, wherein the engineered T cells comprise:

(i) a disrupted TRAC gene, wherein the disrupted TRAC gene comprises a nucleic acid encoding a CAR, wherein the nucleic acid sequence is at least 90% identical to SEQ ID NO: 56 and encodes the CAR of SEQ ID NO:104; and

(ii) a disrupted β2M gene.

E71. The population of cells of embodiment 70 further comprising a disrupted CD33 gene.
E72. A population of cells comprising engineered T cells, wherein the engineered T cells comprise:

(i) a disrupted TRAC gene, wherein the disrupted TRAC gene comprises the nucleic acid sequence of SEQ ID NO: 55; and

(ii) a disrupted β2M gene.

E73. The population of cells of embodiment 72 further comprising a disrupted CD33 gene.
E74. An engineered T cell comprising:

(i) a disrupted TRAC gene;

(ii) a disrupted β2M gene; and

(iii) a nucleic acid encoding a CAR comprising an anti-CD33 antigen-binding fragment.

E75. The engineered T cell of embodiment 74, wherein the CAR comprises (a) an ectodomain that comprises an anti-CD33 antigen-binding fragment, (b) a CD8 transmembrane domain, and (c) an endodomain that comprises a 41BB co-stimulatory domain and a CD3ζ co-stimulatory domain.
E76. The engineered T cell of embodiment 74 or embodiment 75, wherein the disrupted TRAC gene comprises the nucleic acid encoding the CAR.
E77. The engineered T cell of any one of embodiments 74-76 further comprising a disrupted CD33 gene.
E78. An engineered T cell comprising:

(i) a disrupted TRAC gene, wherein the disrupted TRAC gene comprises a nucleic acid encoding a CAR comprising (a) an ectodomain that comprises an anti-CD33 antigen-binding fragment, (b) a CD8 transmembrane domain, and (c) an endodomain that comprises a 41BB co-stimulatory domain and a CD3ζ co-stimulatory domain; and

(ii) a disrupted β2M gene.

E79. The engineered T cell of embodiment 75 further comprising a disrupted CD33 gene.
E80. An engineered T cell comprising:

(i) a disrupted TRAC gene, wherein the disrupted TRAC gene comprises a nucleic acid encoding a CAR comprising the amino acid sequence of SEQ ID NO: 104; and

(ii) a disrupted β2M gene.

E81. The engineered T cell of embodiment 77 further comprising a disrupted CD33 gene.
E82. An engineered T cell comprising:

(i) a disrupted TRAC gene, wherein the disrupted TRAC gene comprises a nucleic acid encoding a CAR, wherein the nucleic acid sequence is at least 90% identical to SEQ ID NO: 56 and encodes the CAR of SEQ ID NO:104; and

(ii) a disrupted β2M gene.

E83. The engineered T cell of embodiment 82 further comprising a disrupted CD33 gene.
E84. An engineered T cell comprising:

(i) a disrupted TRAC gene, wherein the disrupted TRAC gene comprises the nucleic acid sequence of SEQ ID NO: 55; and

(ii) a disrupted β2M gene.

E85. The engineered T cell of embodiment 84 further comprising a disrupted CD33 gene.
E86. The engineered T cell of any one of embodiments 1-21 and 74-85, wherein the T cell is a human T cell.
E87. A method of treating cancer in a subject, comprising administering to the subject the population of cells of any one of embodiments 62-73.
E88. The method of embodiment 87, wherein the cancer is a leukemia, optionally acute lymphoblastic leukemia (ALL), acute myeloid leukemia (AML), chronic lymphocytic leukemia (CLL) and chronic myeloid leukemia (CML).
E89. The method of embodiment 87 or embodiment 88, wherein the cancer comprises cells expressing CD33.
E90. An engineered T cell produced by any one of the methods of embodiments 43-59.
E91. A population of cells produced by any one of the methods of embodiments 43-59.
E92. An engineered T cell of any one of embodiments of 1-21, wherein the CAR comprises an amino acid sequence of SEQ ID NO: 104, 105, 107, 111, 114, 117, or 120.
E93. An engineered T cell of any one of embodiments of 1-21, wherein the CAR comprises an amino acid sequence of SEQ ID NO: 104.
E94. An engineered T cell of any one of embodiments of 1-21, wherein the edited CD33 gene comprises a nucleotide sequence of GGATCCAAATTCTGGCTGC (SEQ ID NO:175), GGATCCAAATTTTCTGGCTGC (SEQ ID NO:176), GGATCCTGGCTGC (SEQ ID NO: 177), GGATCCAATTCTGGCTGC (SEQ ID NO: 178), TCCTGGCTGC (SEQ ID NO: 179), GGATCTGGCTGC (SEQ ID NO: 180), GGATCC, and/or GGATCCATTCTGGCTGC (SEQ ID NO: 181).
E95. An engineered T cell of any one of embodiments of 1-21, wherein the edited CD33 gene lacks a fragment comprising GGATCCAAATTTCTGGCTGC (SEQ ID NO: 174).
E96. An engineered T cell of any one of embodiments of 1-21, wherein the edited CD33 gene lacks a fragment, the 3′ segment of which comprises the nucleotide sequence of GGATCCAAATTTC (SEQ ID NO: 182), GGATCCAAATT (SEQ ID NO: 183), or GGATCCAAATTT (SEQ ID NO: 185).
E97. An engineered T cell of any one of embodiments of 1-21, wherein the edited CD33 gene comprises a nucleotide sequence of ACTCCCCAGTTTCATGGTTAC (SEQ ID NO: 197), ACTCCCCAGTCATGGTTAC (SEQ ID NO: 198), ACTCCCCATGGTTAC (SEQ ID NO: 199), ACTCCCCAGTTAC (SEQ ID NO: 200), ACTCATGGTTAC (SEQ ID NO: 201), ACTCCCCATCATGGTTAC (SEQ ID NO: 202), ACTCCCCATTCATGGTTAC (SEQ ID NO: 203), ACTCCCCAGTGTCATGGTTAC (SEQ ID NO: 204), and/or ACTCCCCAGTCTCATGGTTAC (SEQ ID NO: 205).
E98. An engineered T cell of any one of embodiments of 1-21, wherein the edited CD33 gene lacks a fragment comprising ACTCCCCAGTTCATGGTTAC (SEQ ID NO: 196).
E99. An engineered T cell of any one of embodiments of 1-21, wherein the edited CD33 gene lacks a fragment, the 3′ segment of which comprises the nucleotide sequence of ACTCCCCAGTTCATGGTT (SEQ ID NO: 206).
E100. An engineered T cell of any one of embodiments of 1-21, wherein the edited CD33 gene comprises a nucleotide sequence of AGCCATTATCCAGGGACT (SEQ ID NO: 208), AGCCAGGGACT (SEQ ID NO: 209), AGCCATTATTCCAGGGACT (SEQ ID NO: 210), AGTCCAGGGACT (SEQ ID NO: 211), AGCCATTATAATCCAGGGACT (SEQ ID NO: 212), AGCCATTATCCGGGGACT (SEQ ID NO: 213), AGCCATTATACAGGGACT (SEQ ID NO: 214), AGCCATTATTCCGGGGACT (SEQ ID NO: 216), and/or AGCCATTATAATCCGGGGACT (SEQ ID NO: 217).
E101. An engineered T cell of any one of embodiments of 1-21, wherein the edited CD33 gene lacks a fragment comprising AGCCATTATATCCAGGGACT (SEQ ID NO: 207).
E102. An engineered T cell of any one of embodiments of 1-21, wherein the edited CD33 gene lacks a fragment, the 3′ segment of which comprises the nucleotide sequence of AGCCATTATATCCA (SEQ ID NO: 218) or AGCCATTATA (SEQ ID NO: 219).
E103. An engineered T cell of any one of embodiments of 1-21, wherein the edited CD33 gene comprises a nucleotide sequence of TCAGTGACAGGAGGG (SEQ ID NO: 221), TCAGTGACGTACAGGAGGG (SEQ ID NO: 222), TCAGGAGGG (SEQ ID NO: 223), TCAGTGACGGAGGG (SEQ ID NO: 224), TCAGTGACGGGAGGG (SEQ ID NO: 226), TCAGTGACGGTTACAGGAGGG (SEQ ID NO: 227), TCAGTGACGGACAGGAGGG (SEQ ID NO: 228), TCAGTGACGGGTACAGGAGGG (SEQ ID NO: 229), TCAGTACAGGAGGG (SEQ ID NO: 230), TCAGTGACTACAGGAGGG (SEQ ID NO: 231), TCAGTGACGGG (SEQ ID NO: 232), TCAGTGACGG (SEQ ID NO: 233), TCAGTGACGGCAGGAGGG (SEQ ID NO: 234),TCAGTGACGGAGGAGGG (SEQ ID NO: 235), TCAGTGATACAGGAGGG (SEQ ID NO: 236), TCAGTGTACAGGAGGG (SEQ ID NO: 237), and/or TCATACAGGAGGG (SEQ ID NO: 238).
E104. An engineered T cell of any one of embodiments of 1-21, wherein the edited CD33 gene lacks a fragment comprising TCAGTGACGGTACAGGAGGG (SEQ ID NO: 220).
E105. An engineered T cell of any one of embodiments of 1-21, wherein the edited CD33 gene lacks a fragment, the 3′ segment of which comprises the nucleotide sequence of TCAGTGACGGTA (SEQ ID NO: 239) or TCAGTGACG.
E106. An engineered T cell of any one of embodiments of 1-21, wherein the edited CD33 gene lacks a fragment, the 5′ segment of which comprises the nucleotide sequence of GTGACGGTACAGGAGGG (SEQ ID NO: 242).
E107. An engineered T cell of any one of embodiments of 1-21, wherein the edited CD33 gene comprises a nucleotide sequence of AGCTGGAGCT (SEQ ID NO: 244), AGGTGAAGCTGGAGCT (SEQ ID NO: 245), AGGTGAAGCT (SEQ ID NO: 246), AGGTGAAGTTGGAGCT (SEQ ID NO: 247), AGGTGAAGTCGCTGGAGCT (SEQ ID NO: 248), AGGTGGAGCT (SEQ ID NO: 249), AGGTGAAGCGCTGGAGCT (SEQ ID NO: 250), AGGTGACGCTGGAGCT (SEQ ID NO: 252), and/or AGGTGAAGTTTCGCTGGAGCT (SEQ ID NO: 253).
E108. An engineered T cell of any one of embodiments of 1-21, wherein the edited CD33 gene lacks a fragment comprising AGGTGAAGTTCGCTGGAGCT (SEQ ID NO: 243).
E109. An engineered T cell of any one of embodiments of 1-21, wherein the edited CD33 gene lacks a fragment, the 3′ segment of which comprises the nucleotide sequence of AGGTGAAGTTCG (SEQ ID NO: 256), AGGTGAAGTTCGCTGGAG (SEQ ID NO: 259), AGGTGAAGTTCGCTGG (SEQ ID NO: 260), or AGGTGAAGTT (SEQ ID NO: 261).

E110. An engineered T cell of any one of embodiments of 1-21, wherein the edited CD33 gene lacks a fragment, the 5′ segment of which comprises the nucleotide sequence of GGTGAAGTTCGCTGGAGCT (SEQ ID NO: 262).

E111. An engineered T cell of any one of embodiments of 1-21, wherein the edited CD33 gene comprises a nucleotide sequence of AGTTCGCTGGTGTG (SEQ ID NO: 264) and/or AGTTCGCTGAGCTGGTGTG (SEQ ID NO: 266).
E112. An engineered T cell of any one of embodiments of 1-21, wherein the edited CD33 gene lacks a fragment comprising AGTTCGCTGGAGCTGGTGTG (SEQ ID NO: 263).
E113. An engineered T cell of any one of embodiments of 1-21, wherein the edited CD33 gene lacks a fragment, the 3′ segment of which comprises the nucleotide sequence of AGTTCGCTGG (SEQ ID NO: 267).
E114. An engineered T cell of any one of embodiments of 1-21, wherein the edited CD33 gene comprises a nucleotide sequence of ACTACTCACTTCCTCGGTGCT (SEQ ID NO: 269), ACTACTCGGTGCT (SEQ ID NO: 270), ACTACTCATCCTCGGTGCT (SEQ ID NO: 271), ACTACT, ACTACTCACCCTCGGTGCT (SEQ ID NO: 272), ACTACTCCTCGGTGCT (SEQ ID NO: 273), ACTACTCACCTCGGTGCT (SEQ ID NO: 275), ACTACTCACTCGGTGCT (SEQ ID NO: 276), ACTACTCTCCTCGGTGCT (SEQ ID NO: 277), ACTACTTCCTCGGTGCT (SEQ ID NO: 278), ACTACTCACTTCGGTGCT (SEQ ID NO: 279), and/or ACTATCCTCGGTGCT (SEQ ID NO: 280).
E115. An engineered T cell of any one of embodiments of 1-21, wherein the edited CD33 gene lacks a fragment comprising ACTACTCACTCCTCGGTGCT (SEQ ID NO: 268).

E116. An engineered T cell of any one of embodiments of 1-21, wherein the edited CD33 gene lacks a fragment, the 3′ segment of which comprises the nucleotide sequence of ACTACTCACT (SEQ ID NO: 282), ACTACTCACTCCTC (SEQ ID NO: 283), or ACTACTCACTCCTCGGT (SEQ ID NO: 284).

E117. An engineered T cell of any one of embodiments of 1-21, wherein the edited CD33 gene comprises a nucleotide sequence of CCCGATCTTCCTGGTTGT (SEQ ID NO: 286), CCCGATCCTGGTTGT (SEQ ID NO: 287), CCCGATCTGGTTGT (SEQ ID NO: 288), CCCTGGTTGT (SEQ ID NO: 289), CCCGATCTTCTGGTTGT (SEQ ID NO: 290), CCCGATCTTGGTTGT (SEQ ID NO: 291), CCCGATCTCCTGGTTGT (SEQ ID NO: 292), CCCGATCTTCCCTGGTTGT (SEQ ID NO: 293), and/or CCCGAT.
E118. An engineered T cell of any one of embodiments of 1-21, wherein the edited CD33 gene lacks a fragment comprising CCCGATCTTCTCCTGGTTGT (SEQ ID NO: 285).
E119. An engineered T cell of any one of embodiments of 1-21, wherein the edited CD33 gene lacks a fragment, the 3′ segment of which comprises the nucleotide sequence of CCCGATCTTCT (SEQ ID NO: 295).
E120. An engineered T cell of any one of embodiments of 1-21, wherein the edited CD33 gene lacks a fragment, the 5′ segment of which comprises the nucleotide sequence of TCCTGGTTGT (SEQ ID NO: 298).

### EXAMPLES

### Example 1: Generation of TRAC−/β2M−/Anti-CD33 CAR+ T Cells

This example describes the production of allogeneic human T cells that lack expression of the T cell receptor (TCR) gene (gene edited in the TCR Alpha Constant (TRAC) region), the β2-microglobulin (β2M) gene, and that express a chimeric antigen receptor (CAR) targeting CD33 and CD33⁺ cancers. Four unique anti-CD33 CARs (CTX-965, CTX-964, CTX-969 and CTX-970) comprising CD28 co-stimulatory domains were separately expressed in TRAC⁻/β2M⁻ T cells for experimentation and evaluation. Additional anti-CD33 CARs (CTX-965b, CTX-964b, CTX-969b, and CTX-970b) were also generated with a 4-1BB co-stimulatory domain in place of CD28. Additional CARs may be generated as shown in Table 6.

TABLE 6

| | | | | SEQ |
|---|---|---|---|---|
| CAR | Antibody | scEV | CAR structure | ID NO: |
| CTX-964 | Antibody A (VH: SEQ | VH-VL | CD8[signal peptide]-VH-linker-VL- | 101 |
| | ID NO: 65; VL: SEQ ID | | CD8[hinge]-CD8[tm]-CD28[co- | |
| | NO: 66) | | stimulatory domain]-CD3z | |
| CTX-964b | Antibody A (VH: SEQ | VH-VL | CD8[signal peptide]-VH-linker-VL- | 102 |
| | ID NO: 65; VL: SEQ ID | | CD8[hinge]-CD8[tm]-41BB[co-stimulatory | |
| | NO: 66) | | domain]-CD3z | |
| CTX-965 | Antibody A (VH: SEQ | VL-VH | CD8[signal peptide]-VL-linker-VH- | 103 |
| | ID NO: 65; VL: SEQ ID | | CD8[hinge]-CD8[tm]-CD28[co- | |
| | NO: 66) | | stimulatory domain]-CD3z | |
| CTX-965b | Antibody A (VH: SEQ | VL-VH | CD8[signal peptide]-VL-linker-VH- | 104 |
| | ID NO: 65; VL: SEQ ID | | CD8[hinge]-CD8[tm]-41BB[co-stimulatory | |
| | NO: 66) | | domain]-CD3z | |
| CTX-969 | Antibody B (VH: SEQ | VH-VL | CD8[signal peptide]-VH-linker-VL- | 105 |
| | ID NO: 77; VL: SEQ ID | | CD8[hinge]-CD8[tm]-CD28[co- | |
| | NO: 78) | | stimulatory domain]-CD3z | |
| CTX-969b | Antibody B (VH: SEQ | VH-VL | CD8[signal peptide]-VH-linker-VL- | 106 |
| | ID NO: 77; VL: SEQ ID | | CD8[hinge]-CD8[tm]-41BB[co-stimulatory | |
| | NO: 78) | | domain]-CD3z | |
| CTX-970 | Antibody B (VH: SEQ | VL-VH | CD8[signal peptide]-VL-linker-VH- | 107 |
| | ID NO: 77; VL: SEQ ID | | CD8[hinge]-CD8[tm]-CD28[co- | |
| | NO: 78) | | stimulatory domain]-CD3z | |
| CTX-970b | Antibody B (VH: SEQ | VL-VH | CD8[signal peptide]-VL-linker-VH- | 108 |
| | ID NO: 77; VL: SEQ ID | | CD8[hinge]-CD8[tm]-41BB[co-stimulatory | |
| | NO: 78) | | domain]-CD3z | |
| CTX-981 | Antibody C (VH: SEQ | VL-VH | CD8[signal peptide]-VL-linker-VH- | 111 |
| | ID NO: 89; VL: SEQ ID | | CD8[hinge]-CD8[tm]-CD28[co- | |
| | NO: 90) | | stimulatory domain]-CD3z | |
| CTX-981b | Antibody C (VH: SEQ | VL-VH | CD8[signal peptide]-VL-linker-VH- | 114 |
| | ID NO: 89; VL: SEQ ID | | CD8[hinge]-CD8[tm]-41BB[co-stimulatory | |
| | NO: 90) | | domain]-CD3z | |
| CTX-982 | Antibody C (VH: SEQ | VH-VL | CD8[signal peptide]-VH-linker-VL- | 117 |
| | ID NO: 89; VL: SEQ ID | | CD8[hinge]-CD8[tm]-CD28[co- | |
| | NO: 90) | | stimulatory domain]-CD3z | |
| CTX-982b | Antibody C (VH: SEQ | VH-VL | CD8[signal peptide]-VH-linker-VL- | 120 |
| | ID NO: 89; VL: SEQ ID | | CD8[hinge]-CD8[tm]-41BB[co-stimulatory | |
| | NO: 90) | | domain]-CD3z | |

Activated primary human T cells were electroporated with Cas9:gRNA RNP complexes and infected with adeno-associated adenoviral vectors (AAVs) containing anti-CD33 CAR donor template with homology to the TRAC locus to generate TRAC⁻/β2M⁻/anti-CD33 CAR⁺ T cells. Recombinant AAV serotype 6 (AAV6) comprising a CAR donor template (SEQ ID NOS: 49, 51, 53, 55, 57, 59, 61, 63, 109, 112, 115, or 118) were delivered with Cas9:sgRNA RNPs (1 μM Cas9, 5 μM gRNA) to activated human T cells. The following sgRNAs were used: TRAC (SEQ ID NO: 28) and β2M (SEQ ID NO: 30). The unmodified versions (or other modified versions) of the gRNAs may also be used (e.g., SEQ ID NO: 18 or SEQ ID NO: 20). See also Table 4.

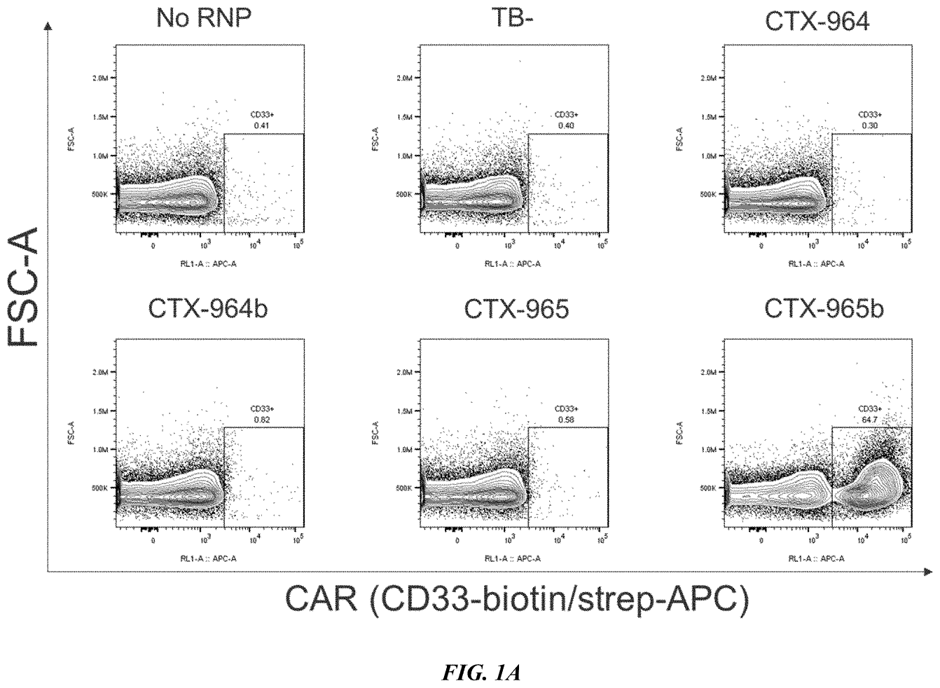

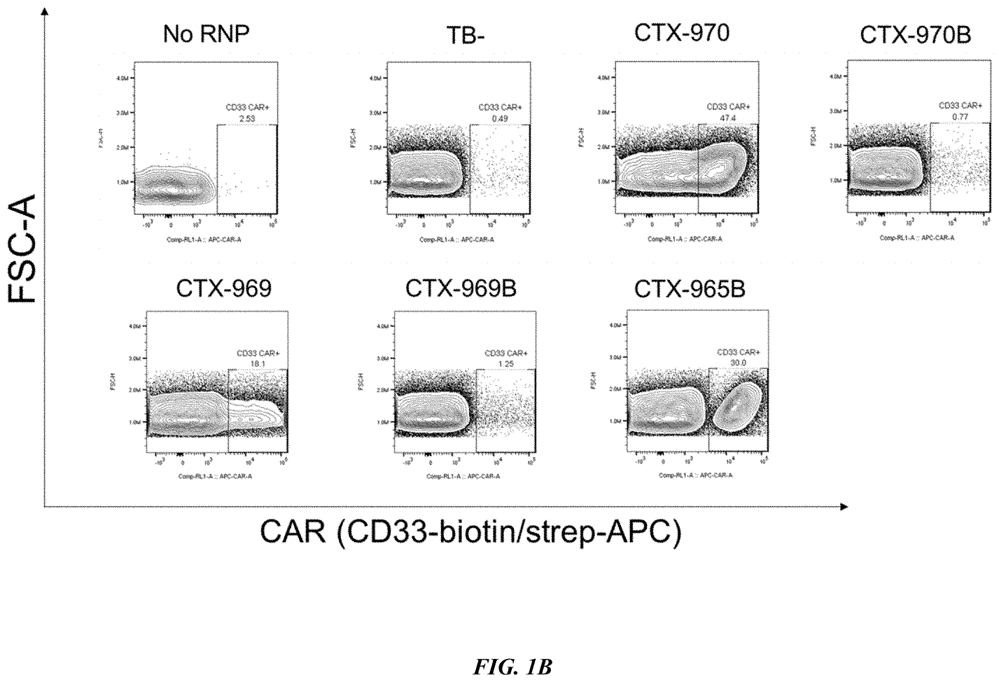

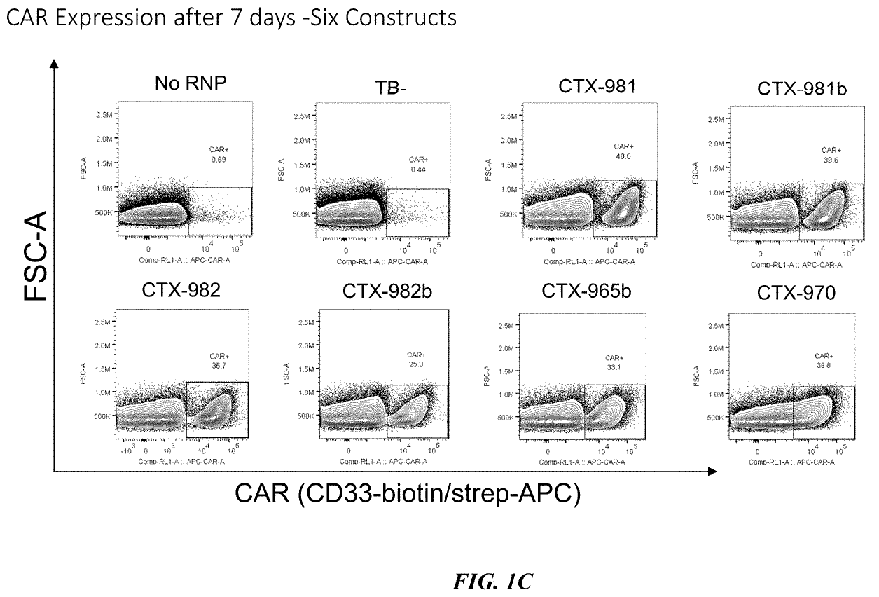

About one (1) week post electroporation, gene-edited T cells were analyzed by flow cytometry to assess the percentage of the cell population that expressed an anti-CD33 CAR. Labeling of cell-surface anti-CD33 CAR was done with a combination of a CD33 protein (AcroBiosystems) conjugated to biotin and a streptavidin-APC secondary reagent (FIG. 1A, FIG. 1B and FIG. 1C). Control cells, including T cells treated with no RNP (e.g., ‘No RNP’) and T cells with a TRAC and B2M gene disruption but no CAR insertion (e.g., TRAC−/B2M− or ‘TB-’), showed no expression of an anti-CD33 CAR surface protein as expected. In contrast, certain T cells generated with a TRAC and B2M gene disruption and a CAR construct did result in a high percentage of the population that expressed an anti-CD33 CAR. This was true for T cells generated with a CTX-965b, CTX-969, or CTX-970 CAR construct. However, not all T cells prepared with a CAR construct had CAR expression. For example, T cells generated with a CTX-964, CTX-964b, CTX-965, CTX 970b, or CTX-969b CAR construct demonstrated no increase in CAR-expressing T cells compared to controls (Table 8; FIG. 1A and FIG. 1B). Surprisingly, for CAR T cells with a given scFv, the co-stimulatory domain used (e.g., CD28 vs. 41BB) had an effect on the percentage of T cells that were positive for CAR expression. For example, while the CTX-965b and CTX-965 CAR constructs had the same scFv, the CTX-965b construct that had a 4-1BB costimulatory domain resulted in significantly more CAR-positive cells than the CTX-965 construct that had a CD28 costimulatory domain (FIG. 1A). Additionally, the orientation of the scFv had an effect on the relative quantities of CAR-expressing cells. For example, CTX-964 resulted in essentially no cells with CAR expression despite being comprised of the same VH and VL domains as CTX-965. Indeed, this outcome was not altered by switching the CAR co-stimulatory domain (e.g., CTX-964 compared to CTX-964b).

Additional TRAC⁻/β2M⁻/anti-CD33 CAR⁺ T cells were generated with CTX-981, CTX-981b, CTX-982 and CTX-982b. For this set of CARs, expression did not appear to be affected by the orientation of the heavy and light chain or the co-stimulatory domain used. Instead, each of the T cell populations generated with this set of CAR constructs resulted in a high proportion of the population that were positive for CAR expression at the cell-surface (FIG. 1C).

Out of the twelve CAR constructs evaluated for CAR expression, three were selected for additional analysis in the examples below:

TRAC−/β2M−/anti-CD33 CAR+ T cells, expressing CTX-965b CAR

TRAC−/β2M−/anti-CD33 CAR+ T cells, expressing CTX-970 CAR

TRAC−/β2M−/anti-CD33 CAR+ T cells. expressing CTX-982b CAR

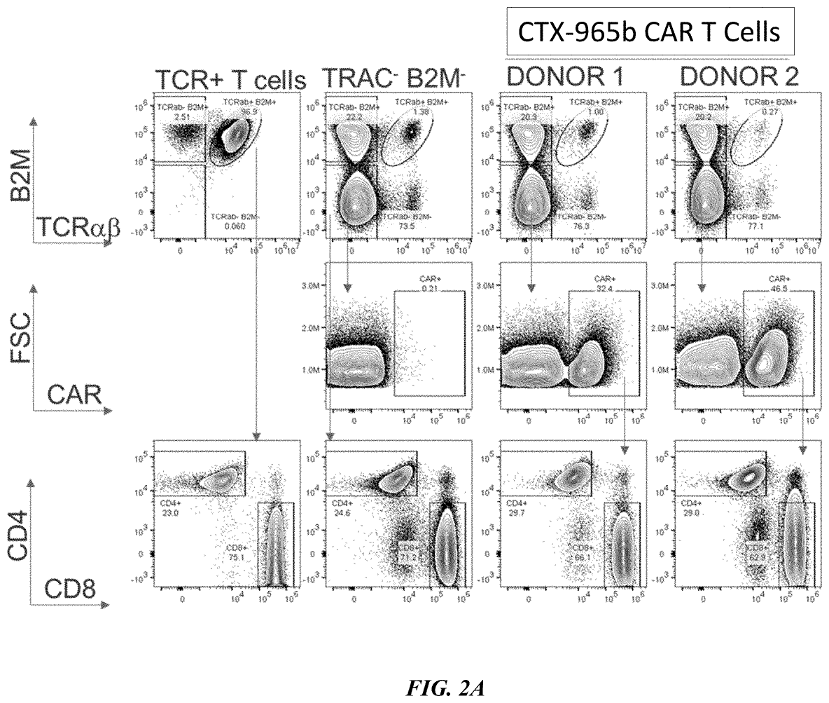

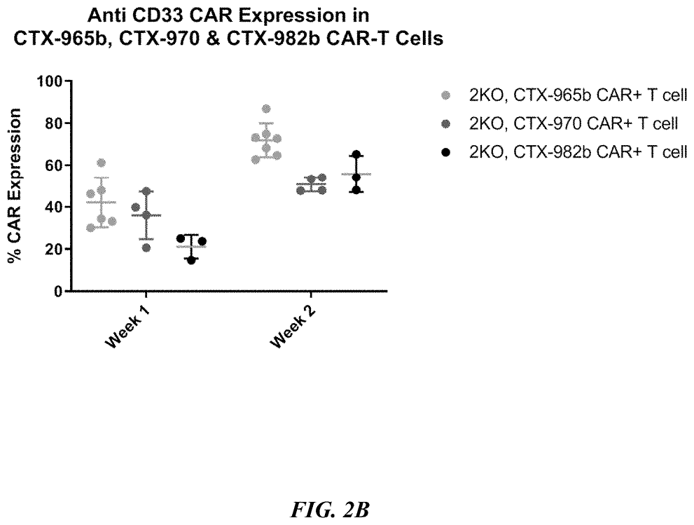

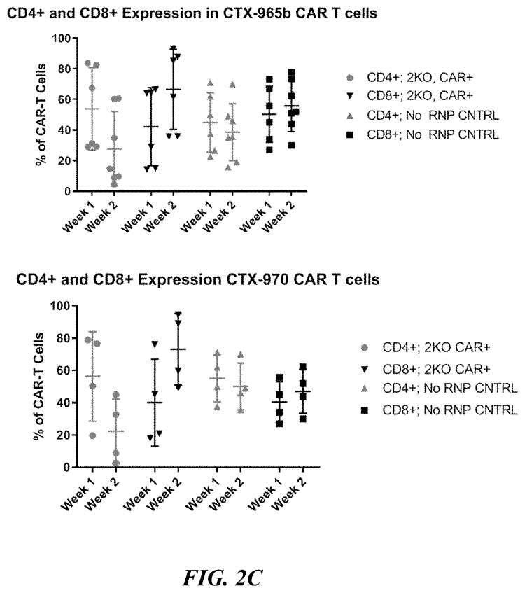

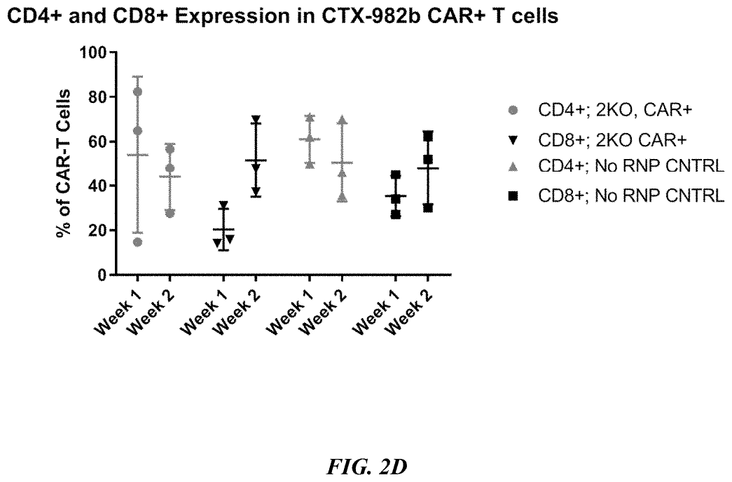

To demonstrate that CAR expression is achieved regardless of the human donor used to generate the gene-edited T cells, anti-CD33 CAR-T cells were prepared using the CTX-965b CAR construct from two additional primary T cell donors according to the gene-editing protocol above (to generate TRAC−/B2M−/anti-CD33 CAR+ T cells referred to as CTX-965b CAR T cells). About one (1) week post-electroporation, cells were analyzed by flow cytometry to assess TRAC (using PE-anti-human TCRαβ, clone BW242/412 from Miltenyi Biotech, Auburn, Calif.), β2M (using PE-Cy7-anti-human β2M, clone 2M2 from Biolegend), CD4 (using APC-Cy7-anti-human CD4, clone RPA-T4 from Biolegend), CD8 (using Pacific Blue-anti-human CD8, clone SK1 from Biolegend) and anti-CD33 CAR (using CD33 protein conjugated to biotin (AcroBiosystems) and streptavidin-APC) expression levels at the cell surface of the edited cell population (FIG. 2A). For cells edited with CTX-965b, a high proportion of the population had positive expression of anti-CD33 CAR and depleted expression of TCR and β2M. Comparable CAR expression was seen for both donor sources, indicating that efficient gene-editing is achieved even when the donor source from which the T cells are derived is varied. Additionally, the levels of CD4 and CD8 on the cell-surface of TRAC⁻/β2M⁻/CAR⁺ cells was found to be comparable to that on control cells (TRAC⁺ T cells and TRAC⁻/β2M⁻ T cells), indicating that the insertion of a CAR is not altering expression of these key phenotypic markers (FIG. 2A).

The following antibodies were used for flow cytometry (Table 7):

TABLE 7

| | Clone | Fluor | Catalogue # | Dilution |
|---|---|---|---|---|
| Antibody | | | | |
| TCRαβ | BW242/412 | PE | 130-099-661 | 1:100 |
| | | | (Miltenyi) | |
| β2M | 2M2 | PE-Cy7 | 316304 | 1:100 |
| | | | (Biolegend) | |
| CD4 | RPA-T4 | APC-Cy7 | Biolegend | 1:100 |
| CD8 | SK1 | Pacific | Biolegend | 1:100 |
| | | Blue | | |
| CD33 | P67.6 | PE | Biolegend | 1:100 |
| | | | 366608 | |
| Strepta- | N/A | APC | 17-4317-82 | 1:100 |
| vidin- | | | (eBioscience | |
| APC | | | through | |
| (SA-APC) | | | ThermoFisher) | |
| Protein | | | | |
| CD33 | | Biotinyl- | CD3-H82E7 | 2 μg/μL |
| protein | | ated; | ACRO | |
| | | Detected | Bioscience | |
| | | with | | |
| | | SA-APC | | |

CTX-965b CAR-T cells were produced from 7 T cell donors in total. In addition, the CTX-970 CAR and CTX-982b CAR was used to generate TRAC-B2M−/anti-CD33 CAR+ T cells (also referred to as CTX-970 CAR T cells and CTX-982b CAR T cells) from 4 T cell donors.

In addition to CAR expression, the edited T cell populations were analyzed for the percentage of cells with depleted surface expression of TCRαβ and β2M due to gene disruption by CRISPR/Cas9 (Table 8). As above, about one (1) week post electroporation, cells were processed for flow cytometry to assess anti-CD33 CAR expression levels on the cell surface of the edited cell population. TCRαβ and β2M expression were also assessed by staining cells with antibodies as described above. A high percentage of edited cells demonstrated depletion of both TCRαβ and β2M surface expression (Table 8). Additionally, depending upon the CAR construct used, a high percentage of edited cells were positive for expression of an andi-CD33 CAR. These data show that T cells from healthy donors can be edited with CRISPR/Cas9 to produce TRAC and B2M disruption and gene insertion resulting in expression of an anti-CD33 CAR.

TABLE 8
Percentage of TCR⁻, B2M⁻ and anti-CD33 CARP cells
in the gene edited cell population

| | N | | | | % |
|---|---|---|---|---|---|
| | (# of T | | | | TRAC−/ |
| CTX-cell | cell | % | % | | B2M−/ |
| population | donors) | TCRab− | B2M− | % CAR+ | CAR− |
| CTX-964 | 1 | NA | NA | 0.3 | NA |
| CTX-964b | 1 | NA | NA | 0.82 | NA |
| CTX-965 | 1 | NA | NA | 0.58 | NA |
| CTX-965b | 6 | 96.8 ± 2.9 | 73.4 ± 8.9 | 40 ± 14 | 41.2 ± 13.6 |
| CTX-969 | 1 | 93.6 | 75.5 | 15.9 | 18.1 |
| CTX-969b | 1 | 92.8 | 73.5 | 1.8 | 1.3 |
| CTX-970 | 4 | 97.5 ± 2.1 | 73.4 ± 11.7 | 36.9 ± 8.4 | 40.2 ± 8.4 |
| CTX-970b | 1 | 93.2 | 73.7 | 0.9 | 0.8 |
| CTX-981 | 1 | 93.6 | 55.6 | 32.4 | 36.9 |
| CTX-981b | 1 | 93.1 | 55.5 | 32.9 | 36.5 |
| CTX-982 | 1 | 93.3 | 56.4 | 28.5 | 33.1 |
| CTX-982b | 3 | 96.3 ± 3.4 | 71.2 ± 13.9 | 20.4 ± 4.8 | 21.8 ± 5.0 |

Surprisingly and unexpectedly, the proportion of cells expressing anti-CD33-CAR increased at 2 weeks of culture relative to cells analyzed at 1 week post electroporation/rAAV infection. This trend was consistent for populations of T cells acquired from different donors and transfected with different CAR constructs: CTX-965b (6 donors), CTX-970 (4 donors) or CTX-982b (3 donors) (FIG. 2B).

T cell Proportion Assay. The proportion of gene-edited T cells that were CD4+ T cells or CD8+ T cells was assessed by flow cytometry at one and two weeks (i.e., 7 days and 14 days) post-editing using the CD4⁺ and CD8⁺ labeling antibodies listed in Table 7. Surprisingly, the percentage of CD4⁺ cells in the edited cell population decreased at 2 weeks post-editing relative to 1 week post-editing. This depletion of CD4 T cells was not observed in control T cells that lacked expression of an anti-CD33 CAR (e.g.: no RNP) (FIG. 2C). For CAR-expressing T cells however, this depletion of CD4 T cells over time was observed for T cells edited with different CAR constructs and acquired from different donors: CTX-965b (FIG. 2C), CTX-970 (FIG. 2C), or CTX-982b (FIG. 2D).

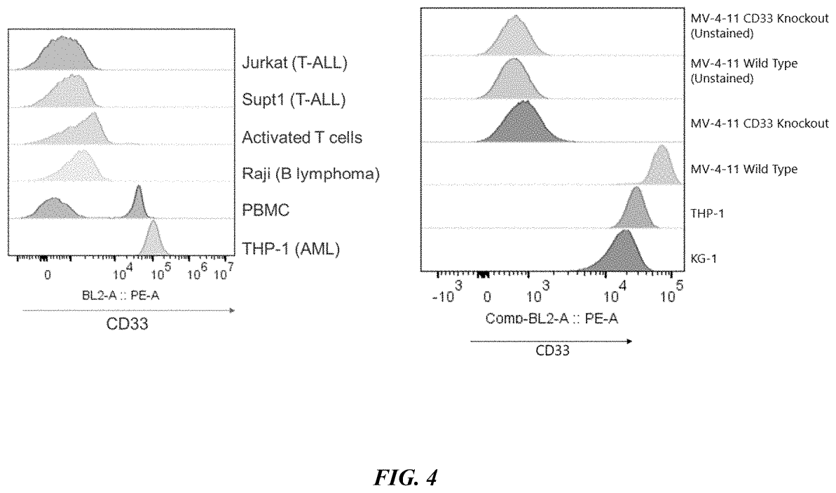

CD33 is expressed on activated cultured T cells (FIG. 4, left panel). Additionally, a higher proportion of CD4C T cells are positive for CD33 expression compared to CD8⁺ T cells (58% vs. 31%, respectively).

The expansion of CAR⁺ cells and the decrease in CD4C cells in anti-CD33 CAR-T cultures suggests that the anti-CD33 CAR-T cells may be reacting against CD33-expressing T cells in the same culture. Indeed, complete loss of surface CD33 expression is observed in anti-CD33 CAR-T (e.g., CTX-965b) cultures relative to controls (36% of cells express CD33 in T cells transfected with no RNP, 30% in TRAC−B2M− cultures, and only 0.21% in CTX-965b cultures), suggesting that CD33-expressing T cells are eliminated by anti-CD33 CAR-expressing T cells. Additionally, given that CD4C T cells express higher levels of CD33, they may be more susceptible to self-reactive killing by CAR-expressing T cells. This self-reactivity may explain the observed decrease in the percentage of CD4⁺ T cells in the T cell culture over time (FIG. 2C). Self-reactive killing may also function to stimulate and activate CAR+ T cells, leading to the observed expansion of CAR+ T cells over time (FIG. 2B). Based upon this data, genetic disruption of CD33 in the T cell culture may prevent self-reactive killing and enable maintenance of balanced CD4/CD8 CAR-T ratios. Genetic disruption of CD33 in T cells can be performed using sgRNA sequences, as shown below.

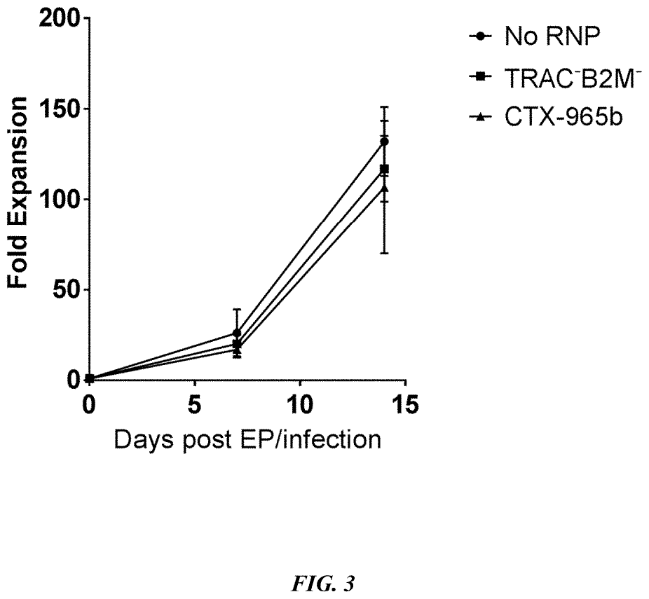

CTX-965b gene-edited T cells were then expanded for two weeks in vitro to demonstrate that the gene edited T cells could grow and expand comparably to control cells (TRAC⁺ T cells and TRAC⁻/β2M⁻ T cells). CTX-965b cells expanded ˜100 fold, similarly to control cells (FIG. 3).

### Example 2: Functional Capabilities of Anti-CD33 CAR+ T Cells

TRAC−/β2M−/anti-CD33 CAR+ T cells (e.g.: CTX-965b CAR T cells or CTX-970 CAR T cells) were generated as described in Example 1. Gene-edited CAR T cells populations were generated from multiple T cells donors. Populations of TCR+ T cells and TRAC−/β2M− T cells were similarly generated for use as controls.

Following preparation of the edited T cells by transfection, the functional activity of the CAR T cells was verified using a flow cytometry-based cytotoxicity assay. The CAR T cells or control T cells (TCR+ T cells and TCR-B2M− T cells) were co-cultured with a CD33-expressing cancer cell line. Three different human AML-derived cell lines (referred to as the “target cells”) were tested: THP-1 (ATCC TIB-202), KG-1 (ATCC CCL-246), or MV4-11 (ATCC CRL-9591). The target cells were labeled with 5 μM efluor670 (eBiosciences), washed and incubated in co-cultures with the CTX-965b CAR T cells (TRAC−/B2M−/anti-CD33 CAR+) or CTX-970 CAR T cells (TRAC−/B2M−/anti-CD33 CAR+) at varying ratios (from 0.05:1 to 1:1 T cells:target cells). The target cells were seeded at 50,000 cells per well in a 96-well, U-bottom plate. The co-culture was incubated overnight. On the following day, wells were washed and media was replaced with 200 μL of media containing a 1:500 dilution of 5 mg/mL DAPI (Molecular Probes). 25 μL of CountBright beads (Life Technologies) were then added to each well and the cell cultures were analyzed for cell viability by flow cytometry (i.e., viable cells being negative for DAPI staining).

Percent cell lysis of the target cells (e.g., THP-1, KG-1 or MV4-11) was then determined using the following formula:

Percent cell lysis=(1−((total number of target cells in a test sample)+(total number of target cells in a control sample))×100;

wherein a test sample was target cells (e.g., THP-1 cells) co-cultured with 1) CTX-965b CAR T cells; 2) CTX-970 CAR T cells; 3) TRAC⁺ T cells; or 4) TRAC⁻/β2M⁻ T cells, and

a control sample was target cells alone that had not been co-cultured.

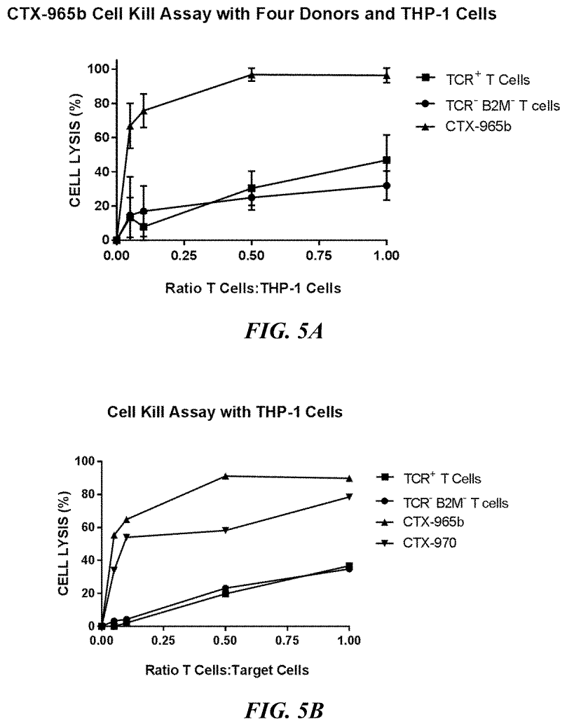

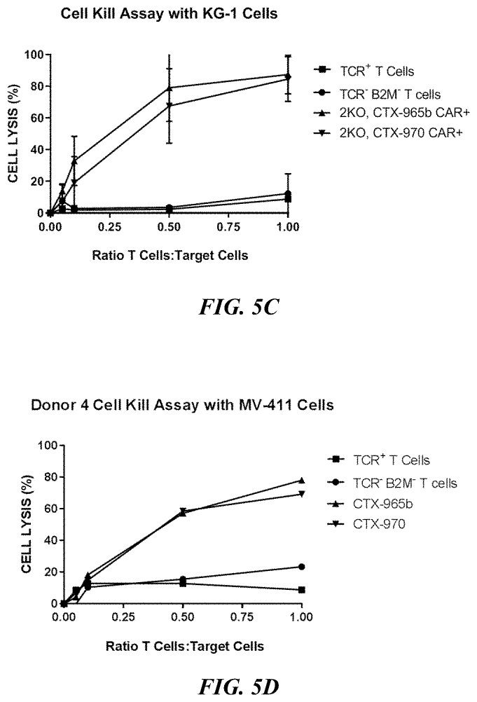

CTX-965b CAR T cells were effective at killing THP-1 cells (i.e., inducing cytotoxicity) at all ratios of CTX-965b T cells to THP-1 cells that were tested (FIG. 5A). CTX-965b CAR T cells were also effective at killing other AML cancer cells (KG-1 and MV4-11) (FIG. 5C and FIG. 5D) In addition, an alternate anti-CD33 CAR, CTX-970, was also effective at killing all of the AML cancer cell lines tested (FIG. 5B-FIG. 5D). These data demonstrate that anti-CD33 CAR T cells as described herein induce potent cell lysis of CD33-expressing AML cell lines. These results are reproducible even when the allogeneic CAR-T cell populations are produced from different donor sources (FIG. 5A).

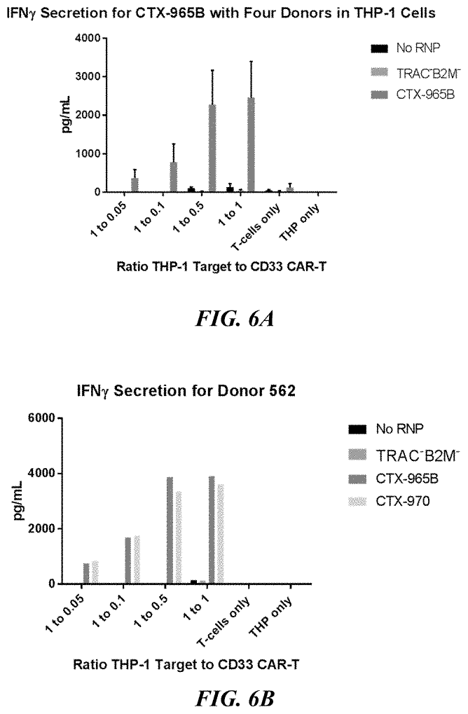

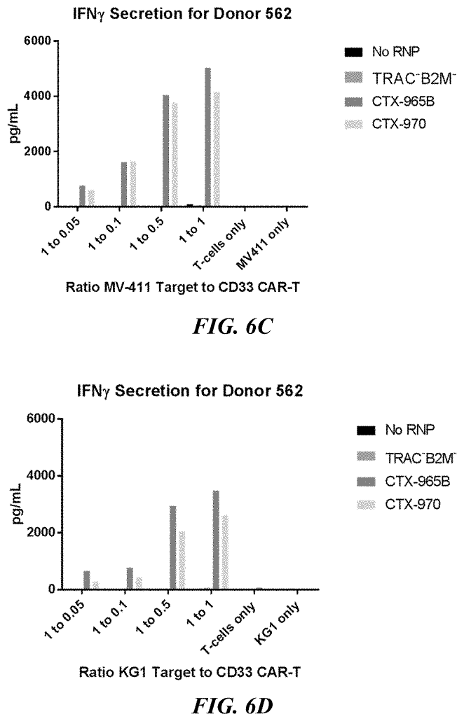

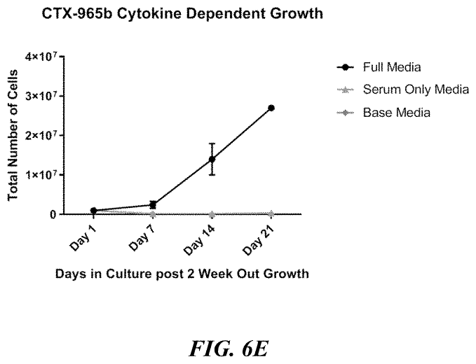

Cytokine Release Assay. The functional ability of TRAC−/B2M−/anti-CD33 CAR+ T cells (e.g., CTX-965b CAR T cells and CTX-970 CAR T cells) to induce cytokine secretion/release in the presence of THP-1, KG-1 or MV4-11 target cells was tested in comparison to control T cells (e.g., TCR+ T cells and TRAC⁻/β2M⁻ T cells). To measure cytokine release, T cells were co-cultured with the target cells for 24 hours. Supernatant media was collected for measurement of IFNγ or IL-2 cytokines by ELISA-based assays (RD Systems) according to manufacturer's instructions (RD Systems). CTX-965b CAR T cells and CTX-970 CAR T cells produced much higher levels of IFNγ relative to controls in response to THP-1 cells, even at low ratios of CAR T cells to target cells (FIG. 6A-FIG. 6D). These results indicate that expression of the anti-CD33 CAR, introduced using either the CTX-965b or CTX-970 CAR construct, confers effector functions on T cells in the presence of CD33-expressing target cells.

Cytokine Dependency. To determine whether gene-editing resulted in unwanted off-target editing that could generate cells with adverse properties, such as uncontrolled cell growth, the ability of TRAC⁻/β2M⁻/anti-CD33 CAR⁺ cells to grow in the absence of cytokines and/or serum was assessed. 1×10⁶ TRAC⁻/β2M⁻/anti-CD33 CAR⁺ T cells were plated on day 0, approximately two weeks post gene editing. The number of viable cells were enumerated 7, 14 and 21 days post plating in either full media, 5% human serum without cytokines (IL-2 and IL-7), or base media lacking serum and cytokines. TRAC⁻/β2M⁻/anti-CD33 CAR⁺ T cells generated from 2 donors were tested. No cell expansion was detected at 14 or 21 days when plated in the media that lacked cytokines, suggesting that any potential off-target effects due to genome editing did not induce growth factor independent expansion activity to the cells. The cells only expanded in the presence of cytokines (full media that contains cytokines) and did not expand in the presence of serum alone, as shown in FIG. 6E. Thus, in vivo, TRAC⁻/β2M⁻/anti-CD33 CAR⁺ cells are not expected to grow in the absence of cytokine, growth factor or antigen stimulation due to any off-target genome editing.

### Example 3: Efficacy of TRAC−/B2M−/Anti-CD33 CAR+ T Cells in an AML Xenograft Model in NOG Mice

CRISPR/Cas9 and AAV6 were used as above (see for example, Example 1) to create human T cells that lacked expression of the TCR and β2M with concomitant expression of a CAR construct targeting CD33 (CTX-965b; SEQ ID NO: 56 (nucleic acid); SEQ ID NO: 104 (amino acid)) that was inserted into the TRAC locus. Activated T cells were first electroporated with 2 distinct Cas9:sgRNA RNP complexes, one containing sgRNAs targeting TRAC (SEQ ID NO: 28) and the other containing sgRNAs targeting β2M (SEQ ID NO: 30). The DNA double stranded break at the TRAC locus was repaired by homology directed repair with an AAV6-delivered DNA template (SEQ ID NO: 55) (encoding anti-CD33 CAR CTX-965b comprising the amino acid sequence of SEQ ID NO: 104) containing right and left homology arms to the TRAC locus flanking a chimeric antigen receptor cassette (−/+ regulatory elements for gene expression). The resulting modified T cells are 2×KO (TRAC−/β2M−), anti-CD33 CAR+ T cells (CTX-965b T cells).

Treatment in Small Tumor Model

The ability of the modified anti-CD33 CAR+ T cells (2×KO (TRAC−/β2M−), anti-CD33 CAR+ T cells) to ameliorate disease caused by a CD33+ cancer cell line was evaluated in NOG mice using methods employed by Translational Drug Development, LLC (Scottsdale, Ariz.). In brief, twelve 5-8 week old female, CIEA NOG (NOD.Cg-Prkdc^{scid}I12rg^{tm1Sug}/JicTac) mice were individually housed in ventilated microisolator cages and maintained under pathogen-free conditions for 5-7 days prior to the start of the study. Mice received a subcutaneous inoculation of 5×10⁶ THP-1, human AML derived cells/mouse in the right hind flank. Once mean tumor size had reached 25-75 mm³ (target of ˜50 mm³), the mice were further divided into 3 treatment groups as shown in Table 9. On Day 1, treatment groups 2 and 3 received a single 200 μl intravenous dose of anti-CD33 CAR+ T cells (CTX-965b T cells) according to Table 9.

TABLE 9
Treatment groups

| | | THP-1 | T cell treatment | |
|---|---|---|---|---|
| Group | CAR-T | cells | (i.v.) | N |
| 1 | None | 5 × 10⁶ | None | 5 |
| | | cells/mouse | | |
| 2 | 2X KO, anti-CD33 | 5 × 10⁶ | 1 × 10⁷ | 5 |
| | CAR + T cells | cells/mouse | cells/mouse | |
| 3 | 2X KO, anti-CD33 | 5 × 10⁶ | 2 × 10⁷ | 3 |
| | CAR + T cells | cells/mouse | cells/mouse | (5*) |

*N = 5 for Group 3 in large tumor model

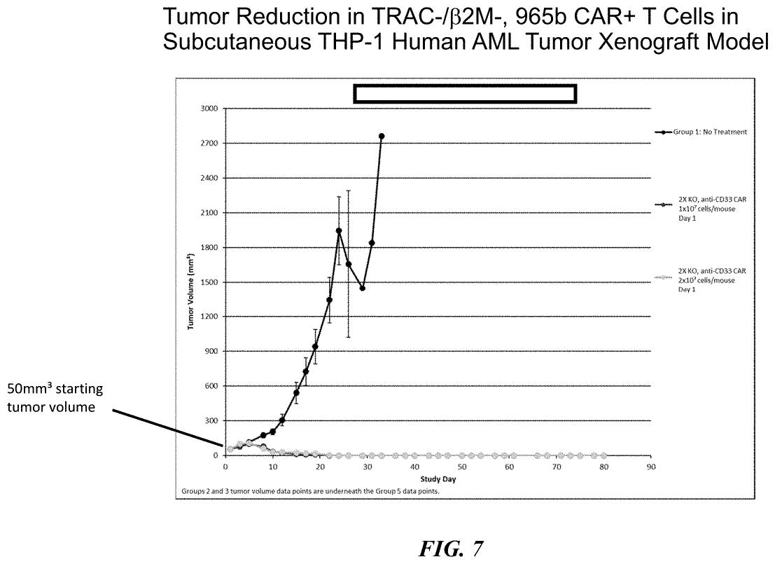

Tumor volume was measured 3 times weekly starting on the day of treatment initiation. By day 5, all animals treated with anti-CD33 CAR+ T cells (Treatment groups 2 and 3) began to show a decrease in tumor volume. By days 6-8, animals treated with anti-CD33 CAR T cells (Treatment groups 2 and 3) demonstrated a significant reduction in tumor growth compared to animals that were untreated (Treatment group 1) (FIG. 7). These data demonstrate that allogeneic anti-CD33 CAR+ T cells can quickly and effectively reduce the growth and expansion of CD33+ AML in vivo. Complete regression of the CD33+ AML tumors due to treatment with allogeneic anti-CD33 CAR+ T cells was achieved by and persisted for the length of the study. For group 2 all mice had 0 mm3 tumors at day 22, for group 3 its day 24.

Treatment in Large Tumor Model

The ability of the modified anti-CD33 CAR+ T cells (2×KO (TRAC−/β2M−), anti-CD33 CAR+ T cells) to reduce large tumors caused by a CD33+ cancer cell line was evaluated in NOG mice using methods employed by Translational Drug Development, LLC (Scottsdale, Ariz.). In brief, twelve 5-8 week old female mice, CIEA NOG (NOD.Cg-Prkdc^{scid}I12rg^{tm1Sug}/JicTac) were individually housed in ventilated microisolator cages and maintained under pathogen-free conditions for 5-7 days prior to the start of the study. Mice received a subcutaneous inoculation of 5×10⁶ THP-1, human AML derived cells/mouse. Once mean tumor size reached 125-175 mm³ (target of ˜150 mm³), the mice were further divided into 3 treatment groups as shown in Table 9.

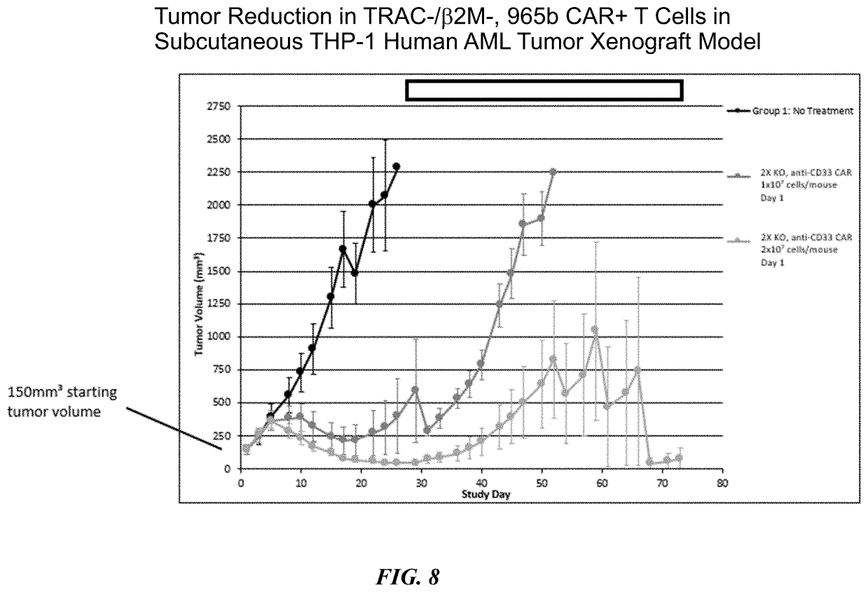

On Day 1, treatment groups 2 and 3 received a single intravenous dose of anti-CD33 CAR+ T cells according to Table 9. Tumor volume was measured 3 times weekly starting with the day of treatment initiation. By day 5, all mice treated with anti-CD33 CAR+ T cells (Treatment groups 2 and 3) began to show an initial decrease in tumor volume (FIG. 8). This reduction was sustained in most of the animals receiving the moderate dose of CAR-T cells for up to day 30 of the study. Tumor growth was substantially delayed in group 3 that received the high dose of anti-CD33 CAR+ T cells. Indeed, 2 of the 5 animals that received the higher dose of anti-CD33 CAR T cells showed low to no tumor growth from the administration of CAR+ T cells through the end of the observation period (day 73). Specifically, one animal had a low tumor volume of 157 mm³ on day 73, and a second animal demonstrated a complete elimination of the tumor by day 43 that continued to the end of the observation period (day 73). These data demonstrate that allogeneic anti-CD33 CAR+ T cells can significantly inhibit or reduce the growth and expansion of large CD33+ AML tumors in vivo.

### Example 4: Design of gRNAs for CD33 Gene Editing

This example describes efficient editing of the CD33 gene in primary human T cells ex vivo using CRISPR/Cas9 gene editing for the purpose of eliminating the risk of self-reactivity in anti-CD33 CAR+ T cells. Genomic segments of the CD33 gene containing the first two protein coding exons (exons 2 and 3) of the CD33 gene were used as input into gRNA design software. Desired gRNAs were those that would introduce insertions or deletions in the coding sequence and thereby produce an out-of-frame mutation or premature stop codon that impairs gene expression (i.e., giving an out-of-frame/loss-of-function allele that is referred to as a “CD33 knockout”). Ten (10) in silico-identified gRNA spacer sequences targeting the CD33 gene were selected based upon a predicted low-risk of off-target gene editing (SEQ ID NO: 132-141). The gRNAs were synthesized and specifically modified as indicated in Table 10. While the gRNAs in Table 10 were modified with 2′-O-methyl phosphorothioate modifications, unmodified gRNAs, or gRNAs with other modifications, may also be used.

TABLE 10
CD33 gRNA Sequences and Target Sequences
gRNA Sequences

| Name | Unmodified Sequence | Modified Sequence |
|---|---|---|
| CD33-1 | UGGCUAUGGAUCCAAAUUUC | U*G*G*CUAUGGAUCCAAAUUUCguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 142) |
| | (SEQ ID NO: 132) | |
| CD33-2 | ACUCCCCAGUUCAUGGUUAC | A*C*U*CCCCAGUUCAUGGUUACguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 143) |
| | (SEQ ID NO: 133) | |
| CD33-3 | GACAAGAACUCCCCAGUUCA | G*A*C*AAGAACUCCCCAGUUCAguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 144) |
| | (SEQ ID NO: 134) | |
| CD33-4 | GGGAAGGAGCCAUUAUAUCC | G*G*G*AAGGAGCCAUUAUAUCCguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 145) |
| | (SEQ ID NO: 135) | |
| CD33-5 | GCAGGAGUCAGUGACGGUAC | G*C*A*GGAGUCAGUGACGGUACguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 146) |
| | (SEQ ID NO: 136) | |
| CD33-6 | ACCUGUCAGGUGAAGUUCGC | A*C*C*UGUCAGGUGAAGUUCGCguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 147) |
| | (SEQ ID NO: 137) | |
| CD33-7 | CAGGUGAAGUUCGCUGGAGC | C*A*G*GUGAAGUUCGCUGGAGCguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 148) |
| | (SEQ ID NO: 138) | |
| CD33-8 | CCCCAGGACUACUCACUCCU | C*C*C*CAGGACUACUCACUCCUguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 149) |
| | (SEQ ID NO: 139) | |
| CD33-9 | GAACACCCCCGAUCUUCUCC | G*A*A*CACCCCCGAUCUUCUCCguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | (SEQ ID NO: 150) |
| | (SEQ ID NO: 140) | |
| CD33-10 | AGGCCCAAAAUCCUCAUCCC | A*G*G*CCCAAAAUCCUCAUCCCguuu |
| | guuuuagagcuagaaauagc | uagagcuagaaauagcaaguuaaaaua |
| | aaguuaaaauaaggcuaguc | aggcuaguccguuaucaacuugaaaaa |
| | cguuaucaacuugaaaaagu | guggcaccgagucggugcU*U*U*U |
| | ggcaccgagucggugcUUUU | NO: 151) |
| | (SEQ ID NO: 141) | |

Target Sequences

| Guide | |
|---|---|
| Name | Target Sequence (PAM) |
| CD33-1 | TGGCTATGGATCCAAATTTC (TGG) (SEQ ID NO: 152) |
| CD33-2 | ACTCCCCAGTTCATGGTTAC (TGG) (SEQ ID NO: 153) |
| CD33-3 | GACAAGAACTCCCCAGTTCA (TGG) (SEQ ID NO: 154) |
| CD33-4 | GGGAAGGAGCCATTATATCC (AGG) (SEQ ID NO: 155) |
| CD33-5 | GCAGGAGTCAGTGACGGTAC (AGG) (SEQ ID NO: 156) |
| CD33-6 | ACCTGTCAGGTGAAGTTCGC (TGG) (SEQ ID NO: 157) |
| CD33-7 | CAGGTGAAGTTCGCTGGAGC (TGG) (SEQ ID NO: 158) |
| CD33-8 | CCCCAGGACTACTCACTCCT (CGG) (SEQ ID NO: 159) |
| CD33-9 | GAACACCCCCGATCTTCTCC (TGG) (SEQ ID NO: 160) |
| CD33-10 | AGGCCCAAAATCCTCATCCC (TGG) (SEQ ID NO: 161) |

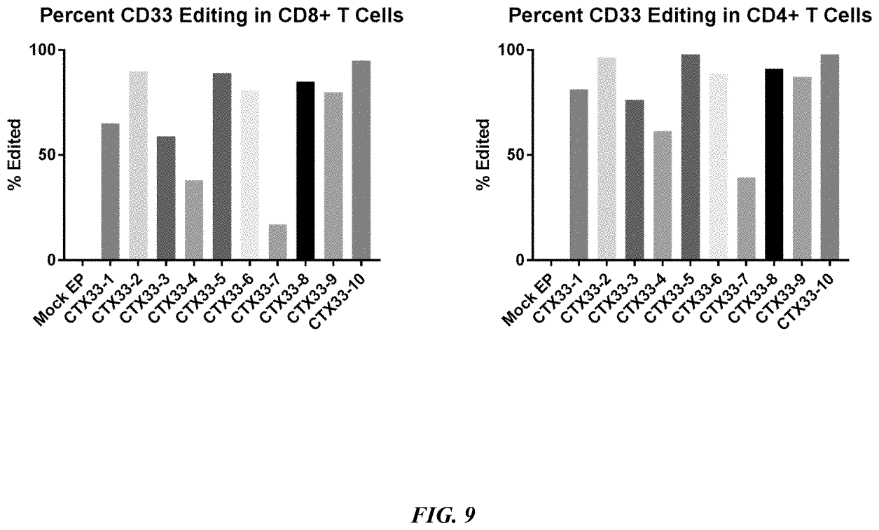

Primary human T cells were transfected (electroporated) with a ribonucleoprotein particle (RNP) containing Cas9 nuclease and a synthetic modified sgRNA targeting the CD33 gene (sequences in Table 10) or controls (no Cas9, no gRNA). Four to six (4-6) days post transfection, cells were processed by flow cytometry (primary antibody: anti-human CD33 antibody, Biolegend cat #366608) to assess CD33 expression levels at the cell surface. For a portion of the gRNAs tested, nearly all of the T cells had loss of surface CD33 expression indicative of gene disruption (i.e., CD33-negative) (e.g., CTX33-2, CTX33-5, and CTX33-10 as shown in FIG. 9)

Three (3) gRNAs were further tested by TIDE analysis, as indicated in Table 11. Of these, two (2) gRNAs achieved high efficiency editing, with editing frequencies above 97%.

TABLE 11
CD33 gRNA spacer sequences, cutting efficiencies,
and CD33 surface protein expression in gene
edited T cells.

| | | | | Protein | Protein |
|---|---|---|---|---|---|
| | | | | expression | expression |
| | | | | in | in |
| | | | | CD4+ cells | CD8+ cells |
| | | | | (knockdown | (knockdown |
| | gRNA spacer | Indel | R2 | %) | %) |
| CD33-1 | UGGCUAUGGAUCCAAAUUUC | | | 81% | 65% |
| | (SEQ ID NO: 164) | | | | |
| CD33-2 | ACUCCCCAGUUCAUGGUUAC | 97.2 | 98 | 97% | 90% |
| | (SEQ ID NO: 165) | | | | |
| CD33-3 | GACAAGAACUCCCCAGUUCA | | | 76% | 59% |
| | (SEQ ID NO: 166) | | | | |
| CD33-4 | GGGAAGGAGCCAUUAUAUCC | | | 61% | 38% |
| | (SEQ ID NO: 167) | | | | |
| CD33-5 | GCAGGAGUCAGUGACGGUAC | 57.5 | 93 | 98% | 89% |
| | (SEQ ID NO: 168) | | | | |
| CD33-6 | ACCUGUCAGGUGAAGUUCGC | | | 89% | 81% |
| | (SEQ ID NO: 169) | | | | |
| CD33-7 | CAGGUGAAGUUCGCUGGAGC | | | 39% | 17% |
| | (SEQ ID NO: 170) | | | | |
| CD33-8 | CCCCAGGACUACUCACUCCU | | | 91% | 85% |
| | (SEQ ID NO: 171) | | | | |
| CD33-9 | GAACACCCCCGAUCUUCUCC | | | 87% | 80% |
| | (SEQ ID NO: 172) | | | | |
| CD33-10 | AGGCCCAAAAUCCUCAUCCC | 97.6 | 97 | 98% | 95% |
| | (SEQ ID NO: 173) | | | | |

Initial experiments were carried out using the guide exhibiting the highest % indel and % knockdown of protein expression.

### Analysis of Off-Target and On-Target Profiles in T Cells.

A homology-dependent assessment of the CD33 gRNAs of Table 12 showed that CD33-2 (SEQ ID NO: 165) had an averaged on-target indel frequency of 88% and no off-target sites with an indel frequency greater than 0.2%. In contrast, other CD33 gRNAs with high average on-target indel frequency greater than 90% (e.g., CD33-5, CD33-8 and CD33-10) produced numerous off-target indels >1% and were deprioritized. The analysis was completed with hybrid capture combined with next-generation sequencing, starting from T cells gene edited with each of the ten guides listed in Table 12. Two cell populations of edited cells were generated from two different donor T cells (termed 1 and 2) and used for this assay. The off-target data guided selection of this particular CD33 gRNA (CD33-2) for further analysis.

TABLE 12
CD33 on-target genomic editing efficiency and off-target editing results in
gene edited T cells.

| | On-target mean | |
|---|---|---|
| Guide | editing (%) ^{a} | Off-target summary |
| CD33-1 | 48.2 | One 4% off-target, one < 1% off-target |
| CD33-2 | 88 | No identified off-targets |
| CD33-3 | 64.3 | One < 1% off target |
| CD33-4 | 29 | Two < 1% off-targets |
| CD33-5 | 91.4 | Two > 70% off-targets, one < 100 off-target |
| CD33-6 | 48.6 | One < 100 off target |
| CD33-7 | 25 | One 2% off-target |
| CD33-8 | 93.9 | Three 1-2% off-targets, six < 100 off-targets |
| CD33-9 | 64 | One 2% off-target |
| CD33-10 | 92.8 | Two > 40% off-targets, four 1-10% off-targets, |
| | | five < 100 off-targets |

^{a} Average across donors 1 and 2.

### Analysis of On-Target Indel Profiles in T Cells.

The data used to quantify off-target editing were also used to quantify and summarize the most frequent on-target indels for all CD33 guides listed in Table 12. This data was generated from hybrid capture of the CD33 locus combined with next-generation sequencing in two donors (termed 1 and 2).

Following gene editing, hybrid capture analysis of the CD33 locus in a population of T cells following CRISPR/Cas9 gene editing to produce CD33˜ T cells results in specific indel frequencies and edited gene sequences at the CD33 locus (Tables 13-22; deletions as dashes and insertions in bold).

For the purposes of individual sequence quantification from hybrid capture data, sequence reads aligning across the CD33 on-target site, 20 bp upstream and downstream of the cut site, were selected and considered for indel sequence quantification. From the selected reads, the sequence within 10 bp upstream and downstream of each putative cut site (˜3 bp upstream of the PAM (Jinek, et al., Science 2012) was quantified as a representative region of on-target non-homologous end joining (NHEJ) editing. The data on these on-target gene edited sequences is presented in the tables below, with the frequencies of these sequences representing the percent of all sequences spanning the on-target site within 20 bp upstream and downstream of each cut site. The indels for each guide are shown relative to an on-target reference sequence in Tables 13-22. The reference sequence is centered on the cleavage site with 10 bp in either direction, ending 4 bp 3′ of the PAM.

TABLE 13
On-target gene edited sequences >1% frequency in
at least one CD33-1 gene edited T cell donor.
Reference on-target sequence^{a}:
GGATCCAAATTTC(TGG)CTGC (SEQ ID NO: 174)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| 175 | GGATCCAAA-TTCTGGCTGC | | T | 6.2 | 6 | 6.1 | 0.1 |
| 176 | GGATCCAAATTTTCTGGCTGC | | | 5 | 6.4 | 5.7 | 1 |
| | -------------TGGCTGC | 182 | GGATCCAAATTTC | 2.8 | 2.1 | 2.5 | 0.5 |
| 177 | GGATC-------CTGGCTGC | | CAAATTT | 2.5 | 2.4 | 2.4 | 0 |
| 178 | GGATCCAA--TTCTGGCTGC | | AT | 2 | 2.7 | 2.3 | 0.6 |
| 179 | ----------TCCTGGCTGC | 183 | GGATCCAAATT | 0 | 3.8 | 1.9 | 2.7 |
| 180 | GGA--------TCTGGCTGC | | TCCAAATT | 2.2 | 1.2 | 1.7 | 0.7 |
| | GGATC--------------C | 184 | CAAATTTCTGGCTG | 1.9 | 0.9 | 1.4 | 0.7 |
| | -------------------- | 174 | GGATCCAAATTTCTGGCTGC | 1.2 | 1.3 | 1.3 | 0.1 |
| | ------------CTGGCTGC | 185 | GGATCCAAATTT | 2 | 0.4 | 1.2 | 1.2 |
| 181 | GGATCCA---TTCTGGCTGC | | AAT | 1.4 | 0.8 | 1.1 | 0.4 |

^{a}On-target sequence centered on cleavage site, with 10 bp in either direction. For comparison, the portion of the CD3 3-1 gRNA target sequence aligning with the Reference on-target sequence is underlined and the PAM is indicated by parenthesis.

TABLE 14
On-target gene edited sequences >1% frequency in
at least one CD33-2 gene edited T cell donor.
Reference on-target sequence^{a}:
AGTTCATGGTTAC(TGG)TTCC (SEQ ID NO: 186)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| 187 | AGTTCATGG-TACTGGTTCC | | T | 24.7 | 25.5 | 25.1 | 0.6 |
| 188 | AGTTCA-------TGGTTCC | | TGGTTAC | 15.6 | 13.1 | 14.3 | 1.8 |
| 189 | AGTTCATG--TACTGGTTCC | | GT | 12.2 | 15 | 13.6 | 2 |
| 190 | AGTTCATGGTTTACTGGTTCC | | | 6 | 5.3 | 5.7 | 0.5 |
| | A--------------GTTCC | 195 | GTTCATGGTTACTG | 2.1 | 3 | 2.5 | 0.6 |
| 191 | AG--------TACTGGTTCC | | TTCATGGT | 1.9 | 2.1 | 2 | 0.1 |
| 192 | AGTTCA----TACTGGTTCC | | TGGT | 1.4 | 2 | 1.7 | 0.4 |
| 193 | AGTTCATGGTATACTGGTTCC | | | 1.5 | 1.3 | 1.4 | 0.1 |
| | -------------------- | 186 | AGTTCATGGTTACTGGTTCC | 1.2 | 1.2 | 1.2 | 0 |
| 194 | A-------GTTACTGGTTCC | | GTTCATG | 1.3 | 1 | 1.1 | 0.2 |

^{a}On-target sequence centered on cleavage site, with 10 bp in either direction. For comparison, the portion of the CD3 3-2 gRNA target sequence aligning with the Reference on-target sequence is underlined and the PAM is indicated by parenthesis.

TABLE 15
On-target gene edited sequences >1% frequency in
at least one CD33-3 gene edited T cell donor.
Reference on-target sequence^{a}:
ACTCCCCAGTTCA(TGG)TTAC (SEQ ID NO: 196)

| | Gene Edited Sequence: | | | | | | |
|---|---|---|---|---|---|---|---|
| | (Deletions indicated | | | | | | |
| SEQ | by dashes (-); | | | | | | |
| ID | insertions indicated | SEQ ID | | Donor 1 | Donor 2 | | Std. |
| NO: | by bold) | NO: | Deleted Sequence: | (%) | (%) | Mean | Dev. |
| 197 | ACTCCCCAGTTTCATGGTTAC | | | 15.9 | 15.2 | 15.5 | 0.5 |
| 198 | ACTCCCCAG-TCATGGTTAC | | T | 9 | 8.8 | 8.9 | 0.1 |
| 199 | ACTCCC-----CATGGTTAC | | CAGTT | 2.7 | 2.7 | 2.7 | 0 |
| 200 | ACTCCCCA-------GTTAC | | GTTCATG | 2.1 | 2 | 2 | 0 |
| 201 | AC--------TCATGGTTAC | | TCCCCAGT | 2 | 1.8 | 1.9 | 0.1 |
| 202 | ACTCCCCA--TCATGGTTAC | | GT | 1.8 | 1.5 | 1.6 | 0.2 |
| 203 | ACTCCCCA-TTCATGGTTAC | | G | 1.5 | 1.6 | 1.6 | 0.1 |
| 204 | ACTCCCCAGTGTCATGGTTAC | | | 1.3 | 1.5 | 1.4 | 0.1 |
| | ------------------AC | 206 | ACTCCCCAGTTCATGGTT | 1.1 | 1.3 | 1.2 | 0.1 |
| | -------------------- | 196 | ACTCCCCAGTTCATGGTTAC | 1 | 1.4 | 1.2 | 0.2 |
| 205 | ACTCCCCAGTCTCATGGTTAC | | | 1 | 0.8 | 0.9 | 0.2 |

^{a}On-target sequence centered on cleavage site, with 10 bp in either direction. For comparison, the portion of the CD3 3-3 gRNA target sequence aligning with the
Reference on-target sequence is underlined and the PAM is indicated by parenthesis.

TABLE 16
On-target gene edited sequences >1% frequency in
at least one CD33-4 gene edited T cell donor.
Reference on-target sequence^{a}:
AGCCATTATATCC(AGG)GACT (SEQ ID NO: 207)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| 208 | AGCCAT--TATCCAGGGACT | | TA | 0.8 | 4.6 | 2.7 | 2.7 |
| | --------------GGGACT | 218 | AGCCATTATATCCA | 4.2 | 0.5 | 2.3 | 2.6 |
| 209 | AG---------CCAGGGACT | | CCATTATAT | 1.4 | 3 | 2.2 | 1.2 |
| 210 | AGCCATTAT-TCCAGGGACT | | A | 0.7 | 2.4 | 1.6 | 1.2 |
| | -------------------- | 207 | AGCCATTATATCCAGGGACT | 1.1 | 1.4 | 1.3 | 0.2 |
| 211 | AG--------TCCAGGGACT | | CCATTATA | 1.1 | 1.4 | 1.3 | 0.2 |
| 212 | AGCCATTATAATCCAGGGACT | | | 0.8 | 1.7 | 1.3 | 0.7 |
| 213 | AGCCAT--TATCCGGGGACT | | TA | 1.9 | 0 | 1 | 1.4 |
| 214 | AGCCATTATA--CAGGGACT | | TC | 0.7 | 1 | 0.9 | 0.3 |
| 215 | ----------TCCAGGGACT | 219 | AGCCATTATA | 0.4 | 1.3 | 0.8 | 0.6 |
| 216 | AGCCATTAT-TCCGGGGACT | | A | 1.2 | 0 | 0.6 | 0.8 |
| 217 | AGCCATTATAATCCGGGGACT | | | 1.1 | 0 | 0.6 | 0.8 |

^{a}On-target sequence centered on cleavage site, with 10 bp in either direction. For comparison, the portion of the CD3 3-4 gRNA target sequence aligning with the Reference on-target sequence is underlined and the PAM is indicated by parenthesis.

TABLE 17
On-target gene edited sequences >1% frequency in
at least one CD33-5 gene edited T cell donor.
Reference on-target sequence^{a}:
TCAGTGACGGTAC(AGG)AGGG (SEQ ID NO: 220)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| | ------------CAGGAGGG | 239 | TCAGTGACGGTA | 13.8 | 14 | 13.9 | 0.1 |
| 221 | TCAGTG-----ACAGGAGGG | | ACGGT | 8.6 | 7.5 | 8 | 0.8 |
| 222 | TCAGTGAC-GTACAGGAGGG | | G | 5.6 | 7.3 | 6.5 | 1.3 |
| 223 | T-----------CAGGAGGG | 240 | CAGTGACGGTA | 6 | 5.3 | 5.6 | 0.5 |
| 224 | TCAGTGAC------GGAGGG | | GGTACA | 4.3 | 4.6 | 4.4 | 0.3 |
| 225 | ---------GTACAGGAGGG | | TCAGTGACG | 3.5 | 2.6 | 3 | 0.6 |
| 226 | TCAGTGACG-----GGAGGG | | GTACA | 2.4 | 2.6 | 2.5 | 0.2 |
| 227 | TCAGTGACGGTTACAGGAGGG | | | 2.3 | 2.4 | 2.4 | 0 |
| 228 | TCAGTGACGG-ACAGGAGGG | | T | 2.2 | 2.3 | 2.2 | 0.1 |
| 229 | TCAGTGACGGGTACAGGAGGG | | | 1.8 | 2.2 | 2 | 0.2 |
| 230 | TCA------GTACAGGAGGG | | GTGACG | 1.4 | 2.3 | 1.9 | 0.6 |
| 231 | TCAGTGAC--TACAGGAGGG | | GG | 1.1 | 2.1 | 1.6 | 0.7 |
| 232 | TCAGTGAC---------GGG | | GGTACAGGA | 1.4 | 1.6 | 1.5 | 0.2 |
| 233 | TCAGTGAC----------GG | 241 | GGTACAGGAG | 1.7 | 1.2 | 1.4 | 0.3 |
| | TCA----------------- | 242 | GTGACGGTACAGGAGGG | 1 | 1.7 | 1.3 | 0.5 |
| 234 | TCAGTGACGG--CAGGAGGG | | TA | 1.8 | 0.7 | 1.2 | 0.8 |
| 235 | TCAGTGACGG---AGGAGGG | | TAC | 0.6 | 1.8 | 1.2 | 0.8 |
| 236 | TCAGTGA---TACAGGAGGG | | CGG | 1.2 | 1.1 | 1.1 | 0.1 |
| 237 | TCAGT----GTACAGGAGGG | | GACG | 1 | 1.1 | 1.1 | 0 |
| | -------------------- | 220 | TCAGTGACGGTACAGGAGGG | 0.7 | 1.1 | 0.9 | 0.3 |
| 238 | TCA-------TACAGGAGGG | | GTGACGG | 0.6 | 1 | 0.8 | 0.3 |

^{a}On-target sequence centered on cleavage site, with 10 bp in either direction. For comparison, the portion of the CD3 3-5 gRNA target sequence aligning with the Reference on-target sequence is underlined and the PAM is indicated by parenthesis.

TABLE 18
On-target gene edited sequences >1% frequency in
at least one CD33-6 gene edited T cell donor.
Reference on-target sequence^{a}:
AGGTGAAGTTCGC(TGG)AGCT (SEQ ID NO: 243)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| 244 | A----------GCTGGAGCT | 254 | GGTGAAGTTC | 3.9 | 4.2 | 4.1 | 0.2 |
| 245 | AGGTGAA----GCTGGAGCT | | GTTC | 4.8 | 3.2 | 4 | 1.1 |
| 246 | AGGTGA----------AGCT | 255 | AGTTCGCTGG | 2.6 | 2.3 | 2.4 | 0.2 |
| | -------------TGGAGCT | 256 | AGGTGAAGTTCG | 1.5 | 3 | 2.3 | 1.1 |
| 247 | AGGTGAAGT----TGGAGCT | | TCGC | 2.1 | 2.2 | 2.2 | 0.1 |
| | -------------------- | 243 | AGGTGAAGTTCGCTGGAGCT | 1.6 | 2.2 | 1.9 | 0.4 |
| | A-------------GGAGCT | 257 | GGTGAAGTTCGCT | 1.8 | 1.3 | 1.5 | 0.3 |
| 248 | AGGTGAAG-TCGCTGGAGCT | | T | 0.9 | 1.9 | 1.4 | 0.8 |
| 249 | AGG----------TGGAGCT | 258 | TGAAGTTCGC | 1.5 | 1.2 | 1.3 | 0.2 |
| 250 | AGGTGAAG--CGCTGGAGCT | | TT | 1.1 | 1.5 | 1.3 | 0.3 |
| | ------------------CT | 259 | AGGTGAAGTTCGCTGGAG | 1.2 | 1.2 | 1.2 | 0 |
| | ----------------AGCT | 260 | AGGTGAAGTTCGCTGG | 1.1 | 1.2 | 1.1 | 0 |
| 251 | ----------CGCTGGAGCT | 261 | AGGTGAAGTT | 1 | 1 | 1 | 0 |
| 252 | AGGTGA----CGCTGGAGCT | | AGTT | 0.9 | 1.1 | 1 | 0.2 |
| 253 | AGGTGAAGTTTCGCTGGAGCT | | | 1.2 | 0.8 | 1 | 0.2 |
| | A------------------- | 262 | GGTGAAGTTCGCTGGAGCT | 0.6 | 1.2 | 0.9 | 0.4 |

^{a}On-target sequence centered on cleavage site, with 10 bp in either direction. For comparison, the portion of the CD3 3-6 gRNA target sequence aligning with the Reference on-target sequence is underlined and the PAM is indicated by parenthesis.

TABLE 19
On-target gene edited sequences >1% frequency in
at least one CD33-7 gene edited T cell donor.
Reference on-target sequence^{a}:
AGTTCGCTGGAGC(TGG)TGTG (SEQ ID NO: 263)

| | Gene Edited Sequence: | | | | | | |
|---|---|---|---|---|---|---|---|
| | (Deletions indicated | | | | | | |
| SEQ | by dashes (-); | | | | | | |
| ID | insertions indicated | | | Donor 1 | Donor 2 | | Std. |
| NO: | by bold) | | Deleted Sequence: | (%) | (%) | Mean | Dev. |
| 264 | AGTTC------GCTGGTGTG | | GCTGGA | 21.5 | 6.6 | 14.1 | 10.5 |
| 265 | ----------AGCTGGTGTG | 267 | AGTTCGCTGG | 2.1 | 1.1 | 1.6 | 0.7 |
| 266 | AGTTCGCT-GAGCTGGTGTG | | G | 1.7 | 0.9 | 1.3 | 0.6 |
| | -------------------- | 263 | AGTTCGCTGGAGCTGGTGTG | 1.7 | 0.3 | 1 | 1 |

^{a}On-target target sequence aligning with the sequence centered on cleavage site, with 10 bp in either direction. For comparison, the portion of the CD3 3-7 gRNA Reference on-target sequence is underlined and the PAM is indicated by parenthesis.

TABLE 20
On-target gene edited sequences >1% frequency in
at least one CD33-8 gene edited T cell donor.
Reference on-target sequence^{a}:
ACTACTCACTCCT(CGG)TGCT (SEQ ID NO: 268)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| 269 | ACTACTCACTTCCTCGGTGCT | | | 29.9 | 35.4 | 32.7 | 3.9 |
| 270 | ACTA-------CTCGGTGCT | | CTCACTC | 8.3 | 6.1 | 7.2 | 1.5 |
| 271 | ACTACTCA-TCCTCGGTGCT | | C | 6.6 | 6.6 | 6.6 | 0 |
| | ACTA--------------CT | 281 | CTCACTCCTCGGTG | 5.3 | 2.6 | 4 | 1.9 |
| 272 | ACTACTCAC-CCTCGGTGCT | | T | 3.9 | 3.8 | 3.9 | 0 |
| 273 | ACT----ACTCCTCGGTGCT | | ACTC | 2.4 | 3.1 | 2.7 | 0.5 |
| | -------------------- | 268 | ACTACTCACTCCTCGGTGCT | 2.3 | 2.8 | 2.5 | 0.4 |
| 274 | ----------CCTCGGTGCT | 282 | ACTACTCACT | 2.7 | 2.1 | 2.4 | 0.4 |
| | --------------GGTGCT | 283 | ACTACTCACTCCTC | 2.4 | 2.1 | 2.3 | 0.2 |
| 275 | ACTACTCA--CCTCGGTGCT | | CT | 2.3 | 1.9 | 2.1 | 0.3 |
| 276 | ACTACTCA---CTCGGTGCT | | CTC | 1.9 | 1.6 | 1.8 | 0.2 |
| 277 | ACTACT--CTCCTCGGTGCT | | CA | 1.7 | 1.7 | 1.7 | 0 |
| 278 | ACTACT---TCCTCGGTGCT | | CAC | 1.1 | 1.7 | 1.4 | 0.5 |
| 279 | ACTACTCACT--TCGGTGCT | | CC | 1.1 | 1.3 | 1.2 | 0.1 |
| | -----------------GCT | 284 | ACTACTCACTCCTCGGT | 1 | 1.2 | 1.1 | 0.2 |
| 280 | ACTA-----TCCTCGGTGCT | | CTCAC | 1.2 | 0.5 | 0.8 | 0.5 |

^{a}On-target sequence centered on cleavage site, with 10 bp in either direction. For comparison, the portion of the CD33-8 gRNA target sequence aligning with the Reference on-target sequence is underlined and the PAM is indicated by parenthesis.

TABLE 21
On-target gene edited sequences >1% frequency in
at least one CD33-9 gene edited T cell donor.
Reference on-target sequence^{a}:
CCCGATCTTCTCC(TGG)TTGT (SEQ ID NO: 285)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| 286 | CCCGATCT--TCCTGGTTGT | | TC | 10.7 | 13.2 | 12 | 1.8 |
| 287 | CCCGA-----TCCTGGTTGT | | TCTTC | 12.6 | 10.9 | 11.7 | 1.2 |
| 288 | CCCGAT------CTGGTTGT | | CTTCTC | 5.4 | 6.2 | 5.8 | 0.5 |
| | -------------------- | 285 | CCCGATCTTCTCCTGGTTGT | 3.3 | 3.6 | 3.4 | 0.3 |
| | -----------CCTGGTTGT | 295 | CCCGATCTTCT | 2.4 | 2.4 | 2.4 | 0 |
| 289 | C----------CCTGGTTGT | 296 | CCGATCTTCT | 2.3 | 2.5 | 2.4 | 0.1 |
| 290 | CCCGATCTT---CTGGTTGT | | CTC | 1.9 | 1.5 | 1.7 | 0.3 |
| 291 | CCCGATCT-----TGGTTGT | | TCTCC | 2 | 1.4 | 1.7 | 0.4 |
| 292 | CCCGA---TCTCCTGGTTGT | | TCT | 1.8 | 1.3 | 1.6 | 0.3 |
| 293 | CCCGATCTTC-CCTGGTTGT | | T | 1.1 | 1 | 1.1 | 0.1 |
| | CCCGA--------------T | 297 | TCTTCTCCTGGTTG | 0.5 | 1 | 0.8 | 0.4 |
| 294 | CCCGATCTTC---------- | 298 | TCCTGGTTGT | 0.5 | 1 | 0.7 | 0.4 |

^{a}On-target sequence centered on cleavage site, with 10 bp in either direction. For comparison, the portion of the CD3 3-9 gRNA target sequence aligning with the Reference on-target sequence is underlined and the PAM is indicated by parenthesis.

TABLE 22
On-target gene edited sequences >1% frequency in
at least one CD33-10 gene edited T cell donor.
Reference on-target sequence^{a}:
AAATCCTCATCCC(TGG)CACT (SEQ ID NO: 299)

| | Gene Edited Sequence: | | | | | | |
|---|---|---|---|---|---|---|---|
| | (Deletions indicated | | | | | | |
| SEQ | by dashes (-); | SEQ | | | | | |
| ID | insertions indicated | ID | | Donor 1 | Donor 2 | | Std. |
| NO: | by bold) | NO: | Deleted Sequence: | (%) | (%) | Mean | Dev. |
| 300 | AAAT-------CCTGGCACT | | CCTCATC | 17.7 | 12.1 | 14.9 | 4 |
| 301 | AA------ATCCCTGGCACT | | ATCCTC | 9.4 | 6.2 | 7.8 | 2.3 |
| 302 | AAATCCTCATTCCCTGGCACT | | | 7.2 | 8.1 | 7.6 | 0.6 |
| 303 | ----------CCCTGGCACT | 317 | AAATCCTCAT | 7.3 | 5.3 | 6.3 | 1.5 |
| 304 | AAATCCTCA-CCCTGGCACT | | T | 4.7 | 5.7 | 5.2 | 0.7 |
| 305 | AAATCCTC--CCCTGGCACT | | AT | 3.2 | 3.9 | 3.5 | 0.5 |
| | --------------GGCACT | 318 | AAATCCTCATCCCT | 2.5 | 3.2 | 2.8 | 0.5 |
| 306 | AAATCC---TCCCTGGCACT | | TCA | 2.6 | 2.7 | 2.6 | 0.1 |
| 307 | AAATC-----CCCTGGCACT | | CTCAT | 2.1 | 2.8 | 2.5 | 0.5 |
| 308 | ACATCCTCATTCCCTGGCACT | | | 1.1 | 2.5 | 1.8 | 1 |
| 309 | ACAT-------CCTGGCACT | | CCTCATC | 2.3 | 1.2 | 1.8 | 0.8 |
| 310 | AAATCCTC-TCCCTGGCACT | | A | 1.2 | 2.1 | 1.6 | 0.6 |
| | -------------------- | 299 | AAATCCTCATCCCTGGCACT | 1.6 | 1.7 | 1.6 | 0.1 |
| 311 | AAATCCTCAT--CTGGCACT | | CC | 0.9 | 1.9 | 1.4 | 0.7 |
| | AAATC-------------CT | 319 | CTCATCCCTGGCA | 1.3 | 1.5 | 1.4 | 0.1 |
| 312 | AAA-------CCCTGGCACT | | ATCCTCATC | 1 | 1.7 | 1.3 | 0.5 |
| | ----------------CACT | 320 | AAATCCTCATCCCTGG | 1.6 | 1.1 | 1.3 | 0.3 |
| 313 | AAATCCT-----CTGGCACT | | CATCC | 1.6 | 0.5 | 1.1 | 0.8 |
| 314 | AAATCC----CCCTGGCACT | | TCAT | 0.8 | 1.2 | 1 | 0.3 |
| 315 | AAATCCT---------CACT | | CATCCCTGG | 1.1 | 0.7 | 0.9 | 0.3 |
| 316 | A------CATCCCTGGCACT | | AATCCT | 0.4 | 1.3 | 0.9 | 0.6 |
| | AAA----------------T | 321 | TCCTCATCCCTGGCAC | 0.6 | 1.1 | 0.9 | 0.3 |
| | -----------CCTGGCACT | 322 | AAATCCTCATC | 1.4 | 0.4 | 0.9 | 0.7 |
| | AAATC--------------- | 323 | CTCATCCCTGGCACT | 0.4 | 1.2 | 0.8 | 0.6 |
| | ------------------CT | 324 | AAATCCTCATCCCTGGCA | 0.3 | 1.1 | 0.7 | 0.6 |

^{a}On-target sequence centered on cleavage site, with 10 bp m either direction For comparison, the portion of the CD33*10gRNA target sequence aligning with the Reference on-target sequence is underlined and the PAM is indicated by parenthesis

### Example 5. Stabilization of CAR Expression and CD4/CD8 Cell Populations with CD33 Knock-Out

This example describes the surprising beneficial effect of editing the CD33 gene in primary human T cells ex vivo using CRISPR/Cas9 gene editing. CRISPR/Cas9 and AAV6 were used as above (see for example, Examples 1-3) to create human T cells that lacked expression of the TCR, β2M and CD33 with concomitant expression from the TRAC locus using a CAR construct targeting CD33.

Activated T cells were first electroporated with 3 distinct Cas9:sgRNA RNP complexes, one containing sgRNAs targeting the TRAC locus (SEQ ID NO: 28), the second containing sgRNAs targeting the β2M locus (SEQ ID NO: 30) and the third containing the sgRNAs targeting CD33 (CD33-10: SEQ ID NO: 151). The DNA double stranded break at the TRAC locus was repaired by homology directed repair with an AAV6-delivered DNA template (e.g., SEQ ID NOS: 49, 51, 53, 55, 57, 59, 61, 63, 109, 112, 115, or 118) (encoding an anti-CD33 CAR comprising the amino acid sequence of SEQ ID NO: 101, 102, 103, 104, 105, 106, 107, 108, 111, 114, 117, 120). The anti-CD33 CAR construct was comprised of right and left homology arms corresponding to the TRAC locus that flanked a chimeric antigen receptor cassette (−/+ regulatory elements for gene expression). The resulting modified T cells are 3× KO (TRAC−/β2M−/CD33−), anti-CD33 CAR+ T cells. The 3×KO anti-CD33 CAR T cells were compared to the 2× KO (TRAC−/β2M−), anti-CD33 CAR T cells generated as described above.

Anti-CD33 CAR expression and CD4/CD8 cell populations were assessed as described in Example 2.

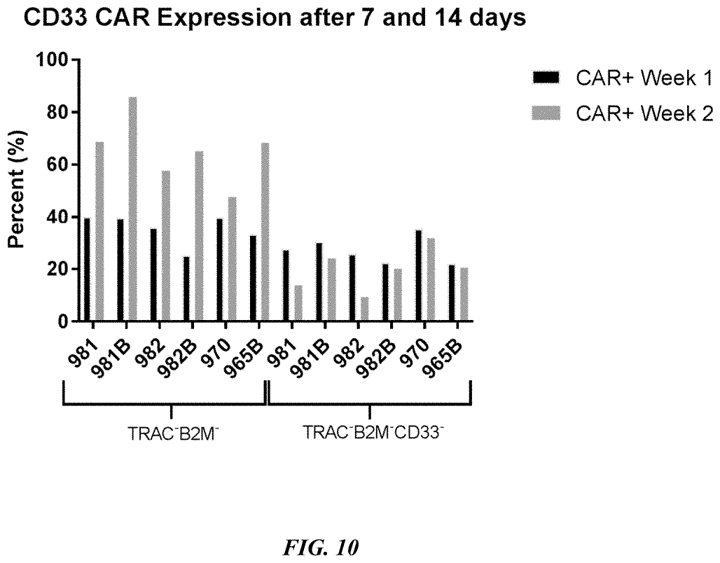

As described in Example 1, TRAC−/B2M−/CAR+ (2× KO, CAR+) edited T cells demonstrated an increase in the percentage of cells that were CAR+ over time (FIG. 2B). To evaluate if knocking out CD33 would stabilize the CAR+ T cell population, T cells were edited with six different CAR constructs (CTX-981, CTX-981b, CTX-982, CTX-982b, CTX-970, CTX-965b) and evaluated for the ability of CD33 KO to stabilize CAR expression. FIG. 10 shows that the percentage of cells positive for CAR expression increased between 7 and 14 days for T cells that were edited with 2× KO (TRAC−/β2M−) and anti-CD33 CAR. However, for T cells that were edited with 3× KO (TRAC−/β2M−/CD33−) and anti-CD33 CAR, no such increase in the percentage of CAR-expressing cells was observed between 7 and 14 days.

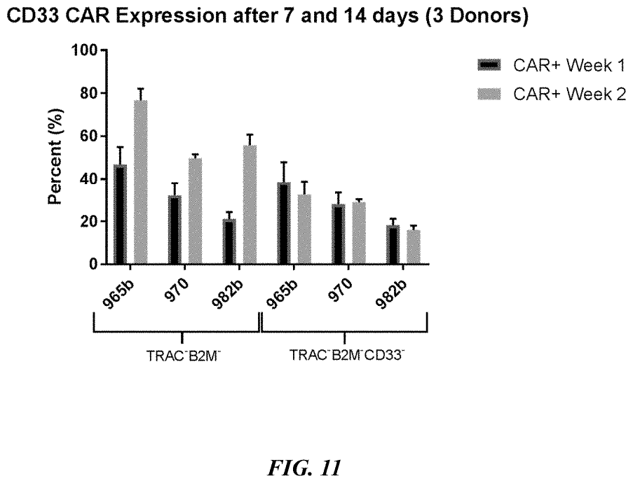

The study was repeated with anti-CD33 CAR T cells generated from 3 different primary T cell donors. The 2× KO, anti-CD33 CAR T cells (CTX-965b, CTX-970 and CTX-982b) again showed an increase in the proportion of cells that are CAR+ between 7 days and 14 days which was not observed in 3× KO, anti-CD33 CAR+(TRAC−/β2M−/CD33−/CAR+) T cells (FIG. 11). Instead, 3KO CAR+ T cells maintained a similar level of CAR-expressing cells at 7 and 14 days.

These results demonstrate that knocking out the CD33 gene in T cells edited to express an anti-CD33 CAR prevents expansion of the portion of edited cells that are CAR+. This stabilization of CAR-expressing cells in the edited T cell population enables the generation of a reproducible CAR T cell product.

Proportion of CD4 and CD8 T Cells is Stabilized with CD33 Knock-Out.

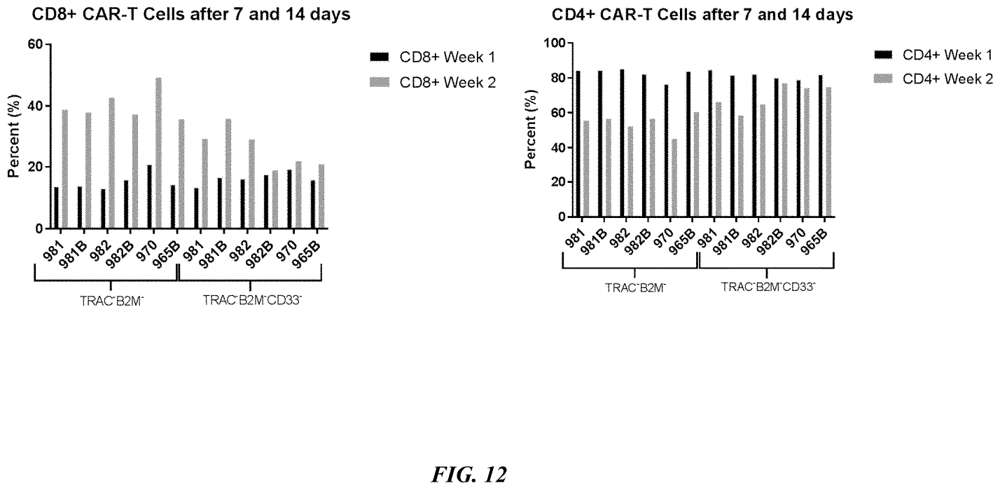

Previously, T cells edited to be 2× KO (TRAC−/β2M−) and anti-CD33 CAR+ showed a decrease in the proportion of CD4+ T cells and an increase in the proportion of CD8+ T cells between 7 and 14 days (FIG. 2C). To determine the effect of a CD33 knockout on the ratio of CD4 and CD8 cells over time, 3× KO (TRAC−/β2M−/CD33−), anti-CD33 CAR+ T cells were evaluated by flow cytometry on day 7 and day 14. The proportion of CD4+ and CD8+ T cells was determined for cells edited with different CAR constructs (CTX-981, CTX-981b, CTX-982, CTX-982b, CTX-970, CTX-965b). The same measurement was performed for T cells edited to be 2× KO (TRAC−/β2M−) and anti-CD33 CAR+. While an increase in CD4+ T cells and decrease in CD8+ cells was again observed for 2× KO CAR+ T cells between 7 and 14 days, no such change was observed for 3× KO CAR+ T cells (FIG. 12). In particular, 3× KO CAR+ T cells edited with a CTX-982b, CTX-970, or CTX-965b CAR construct demonstrated little change in the proportion of cells that were CD4+ and that were CD8+. These data demonstrate that knocking out CD33 stabilizes the ratio of CD4+ T cells to CD8+ T cells in the edited T cell population over time.

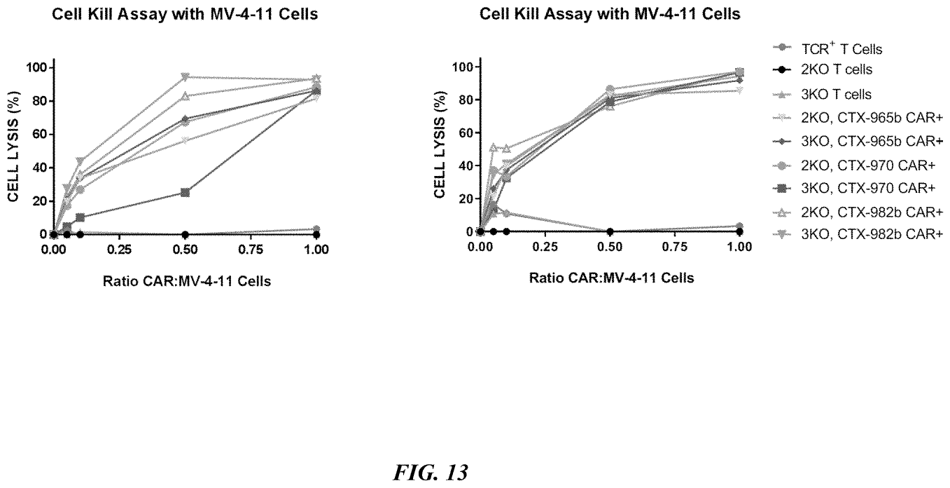

Target cell killing and cytokine secretion by 3× KO (TRAC−/β2M−/CD33−), anti-CD33 CAR+ T cells The ability of 2× KO (TRAC−/B2M−) CAR+ T cells to kill cancer cells was evaluated as described in Example 2 above. Likewise, the ability of 3× KO (TRAC−/B2M−/CD33−) CAR+ T cells to eliminate CD33+ tumor cells was determined. This was done as described above wherein CAR+ T cells were co-cultured with the CD33-expressing AML cell line MV4-11 (ATCC CRL-9591). A direct comparison of 2× KO CAR+ T cells and 3× KO CAR+ T cells was performed. In each case the T cells were generated from two different human donors and were edited with CAR construct CTX-965b, CTX-970 or CTX-982. The T cells were incubated with MV4-11 cells at a ratio of CAR T cell to target cell of 0.05:1-1:1 and the percentage of cell lysis among MV4-11 cells was determined as described above. Both 2× KO CAR+ T cells and 3× KO CAR+ T cells demonstrated efficient killing of MV4-11 cells, even at low CAR T cell: target cell ratios (FIG. 13). This result was consistent for T cells generated from different donors. Regardless of the donor, both 2× KO and 3× KO CAR+ T cells achieved nearly complete killing of target cells when seeded at a 1:1 ratio.

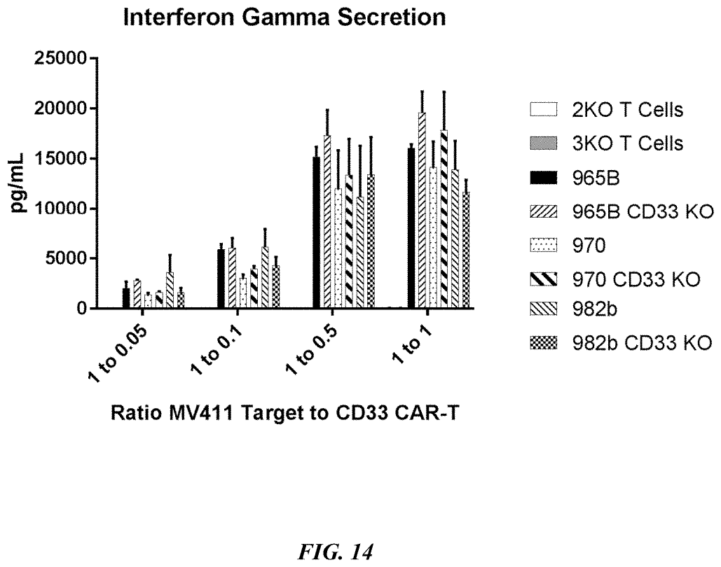

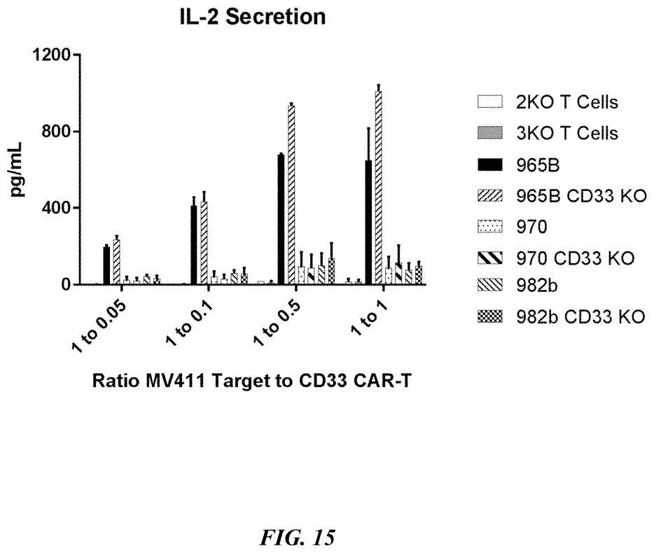

The functional ability of TRAC−/B2M−/anti-CD33 CAR+ T cells (e.g.: CTX-965b CAR T cells and CTX-970 CAR T cells) to induce cytokine secretion in the presence of target cells was evaluated according to the procedure described in Example 2. To measure cytokine release, 3× KO CAR+ T cells were co-cultured with CD33-expressing MV4-11 target cells for 24 hours at a ratio of anti-CD33 CAR-T to MV411 ranging from 0.05:1 to 1:1. Supernatant media was collected and cytokines (e.g., IFNγ and IL-2) present in the supernatant were quantified using IFNγ or IL-2 ELISA assays (RD Systems) following manufacturer's instructions (RD Systems). Cytokine production by 3× KO CAR+ T cells was compared to cytokine production by 2× KO (TRAC−/β2M−), CAR+ T cells and control T cells (e.g., TRAC+ T cells and TRAC−/β2M− T cells). Quantification of IFNγ production indicated that 3× KO CAR+ T cells induced high levels of IFNγ compared to control T cells and comparable levels of IFNγ production relative to 2× KO CAR+ T cells for each CAR construct tested (FIG. 14). Thus, the CD33 knock-out did not appear to alter the ability of the CAR+ T cells to secrete IFNγ. Similarly, the ability of CAR+ T cells to produce IL-2 in response to target cell stimulation was evaluated. The level of IL-2 production was dependent upon the CAR construct used to generate the T cells. The anti-CD33 CAR+ T cells generated with CTX-965b CAR produced high levels of IL-2 relative to control T cells, while those generated using the CTX-970 or CTX-982b CAR constructs produced levels of IL-2 that were only slightly higher than control T cells (FIG. 15). The low IL-2 production observed for T cells generated with the CTX-970 or CTX-982b CAR constructs was observed for both 2× KO CAR+ T cells and 3× KO CAR+ T cells, indicating that the CD33 knockout did not rescue IL-2 production. However, for T cells generated with the CTX-965b CAR, T cells that had the 3× KO (TRAC−/β2M−/CD33−) produced higher levels of IL-2 than T cells that had only the 2× KO (TRAC−/β2M−).

### Example 6. Selectivity of the Anti-CD33 CAR+ T Cells

This example describes the generation of a CD33-deficient cancer cell line and the use of this cell line to evaluate the ability of gene-edited CAR+ T cells to achieve selective killing of CD33-expressing target cells.

### Generation of CD33 KO MV4-11 AML Cell Line

To generate a CD33-deficient cancer cell line, MV4-11 cells were electroporated with Cas9:sgRNA targeting CD33 (CD33-10; SEQ ID NO: 151). Cells were plated at ˜1 cell per well and allowed to expand for three weeks. Several clonal lines were then tested for CD33 surface expression using flow cytometry and an anti-CD33 antibody (PE-anti-human-CD33, Biolegend catalog #366608) Wild type MV4-11 (WT MV4-11) had high level of surface CD33, while one particular clonal line demonstrated no CD33 staining above background. This clonal line (referred to as CD33 KO MV4-11 cells) was subsequently used to evaluate CD33-selective killing by anti-CD33 CAR+ T cells.

Selective-killing by anti-CD33 CAR+ T cells was evaluated by measuring target cell lysis and cytokine production in the presence of WT MV4-11 or CD33 KO MV4-11. CAR+ T cells that were defined as selective were those that induced cell lysis and cytokine production in the presence of CD33-expressing WT MV4-11 cells, but exhibited no response in the presence of CD33 KO MV4-11. This result indicates that the anti-CD33 CAR+ T cell requires recognition of a CD33 antigen on the surface of the target cell to mediate cell killing. Selective killing was evaluated by measuring target cell lysis and CAR+ T cell cytokine production as described in example 2. Selectivity was measured for CAR+ T cells generated with three different CAR constructs (CTX-965b, CTX-970, and CTX-982b) and for both 2× KO CAR+ T cells and 3× KO CAR+ T cells. Thus, 6 CAR+ T cells were evaluated for selective target cell lysis and cytokine production when co-cultured for 24 hours with either WT MV4-11 or CD33 KO MV4-11 cells at a CAR+ T cell:target cell ratio of 1:1. The CAR+ T cells evaluated were as follows:

2× KO (TRAC−/β2M−), anti-CD33 CAR+ T cells, expressing CTX-965b CAR

2× KO (TRAC−/β2M−), anti-CD33 CAR+ T cells, expressing CTX-970 CAR

2× KO (TRAC−/β2M−), anti-CD33 CAR+ T cells, expressing CTX-982b CAR

3× KO (TRAC−/β2M−/CD33−), anti-CD33 CAR+ T cells, expressing CTX-965b CAR

3× KO (TRAC−/β2M−/CD33−), anti-CD33 CAR+ T cells. expressing CTX-970 CAR

3× KO (TRAC−/β2M−/CD33−), anti-CD33 CAR+ T cells. expressing CTX-982b CAR

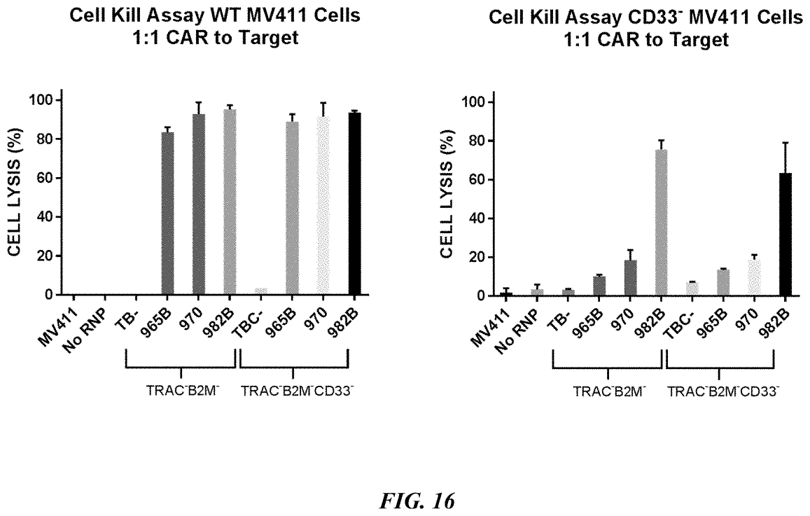

Both 2× KO CAR+ T cells and 3× KO CAR+ T cells achieved near-complete killing of WT MV4-11 target cells (FIG. 16, left panel). In contrast, no killing of WT MV4-11 was seen for control groups (e.g., either MV4-11 alone or MV4-11 co-cultured with no RNP T cells, TRAC−/B2M− T cells, or TRAC−/B2M−/CD33− T cells). The ability of CAR+ T cells to kill CD33 KO MV4-11 cells was also evaluated. CAR+ T cells generated with a CTX-965b CAR or a CTX-970 CAR induced negligible to low levels of cell killing compared to control groups (FIG. 16, right panel). This was true for both 2× KO CAR+ and 3× KO CAR+ T cells. In contrast, both 2× KO CAR+ and 3× KO CAR+ T cells generated with a CTX-982b CAR induced killing of CD33 KO MV4-11 cells at levels higher than 60%. Together, these results indicate that 2× KO or 3× KO anti-CD33 CAR+ T cells generated with a CTX-965b or CTX-970 CAR are selective for killing CD33-expressing target cells, while T cells generated with a CAR-982b are non-selective and induce undesirable killing of cells deficient in CD33 expression (i.e., off-target cells).

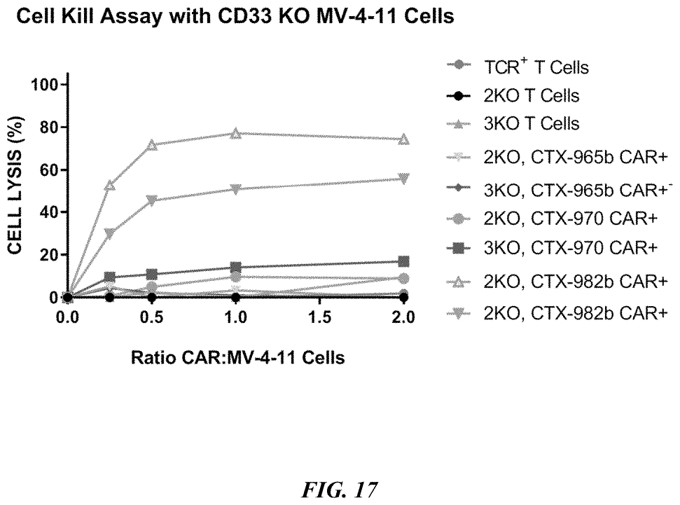

To determine if selective killing by CAR+ T cells expressing the CTX-965b CAR or the CTX-970 CAR is maintained at high ratios of CAR+ T cell to cancer cells, a range of ratios was evaluated. CAR+ T cells were co-cultured with CD33 KO MV4-11 cells at a ratio ranging from 0.25:1 to 2:1. While CAR+ T cells generated with a CTX-965 CAR or a CTX-970 CAR demonstrated low or negligible levels of cell lysis even at the highest ratio tested, CAR+ T cells generated with a CTX-982b CAR induced high levels of cell killing at all ratios tested (FIG. 17). These results demonstrate that CTX-965 or CTX-970 CAR T cells are selective even when cultured at high abundance relative to cancer cells, while CTX-982b CAR T cells are not selective at any seeding ratio tested.

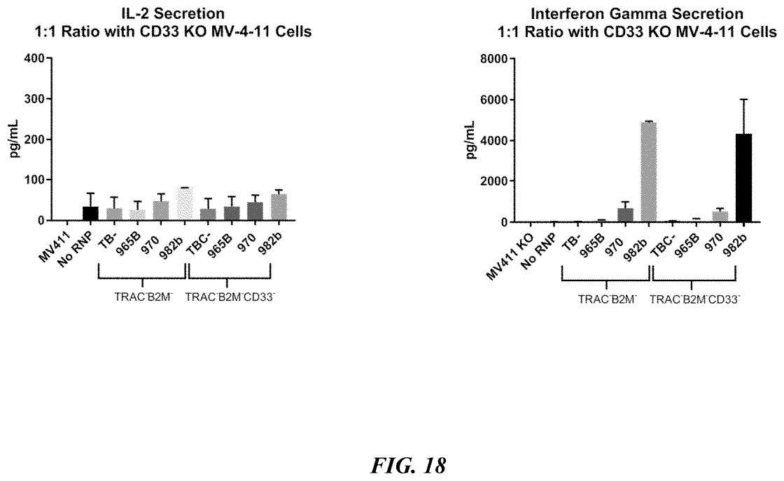

To provide an additional measure of selectivity, cytokine production by CAR+ T cells in the presence of CD33 KO MV4-11 cells was evaluated. Given that CAR+ T cells produce cytokines upon recognition of an antigen on a target cell, selective CAR+ T cells are expected to only produce cytokines when a target cell expressing a CAR-specific antigen is present. Given that CAR+ T cells with a CTX-965 CAR or a CTX-970 CAR were selective for inducing lysis of CD33-expressing target cells (FIG. 16), it was expected that likewise, they would only produce cytokines in the presence of CD33-expressing target cells and not in the presence of CD33-deficient cells. To determine if this was the case, CAR+ T cells were co-cultured with CD33 KO MV4-11 cells and IL-2 and IFNγ in the supernatant were measured by ELISA as described in example 2. Interestingly, none of the CAR+ T cells produced IL-2 when co-cultured with CD33 KO MV4-11 cells (FIG. 18, left panel). This was true even for CTX-982b CAR T cells that were identified as non-selective killers as described above. However, CTX-982b CAR T cells induced significant levels of IFNγ when incubated with CD33 KO MV4-11 cells (FIG. 18, right panel). This was true for both 2× KO CAR+ and 3× KO CAR+ T cells prepared with a CTX-982b CAR. In contrast, 2× KO CAR+ and 3× KO CAR+ T cells prepared with a CTX-965 or CTX-970 CAR induced no IFNγ when incubated with CD33 KO MV4-11 cells. These differences in IFNγ production in the presence of CD33-deficient cells substantiate the conclusion that while CAR+ T cells generated with a CTX-982b CAR are non-selective, CAR+ T cells generated with a CTX-965 or CTX-970 CAR are able to achieve selective activation and killing only in the presence of CD33-expressing target cells or cancer cells.

### Example 7. Effect of Anti-CD33 CAR T Cells In Vivo

This example describes studies performed to evaluate therapeutic effects of anti-CD33 CAR+ T cells in an animal model of acute myeloid leukemia (AMLXMV-4-11 NSG model).

The MV-4-11 human AML derived cell line was made to express both luciferase and mCherry (MV-4-11-Luc-mCh-Puro) genes for in vivo imaging studies. Bioluminescence imaging (BLI) correlates with the amount of tumor burden, and therefore BLI was quantitated to assess tumor burden. To measure BLI, mice were injected with luciferin prior to measuring luciferase using IVIS S5 Lumina (Perkin Elmer). On Day 0, MV-4-11-Luc-mCh-Puro cells were injected into 5-6 week old female NSG mice (The Jackson Laboratory) (2×10⁶ cells/mouse). Anti-CD33 CAR-T cells (CTX-965b) were tested at three doses (1.5×10⁶ cells/mouse, 3.0×10⁶ cells/mouse, and 6.0×10⁶ cells/mouse) in the MV-4-11 NSG model. Anti-CD33 CAR-T cells (CTX-965b) were injected into mice at Day 5. Clinical observations and body weight measurements were performed every 2-4 days and BLI was measured weekly.

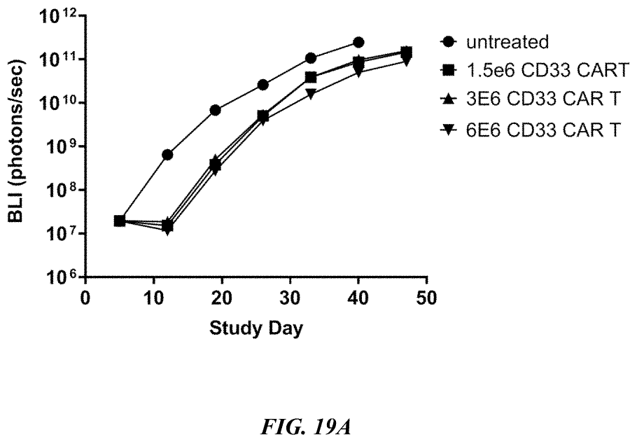

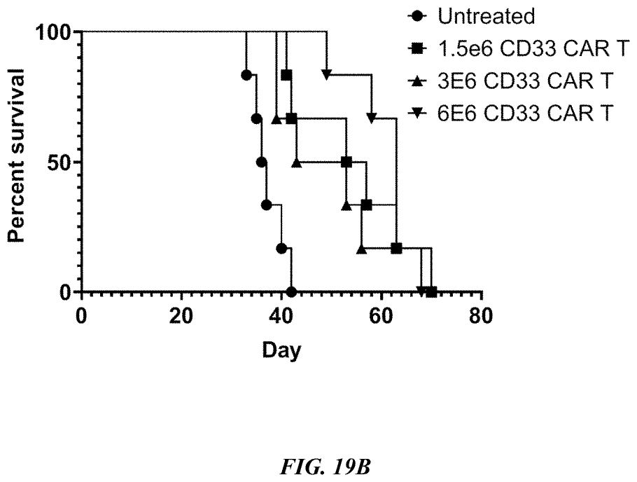

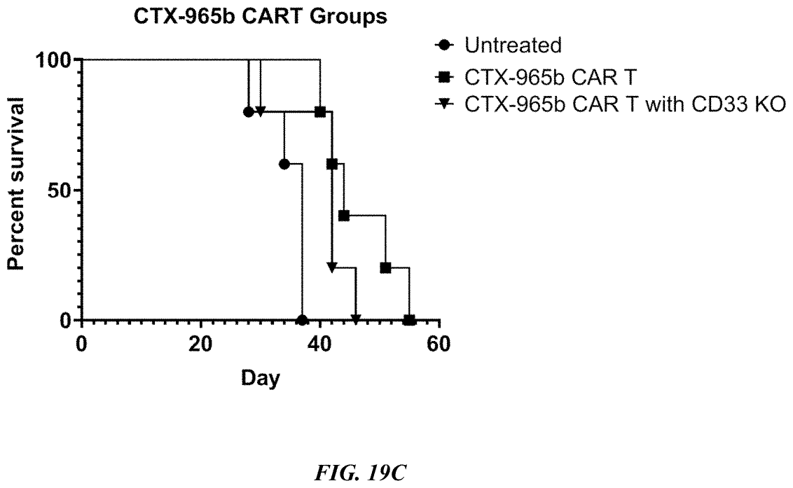

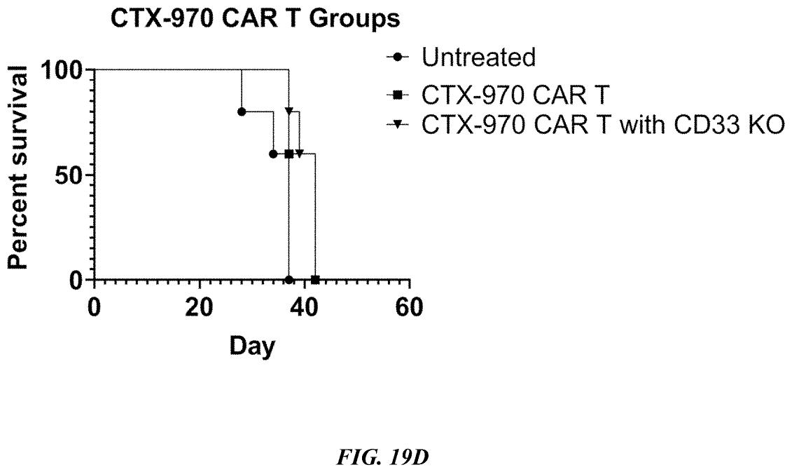

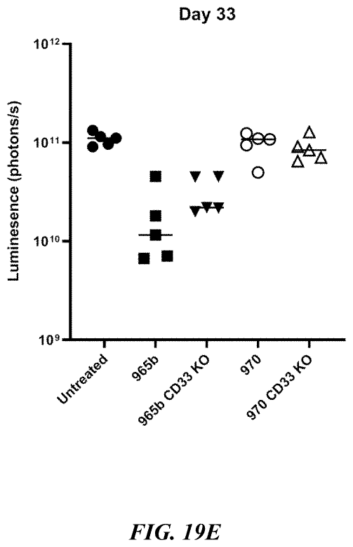

All three doses reduced tumor burden (FIG. 19A), delayed the onset of tumor growth, and led to an increase in mouse survival (FIG. 19B and Table 23) relative to untreated mice.

TABLE 23
Median survival of AML mice.

| | Median Survival | p value (compared to |
|---|---|---|
| Group | (Days) | untreated) Log-rank |
| Untreated | 36.5 | |
| 1.5E6 CAR-T+ | 55 | 0.0033 |
| 3E6 CAR-T+ | 48 | 0.01 |
| 6E6 CAR-T+ | 63 | 0.0005 |

Additional studies were performed to evaluate therapeutic effects of anti-CD33 CAR+ T cells with or without knockout of the CD33 gene (guide CD33-2) in the MV-4-11 NSG mouse model of AML. In these studies, mice were injected with CTX-965b or CTX-970 with or without knockout of the CD33 gene at a dose of 3×10⁶ cells/mouse. Mice in each treatment group displayed increased survival compared to mice in the untreated control group (FIGS. 19C-19D and Table 24).

TABLE 24
Median survival of AML mice.

| | Median Survival | p value (compared to |
|---|---|---|
| Group | (Days) | untreated) Log-rank |
| Untreated | 37 | |
| CTX-965b CAR T | 44 | 0.0034 |
| CTX-965b CAR T + CD33 | 42 | 0.041 |
| KO | | |
| CTX-970 CAR T | 42 | 0.029 |
| CTX-970 CAR T + CD33 KO | 37 | 0.012 |

CTX-965b CAR+ T cells with or without CD33 knockout appeared to be more efficacious in terms of tumor burden control, as indicated by the lower luminescence measurements detected on Day 33 for the CTX-965b groups compared to those of the CTX-970 groups (FIG. 19E and Table 25).

TABLE 25
Statistical analysis of luminescence measurements detected on Day 33.

| Ordinary one-way ANOVA | |
|---|---|
| Tukey's multiple comparisons test | Adjusted P Value |
| Untreated vs. 965b | <0.0001 |
| Untreated vs. 965b CD33 KO | <0.0001 |
| 965b vs. 970 | <0.0001 |
| 965b vs. 970 CD33 KO | 0.0003 |
| 965b CD33 KO vs. 970 | 0.0005 |
| 965b CD33 KO vs. 970 CD33 KO | 0.0026 |

In sum, these results demonstrate that anti-CD33 CAR+ T cells have therapeutic effects in a mouse model of AML. Such therapeutic effects were provided by anti-CD33 CAR+ T cells with or without knockout of the CD33 gene.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1-89. (canceled)

90. A population of engineered T cells, wherein the engineered T cells comprise:
(i) a nucleic acid encoding a CAR comprising (a) an ectodomain that an anti-CD33 antigen-binding fragment; and
(ii) a disrupted TRAC gene; a disrupted β2M gene, or a combination thereof.

91. The population of engineered T cells of claim 90, wherein the engineered T cells comprise the disrupted TRAC gene, in which the nucleic acid encoding the CAR is inserted.

92. The population of engineered T cells of claim 90, wherein the anti-CD33 antigen-binding fragment is an anti-CD33 single-chain variable fragment (scFv).

93. The population of engineered T cells of claim 92, wherein the anti-CD33 scFv comprises the same heavy chain variable domain (VH) complementarity determining regions (CDRs) and the same light chain variable domain (VL) CDRs as a reference antibody, wherein the reference antibody comprises:
(i) a VH set forth as SEQ ID NO: 65 and a VL set forth as SEQ ID NO: 66,
(ii) a VH set forth as SEQ ID NO: 77 and a VL set forth as SEQ ID NO: 78, or
(iii) a VH set forth as SEQ ID NO: 89 and a VL set forth as SEQ ID NO: 90.

94. The population of engineered T cells of claim 93, wherein the anti-CD33 scFv comprises the same VH and the same VL as the reference antibody.

95. The population of engineered T cells of claim 94, wherein the anti-CD33 scFv comprises the amino acid sequence selected from the group consisting of SEQ ID NOs: 73, 75, 85, 87, 97, and 99.

96. The population of engineered T cells of claim 90, wherein the CAR further comprises an endodomain that comprises (i) a CD28 co-stimulatory domain or a 41BB co-stimulatory domain, and (ii) a CD3ζ co-stimulatory domain.

97. The population of engineered T cells of claim 96, wherein the CAR further comprises a CD8 transmembrane domain.

98. The population of engineered T cells of claim 90, wherein the CAR comprises an amino acid sequence selected from the group consisting of SEQ ID NOs: 100-108.

99. The population of engineered T cells of claim 91, wherein the disrupted TRAC gene comprises a nucleotide sequence selected from the group consisting of SEQ ID NOs: 49, 51, 53, 55, 57, 59, 61, 63, 109, 112, 115, and 118.

100. The population of engineered T cells of claim 90, wherein the engineered T cells comprise the disrupted 62M gene, which comprises at least one nucleotide sequence selected from any one of SEQ ID NOs: 9-14.

101. The population of engineered T cells of claim 90, wherein the engineered T cells comprise a wild-type CD33 gene.

101. The population of engineered T cells of claim 90, wherein the engineered T cells comprise a disrupted CD33 gene.

102. The population of engineered T cells of claim 90, wherein the engineered T cells comprise (i) the nucleic acid encoding the CAR, wherein the CAR comprises the amino acid sequence of SEQ ID NO:104, (ii) the disrupted TRAC gene, and (iii) the disrupted β2M gene.

103. The population of engineered T cells of claim 102, wherein the nucleic acid encoding the CAR comprises the nucleotide sequence of SEQ ID NO:56.

104. The population of engineered T cells of claim 103, wherein the disrupted TRAC gene comprises the nucleotide sequence of SEQ ID NO: 55.

105. The population of engineered T cells of claim 90, wherein:
(a) at least 50% of engineered T cells of the population do not express a detectable level of T cell receptor (TCR) protein;
(b) at least 50% of engineered T cells of the population do not express a detectable level of β2M protein;
(c) at least 70% of engineered T cells of the population express the CAR; or
(d) a combination of any one of (a)-(c).

106. The population of engineered T cells of claim 103, wherein at least 50% of engineered T cells of the population do not express a detectable level of CD33 protein.

107. A method for treating cancer, the method comprising administering an effective amount of the population of engineered T cells of claim 90 to a subject in need thereof.

108. The method of claim 107, wherein the subject is a human patient having leukemia.

109. The method of claim 108, wherein the leukemia is acute lymphoblastic leukemia (ALL), acute myeloid leukemia (AML), chronic lymphocytic leukemia (CLL) or chronic myeloid leukemia (CML).

110. A method for producing the population of engineered T cells of claim 90, the method comprising:
(a) delivering to a plurality of T cells
(i) a RNA-guided nuclease,
(ii) a gRNA targeting a TRAC gene, a gRNA targeting β2M gene, or a combination thereof, and
(iii) a vector comprising a donor template that comprises the nucleic acid encoding the CAR; and
(b) producing the population of engineered T cells.


SEQUENCE LISTING (325 sequences)

SEQ ID NO: 1 - DNA, 19 bp, Artificial Sequence
  SYNTHESIZED POLYNUCLEOTIDE
aagagcaacaaatctgact

SEQ ID NO: 2 - DNA, 58 bp, Artificial Sequence
  SYNTHESIZED POLYNUCLEOTIDE
aagagcaacagtgctgtgcctggagcaacaaatctgactaagagcaacaaatctgact

SEQ ID NO: 3 - DNA, 52 bp, Artificial Sequence
  SYNTHESIZED POLYNUCLEOTIDE
aagagcaacagtgctggagcaacaaatctgactaagagcaacaaatctgact

SEQ ID NO: 4 - DNA, 53 bp, Artificial Sequence
  SYNTHESIZED POLYNUCLEOTIDE
aagagcaacagtgcctggagcaacaaatctgactaagagcaacaaatctgact

SEQ ID NO: 5 - DNA, 38 bp, Artificial Sequence
  SYNTHESIZED POLYNUCLEOTIDE
aagagcaacagtgctgactaagagcaacaaatctgact

SEQ ID NO: 6 - DNA, 60 bp, Artificial Sequence
  SYNTHESIZED POLYNUCLEOTIDE
aagagcaacagtgctgtgggcctggagcaacaaatctgactaagagcaacaaatctgact

SEQ ID NO: 7 - DNA, 57 bp, Artificial Sequence
  SYNTHESIZED POLYNUCLEOTIDE
aagagcaacagtgctggcctggagcaacaaatctgactaagagcaacaaatctgact

SEQ ID NO: 8 - DNA, 60 bp, Artificial Sequence
  SYNTHESIZED POLYNUCLEOTIDE
aagagcaacagtgctgtgtgcctggagcaacaaatctgactaagagcaacaaatctgact

SEQ ID NO: 9 - DNA, 79 bp, Artificial Sequence
  SYNTHESIZED POLYNUCLEOTIDE
cgtggccttagctgtgctcgcgctactctctctttctgcctggaggctatccagcgtgagtctctcctaccctcccgct

SEQ ID NO: 10 - DNA, 78 bp, Artificial Sequence
  SYNTHESIZED POLYNUCLEOTIDE
cgtggccttagctgtgctcgcgctactctctctttcgcctggaggctatccagcgtgagtctctcctaccctcccgct

SEQ ID NO: 11 - DNA, 75 bp, Artificial Sequence
  SYNTHESIZED POLYNUCLEOTIDE
cgtggccttagctgtgctcgcgctactctctctttctggaggctatccagcgtgagtctctcctaccctcccgct

SEQ ID NO: 12 - DNA, 84 bp, Artificial Sequence
  SYNTHESIZED POLYNUCLEOTIDE
cgtggccttagctgtgctcgcgctactctctctttctggatagcctggaggctatccagcgtgagtctctcctaccctcccgct

SEQ ID NO: 13 - DNA, 55 bp, Artificial Sequence
  SYNTHESIZED POLYNUCLEOTIDE
cgtggccttagctgtgctcgcgctatccagcgtgagtctctcctaccctcccgct

SEQ ID NO: 14 - DNA, 82 bp, Artificial Sequence
  SYNTHESIZED POLYNUCLEOTIDE
cgtggccttagctgtgctcgcgctactctctctttctgtggcctggaggctatccagcgtgagtctctcctaccctcccgct

SEQ ID NO: 15 - RNA, 100 nt, Artificial Sequence
  SYNTHESIZED POLYNUCLEOTIDE
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 16 - RNA, 96 nt, Artificial Sequence
  SYNTHESIZED POLYNUCLEOTIDE
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 17 - RNA, 114 nt, Artificial Sequence
  SYNTHESIZED POLYNUCLEOTIDE
  misc_feature (1)..(1): n is a, c, g, or u
  misc_feature (2)..(8): n at positions 2-8 may be absent
  misc_feature (9)..(17): n is a, c, g, or u
  misc_feature (18)..(30): n at positions 18-30 may be absent
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 18 - RNA, 100 nt, Artificial Sequence
  SYNTHESIZED POLYNUCLEOTIDE
agagcaacagugcuguggccguuuuagagcuagaaauagcaaguuaaaauaaggcuaguccguuaucaacuugaaaaaguggcaccgagucggugcuuuu

SEQ ID NO: 19 - RNA, 20 nt, Artificial Sequence
  SYNTHESIZED POLYNUCLEOTIDE
agagcaacagugcuguggcc

SEQ ID NO: 20 - RNA, 100 nt, Artificial Sequence
  SYNTHESIZED POLYNUCLEOTIDE
gcuacucucucuuucuggccguuuuagagcuagaaauagcaaguuaaaauaaggcuaguccguuaucaacuugaaaaaguggcaccgagucggugcuuuu

SEQ ID NO: 21 - RNA, 20 nt, Artificial Sequence
  SYNTHESIZED POLYNUCLEOTIDE
gcuacucucucuuucuggcc

SEQ ID NO: 22 - RNA, 100 nt, Artificial Sequence
  SYNTHESIZED POLYNUCLEOTIDE
cugcagcuucuccaacacauguuuuagagcuagaaauagcaaguuaaaauaaggcuaguccguuaucaacuugaaaaaguggcaccgagucggugcuuuu

SEQ ID NO: 23 - RNA, 20 nt, Artificial Sequence
  SYNTHESIZED POLYNUCLEOTIDE
cugcagcuucuccaacacau

SEQ ID NO: 24 - RNA, 100 nt, Artificial Sequence
  SYNTHESIZED POLYNUCLEOTIDE
gcuuuggucccauuggucgcguuuuagagcuagaaauagcaaguuaaaauaaggcuaguccguuaucaacuugaaaaaguggcaccgagucggugcuuuu

SEQ ID NO: 25 - RNA, 20 nt, Artificial Sequence
  SYNTHESIZED POLYNUCLEOTIDE
gcuuuggucccauuggucgc

SEQ ID NO: 26 - RNA, 100 nt, Artificial Sequence
  SYNTHESIZED POLYNUCLEOTIDE
gcccgcaggacgcacccauaguuuuagagcuagaaauagcaaguuaaaauaaggcuaguccguuaucaacuugaaaaaguggcaccgagucggugcuuuu

SEQ ID NO: 27 - RNA, 20 nt, Artificial Sequence
  SYNTHESIZED POLYNUCLEOTIDE
gcccgcaggacgcacccaua

SEQ ID NO: 28 - RNA, 100 nt, Artificial Sequence; identical to SEQ ID NO: 18
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 29 - RNA, 20 nt, Artificial Sequence
  SYNTHESIZED POLYNUCLEOTIDE
  modified_base (1)..(3): 2′-O-methyl phosphorothioate
agagcaacagugcuguggcc

SEQ ID NO: 30 - RNA, 100 nt, Artificial Sequence; identical to SEQ ID NO: 20
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 31 - RNA, 20 nt, Artificial Sequence
  SYNTHESIZED POLYNUCLEOTIDE
  modified_base (1)..(3): 2′-O-methyl phosphorothioate
gcuacucucucuuucuggcc

SEQ ID NO: 32 - RNA, 100 nt, Artificial Sequence; identical to SEQ ID NO: 22
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 33 - RNA, 20 nt, Artificial Sequence
  SYNTHESIZED POLYNUCLEOTIDE
  modified_base (1)..(3): 2′-O-methyl phosphorothioate
cugcagcuucuccaacacau

SEQ ID NO: 34 - RNA, 100 nt, Artificial Sequence; identical to SEQ ID NO: 24
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 35 - RNA, 20 nt, Artificial Sequence
  SYNTHESIZED POLYNUCLEOTIDE
  modified_base (1)..(3): 2′-O-methyl phosphorothioate
gcuuuggucccauuggucgc

SEQ ID NO: 36 - RNA, 100 nt, Artificial Sequence; identical to SEQ ID NO: 26
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 37 - RNA, 20 nt, Artificial Sequence
  SYNTHESIZED POLYNUCLEOTIDE
  modified_base (1)..(3): 2′-O-methyl phosphorothioate
gcccgcaggacgcacccaua

SEQ ID NO: 38 - DNA, 23 bp, Artificial Sequence
  SYNTHESIZED POLYNUCLEOTIDE
gctttggtcccattggtcgcggg

SEQ ID NO: 39 - DNA, 23 bp, Artificial Sequence
  SYNTHESIZED POLYNUCLEOTIDE
gcccgcaggacgcacccataggg

SEQ ID NO: 40 - DNA, 23 bp, Artificial Sequence
  SYNTHESIZED POLYNUCLEOTIDE
agagcaacagtgctgtggcctgg

SEQ ID NO: 41 - DNA, 23 bp, Artificial Sequence
  SYNTHESIZED POLYNUCLEOTIDE
gctactctctctttctggcctgg

SEQ ID NO: 42 - DNA, 23 bp, Artificial Sequence
  SYNTHESIZED POLYNUCLEOTIDE
ctgcagcttctccaacacatcgg

SEQ ID NO: 43 - DNA, 126 bp, Artificial Sequence
  SYNTHESIZED POLYNUCLEOTIDE
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 44 - protein, 42 aa, Artificial Sequence
  SYNTHESIZED POLYPEPTIDE
KRGRKKLLYIFKQPFMRPVQTTQEEDGCSCRFPEEEEGGCEL

SEQ ID NO: 45 - DNA, 120 bp, Artificial Sequence
  SYNTHESIZED POLYNUCLEOTIDE
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 46 - protein, 40 aa, Artificial Sequence
  SYNTHESIZED POLYPEPTIDE
SKRSRLLHSDYMNMTPRRPGPTRKHYQPYAPPRDFAAYRS

SEQ ID NO: 47 - DNA, 336 bp, Artificial Sequence
  SYNTHESIZED POLYNUCLEOTIDE
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 48 - protein, 112 aa, Artificial Sequence
  SYNTHESIZED POLYPEPTIDE
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 4361 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtacaactccaacaacccggagctgaggttgtaaaaccaggtgcgtcagtcaagatgagttgcaaagccagtggatatacttttacttcctattacattcattggatcaagcagactccaggtcaggggctcgagtgggtaggcgtgatctaccccggtaacgacgacatttcatacaaccaaaaatttcaggggaaagcgacgctgactgctgacaagagtagcacgaccgcatatatgcaactctcatcacttacgtctgaggattctgcagtttattattgcgctcgggaagttcggcttcgatatttcgatgtgtggggtcagggcacgaccgtaacggtgagcagtggtggcggtggcgggtccgggggcggtggatcaggtggtggggggagtgagatagtgttgacccagtcaccggggtccctcgcagtttcaccgggagagagggtcacaatgtcctgcaaatcctcccaatcagtgttcttctcttccagccaaaaaaactaccttgcgtggtatcaacagataccgggacagtctcctcgcctcctgatctactgggcatctacccgagaaagcggtgttccggataggtttaccggttccgggtctgggaccgattttacgttgacaatatccagcgtacagccggaagaccttgctatctattactgtcaccagtacctttccagccggacgttcgggcagggcacgaagctggagattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 50 - DNA, 1579 bp, Artificial Sequence
  SYNTHESIZED POLYNUCLEOTIDE
ccaccatggcgcttccggtgacagcactgctcctccccttggcgctgttgctccacgcagcaaggccgcaggtacaactccaacaacccggagctgaggttgtaaaaccaggtgcgtcagtcaagatgagttgcaaagccagtggatatacttttacttcctattacattcattggatcaagcagactccaggtcaggggctcgagtgggtaggcgtgatctaccccggtaacgacgacatttcatacaaccaaaaatttcaggggaaagcgacgctgactgctgacaagagtagcacgaccgcatatatgcaactctcatcacttacgtctgaggattctgcagtttattattgcgctcgggaagttcggcttcgatatttcgatgtgtggggtcagggcacgaccgtaacggtgagcagtggtggcggtggcgggtccgggggcggtggatcaggtggtggggggagtgagatagtgttgacccagtcaccggggtccctcgcagtttcaccgggagagagggtcacaatgtcctgcaaatcctcccaatcagtgttcttctcttccagccaaaaaaactaccttgcgtggtatcaacagataccgggacagtctcctcgcctcctgatctactgggcatctacccgagaaagcggtgttccggataggtttaccggttccgggtctgggaccgattttacgttgacaatatccagcgtacagccggaagaccttgctatctattactgtcaccagtacctttccagccggacgttcgggcagggcacgaagctggagattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 51 - DNA, 4367 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtacaactccaacaacccggagctgaggttgtaaaaccaggtgcgtcagtcaagatgagttgcaaagccagtggatatacttttacttcctattacattcattggatcaagcagactccaggtcaggggctcgagtgggtaggcgtgatctaccccggtaacgacgacatttcatacaaccaaaaatttcaggggaaagcgacgctgactgctgacaagagtagcacgaccgcatatatgcaactctcatcacttacgtctgaggattctgcagtttattattgcgctcgggaagttcggcttcgatatttcgatgtgtggggtcagggcacgaccgtaacggtgagcagtggtggcggtggcgggtccgggggcggtggatcaggtggtggggggagtgagatagtgttgacccagtcaccggggtccctcgcagtttcaccgggagagagggtcacaatgtcctgcaaatcctcccaatcagtgttcttctcttccagccaaaaaaactaccttgcgtggtatcaacagataccgggacagtctcctcgcctcctgatctactgggcatctacccgagaaagcggtgttccggataggtttaccggttccgggtctgggaccgattttacgttgacaatatccagcgtacagccggaagaccttgctatctattactgtcaccagtacctttccagccggacgttcgggcagggcacgaagctggagattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 52 - DNA, 1585 bp, Artificial Sequence
  SYNTHESIZED POLYNUCLEOTIDE
ccaccatggcgcttccggtgacagcactgctcctccccttggcgctgttgctccacgcagcaaggccgcaggtacaactccaacaacccggagctgaggttgtaaaaccaggtgcgtcagtcaagatgagttgcaaagccagtggatatacttttacttcctattacattcattggatcaagcagactccaggtcaggggctcgagtgggtaggcgtgatctaccccggtaacgacgacatttcatacaaccaaaaatttcaggggaaagcgacgctgactgctgacaagagtagcacgaccgcatatatgcaactctcatcacttacgtctgaggattctgcagtttattattgcgctcgggaagttcggcttcgatatttcgatgtgtggggtcagggcacgaccgtaacggtgagcagtggtggcggtggcgggtccgggggcggtggatcaggtggtggggggagtgagatagtgttgacccagtcaccggggtccctcgcagtttcaccgggagagagggtcacaatgtcctgcaaatcctcccaatcagtgttcttctcttccagccaaaaaaactaccttgcgtggtatcaacagataccgggacagtctcctcgcctcctgatctactgggcatctacccgagaaagcggtgttccggataggtttaccggttccgggtctgggaccgattttacgttgacaatatccagcgtacagccggaagaccttgctatctattactgtcaccagtacctttccagccggacgttcgggcagggcacgaagctggagattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 53 - DNA, 4361 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 54 - DNA, 1579 bp, Artificial Sequence
  SYNTHESIZED POLYNUCLEOTIDE
ccaccatggcgcttccggtgacagcactgctcctccccttggcgctgttgctccacgcagcaaggccggaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 55 - DNA, 4367 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 56 - DNA, 1585 bp, Artificial Sequence
  SYNTHESIZED POLYNUCLEOTIDE
ccaccatggcgcttccggtgacagcactgctcctccccttggcgctgttgctccacgcagcaaggccggaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 57 - DNA, 4343 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaagtgcagcttgtgcaatctggcgccgaagttaagaaaccaggcgcatctgtgaaggtgagttgtaaagcgtccggctatacttttacgaactacgacattaactgggttaggcaagctccagggcagggtctggagtggataggttggatatatccaggtgacgggtctactaaatataatgaaaaattcaaggctaaggccactttgaccgccgacacctctacatcaactgcatacatggagttgagaagccttcgcagcgatgacacggcggtatattattgtgccagtggttatgaagacgctatggactattgggggcaggggacgacggtaaccgtatcaagcggaggcggagggggatcaggcgggggcggatcaggggggggcggtagtgatatacaaatgacacaatctccaagctcactttctgccagcgttggtgatcgcgtaactattaactgcaaggcctcacaggacattaacagttatcttagctggttccagcaaaaacctgggaaagctccaaaaactttgatctatcgagcgaataggctggtcgacggagtcccatctcgcttctccggcagtggctcaggccaggactatacgctgacaataagtagtttgcaaccagaggacttcgcaacctattactgtttgcaatacgatgagttcccactcacattcggtgggggtactaaagtagaaataaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 58 - DNA, 1561 bp, Artificial Sequence
  SYNTHESIZED POLYNUCLEOTIDE
ccaccatggcgcttccggtgacagcactgctcctccccttggcgctgttgctccacgcagcaaggccgcaagtgcagcttgtgcaatctggcgccgaagttaagaaaccaggcgcatctgtgaaggtgagttgtaaagcgtccggctatacttttacgaactacgacattaactgggttaggcaagctccagggcagggtctggagtggataggttggatatatccaggtgacgggtctactaaatataatgaaaaattcaaggctaaggccactttgaccgccgacacctctacatcaactgcatacatggagttgagaagccttcgcagcgatgacacggcggtatattattgtgccagtggttatgaagacgctatggactattgggggcaggggacgacggtaaccgtatcaagcggaggcggagggggatcaggcgggggcggatcaggggggggcggtagtgatatacaaatgacacaatctccaagctcactttctgccagcgttggtgatcgcgtaactattaactgcaaggcctcacaggacattaacagttatcttagctggttccagcaaaaacctgggaaagctccaaaaactttgatctatcgagcgaataggctggtcgacggagtcccatctcgcttctccggcagtggctcaggccaggactatacgctgacaataagtagtttgcaaccagaggacttcgcaacctattactgtttgcaatacgatgagttcccactcacattcggtgggggtactaaagtagaaataaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 59 - DNA, 4349 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaagtgcagcttgtgcaatctggcgccgaagttaagaaaccaggcgcatctgtgaaggtgagttgtaaagcgtccggctatacttttacgaactacgacattaactgggttaggcaagctccagggcagggtctggagtggataggttggatatatccaggtgacgggtctactaaatataatgaaaaattcaaggctaaggccactttgaccgccgacacctctacatcaactgcatacatggagttgagaagccttcgcagcgatgacacggcggtatattattgtgccagtggttatgaagacgctatggactattgggggcaggggacgacggtaaccgtatcaagcggaggcggagggggatcaggcgggggcggatcaggggggggcggtagtgatatacaaatgacacaatctccaagctcactttctgccagcgttggtgatcgcgtaactattaactgcaaggcctcacaggacattaacagttatcttagctggttccagcaaaaacctgggaaagctccaaaaactttgatctatcgagcgaataggctggtcgacggagtcccatctcgcttctccggcagtggctcaggccaggactatacgctgacaataagtagtttgcaaccagaggacttcgcaacctattactgtttgcaatacgatgagttcccactcacattcggtgggggtactaaagtagaaataaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 60 - DNA, 1567 bp, Artificial Sequence
  SYNTHESIZED POLYNUCLEOTIDE
ccaccatggcgcttccggtgacagcactgctcctccccttggcgctgttgctccacgcagcaaggccgcaagtgcagcttgtgcaatctggcgccgaagttaagaaaccaggcgcatctgtgaaggtgagttgtaaagcgtccggctatacttttacgaactacgacattaactgggttaggcaagctccagggcagggtctggagtggataggttggatatatccaggtgacgggtctactaaatataatgaaaaattcaaggctaaggccactttgaccgccgacacctctacatcaactgcatacatggagttgagaagccttcgcagcgatgacacggcggtatattattgtgccagtggttatgaagacgctatggactattgggggcaggggacgacggtaaccgtatcaagcggaggcggagggggatcaggcgggggcggatcaggggggggcggtagtgatatacaaatgacacaatctccaagctcactttctgccagcgttggtgatcgcgtaactattaactgcaaggcctcacaggacattaacagttatcttagctggttccagcaaaaacctgggaaagctccaaaaactttgatctatcgagcgaataggctggtcgacggagtcccatctcgcttctccggcagtggctcaggccaggactatacgctgacaataagtagtttgcaaccagaggacttcgcaacctattactgtttgcaatacgatgagttcccactcacattcggtgggggtactaaagtagaaataaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 61 - DNA, 4343 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggacatccaaatgacccagtcaccgagttcactgtctgccagtgtaggagatcgcgtcaccattaattgcaaggccagtcaggacataaatagttacctgagctggttccagcaaaaaccaggtaaggccccgaagactcttatctatcgagcgaaccgacttgtagacggtgttccttccagattttccggcagtggcagcggccaagattacactcttactatctcttcactgcaacctgaagatttcgcgacctattactgcctccaatacgacgagttcccactgacgtttggcggcggaacgaaggtagaaatcaagggcgggggaggggggtcaggtggaggcggctctggcggaggtggtagtcaggtccaactcgttcagagcggggcggaggtaaagaagccaggggccagtgtcaaggttagttgtaaagcatctggctataccttcacgaattacgatataaactgggtacgacaagcccctgggcaaggacttgaatggattggatggatctatccaggcgatggatcaaccaaatacaatgagaagtttaaggctaaagccacactcaccgccgatacctccaccagtacagcgtatatggagttgaggtcacttcgctctgatgatactgcggtgtactattgcgcaagtggttacgaggacgctatggactactgggggcaagggacaacagtgaccgtttcttctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 62 - DNA, 1561 bp, Artificial Sequence
  SYNTHESIZED POLYNUCLEOTIDE
ccaccatggcgcttccggtgacagcactgctcctccccttggcgctgttgctccacgcagcaaggccggacatccaaatgacccagtcaccgagttcactgtctgccagtgtaggagatcgcgtcaccattaattgcaaggccagtcaggacataaatagttacctgagctggttccagcaaaaaccaggtaaggccccgaagactcttatctatcgagcgaaccgacttgtagacggtgttccttccagattttccggcagtggcagcggccaagattacactcttactatctcttcactgcaacctgaagatttcgcgacctattactgcctccaatacgacgagttcccactgacgtttggcggcggaacgaaggtagaaatcaagggcgggggaggggggtcaggtggaggcggctctggcggaggtggtagtcaggtccaactcgttcagagcggggcggaggtaaagaagccaggggccagtgtcaaggttagttgtaaagcatctggctataccttcacgaattacgatataaactgggtacgacaagcccctgggcaaggacttgaatggattggatggatctatccaggcgatggatcaaccaaatacaatgagaagtttaaggctaaagccacactcaccgccgatacctccaccagtacagcgtatatggagttgaggtcacttcgctctgatgatactgcggtgtactattgcgcaagtggttacgaggacgctatggactactgggggcaagggacaacagtgaccgtttcttctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 63 - DNA, 4349 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggacatccaaatgacccagtcaccgagttcactgtctgccagtgtaggagatcgcgtcaccattaattgcaaggccagtcaggacataaatagttacctgagctggttccagcaaaaaccaggtaaggccccgaagactcttatctatcgagcgaaccgacttgtagacggtgttccttccagattttccggcagtggcagcggccaagattacactcttactatctcttcactgcaacctgaagatttcgcgacctattactgcctccaatacgacgagttcccactgacgtttggcggcggaacgaaggtagaaatcaagggcgggggaggggggtcaggtggaggcggctctggcggaggtggtagtcaggtccaactcgttcagagcggggcggaggtaaagaagccaggggccagtgtcaaggttagttgtaaagcatctggctataccttcacgaattacgatataaactgggtacgacaagcccctgggcaaggacttgaatggattggatggatctatccaggcgatggatcaaccaaatacaatgagaagtttaaggctaaagccacactcaccgccgatacctccaccagtacagcgtatatggagttgaggtcacttcgctctgatgatactgcggtgtactattgcgcaagtggttacgaggacgctatggactactgggggcaagggacaacagtgaccgtttcttctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 64 - DNA, 1567 bp, Artificial Sequence
  SYNTHESIZED POLYNUCLEOTIDE
ccaccatggcgcttccggtgacagcactgctcctccccttggcgctgttgctccacgcagcaaggccggacatccaaatgacccagtcaccgagttcactgtctgccagtgtaggagatcgcgtcaccattaattgcaaggccagtcaggacataaatagttacctgagctggttccagcaaaaaccaggtaaggccccgaagactcttatctatcgagcgaaccgacttgtagacggtgttccttccagattttccggcagtggcagcggccaagattacactcttactatctcttcactgcaacctgaagatttcgcgacctattactgcctccaatacgacgagttcccactgacgtttggcggcggaacgaaggtagaaatcaagggcgggggaggggggtcaggtggaggcggctctggcggaggtggtagtcaggtccaactcgttcagagcggggcggaggtaaagaagccaggggccagtgtcaaggttagttgtaaagcatctggctataccttcacgaattacgatataaactgggtacgacaagcccctgggcaaggacttgaatggattggatggatctatccaggcgatggatcaaccaaatacaatgagaagtttaaggctaaagccacactcaccgccgatacctccaccagtacagcgtatatggagttgaggtcacttcgctctgatgatactgcggtgtactattgcgcaagtggttacgaggacgctatggactactgggggcaagggacaacagtgaccgtttcttctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 65 - protein, 118 aa, Artificial Sequence
  SYNTHESIZED POLYPEPTIDE
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 66 - protein, 112 aa, Artificial Sequence
  SYNTHESIZED POLYPEPTIDE
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIK

SEQ ID NO: 67 - protein, 5 aa, Artificial Sequence
  SYNTHESIZED POLYPEPTIDE
SYYIH

SEQ ID NO: 68 - protein, 17 aa, Artificial Sequence
  SYNTHESIZED POLYPEPTIDE
VIYPGNDDISYNQKFQG

SEQ ID NO: 69 - protein, 9 aa, Artificial Sequence
  SYNTHESIZED POLYPEPTIDE
EVRLRYFDV

SEQ ID NO: 70 - protein, 17 aa, Artificial Sequence
  SYNTHESIZED POLYPEPTIDE
KSSQSVFFSSSQKNYLA

SEQ ID NO: 71 - protein, 7 aa, Artificial Sequence
  SYNTHESIZED POLYPEPTIDE
WASTRES

SEQ ID NO: 72 - protein, 8 aa, Artificial Sequence
  SYNTHESIZED POLYPEPTIDE
HQYLSSRT

SEQ ID NO: 73 - protein, 246 aa, Artificial Sequence
  SYNTHESIZED POLYPEPTIDE
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSGGGGGSGGGGSGGGGSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIK

SEQ ID NO: 74 - DNA, 738 bp, Artificial Sequence
  SYNTHESIZED POLYNUCLEOTIDE
caggtacaactccaacaacccggagctgaggttgtaaaaccaggtgcgtcagtcaagatgagttgcaaagccagtggatatacttttacttcctattacattcattggatcaagcagactccaggtcaggggctcgagtgggtaggcgtgatctaccccggtaacgacgacatttcatacaaccaaaaatttcaggggaaagcgacgctgactgctgacaagagtagcacgaccgcatatatgcaactctcatcacttacgtctgaggattctgcagtttattattgcgctcgggaagttcggcttcgatatttcgatgtgtggggtcagggcacgaccgtaacggtgagcagtggtggcggtggcgggtccgggggcggtggatcaggtggtggggggagtgagatagtgttgacccagtcaccggggtccctcgcagtttcaccgggagagagggtcacaatgtcctgcaaatcctcccaatcagtgttcttctcttccagccaaaaaaactaccttgcgtggtatcaacagataccgggacagtctcctcgcctcctgatctactgggcatctacccgagaaagcggtgttccggataggtttaccggttccgggtctgggaccgattttacgttgacaatatccagcgtacagccggaagaccttgctatctattactgtcaccagtacctttccagccggacgttcgggcagggcacgaagctggagattaaa

SEQ ID NO: 75 - protein, 246 aa, Artificial Sequence
  SYNTHESIZED POLYPEPTIDE
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKGGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 76 - DNA, 738 bp, Artificial Sequence
  SYNTHESIZED POLYNUCLEOTIDE
gaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagc

SEQ ID NO: 77 - protein, 117 aa, Artificial Sequence
  SYNTHESIZED POLYPEPTIDE
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 78 - protein, 107 aa, Artificial Sequence
  SYNTHESIZED POLYPEPTIDE
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 79 - protein, 5 aa, Artificial Sequence
  SYNTHESIZED POLYPEPTIDE
NYDIN

SEQ ID NO: 80 - protein, 17 aa, Artificial Sequence
  SYNTHESIZED POLYPEPTIDE
WIYPGDGSTKYNEKFKA

SEQ ID NO: 81 - protein, 8 aa, Artificial Sequence
  SYNTHESIZED POLYPEPTIDE
GYEDAMDY

SEQ ID NO: 82 - protein, 11 aa, Artificial Sequence
  SYNTHESIZED POLYPEPTIDE
KASQDINSYLS

SEQ ID NO: 83 - protein, 7 aa, Artificial Sequence
  SYNTHESIZED POLYPEPTIDE
RANRLVD

SEQ ID NO: 84 - protein, 9 aa, Artificial Sequence
  SYNTHESIZED POLYPEPTIDE
LQYDEFPLT

SEQ ID NO: 85 - protein, 239 aa, Artificial Sequence
  SYNTHESIZED POLYPEPTIDE
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 86 - DNA, 720 bp, Artificial Sequence
  SYNTHESIZED POLYNUCLEOTIDE
caagtgcagcttgtgcaatctggcgccgaagttaagaaaccaggcgcatctgtgaaggtgagttgtaaagcgtccggctatacttttacgaactacgacattaactgggttaggcaagctccagggcagggtctggagtggataggttggatatatccaggtgacgggtctactaaatataatgaaaaattcaaggctaaggccactttgaccgccgacacctctacatcaactgcatacatggagttgagaagccttcgcagcgatgacacggcggtatattattgtgccagtggttatgaagacgctatggactattgggggcaggggacgacggtaaccgtatcaagcggaggcggagggggatcaggcgggggcggatcaggggggggcggtagtgatatacaaatgacacaatctccaagctcactttctgccagcgttggtgatcgcgtaactattaactgcaaggcctcacaggacattaacagttatcttagctggttccagcaaaaacctgggaaagctccaaaaactttgatctatcgagcgaataggctggtcgacggagtcccatctcgcttctccggcagtggctcaggccaggactatacgctgacaataagtagtttgcaaccagaggacttcgcaacctattactgtttgcaatacgatgagttcccactcacattcggtgggggtactaaagtagaaataaaa

SEQ ID NO: 87 - protein, 239 aa, Artificial Sequence
  SYNTHESIZED POLYPEPTIDE
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKGGGGSGGGGSGGGGSQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 88 - DNA, 720 bp, Artificial Sequence
  SYNTHESIZED POLYNUCLEOTIDE
gacatccaaatgacccagtcaccgagttcactgtctgccagtgtaggagatcgcgtcaccattaattgcaaggccagtcaggacataaatagttacctgagctggttccagcaaaaaccaggtaaggccccgaagactcttatctatcgagcgaaccgacttgtagacggtgttccttccagattttccggcagtggcagcggccaagattacactcttactatctcttcactgcaacctgaagatttcgcgacctattactgcctccaatacgacgagttcccactgacgtttggcggcggaacgaaggtagaaatcaagggcgggggaggggggtcaggtggaggcggctctggcggaggtggtagtcaggtccaactcgttcagagcggggcggaggtaaagaagccaggggccagtgtcaaggttagttgtaaagcatctggctataccttcacgaattacgatataaactgggtacgacaagcccctgggcaaggacttgaatggattggatggatctatccaggcgatggatcaaccaaatacaatgagaagtttaaggctaaagccacactcaccgccgatacctccaccagtacagcgtatatggagttgaggtcacttcgctctgatgatactgcggtgtactattgcgcaagtggttacgaggacgccatggactactgggggcaagggacaacagtgaccgtttcttct

SEQ ID NO: 89 - protein, 116 aa, Artificial Sequence [sequence omitted]

SEQ ID NO: 90 - protein, 111 aa, Artificial Sequence [sequence omitted]

SEQ ID NO: 91 - protein, 5 aa, Artificial Sequence
  SYNTHESIZED POLYPEPTIDE
DSNIH

SEQ ID NO: 92 - protein, 17 aa, Artificial Sequence
  SYNTHESIZED POLYPEPTIDE
YIYPYNGGTDYNQKFKN

SEQ ID NO: 93 - protein, 7 aa, Artificial Sequence
  SYNTHESIZED POLYPEPTIDE
GNPWLAY

SEQ ID NO: 94 - protein, 15 aa, Artificial Sequence
  SYNTHESIZED POLYPEPTIDE
RASESLDNYGIRFLT

SEQ ID NO: 95 - protein, 7 aa, Artificial Sequence
  SYNTHESIZED POLYPEPTIDE
AASNQGS

SEQ ID NO: 96 - protein, 9 aa, Artificial Sequence
  SYNTHESIZED POLYPEPTIDE
QQTKEVPWS

SEQ ID NO: 97 - protein, 242 aa, Artificial Sequence
  SYNTHESIZED POLYPEPTIDE
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSGGGGSGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSS

SEQ ID NO: 98 - DNA, 726 bp, Artificial Sequence
  SYNTHESIZED POLYNUCLEOTIDE
gatatacagctcacgcagagtccatcaacactgtccgccagtgtcggtgaccgggttactattacgtgccgcgcaagcgaatctctggataattatggtatccggtttctgacatggtttcagcaaaaaccggggaaagctcccaagctgcttatgtacgccgcctctaatcaggggtcaggtgtccctagccggttctccggttccggtagtggcacggaattcactctcacaatcagttcactccagccggatgactttgcaacgtattattgtcaacaaacgaaggaggttccttggtctttcggtcagggaactaaggttgaggttaagggaggaggtggttctggcggaggcggatctggtggcggaggttccgaggtacaacttgtgcaaagtggggctgaggttaaaaaacccggcagctctgtcaaagtttcctgtaaggctagtggttacaccatcactgactccaatatacactgggttagacaggctccagggcagtcacttgagtggataggctacatctatccatacaacggaggtacagactacaaccaaaaatttaaaaacagggcgacgcttacagtcgataaccccacaaatacagcatatatggagctgtcatctttgcgcagcgaagatacagctttctactattgtgtgaatggtaatccctggctggcctattgggggcagggaactcttgtcactgtttccagt

SEQ ID NO: 99 - protein, 242 aa, Artificial Sequence
  SYNTHESIZED POLYPEPTIDE
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 100 - DNA, 726 bp, Artificial Sequence
  SYNTHESIZED POLYNUCLEOTIDE
gaggtccaacttgttcaatccggcgctgaagtgaaaaagccaggaagtagcgtaaaagtaagctgtaaagctagcggttacaccattaccgacagcaacatccattgggtgcggcaggcgccaggacaatccctcgagtggataggttacatctatccttacaacgggggaacagattataatcagaagttcaagaaccgggcaacgctcactgttgacaatcccactaatactgcctatatggagctctccagcctccgcagtgaggacactgcgttttattattgcgtgaatggcaacccgtggcttgcttattggggacagggcacattggttacagtaagttctggtggcggaggttccgggggagggggtagtggtggtggtgggtcagacattcaacttacacaaagtccatcaaccctcagtgcgtctgtaggggatcgggtcacaataacctgccgagccagcgagtctttggacaactacggaataaggttcctcacgtggtttcagcagaaaccgggcaaagcacccaagctccttatgtatgccgcgagcaaccagggttccggagtcccgagccggttttctggttccgggagcggtacggagttcacactcacaatatcttccctgcagcctgatgactttgccacctactattgccagcagactaaagaggttccctggtcctttggtcagggcacgaaagtggaagtcaaa

SEQ ID NO: 101 - protein, 507 aa, Artificial Sequence
  SYNTHESIZED POLYPEPTIDE
MALPVTALLLPLALLLHAARPQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSGGGGGSGGGGSGGGGSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 509 aa, Artificial Sequence
  SYNTHESIZED POLYPEPTIDE
MALPVTALLLPLALLLHAARPQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSGGGGGSGGGGSGGGGSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 507 aa, Artificial Sequence
  SYNTHESIZED POLYPEPTIDE
MALPVTALLLPLALLLHAARPEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKGGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 509 aa, Artificial Sequence
  SYNTHESIZED POLYPEPTIDE
MALPVTALLLPLALLLHAARPEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKGGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 501 aa, Artificial Sequence
  SYNTHESIZED POLYPEPTIDE
MALPVTALLLPLALLLHAARPQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 503 aa, Artificial Sequence
  SYNTHESIZED POLYPEPTIDE
MALPVTALLLPLALLLHAARPQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 501 aa, Artificial Sequence
  SYNTHESIZED POLYPEPTIDE
MALPVTALLLPLALLLHAARPDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKGGGGGSGGGGSGGGGSQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 503 aa, Artificial Sequence
  SYNTHESIZED POLYPEPTIDE
MALPVTALLLPLALLLHAARPDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKGGGGGSGGGGSGGGGSQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - DNA, 4349 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggatatacagctcacgcagagtccatcaacactgtccgccagtgtcggtgaccgggttactattacgtgccgcgcaagcgaatctctggataattatggtatccggtttctgacatggtttcagcaaaaaccggggaaagctcccaagctgcttatgtacgccgcctctaatcaggggtcaggtgtccctagccggttctccggttccggtagtggcacggaattcactctcacaatcagttcactccagccggatgactttgcaacgtattattgtcaacaaacgaaggaggttccttggtctttcggtcagggaactaaggttgaggttaagggaggaggtggttctggcggaggcggatctggtggcggaggttccgaggtacaacttgtgcaaagtggggctgaggttaaaaaacccggcagctctgtcaaagtttcctgtaaggctagtggttacaccatcactgactccaatatacactgggttagacaggctccagggcagtcacttgagtggataggctacatctatccatacaacggaggtacagactacaaccaaaaatttaaaaacagggcgacgcttacagtcgataaccccacaaatacagcatatatggagctgtcatctttgcgcagcgaagatacagctttctactattgtgtgaatggtaatccctggctggcctattgggggcagggaactcttgtcactgtttccagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 110 - DNA, 1509 bp, Artificial Sequence
  SYNTHESIZED POLYNUCLEOTIDE
atggcgcttccggtgacagcactgctcctccccttggcgctgttgctccacgcagcaaggccggatatacagctcacgcagagtccatcaacactgtccgccagtgtcggtgaccgggttactattacgtgccgcgcaagcgaatctctggataattatggtatccggtttctgacatggtttcagcaaaaaccggggaaagctcccaagctgcttatgtacgccgcctctaatcaggggtcaggtgtccctagccggttctccggttccggtagtggcacggaattcactctcacaatcagttcactccagccggatgactttgcaacgtattattgtcaacaaacgaaggaggttccttggtctttcggtcagggaactaaggttgaggttaagggaggaggtggttctggcggaggcggatctggtggcggaggttccgaggtacaacttgtgcaaagtggggctgaggttaaaaaacccggcagctctgtcaaagtttcctgtaaggctagtggttacaccatcactgactccaatatacactgggttagacaggctccagggcagtcacttgagtggataggctacatctatccatacaacggaggtacagactacaaccaaaaatttaaaaacagggcgacgcttacagtcgataaccccacaaatacagcatatatggagctgtcatctttgcgcagcgaagatacagctttctactattgtgtgaatggtaatccctggctggcctattgggggcagggaactcttgtcactgtttccagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 111 - protein, 503 aa, Artificial Sequence
  SYNTHESIZED POLYPEPTIDE
MALPVTALLLPLALLLHAARPDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSGGGGSGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - DNA, 4355 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggatatacagctcacgcagagtccatcaacactgtccgccagtgtcggtgaccgggttactattacgtgccgcgcaagcgaatctctggataattatggtatccggtttctgacatggtttcagcaaaaaccggggaaagctcccaagctgcttatgtacgccgcctctaatcaggggtcaggtgtccctagccggttctccggttccggtagtggcacggaattcactctcacaatcagttcactccagccggatgactttgcaacgtattattgtcaacaaacgaaggaggttccttggtctttcggtcagggaactaaggttgaggttaagggaggaggtggttctggcggaggcggatctggtggcggaggttccgaggtacaacttgtgcaaagtggggctgaggttaaaaaacccggcagctctgtcaaagtttcctgtaaggctagtggttacaccatcactgactccaatatacactgggttagacaggctccagggcagtcacttgagtggataggctacatctatccatacaacggaggtacagactacaaccaaaaatttaaaaacagggcgacgcttacagtcgataaccccacaaatacagcatatatggagctgtcatctttgcgcagcgaagatacagctttctactattgtgtgaatggtaatccctggctggcctattgggggcagggaactcttgtcactgtttccagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 113 - DNA, 1515 bp, Artificial Sequence
  SYNTHESIZED POLYNUCLEOTIDE
atggcgcttccggtgacagcactgctcctccccttggcgctgttgctccacgcagcaaggccggatatacagctcacgcagagtccatcaacactgtccgccagtgtcggtgaccgggttactattacgtgccgcgcaagcgaatctctggataattatggtatccggtttctgacatggtttcagcaaaaaccggggaaagctcccaagctgcttatgtacgccgcctctaatcaggggtcaggtgtccctagccggttctccggttccggtagtggcacggaattcactctcacaatcagttcactccagccggatgactttgcaacgtattattgtcaacaaacgaaggaggttccttggtctttcggtcagggaactaaggttgaggttaagggaggaggtggttctggcggaggcggatctggtggcggaggttccgaggtacaacttgtgcaaagtggggctgaggttaaaaaacccggcagctctgtcaaagtttcctgtaaggctagtggttacaccatcactgactccaatatacactgggttagacaggctccagggcagtcacttgagtggataggctacatctatccatacaacggaggtacagactacaaccaaaaatttaaaaacagggcgacgcttacagtcgataaccccacaaatacagcatatatggagctgtcatctttgcgcagcgaagatacagctttctactattgtgtgaatggtaatccctggctggcctattgggggcagggaactcttgtcactgtttccagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 114 - protein, 505 aa, Artificial Sequence
  SYNTHESIZED POLYPEPTIDE
MALPVTALLLPLALLLHAARPDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKGGGGSGGGGSGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - DNA, 4349 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggaggtccaacttgttcaatccggcgctgaagtgaaaaagccaggaagtagcgtaaaagtaagctgtaaagctagcggttacaccattaccgacagcaacatccattgggtgcggcaggcgccaggacaatccctcgagtggataggttacatctatccttacaacgggggaacagattataatcagaagttcaagaaccgggcaacgctcactgttgacaatcccactaatactgcctatatggagctctccagcctccgcagtgaggacactgcgttttattattgcgtgaatggcaacccgtggcttgcttattggggacagggcacattggttacagtaagttctggtggcggaggttccgggggagggggtagtggtggtggtgggtcagacattcaacttacacaaagtccatcaaccctcagtgcgtctgtaggggatcgggtcacaataacctgccgagccagcgagtctttggacaactacggaataaggttcctcacgtggtttcagcagaaaccgggcaaagcacccaagctccttatgtatgccgcgagcaaccagggttccggagtcccgagccggttttctggttccgggagcggtacggagttcacactcacaatatcttccctgcagcctgatgactttgccacctactattgccagcagactaaagaggttccctggtcctttggtcagggcacgaaagtggaagtcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 116 - DNA, 1509 bp, Artificial Sequence
  SYNTHESIZED POLYNUCLEOTIDE
atggcgcttccggtgacagcactgctcctccccttggcgctgttgctccacgcagcaaggccggaggtccaacttgttcaatccggcgctgaagtgaaaaagccaggaagtagcgtaaaagtaagctgtaaagctagcggttacaccattaccgacagcaacatccattgggtgcggcaggcgccaggacaatccctcgagtggataggttacatctatccttacaacgggggaacagattataatcagaagttcaagaaccgggcaacgctcactgttgacaatcccactaatactgcctatatggagctctccagcctccgcagtgaggacactgcgttttattattgcgtgaatggcaacccgtggcttgcttattggggacagggcacattggttacagtaagttctggtggcggaggttccgggggagggggtagtggtggtggtgggtcagacattcaacttacacaaagtccatcaaccctcagtgcgtctgtaggggatcgggtcacaataacctgccgagccagcgagtctttggacaactacggaataaggttcctcacgtggtttcagcagaaaccgggcaaagcacccaagctccttatgtatgccgcgagcaaccagggttccggagtcccgagccggttttctggttccgggagcggtacggagttcacactcacaatatcttccctgcagcctgatgactttgccacctactattgccagcagactaaagaggttccctggtcctttggtcagggcacgaaagtggaagtcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 117 - protein, 503 aa, Artificial Sequence
  SYNTHESIZED POLYPEPTIDE
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - DNA, 4355 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggaggtccaacttgttcaatccggcgctgaagtgaaaaagccaggaagtagcgtaaaagtaagctgtaaagctagcggttacaccattaccgacagcaacatccattgggtgcggcaggcgccaggacaatccctcgagtggataggttacatctatccttacaacgggggaacagattataatcagaagttcaagaaccgggcaacgctcactgttgacaatcccactaatactgcctatatggagctctccagcctccgcagtgaggacactgcgttttattattgcgtgaatggcaacccgtggcttgcttattggggacagggcacattggttacagtaagttctggtggcggaggttccgggggagggggtagtggtggtggtgggtcagacattcaacttacacaaagtccatcaaccctcagtgcgtctgtaggggatcgggtcacaataacctgccgagccagcgagtctttggacaactacggaataaggttcctcacgtggtttcagcagaaaccgggcaaagcacccaagctccttatgtatgccgcgagcaaccagggttccggagtcccgagccggttttctggttccgggagcggtacggagttcacactcacaatatcttccctgcagcctgatgactttgccacctactattgccagcagactaaagaggttccctggtcctttggtcagggcacgaaagtggaagtcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 119 - DNA, 1515 bp, Artificial Sequence
  SYNTHESIZED POLYNUCLEOTIDE
atggcgcttccggtgacagcactgctcctccccttggcgctgttgctccacgcagcaaggccggaggtccaacttgttcaatccggcgctgaagtgaaaaagccaggaagtagcgtaaaagtaagctgtaaagctagcggttacaccattaccgacagcaacatccattgggtgcggcaggcgccaggacaatccctcgagtggataggttacatctatccttacaacgggggaacagattataatcagaagttcaagaaccgggcaacgctcactgttgacaatcccactaatactgcctatatggagctctccagcctccgcagtgaggacactgcgttttattattgcgtgaatggcaacccgtggcttgcttattggggacagggcacattggttacagtaagttctggtggcggaggttccgggggagggggtagtggtggtggtgggtcagacattcaacttacacaaagtccatcaaccctcagtgcgtctgtaggggatcgggtcacaataacctgccgagccagcgagtctttggacaactacggaataaggttcctcacgtggtttcagcagaaaccgggcaaagcacccaagctccttatgtatgccgcgagcaaccagggttccggagtcccgagccggttttctggttccgggagcggtacggagttcacactcacaatatcttccctgcagcctgatgactttgccacctactattgccagcagactaaagaggttccctggtcctttggtcagggcacgaaagtggaagtcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 120 - protein, 505 aa, Artificial Sequence
  SYNTHESIZED POLYPEPTIDE
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 21 aa, Artificial Sequence
  SYNTHESIZED POLYPEPTIDE
MALPVTALLLPLALLLHAARP

SEQ ID NO: 122 - DNA, 261 bp, Artificial Sequence
  SYNTHESIZED POLYNUCLEOTIDE
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 123 - protein, 88 aa, Artificial Sequence
  SYNTHESIZED POLYPEPTIDE
SAAAFVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 124 - DNA, 252 bp, Artificial Sequence
  SYNTHESIZED POLYNUCLEOTIDE
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 125 - protein, 84 aa, Artificial Sequence
  SYNTHESIZED POLYPEPTIDE
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 126-127 - skipped (2 entries)

SEQ ID NO: 128 - DNA, 800 bp, Artificial Sequence
  SYNTHESIZED POLYNUCLEOTIDE
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 129 - DNA, 1178 bp, Artificial Sequence
  SYNTHESIZED POLYNUCLEOTIDE
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 130 - DNA, 49 bp, Artificial Sequence
  SYNTHESIZED POLYNUCLEOTIDE
aataaaatcgctatccatcgaagatggatgtgtgttggttttttgtgtg

SEQ ID NO: 131 - DNA, 804 bp, Artificial Sequence
  SYNTHESIZED POLYNUCLEOTIDE
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 132 - RNA, 99 nt, Artificial Sequence
  SYNTHESIZED POLYNUCLEOTIDE
uggcuauggauccaaauuucguuuuagagcuagaaauagcaaguuaaaauaaggcuaguccguuaucaacuugaaaaaguggcaccgagucggugcuuu

SEQ ID NO: 133 - RNA, 100 nt, Artificial Sequence
  SYNTHESIZED POLYNUCLEOTIDE
acuccccaguucaugguuacguuuuagagcuagaaauagcaaguuaaaauaaggcuaguccguuaucaacuugaaaaaguggcaccgagucggugcuuuu

SEQ ID NO: 134 - RNA, 100 nt, Artificial Sequence
  SYNTHESIZED POLYNUCLEOTIDE
gacaagaacuccccaguucaguuuuagagcuagaaauagcaaguuaaaauaaggcuaguccguuaucaacuugaaaaaguggcaccgagucggugcuuuu

SEQ ID NO: 135 - RNA, 99 nt, Artificial Sequence
  SYNTHESIZED POLYNUCLEOTIDE
gggaaggagccauuauauccguuuuagagcuagaaauagcaaguuaaaauaaggcuaguccguuaucaacuugaaaaaguggcaccgagucggugcuuu

SEQ ID NO: 136 - RNA, 100 nt, Artificial Sequence
  SYNTHESIZED POLYNUCLEOTIDE
gcaggagucagugacgguacguuuuagagcuagaaauagcaaguuaaaauaaggcuaguccguuaucaacuugaaaaaguggcaccgagucggugcuuuu

SEQ ID NO: 137 - RNA, 100 nt, Artificial Sequence
  SYNTHESIZED POLYNUCLEOTIDE
accugucaggugaaguucgcguuuuagagcuagaaauagcaaguuaaaauaaggcuaguccguuaucaacuugaaaaaguggcaccgagucggugcuuuu

SEQ ID NO: 138 - RNA, 100 nt, Artificial Sequence
  SYNTHESIZED POLYNUCLEOTIDE
caggugaaguucgcuggagcguuuuagagcuagaaauagcaaguuaaaauaaggcuaguccguuaucaacuugaaaaaguggcaccgagucggugcuuuu

SEQ ID NO: 139 - RNA, 100 nt, Artificial Sequence
  SYNTHESIZED POLYNUCLEOTIDE
ccccaggacuacucacuccuguuuuagagcuagaaauagcaaguuaaaauaaggcuaguccguuaucaacuugaaaaaguggcaccgagucggugcuuuu

SEQ ID NO: 140 - RNA, 100 nt, Artificial Sequence
  SYNTHESIZED POLYNUCLEOTIDE
gaacacccccgaucuucuccguuuuagagcuagaaauagcaaguuaaaauaaggcuaguccguuaucaacuugaaaaaguggcaccgagucggugcuuuu

SEQ ID NO: 141 - RNA, 100 nt, Artificial Sequence
  SYNTHESIZED POLYNUCLEOTIDE
aggcccaaaauccucaucccguuuuagagcuagaaauagcaaguuaaaauaaggcuaguccguuaucaacuugaaaaaguggcaccgagucggugcuuuu

SEQ ID NO: 142 - RNA, 100 nt, Artificial Sequence
  SYNTHESIZED POLYNUCLEOTIDE
  modified_base (1)..(3): 2′-O-methyl phosphorothioate
  modified_base (97)..(99): 2′-O-methyl phosphorothioate
uggcuauggauccaaauuucguuuuagagcuagaaauagcaaguuaaaauaaggcuaguccguuaucaacuugaaaaaguggcaccgagucggugcuuuu

SEQ ID NO: 143 - RNA, 100 nt, Artificial Sequence; identical to SEQ ID NO: 133
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 144 - RNA, 100 nt, Artificial Sequence; identical to SEQ ID NO: 134
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 145 - RNA, 100 nt, Artificial Sequence
  SYNTHESIZED POLYNUCLEOTIDE
  modified_base (1)..(3): 2′-O-methyl phosphorothioate
  modified_base (97)..(99): 2′-O-methyl phosphorothioate
gggaaggagccauuauauccguuuuagagcuagaaauagcaaguuaaaauaaggcuaguccguuaucaacuugaaaaaguggcaccgagucggugcuuuu

SEQ ID NO: 146 - RNA, 100 nt, Artificial Sequence; identical to SEQ ID NO: 136
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 147 - RNA, 100 nt, Artificial Sequence; identical to SEQ ID NO: 137
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 148 - RNA, 100 nt, Artificial Sequence; identical to SEQ ID NO: 138
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 149 - RNA, 100 nt, Artificial Sequence; identical to SEQ ID NO: 139
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 150 - RNA, 100 nt, Artificial Sequence; identical to SEQ ID NO: 140
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 151 - RNA, 100 nt, Artificial Sequence; identical to SEQ ID NO: 141
  SYNTHESIZED POLYNUCLEOTIDE
  modified_base (1)..(3): 2′-O-methyl phosphorothioate
  modified_base (97)..(99): 2′-O-methyl phosphorothioate

SEQ ID NO: 152 - DNA, 23 bp, Artificial Sequence
  SYNTHESIZED POLYNUCLEOTIDE
tggctatggatccaaatttctgg

SEQ ID NO: 153 - DNA, 23 bp, Artificial Sequence
  SYNTHESIZED POLYNUCLEOTIDE
actccccagttcatggttactgg

SEQ ID NO: 154 - DNA, 23 bp, Artificial Sequence
  SYNTHESIZED POLYNUCLEOTIDE
gacaagaactccccagttcatgg

SEQ ID NO: 155 - DNA, 23 bp, Artificial Sequence
  SYNTHESIZED POLYNUCLEOTIDE
gggaaggagccattatatccagg

SEQ ID NO: 156 - DNA, 23 bp, Artificial Sequence
  SYNTHESIZED POLYNUCLEOTIDE
gcaggagtcagtgacggtacagg

SEQ ID NO: 157 - DNA, 23 bp, Artificial Sequence
  SYNTHESIZED POLYNUCLEOTIDE
acctgtcaggtgaagttcgctgg

SEQ ID NO: 158 - DNA, 23 bp, Artificial Sequence
  SYNTHESIZED POLYNUCLEOTIDE
caggtgaagttcgctggagctgg

SEQ ID NO: 159 - DNA, 23 bp, Artificial Sequence
  SYNTHESIZED POLYNUCLEOTIDE
ccccaggactactcactcctcgg

SEQ ID NO: 160 - DNA, 23 bp, Artificial Sequence
  SYNTHESIZED POLYNUCLEOTIDE
gaacacccccgatcttctcctgg

SEQ ID NO: 161 - DNA, 23 bp, Artificial Sequence
  SYNTHESIZED POLYNUCLEOTIDE
aggcccaaaatcctcatccctgg

SEQ ID NO: 162 - protein, 22 aa, Artificial Sequence
  SYNTHESIZED POLYPEPTIDE
MLLLVTSLLLCELPHPAFLLIP

SEQ ID NO: 163 - protein, 23 aa, Artificial Sequence
  SYNTHESIZED POLYPEPTIDE
IYIWAPLAGTCGVLLLSLVITLY

SEQ ID NO: 164 - RNA, 20 nt, Artificial Sequence
  SYNTHESIZED POLYNUCLEOTIDE
uggcuauggauccaaauuuc

SEQ ID NO: 165 - RNA, 20 nt, Artificial Sequence
  SYNTHESIZED POLYNUCLEOTIDE
acuccccaguucaugguuac

SEQ ID NO: 166 - RNA, 20 nt, Artificial Sequence
  SYNTHESIZED POLYNUCLEOTIDE
gacaagaacuccccaguuca

SEQ ID NO: 167 - RNA, 20 nt, Artificial Sequence
  SYNTHESIZED POLYNUCLEOTIDE
gggaaggagccauuauaucc

SEQ ID NO: 168 - RNA, 20 nt, Artificial Sequence
  SYNTHESIZED POLYNUCLEOTIDE
gcaggagucagugacgguac

SEQ ID NO: 169 - RNA, 20 nt, Artificial Sequence
  SYNTHESIZED POLYNUCLEOTIDE
accugucaggugaaguucgc

SEQ ID NO: 170 - RNA, 20 nt, Artificial Sequence
  SYNTHESIZED POLYNUCLEOTIDE
caggugaaguucgcuggagc

SEQ ID NO: 171 - RNA, 20 nt, Artificial Sequence
  SYNTHESIZED POLYNUCLEOTIDE
ccccaggacuacucacuccu

SEQ ID NO: 172 - RNA, 20 nt, Artificial Sequence
  SYNTHESIZED POLYNUCLEOTIDE
gaacacccccgaucuucucc

SEQ ID NO: 173 - RNA, 20 nt, Artificial Sequence
  SYNTHESIZED POLYNUCLEOTIDE
aggcccaaaauccucauccc

SEQ ID NO: 174 - DNA, 20 bp, Artificial Sequence
  SYNTHESIZED POLYNUCLEOTIDE
ggatccaaatttctggctgc

SEQ ID NO: 175 - DNA, 19 bp, Artificial Sequence
  SYNTHESIZED POLYNUCLEOTIDE
ggatccaaattctggctgc

SEQ ID NO: 176 - DNA, 21 bp, Artificial Sequence
  SYNTHESIZED POLYNUCLEOTIDE
ggatccaaattttctggctgc

SEQ ID NO: 177 - DNA, 13 bp, Artificial Sequence
  SYNTHESIZED POLYNUCLEOTIDE
ggatcctggctgc

SEQ ID NO: 178 - DNA, 18 bp, Artificial Sequence
  SYNTHESIZED POLYNUCLEOTIDE
ggatccaattctggctgc

SEQ ID NO: 179 - DNA, 10 bp, Artificial Sequence
  SYNTHESIZED POLYNUCLEOTIDE
tcctggctgc

SEQ ID NO: 180 - DNA, 12 bp, Artificial Sequence
  SYNTHESIZED POLYNUCLEOTIDE
ggatctggctgc

SEQ ID NO: 181 - DNA, 17 bp, Artificial Sequence
  SYNTHESIZED POLYNUCLEOTIDE
ggatccattctggctgc

SEQ ID NO: 182 - DNA, 13 bp, Artificial Sequence
  SYNTHESIZED POLYNUCLEOTIDE
ggatccaaatttc

SEQ ID NO: 183 - DNA, 11 bp, Artificial Sequence
  SYNTHESIZED POLYNUCLEOTIDE
ggatccaaatt

SEQ ID NO: 184 - DNA, 14 bp, Artificial Sequence
  Synthetic
caaatttctggctg

SEQ ID NO: 185 - DNA, 12 bp, Artificial Sequence
  SYNTHESIZED POLYNUCLEOTIDE
ggatccaaattt

SEQ ID NO: 186 - DNA, 20 bp, Artificial Sequence
  SYNTHESIZED POLYNUCLEOTIDE
agttcatggttactggttcc

SEQ ID NO: 187 - DNA, 19 bp, Artificial Sequence
  SYNTHESIZED POLYNUCLEOTIDE
agttcatggtactggttcc

SEQ ID NO: 188 - DNA, 13 bp, Artificial Sequence
  SYNTHESIZED POLYNUCLEOTIDE
agttcatggttcc

SEQ ID NO: 189 - DNA, 18 bp, Artificial Sequence
  SYNTHESIZED POLYNUCLEOTIDE
agttcatgtactggttcc

SEQ ID NO: 190 - DNA, 21 bp, Artificial Sequence
  SYNTHESIZED POLYNUCLEOTIDE
agttcatggtttactggttcc

SEQ ID NO: 191 - DNA, 18 bp, Artificial Sequence
  SYNTHESIZED POLYNUCLEOTIDE
agttccagtactggttcc

SEQ ID NO: 192 - DNA, 16 bp, Artificial Sequence
  SYNTHESIZED POLYNUCLEOTIDE
agttcatactggttcc

SEQ ID NO: 193 - DNA, 21 bp, Artificial Sequence
  SYNTHESIZED POLYNUCLEOTIDE
agttcatggtatactggttcc

SEQ ID NO: 194 - DNA, 13 bp, Artificial Sequence
  SYNTHESIZED POLYNUCLEOTIDE
agttactggttcc

SEQ ID NO: 195 - DNA, 14 bp, Artificial Sequence
  SYNTHESIZED POLYNUCLEOTIDE
gttcatggttactg

SEQ ID NO: 196 - DNA, 20 bp, Artificial Sequence
  SYNTHESIZED POLYNUCLEOTIDE
actccccagttcatggttac

SEQ ID NO: 197 - DNA, 21 bp, Artificial Sequence
  SYNTHESIZED POLYNUCLEOTIDE
actccccagtttcatggttac

SEQ ID NO: 198 - DNA, 19 bp, Artificial Sequence
  SYNTHESIZED POLYNUCLEOTIDE
actccccagtcatggttac

SEQ ID NO: 199 - DNA, 15 bp, Artificial Sequence
  SYNTHESIZED POLYNUCLEOTIDE
actccccatggttac

SEQ ID NO: 200 - DNA, 13 bp, Artificial Sequence
  SYNTHESIZED POLYNUCLEOTIDE
actccccagttac

SEQ ID NO: 201 - DNA, 12 bp, Artificial Sequence
  SYNTHESIZED POLYNUCLEOTIDE
actcatggttac

SEQ ID NO: 202 - DNA, 18 bp, Artificial Sequence
  SYNTHESIZED POLYNUCLEOTIDE
actccccatcatggttac

SEQ ID NO: 203 - DNA, 19 bp, Artificial Sequence
  SYNTHESIZED POLYNUCLEOTIDE
actccccattcatggttac

SEQ ID NO: 204 - DNA, 21 bp, Artificial Sequence
  SYNTHESIZED POLYNUCLEOTIDE
actccccagtgtcatggttac

SEQ ID NO: 205 - DNA, 21 bp, Artificial Sequence
  SYNTHESIZED POLYNUCLEOTIDE
actccccagtctcatggttac

SEQ ID NO: 206 - DNA, 18 bp, Artificial Sequence
  SYNTHESIZED POLYNUCLEOTIDE
actccccagttcatggtt

SEQ ID NO: 207 - DNA, 20 bp, Artificial Sequence
  SYNTHESIZED POLYNUCLEOTIDE
agccattatatccagggact

SEQ ID NO: 208 - DNA, 18 bp, Artificial Sequence
  SYNTHESIZED POLYNUCLEOTIDE
agccattatccagggact

SEQ ID NO: 209 - DNA, 11 bp, Artificial Sequence
  SYNTHESIZED POLYNUCLEOTIDE
agccagggact

SEQ ID NO: 210 - DNA, 19 bp, Artificial Sequence
  SYNTHESIZED POLYNUCLEOTIDE
agccattattccagggact

SEQ ID NO: 211 - DNA, 12 bp, Artificial Sequence
  SYNTHESIZED POLYNUCLEOTIDE
agtccagggact

SEQ ID NO: 212 - DNA, 21 bp, Artificial Sequence
  SYNTHESIZED POLYNUCLEOTIDE
agccattataatccagggact

SEQ ID NO: 213 - DNA, 18 bp, Artificial Sequence
  SYNTHESIZED POLYNUCLEOTIDE
agccattatccggggact

SEQ ID NO: 214 - DNA, 18 bp, Artificial Sequence
  SYNTHESIZED POLYNUCLEOTIDE
agccattatacagggact

SEQ ID NO: 215 - DNA, 10 bp, Artificial Sequence
  SYNTHESIZED POLYNUCLEOTIDE
tccagggact

SEQ ID NO: 216 - DNA, 19 bp, Artificial Sequence
  SYNTHESIZED POLYNUCLEOTIDE
agccattattccggggact

SEQ ID NO: 217 - DNA, 21 bp, Artificial Sequence
  SYNTHESIZED POLYNUCLEOTIDE
agccattataatccggggact

SEQ ID NO: 218 - DNA, 14 bp, Artificial Sequence
  SYNTHESIZED POLYNUCLEOTIDE
agccattatatcca

SEQ ID NO: 219 - DNA, 10 bp, Artificial Sequence
  SYNTHESIZED POLYNUCLEOTIDE
agccattata

SEQ ID NO: 220 - DNA, 20 bp, Artificial Sequence
  SYNTHESIZED POLYNUCLEOTIDE
tcagtgacggtacaggaggg

SEQ ID NO: 221 - DNA, 15 bp, Artificial Sequence
  SYNTHESIZED POLYNUCLEOTIDE
tcagtgacaggaggg

SEQ ID NO: 222 - DNA, 19 bp, Artificial Sequence
  SYNTHESIZED POLYNUCLEOTIDE
tcagtgacgtacaggaggg

SEQ ID NO: 223 - DNA, 9 bp, Artificial Sequence
  SYNTHESIZED POLYNUCLEOTIDE
tcaggaggg

SEQ ID NO: 224 - DNA, 14 bp, Artificial Sequence
  SYNTHESIZED POLYNUCLEOTIDE
tcagtgacggaggg

SEQ ID NO: 225 - DNA, 11 bp, Artificial Sequence
  SYNTHESIZED POLYNUCLEOTIDE
gtacaggaggg

SEQ ID NO: 226 - DNA, 15 bp, Artificial Sequence
  SYNTHESIZED POLYNUCLEOTIDE
tcagtgacgggaggg

SEQ ID NO: 227 - DNA, 21 bp, Artificial Sequence
  SYNTHESIZED POLYNUCLEOTIDE
tcagtgacggttacaggaggg

SEQ ID NO: 228 - DNA, 19 bp, Artificial Sequence
  SYNTHESIZED POLYNUCLEOTIDE
tcagtgacggacaggaggg

SEQ ID NO: 229 - DNA, 21 bp, Artificial Sequence
  SYNTHESIZED POLYNUCLEOTIDE
tcagtgacgggtacaggaggg

SEQ ID NO: 230 - DNA, 14 bp, Artificial Sequence
  SYNTHESIZED POLYNUCLEOTIDE
tcagtacaggaggg

SEQ ID NO: 231 - DNA, 18 bp, Artificial Sequence
  SYNTHESIZED POLYNUCLEOTIDE
tcagtgactacaggaggg

SEQ ID NO: 232 - DNA, 11 bp, Artificial Sequence
  SYNTHESIZED POLYNUCLEOTIDE
tcagtgacggg

SEQ ID NO: 233 - DNA, 10 bp, Artificial Sequence
  SYNTHESIZED POLYNUCLEOTIDE
tcagtgacgg

SEQ ID NO: 234 - DNA, 18 bp, Artificial Sequence
  SYNTHESIZED POLYNUCLEOTIDE
tcagtgacggcaggaggg

SEQ ID NO: 235 - DNA, 16 bp, Artificial Sequence
  SYNTHESIZED POLYNUCLEOTIDE
cagtgacggaggaggg

SEQ ID NO: 236 - DNA, 17 bp, Artificial Sequence
  SYNTHESIZED POLYNUCLEOTIDE
tcagtgatacaggaggg

SEQ ID NO: 237 - DNA, 16 bp, Artificial Sequence
  SYNTHESIZED POLYNUCLEOTIDE
tcagtgtacaggaggg

SEQ ID NO: 238 - DNA, 13 bp, Artificial Sequence
  SYNTHESIZED POLYNUCLEOTIDE
tcatacaggaggg

SEQ ID NO: 239 - DNA, 12 bp, Artificial Sequence
  SYNTHESIZED POLYNUCLEOTIDE
tcagtgacggta

SEQ ID NO: 240 - DNA, 11 bp, Artificial Sequence
  SYNTHESIZED POLYNUCLEOTIDE
cagtgacggta

SEQ ID NO: 241 - DNA, 10 bp, Artificial Sequence
  SYNTHESIZED POLYNUCLEOTIDE
ggtacaggag

SEQ ID NO: 242 - DNA, 17 bp, Artificial Sequence
  SYNTHESIZED POLYNUCLEOTIDE
gtgacggtacaggaggg

SEQ ID NO: 243 - DNA, 20 bp, Artificial Sequence
  SYNTHESIZED POLYNUCLEOTIDE
aggtgaagttcgctggagct

SEQ ID NO: 244 - DNA, 10 bp, Artificial Sequence
  SYNTHESIZED POLYNUCLEOTIDE
agctggagct

SEQ ID NO: 245 - DNA, 16 bp, Artificial Sequence
  SYNTHESIZED POLYNUCLEOTIDE
aggtgaagctggagct

SEQ ID NO: 246 - DNA, 10 bp, Artificial Sequence
  SYNTHESIZED POLYNUCLEOTIDE
aggtgaagct

SEQ ID NO: 247 - DNA, 16 bp, Artificial Sequence
  SYNTHESIZED POLYNUCLEOTIDE
aggtgaagttggagct

SEQ ID NO: 248 - DNA, 19 bp, Artificial Sequence
  SYNTHESIZED POLYNUCLEOTIDE
aggtgaagtcgctggagct

SEQ ID NO: 249 - DNA, 10 bp, Artificial Sequence
  SYNTHESIZED POLYNUCLEOTIDE
aggtggagct

SEQ ID NO: 250 - DNA, 18 bp, Artificial Sequence
  SYNTHESIZED POLYNUCLEOTIDE
aggtgaagcgctggagct

SEQ ID NO: 251 - DNA, 10 bp, Artificial Sequence
  SYNTHESIZED POLYNUCLEOTIDE
cgctggagct

SEQ ID NO: 252 - DNA, 16 bp, Artificial Sequence
  SYNTHESIZED POLYNUCLEOTIDE
aggtgacgctggagct

SEQ ID NO: 253 - DNA, 21 bp, Artificial Sequence
  SYNTHESIZED POLYNUCLEOTIDE
aggtgaagtttcgctggagct

SEQ ID NO: 254 - DNA, 10 bp, Artificial Sequence
  SYNTHESIZED POLYNUCLEOTIDE
ggtgaagttc

SEQ ID NO: 255 - DNA, 10 bp, Artificial Sequence
  SYNTHESIZED POLYNUCLEOTIDE
agttcgctgg

SEQ ID NO: 256 - DNA, 12 bp, Artificial Sequence
  SYNTHESIZED POLYNUCLEOTIDE
aggtgaagttcg

SEQ ID NO: 257 - DNA, 13 bp, Artificial Sequence
  SYNTHESIZED POLYNUCLEOTIDE
ggtgaagttcgct

SEQ ID NO: 258 - DNA, 10 bp, Artificial Sequence
  SYNTHESIZED POLYNUCLEOTIDE
tgaagttcgc

SEQ ID NO: 259 - DNA, 18 bp, Artificial Sequence
  SYNTHESIZED POLYNUCLEOTIDE
aggtgaagttcgctggag

SEQ ID NO: 260 - DNA, 16 bp, Artificial Sequence
  SYNTHESIZED POLYNUCLEOTIDE
aggtgaagttcgctgg

SEQ ID NO: 261 - DNA, 10 bp, Artificial Sequence
  SYNTHESIZED POLYNUCLEOTIDE
aggtgaagtt

SEQ ID NO: 262 - DNA, 19 bp, Artificial Sequence
  SYNTHESIZED POLYNUCLEOTIDE
ggtgaagttcgctggagct

SEQ ID NO: 263 - DNA, 20 bp, Artificial Sequence
  SYNTHESIZED POLYNUCLEOTIDE
agttcgctggagctggtgtg

SEQ ID NO: 264 - DNA, 14 bp, Artificial Sequence
  SYNTHESIZED POLYNUCLEOTIDE
agttcgctggtgtg

SEQ ID NO: 265 - DNA, 10 bp, Artificial Sequence
  SYNTHESIZED POLYNUCLEOTIDE
agctggtgtg

SEQ ID NO: 266 - DNA, 19 bp, Artificial Sequence
  SYNTHESIZED POLYNUCLEOTIDE
agttcgctgagctggtgtg

SEQ ID NO: 267 - DNA, 10 bp, Artificial Sequence
  SYNTHESIZED POLYNUCLEOTIDE
agttcgctgg

SEQ ID NO: 268 - DNA, 20 bp, Artificial Sequence
  SYNTHESIZED POLYNUCLEOTIDE
actactcactcctcggtgct

SEQ ID NO: 269 - DNA, 21 bp, Artificial Sequence
  SYNTHESIZED POLYNUCLEOTIDE
actactcacttcctcggtgct

SEQ ID NO: 270 - DNA, 13 bp, Artificial Sequence
  SYNTHESIZED POLYNUCLEOTIDE
actactcggtgct

SEQ ID NO: 271 - DNA, 19 bp, Artificial Sequence
  SYNTHESIZED POLYNUCLEOTIDE
actactcatcctcggtgct

SEQ ID NO: 272 - DNA, 25 bp, Artificial Sequence
  SYNTHESIZED POLYNUCLEOTIDE
actactactactcaccctcggtgct

SEQ ID NO: 273 - DNA, 16 bp, Artificial Sequence
  SYNTHESIZED POLYNUCLEOTIDE
actactcctcggtgct

SEQ ID NO: 274 - DNA, 10 bp, Artificial Sequence
  SYNTHESIZED POLYNUCLEOTIDE
cctcggtgct

SEQ ID NO: 275 - DNA, 18 bp, Artificial Sequence
  SYNTHESIZED POLYNUCLEOTIDE
actactcacctcggtgct

SEQ ID NO: 276 - DNA, 17 bp, Artificial Sequence
  SYNTHESIZED POLYNUCLEOTIDE
actactcactcggtgct

SEQ ID NO: 277 - DNA, 18 bp, Artificial Sequence
  SYNTHESIZED POLYNUCLEOTIDE
actactctcctcggtgct

SEQ ID NO: 278 - DNA, 17 bp, Artificial Sequence
  SYNTHESIZED POLYNUCLEOTIDE
actacttcctcggtgct

SEQ ID NO: 279 - DNA, 18 bp, Artificial Sequence
  SYNTHESIZED POLYNUCLEOTIDE
actactcacttcggtgct

SEQ ID NO: 280 - DNA, 15 bp, Artificial Sequence
  SYNTHESIZED POLYNUCLEOTIDE
actatcctcggtgct

SEQ ID NO: 281 - DNA, 10 bp, Artificial Sequence
  SYNTHESIZED POLYNUCLEOTIDE
actactcact

SEQ ID NO: 282 - DNA, 10 bp, Artificial Sequence
  SYNTHESIZED POLYNUCLEOTIDE
actactcact

SEQ ID NO: 283 - DNA, 14 bp, Artificial Sequence
  SYNTHESIZED POLYNUCLEOTIDE
actactcactcctc

SEQ ID NO: 284 - DNA, 17 bp, Artificial Sequence
  SYNTHESIZED POLYNUCLEOTIDE
actactcactcctcggt

SEQ ID NO: 285 - DNA, 20 bp, Artificial Sequence
  SYNTHESIZED POLYNUCLEOTIDE
cccgatcttctcctggttgt

SEQ ID NO: 286 - DNA, 18 bp, Artificial Sequence
  SYNTHESIZED POLYNUCLEOTIDE
cccgatcttcctggttgt

SEQ ID NO: 287 - DNA, 15 bp, Artificial Sequence
  SYNTHESIZED POLYNUCLEOTIDE
cccgatcctggttgt

SEQ ID NO: 288 - DNA, 14 bp, Artificial Sequence
  SYNTHESIZED POLYNUCLEOTIDE
cccgatctggttgt

SEQ ID NO: 289 - DNA, 10 bp, Artificial Sequence
  SYNTHESIZED POLYNUCLEOTIDE
ccctggttgt

SEQ ID NO: 290 - DNA, 17 bp, Artificial Sequence
  SYNTHESIZED POLYNUCLEOTIDE
cccgatcttctggttgt

SEQ ID NO: 291 - DNA, 15 bp, Artificial Sequence
  SYNTHESIZED POLYNUCLEOTIDE
cccgatcttggttgt

SEQ ID NO: 292 - DNA, 17 bp, Artificial Sequence
  SYNTHESIZED POLYNUCLEOTIDE
cccgatctcctggttgt

SEQ ID NO: 293 - DNA, 19 bp, Artificial Sequence
  SYNTHESIZED POLYNUCLEOTIDE
cccgatcttccctggttgt

SEQ ID NO: 294 - DNA, 10 bp, Artificial Sequence
  SYNTHESIZED POLYNUCLEOTIDE
cccgatcttc

SEQ ID NO: 295 - DNA, 11 bp, Artificial Sequence
  SYNTHESIZED POLYNUCLEOTIDE
cccgatcttct

SEQ ID NO: 296 - DNA, 10 bp, Artificial Sequence
  Synthetic
ccgatcttct

SEQ ID NO: 297 - DNA, 14 bp, Artificial Sequence
  Synthetic
tcttctcctggttg

SEQ ID NO: 298 - DNA, 10 bp, Artificial Sequence
  SYNTHESIZED POLYNUCLEOTIDE
tcctggttgt

SEQ ID NO: 299 - DNA, 20 bp, Artificial Sequence
  SYNTHESIZED POLYNUCLEOTIDE
aaatcctcatccctggcact

SEQ ID NO: 300 - DNA, 13 bp, Artificial Sequence
  SYNTHESIZED POLYNUCLEOTIDE
aaatcctggcact

SEQ ID NO: 301 - DNA, 14 bp, Artificial Sequence
  SYNTHESIZED POLYNUCLEOTIDE
aaatccctggcact

SEQ ID NO: 302 - DNA, 21 bp, Artificial Sequence
  SYNTHESIZED POLYNUCLEOTIDE
aaatcctcattccctggcact

SEQ ID NO: 303 - DNA, 10 bp, Artificial Sequence
  SYNTHESIZED POLYNUCLEOTIDE
ccctggcact

SEQ ID NO: 304 - DNA, 19 bp, Artificial Sequence
  SYNTHESIZED POLYNUCLEOTIDE
aaatcctcaccctggcact

SEQ ID NO: 305 - DNA, 18 bp, Artificial Sequence
  SYNTHESIZED POLYNUCLEOTIDE
aaatcctcccctggcact

SEQ ID NO: 306 - DNA, 17 bp, Artificial Sequence
  SYNTHESIZED POLYNUCLEOTIDE
aaatcctccctggcact

SEQ ID NO: 307 - DNA, 15 bp, Artificial Sequence
  SYNTHESIZED POLYNUCLEOTIDE
aaatcccctggcact

SEQ ID NO: 308 - DNA, 21 bp, Artificial Sequence
  SYNTHESIZED POLYNUCLEOTIDE
acatcctcattccctggcact

SEQ ID NO: 309 - DNA, 13 bp, Artificial Sequence
  SYNTHESIZED POLYNUCLEOTIDE
acatcctggcact

SEQ ID NO: 310 - DNA, 19 bp, Artificial Sequence
  SYNTHESIZED POLYNUCLEOTIDE
aaatcctctccctggcact

SEQ ID NO: 311 - DNA, 18 bp, Artificial Sequence
  SYNTHESIZED POLYNUCLEOTIDE
aaatcctcatctggcact

SEQ ID NO: 312 - DNA, 20 bp, Artificial Sequence
  SYNTHESIZED POLYNUCLEOTIDE
aaatcctaaaccctggcact

SEQ ID NO: 313 - DNA, 15 bp, Artificial Sequence
  SYNTHESIZED POLYNUCLEOTIDE
aaatcctctggcact

SEQ ID NO: 314 - DNA, 16 bp, Artificial Sequence
  SYNTHESIZED POLYNUCLEOTIDE
aaatccccctggcact

SEQ ID NO: 315 - DNA, 11 bp, Artificial Sequence
  SYNTHESIZED POLYNUCLEOTIDE
aaatcctcact

SEQ ID NO: 316 - DNA, 14 bp, Artificial Sequence
  SYNTHESIZED POLYNUCLEOTIDE
acatccctggcact

SEQ ID NO: 317 - DNA, 10 bp, Artificial Sequence
  SYNTHESIZED POLYNUCLEOTIDE
aaatcctcat

SEQ ID NO: 318 - DNA, 14 bp, Artificial Sequence
  SYNTHESIZED POLYNUCLEOTIDE
aaatcctcatccct

SEQ ID NO: 319 - DNA, 13 bp, Artificial Sequence
  SYNTHESIZED POLYNUCLEOTIDE
ctcatccctggca

SEQ ID NO: 320 - DNA, 16 bp, Artificial Sequence
  SYNTHESIZED POLYNUCLEOTIDE
aaatcctcatccctgg

SEQ ID NO: 321 - DNA, 16 bp, Artificial Sequence
  Synthetic
tcctcatccctggcac

SEQ ID NO: 322 - DNA, 11 bp, Artificial Sequence
  SYNTHESIZED POLYNUCLEOTIDE
aaatcctcatc

SEQ ID NO: 323 - DNA, 15 bp, Artificial Sequence
  SYNTHESIZED POLYNUCLEOTIDE
ctcatccctggcact

SEQ ID NO: 324 - DNA, 18 bp, Artificial Sequence
  SYNTHESIZED POLYNUCLEOTIDE
aaatcctcatccctggca

SEQ ID NO: 325 - DNA, 20 bp, Artificial Sequence
  SYNTHESIZED POLYNUCLEOTIDE
agagcaacagtgctgtggcc